(19) United States
(12) Patent Application Publication
(10) Pub. No.: US 2025/0188188 A1
(43) Pub. Date: Jun. 12, 2025

(54) ANTIBODIES THAT BIND TO C1S AND USES THEREOF

(71) Applicant: DIANTHUS THERAPEUTICS OPCO, INC., New York, NY (US)

(72) Inventors: Vahe Bedian, Waltham, MA (US); Charles A. Omer, Waltham, MA (US)

(21) Appl. No.: 18/781,579

(22) Filed: Jul. 23, 2024

Related U.S. Application Data
(63) Continuation of application No. 18/515,714, filed on Nov. 21, 2023, now Pat. No. 12,110,344.
(60) Provisional application No. 63/384,537, filed on Nov. 21, 2022.
(60) Provisional application No. 63/485,765, filed on Feb. 17, 2023.
(60) Provisional application No. 63/501,807, filed on May 12, 2023.
(60) Provisional application No. 63/590,980, filed on Oct. 17, 2023.

(51) Int. Cl. C07K 16/40; A61K 39/00; A61P 25/28

(52) U.S. Cl. CPC C07K 16/40; A61P 25/28; A61K 39/00

(57) ABSTRACT

Embodiments that are provided for herein relate to antibodies and compositions that bind to C1s. Also provided are methods of producing the antibodies of the present disclosure, as well as uses of the provided antibodies and compositions for the treatment of C1s mediated diseases and disorders.

### CROSS-REFERENCE TO RELATED APPLICATIONS

This application claims the benefit of U.S. Provisional Application No. 63/590,980, filed on Oct. 17, 2023, U.S. Provisional Application No. 63/501,807, filed May 12, 2023, U.S. Provisional Application No. 63/485,765, filed on Feb. 17, 2023, and U.S. Provisional Application No. 63/384,537, filed on Nov. 21, 2022, each of which is hereby incorporated by reference in their entirety.

### REFERENCE TO AN ELECTRONIC SEQUENCE LISTING

This application contains a Sequence Listing which has been submitted electronically in XML format and is hereby incorporated by reference in its entirety. The Sequence Listing is named “DIN002WOsequencelisting.xml”, was created on Nov. 17, 2023, and is 495,158 bytes in size.

### FIELD OF THE INVENTION

The field of the invention is immunology, in particular therapeutic antibodies, and treatment of disease with those antibodies.

### BACKGROUND

The complement system is a well-known effector mechanism of the innate immune response, providing not only protection against pathogens and other harmful agents but also recovery from injury. Complement activation due to autoantibodies and alloantibodies can lead to damage to normal cells or rejection of transplanted tissue. The complement pathway is comprised of a number of proteins that typically exist in the body in an inactive form. Complement is comprised of the alternative, classical and lectin pathways, and are critical for immune defense. The classical complement pathway is triggered by activation of the first component of complement, referred to as the C1 complex, which consists of C1q, C1r, and C1s proteins. Upon binding of C1 to an immune complex or other activator, the C1s component, a diisopropyl fluorophosphate (DFP)-sensitive serine protease, cleaves complement components C4 and C2 to initiate activation of the classical complement pathway. Binding of C1 to antigen-antibody complexes induces cleavage of proenzyme C1s to active C1s enzyme, and the eventual formation of the pore-forming membrane attack complex (MAC) that is responsible for the destruction of the neuromuscular junction. The classical complement pathway appears to play a role in many diseases and disorders.

For example, sutimlimab (TNT009), marketed as EnJaymo®, is an antibody that inhibits C1s for treatment of hemolysis in adults with cold agglutinin disease.

However, sutimlimab binds to both the active form of C1s, and the inactive zymogen proC1s, and due to the lack of specificity of sutimlimab for either form of C1s, a very high dose must be administered to overcome circulating levels of proC1s, which may limit the clinical use of sutimlimab.

Myasthenia gravis (MG) is a rare and chronic autoimmune disease caused by autoantibodies that target proteins at the neuromuscular junction, which lead to impaired neurotransmission, muscle fatigue, and weakness. The most prevalent autoantibody in myasthenia gravis is against the nicotinic acetylcholine receptor (AChR), which is proposed to induce pathology through three different mechanisms, including activation of the complement system. Treatments for MG lack in their effectiveness.

Therefore, there is a need in the art for compounds that treat a complement classical pathway-mediated disease or disorder with specificity for active forms of the complement proteins. The embodiments provided for herein satisfy these needs as well as others.

### SUMMARY

In some embodiments, an antibody, or antigen binding fragment thereof, is provided that binds to the active form of C1s. In some embodiments, an antibody, or antigen binding fragment thereof, is provided that specifically binds to the active form of C1s. In some embodiments, the antibody, or antigen binding fragment thereof, is as provided herein.

In some embodiments, a variant of an antibody, or antigen binding fragment thereof, is provided, wherein the variant has 1-10 substitutions, deletions, or insertions.

In some embodiments, a variant of an antibody, or antigen binding fragment thereof, is provided, wherein the variant has 1-10 conservative substitutions.

In some embodiments, a recombinant antibody, or antigen binding fragment thereof, is provided, wherein the recombinant antibody, or antigen binding fragment thereof, binds to C1s. In some embodiments, the recombinant antibody, or antigen binding fragment thereof, is an antibody, or antigen binding fragment thereof, as provided for herein.

In some embodiments, an isolated nucleic acid molecule is provided. In some embodiments, the isolated nucleic acid molecule encodes an antibody or antigen binding fragment thereof, a heavy chain variable region, a light chain variable region, a heavy chain, a light chain, or a combination thereof. In some embodiments, the antibody or antigen binding fragment thereof, a heavy chain variable region, a light chain variable region, a heavy chain, or light chain are as provided for herein.

In some embodiments, an expression vector is provided. In some embodiments, the expression vector comprises a nucleic acid molecule as provided for herein.

In some embodiments a host cell is provided. In some embodiments, the host cell comprises a nucleic acid molecule or expression vector as provided for herein. In some embodiments, the host cell comprises a nucleic acid molecule as provided for herein. In some embodiments, the host cell comprises an expression vector as provided for herein.

In some embodiments, an antibody, or antigen binding fragment thereof, is provided, wherein the antibody, or antigen binding fragment thereof, is produced by the host cell.

In some embodiments, a pharmaceutical composition is provided. In some embodiments, the pharmaceutical composition comprises an antibody, or antigen binding fragment thereof, as provided for herein.

In some embodiments, a method of producing a polypeptide comprising a heavy chain variable region or a light chain variable region is provided. In some embodiments, the method comprises (a) growing a host cell as provided for herein under conditions so that the host cell expresses the polypeptide comprising the heavy chain variable region or the light chain variable region; and (b) purifying the polypeptide comprising the heavy chain or the light chain variable regions. In some embodiments, the heavy chain variable region is as provided for herein. In some embodiments, the light chain variable region is as provided for herein.

In some embodiments, a method of producing an antibody or an antigen binding fragment of the antibody is provided. In some embodiments, the antibody or antigen binding fragment bind to human C1s. In some embodiments, the method comprises (a) growing a host cell as provided for herein under conditions so that the host cell expresses a polypeptide or polypeptides comprising the immunoglobulin heavy chain variable region and/or the immunoglobulin light chain variable region, thereby producing the antibody or the antigen binding fragment of the antibody; and (b) purifying the antibody or the antigen-binding fragment of the antibody.

In some embodiments, a method of treating a subject with a C1s mediated disorder is provided. In some embodiments, the method comprises administering to the subject an antibody, or antigen binding fragment thereof, as provided for herein, or a pharmaceutical composition as provided for herein.

In some embodiments, an antibody, or antigen binding fragment thereof, or a pharmaceutical composition is provided, wherein the antibody, or antigen binding fragment thereof, or the pharmaceutical composition are for the use in the treatment of a C1s mediated disorder. In some embodiments, the antibody, or antigen binding fragment thereof, is as provided for herein. In some embodiments, the pharmaceutical composition is as provided for herein. In some embodiments, the C1s mediated disorder is as provided for herein. In some embodiments, the C1s mediated disorder is hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the condition can be caused by a stroke or spinal cord injury.

In some embodiments, an antibody, or antigen binding fragment thereof, or a pharmaceutical composition is provided, wherein the antibody, or antigen binding fragment thereof, or the pharmaceutical composition are for the use as a medicament. In some embodiments, the antibody, or antigen binding fragment thereof, is as provided for herein. In some embodiments, the pharmaceutical composition is as provided for herein.

In some embodiments, a use of an antibody, or antigen binding fragment thereof, or a pharmaceutical composition is provided, wherein the use is for the treatment of a C1s mediated disorder. In some embodiments, the antibody, or antigen binding fragment thereof, is as provided for herein. In some embodiments, the pharmaceutical composition is as provided for herein. In some embodiments, the C1s mediated disorder is as provided for herein.

### BRIEF DESCRIPTION OF THE DRAWINGS

FIG. 1 illustrates the amino acid sequence of hProC1s RQ, in which the underlined portion denotes the expressed protein-secreting signal sequence (first 19 amino acids), the bolded portion denotes the sequence in which arginine 437 has been changed to glutamine, and the italicized region denotes the spacer and 10 histidine residues used in the purification of the protein (last 19 amino acids).

FIGS. 2A-2C show binding of hProC1s RQ and human C1s to the antibodies disclosed herein. FIG. 2A shows binding to antibody 4011g and derivatives. FIG. 2B shows binding to antibody 5L3g and derivatives. FIG. 2C shows binding to antibody 191g and derivatives.

FIG. 3 depicts inhibition of the Wieslab complement classical pathway assay in 1% human serum by the antibodies IgG4-MAB17, IgG4-MAB4, and IgG4-MAB9, as disclosed herein.

FIGS. 4A-4C depicts the antibody-mediated inhibition of complement-mediated hRBC lysis in 25% human serum by antibodies IgG4-MAB17, IgG4-MAB4, and IgG4-MAB9 (FIG. 4A), by antibodies 191g, MAB4 and MAB9 (FIG. 4B), and by antibodies MAB2 (US20220380483A1) and MAB9 (FIG. 4C), as disclosed herein.

FIG. 5 depicts the antibody-mediated inhibition of C3b deposition on hRBCs by antibodies IgG4-MAB17, IgG4-MAB4, and IgG4-MAB9, as disclosed herein.

FIG. 6 shows mean concentrations of antibodies after a single IV dose of 3 or 30 mg/kg body weight (mpk) over time in cynomolgus monkeys, by antibodies IgG4-MAB17, IgG4-MAB4, and IgG4-MAB9, as disclosed herein.

FIG. 7 shows the concentration of IgG4-MAB39 in serum diluted by the dilution factor necessary to obtain 50% lysis of sensibilized sheep RBCs (CH50). The serum was obtained from cynomolgus monkeys treated with a single subcutaneous dose of 20 mg/kg body weight (mpk) of IgG4-MAB39. The concentration of IgG4-MAB39 at CH50 was monitored over time for up to 30 days.

FIG. 8 depicts preclinical cell-based model of myasthenia gravis was used to assess the effect of complement classical pathway inhibition with IgG4-MAB39 on neuromuscular junction functionality. Human induced pluripotent stem cell (iPSC)-derived motor neuron and skeletal muscle cells were co-cultured in a two-chamber compartmentalized microfluidic device with motor neuron on one chamber and muscle cells on the second chamber. Sera (10% concentration) from AChR+ MG patients (n=3) supplemented with complement-preserved pooled normal human sera (1%), were added to the skeletal muscle cell reservoir in the absence or presence of IgG4-MAB39 or anti-C5 antibody recombinant ravulizumab (from published patent). The motor neuron chamber received electrical stimulation at 2 Hz for 2 minutes and skeletal muscle contraction was monitored in real-time. The muscle contraction fatigue index score was calculated based on the area under the curve (AUC) of the region of interest (ROI) divided by tetanus response from that myotube (maximum ROI trace height×duration of stimulation). IgG4-MAB39 decreased the muscle contract fatigue index score in AChR+ MG patient sera, an indication of improved neurotransmission, neuromuscular junction functionality, and muscle contraction. The response profile of IgG-MAB39 was similar to that of anti-C5 antibody recombinant ravulizumab. IgG4-MAB39 improved neurotransmission impairment in a physiologically relevant in vitro model of AChR+ MG. This finding provides a scientific rationale for targeting complement classical pathway in AChR+ MG.

FIG. 9 shows the dose-dependent complete inhibition of the Classical Pathway with MAB39 and its pre-clinical precursor MAB9 in the Wieslab assay.

FIG. 10 shows dose-dependent complete inhibition of the Classical Pathway with MAB39 and its pre-clinical precursor MAB9 in the antibody-sensitized sheep red blood cell lysis assay.

FIG. 11 shows percent (%) bacterial survival of N. meningitidis in complement-containing sera and anticapsular antibody with heat-inactivation, anti-C7 antibody, ravulizumab, or MAB39.

### DETAILED DESCRIPTION

Provided herein are binding proteins, e.g., antibodies, or fragments, thereof, that selectively bind to C1s and have low binding to the zymogen proC1s. In some embodiments, the antibodies inhibit activation of the classical complement pathway and can be used in methods to treat complement mediated disorders, such as, but not limited to those provided for herein. In some embodiments, the selectivity for C1s over proC1s can be used to reduce or prevent target mediated clearance of the therapeutic antibody, thus requiring lower doses and a reduction in the frequency of administration of the antibody.

Before the present compositions and methods are described, it is to be understood that the scope of the invention is not limited to the particular processes, compositions, or methodologies described herein, as these may vary. It is also to be understood that the terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the methods and systems disclosed herein, the preferred methods, devices, and materials are now described.

The following explanations of terms and methods are provided to better describe the present disclosure and to guide those of ordinary skill in the art in the practice of the present disclosure.

Those of ordinary skill in the art will understand that given the amino acid sequence of a variable region of the binding molecules (e.g., antibodies) set forth herein can determine the CDR sequences therein by these or any other conventions used to define CDR, such as, for example, IMGT, Kabat, Chothia, or Contact using web-based tools are available for determining the CDRs in such variable regions based on any known convention. Such tools include those found at www.abysis.org/abysis/sequence_input/key_annotation/key_annotation.cgi and www.novoprolabs.com/tools/cdr.

Accordingly, the disclosure herein of a set of three CDRs in a heavy or light chain variable region based upon one CDR convention is considered the equivalent of that same set of CDRs as determined by any other convention, which can be referred to as “convention equivalents” or “convention equivalent CDRs”.

As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements, unless the context clearly indicates otherwise. For example, the phrase “A or B” refers to A alone or B alone. The phrase “A, B, or a combination thereof” refers to A alone, B alone, or a combination of A and B. Similarly, “one or more of A and B” refers to A, B, or a combination of both A and B. The phrase “A and B” refers to a combination of A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless otherwise defined, all technical and scientific terms used herein have the same meaning as commonly understood to one of ordinary skill in the art. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic antibody.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies (single domain antibody) and multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion or fragment of one heavy chain that contains the V_{H} domain and the C_{H}1 domain and also the region between the C_{H}1 and CH2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and CH2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

The term “single-chain Fv” or “scFv” antibody refers to antibody fragments comprising the VH and VL domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “single-domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

In certain embodiments, monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

Typically, a variant antibody or antigen binding fragment of the antibodies provided herein retain at least 10% of its C1s binding activity (when compared to a parental antibody that is modified) when that activity is expressed on a molar basis. In some embodiments, a variant antibody (or antigen fragment thereof), or antigen binding fragment of an antibody provided herein, retains at least 20%, 50%, 70%, 80%, 90%, 95% or 100% or more of the C1s binding affinity as the parental antibody. As described herein, it is also intended that an antibody or antigen binding fragment of the invention can include conservative or non-conservative amino acid substitutions, which can also be referred to as “conservative variants” or “function conserved variants” of the antibody, that do not substantially alter its biologic activity.

“Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to population of substantially homogeneous antibodies, i.e., the antibody molecules comprising the population are identical in amino acid sequence except for possible naturally occurring mutations and/or post-translational modifications that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, a “chimeric antibody” is an antibody having the variable domain from a first antibody and constant domain from a second antibody, where the first and second antibodies are from different species. (U.S. Pat. No. 4,816,567; and Morrison et al., (1984) Proc. Natl. Acad. Sci. USA 81: 6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g. rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In some embodiments, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. However, in bifunctional or bispecific antibodies, the two binding sites are, in general, not the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain; Chothia and Lesk (1987) J. Mol. Biol. 196: 901-917). The CDRs can also be referenced according to the IMGT system for the identification of CDRs, which is described in Lefranc M P. Unique database numbering system for immunogenetic analysis. Immunol Today (1997) 18:509. As used herein, the term “framework” or “FR” residues refers to those variable domain residues other than the hypervariable region residues defined herein as CDR residues. CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived.

As used herein, “specific binding” or “immunospecific binding” or “binds immunospecifically” refer to antibody binding to a predetermined antigen at a much higher affinity than for another antigen(s) (e.g. selectively binds the active form of complement component C1s as compared to inactive C1s, which can also be referred to as proC1s zymogen). In some embodiments, the antibody binds the predetermined antigen with a dissociation constant (K_{D}) of 10⁻⁷ M or less, and such K_{D} is at least two-fold less than its K_{D} for binding to a non-specific antigen (e.g., BSA, casein, or another non-specific polypeptide).

The phrases “an antibody recognizing C1s” and “an antibody specific for C1s” are used interchangeably herein with the term “an antibody which binds immunospecifically to C1s.” In some embodiments, the antibody binds specifically or preferentially to C1s, such as the active form of C1s over other proteins, such as, but not limited to, the inactive form of C1s (proC1s). The degree of specificity necessary for an anti-C1s antibody may depend on the intended use of the antibody, and at any rate is defined by its suitability for use for an intended purpose. In some embodiments, the antibody, or binding compound derived from the antigen-binding site of an antibody, of the contemplated method binds to its antigen (active form of C1s), with an affinity that is at least two-fold greater, at least ten times greater, at least 20-times greater, or at least 100-times greater than the affinity with any other antigen, including, but not limited to inactive C1s.

Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589 601 (1983), which references are entirely incorporated herein by reference.

The term “homolog” means protein sequences having between 40% and 100% sequence identity to a reference sequence. Percent identity between two peptide chains can be determined by pair wise alignment using the default settings of the AlignX module of Vector NTI v.9.0.0 (Invitrogen Corp., Carlsbad, Calif) or other suitable alignment software, such as BLAST. In some embodiments, the antibody, or antigen binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

TABLE 1
Table: Exemplary Conservative Substitutions:

| | Exemplary |
|---|---|
| Original Residue | Conservative Substitutions |
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

The term “in combination with” as used herein means that the described agents can be administered to an animal or subject together in a mixture, concurrently as single agents or sequentially as single agents in any order.

The term “epitope” is meant to refer to that portion of any molecule capable of being recognized by and bound by an antibody at one or more of the Ab's antigen binding regions. Epitopes usually consist of chemically active surface groupings of molecules such as amino acids or sugar side chains and have specific three-dimensional structural characteristics as well as specific charge characteristics.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

In some embodiments, the antibody is a monoclonal antibody which binds to C1s. The sequence of active C1s is as follows (SEQ ID NO: 258): residues 1-15 (SEQ ID NO: 259) constitute the signal peptide which is cleaved during translation, and residues 16-688 (SEQ ID NO: 260 constitute the mature protein, as shown in

(SEQ ID NO: 258)
MWCIVLFSLLAWVYAEPTMYGEILSPNYPQAYPSEVEKSWDIEVP
EGYGIHLYFTHLDIELSENCAYDSVQIISGDTEEGRLCGQRSSNN
PHSPIVEEFQVPYNKLQVIFKSDFSNEERFTGFAAYYVATDINEC
TDFVDVPCSHFCNNFIGGYFCSCPPEYFLHDDMKNCGVNCSGDVF
TALIGEIASPNYPKPYPENSRCEYQIRLEKGFQVVVTLRREDFDV
EAADSAGNCLDSLVFVAGDRQFGPYCGHGFPGPLNIETKSNALDI
IFQTDLTGQKKGWKLRYHGDPMPCPKEDTPNSVWEPAKAKYVFRD
VVQITCLDGFEVVEGRVGATSFYSTCQSNGKWSNSKLKCQPVDCG
IPESIENGKVEDPESTLFGSVIRYTCEEPYYYMENGGGGEYHCAG
NGSWVNEVLGPELPKCVPVCGVPREPFEEKQRIIGGSDADIKNFP
WQVFFDNPWAGGALINEYWVLTAAHVVEGNREPTMYVGSTSVQTS
RLAKSKMLTPEHVFIHPGWKLLEVPEGRTNFDNDIALVRLKDPVK
MGPTVSPICLPGTSSDYNLMDGDLGLISGWGRTEKRDRAVRLKAA
RLPVAPLRKCKEVKVEKPTADAEAYVFTPNMICAGGEKGMDSCKG
DSGGAFAVQDPNDKTKFYAAGLVSWGPQCGTYGLYTRVKNYVDWI
MKTMQENSTPRED;
(SEQ ID NO: 259)
MWCIVLFSLLAWVYA;
(SEQ ID NO: 260)
EPTMYGEILSPNYPQAYPSEVEKSWDIEVPEGYGIHLYFTHLDIE
LSENCAYDSVQIISGDTEEGRLCGQRSSNNPHSPIVEEFQVPYNK
LQVIFKSDFSNEERFTGFAAYYVATDINECTDFVDVPCSHFCNNF
IGGYFCSCPPEYFLHDDMKNCGVNCSGDVFTALIGEIASPNYPKP
YPENSRCEYQIRLEKGFQVVVTLRREDFDVEAADSAGNCLDSLVF
VAGDRQFGPYCGHGFPGPLNIETKSNALDIIFQTDLTGQKKGWKL
RYHGDPMPCPKEDTPNSVWEPAKAKYVFRDVVQITCLDGFEVVEG
RVGATSFYSTCQSNGKWSNSKLKCQPVDCGIPESIENGKVEDPES
TLFGSVIRYTCEEPYYYMENGGGGEYHCAGNGSWVNEVLGPELPK
CVPVCGVPREPFEEKQRIIGGSDADIKNFPWQVFFDNPWAGGALI
NEYWVLTAAHVVEGNREPTMYVGSTSVQTSRLAKSKMLTPEHVFI
HPGWKLLEVPEGRTNFDNDIALVRLKDPVKMGPTVSPICLPGTSS
DYNLMDGDLGLISGWGRTEKRDRAVRLKAARLPVAPLRKCKEVKV
EKPTADAEAYVFTPNMICAGGEKGMDSCKGDSGGAFAVQDPNDKT
KFYAAGLVSWGPQCGTYGLYTRVKNYVDWIMKTMQENSTPRED.

The difference between inactive C1s and active C1s is that inactive proC1s is cleaved at the peptide bond between R437 and 1438 and undergoes a conformational change. The two fragments generated by this cleavage remain associated by a disulfide bond. Without wishing to be bound by a particular theory, proC1s is a single chain 86,000 Da protein that is the native form of C1s proteins (e.g. serine protease). C1s is a subunit of the C1 complex which is the first complement component in the cascade referred to as the classical pathway of complement. ProC1s is an inactive zymogen until C1 is activated. C1 complex binds to and is activated by antigen-antibody complexes (immune complexes) yielding C1r enzyme. C1r enzyme in the C1 complex activates proC1s generating C1s enzyme. C1 complex is a non-covalent calcium-dependent complex of one C1q, two C1r and two C1s molecules. C1q binds through two or more of its six arms to the Fc domains of IgG or IgM. The binding of multiple arms to immune complexes causes the two C1r proteins in the complex (protease zymogens) to activate producing two proteases that cleave and activate the two proC1s in the complex (Morikis, D. and Lambris, J. D. (2005)). This activation of proC1s is caused by cleavage into the two chain C1s enzyme with 58,000 and 28,000 Dalton fragments.

In some embodiments, the antibodies provided herein do not inhibit, or significantly inhibit, the proteolytic cleavage of non-complement substrates. Examples of non-complement substrates, include but are not limited to, collagen, HMC I, HMGB1, IGFBP5, LRP6, NCL, and NPM1. Without being bound to any theory, such substrates are important in the clearance of antigens on apoptotic cells or in the hemostasis of bone, muscle, and connective tissues. Accordingly, an antibody that does not inhibit, or significantly inhibit, the proteolytic cleavage of non-complement substrates, such as those provided for herein, can allow the antibody to be utilized with fewer side effects or at higher doses. In some embodiments, the non-complement substrate is collagen. In some embodiments, the non-complement substrate is HMC I. In some embodiments, the non-complement substrate is HMGB1. In some embodiments, the non-complement substrate is IGFBP5. In some embodiments, the non-complement substrate is LRP6. In some embodiments, the non-complement substrate is NCL. In some embodiments, the non-complement substrate is NPM1.

In some embodiments, the antibody comprises an Fc domain. The Fc domain can be linked to the heavy or light chain of the antibody. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat (“EU Numbering”). Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

Other mutations can be used in the Fc domain, such as those provided for in U.S. Pat. No. 8,394,925, which is hereby incorporated by reference in its entirety. In some embodiments, the Fc region is a variant Fc region comprising amino acid substitutions at positions 428 and 434, wherein the amino acid substitutions are a leucine that is not the wild-type amino acid at position 428 and a serine that is not the wild-type amino acid at position 434, wherein the polypeptide is an antibody and wherein numbering is according to the EU Index in Kabat et al. In some embodiments, the Fc region comprises a S228P, L235E, M428L, or N434S substitution. In some embodiments, the Fc region comprises a M428L substitution. In some embodiments, the Fc region comprises a N434S substitution. In some embodiments, the Fc region comprises a M428L and a N434S substitution (EU Numbering), which can be collectively referred to as “LS mutations” or “LS”. In some embodiments, the Fc region comprises a M252Y, S254T, and/or T256E substitution (EU Numbering). In some embodiments, the Fc region comprises a M252Y, S254T, and T256E substitutions. When all three of the mutations of M252Y, S254T, and T256E are present in the Fc region, it can be referred to as “YTE” or “YTE mutations.”

In some embodiments, the antibody comprises a constant region as set forth below with or without the mutations provided for in the list below:

>IgG4 S228P L235E LS
(SEQ ID NO: 261)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVLHEALHSH
YTQKSLSLSLGK;
>IgG4 S228P L235E YTE
(SEQ ID NO: 262)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLYITR
EPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK.

In some embodiments, the antibody comprises a constant region as provided herein, wherein the C-terminal lysine (K) amino acid has been deleted. In some embodiments, the antibody comprises a constant region as set follows:

>IgG4 S228P L235E LS-trunc
(SEQ ID NO: 263)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVLHEALHSH
YTQKSLSLSLG;
>IgG4 S228P L235E YTE-trunc
(SEQ ID NO: 264)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLYITR
EPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLG.

In some embodiments, the antibody, such as the light chain, comprises a kappa constant region, such as the human constant domain, which can comprise a sequence of:

RTVAAPSVFIFPPSDEQLKSGTASVVCLLNNFYPREAKVQW
KVDNALQSGNSQESVTEQDSKDSTYSLSSTLTLSKADYEKH
KVYACEVTHQGLSSPVTKSFNRGEC
(human kappa constant domain, SEQ ID NO: 265).

As used herein and unless otherwise indicated, the term “about” is intended to mean ±5% of the value it modifies. Thus, about 100 means 95 to 105.

In some embodiments, the antibodies described herein are used to detect the presence of the antigen. The present antibody can be used in any device or method to detect the presence of the antigen. In some embodiments, the antibodies are used to detect the active form of C1s. In some embodiments, the antibodies are used to detect the active form preferentially (specifically) over the inactive form of C1s.

The term “purified” when referenced to an antibody refers to an antibody that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and, at least 95%, 96%, 97%, 98%, 99%, or 100% (w/w) pure. In some embodiments, the antibody is purified.

### Antibody Conjugates

The antibodies provided for herein may also be conjugated to a chemical moiety. The chemical moiety may be, inter alia, a polymer, a radionuclide or a cytotoxic factor. In some embodiments, this can be referred to as an antibody drug conjugate. In some embodiments, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)). Examples of chemical moieties include, but are not limited to, anti-mitotics, such as calicheamicins (e.g. ozogamicin), monomethyl auristatin E, mertansine, and the like. Other examples include, but are not limited to, biologically active anti-microtubule agents, alkylating agents and DNA minor groove binding agents. Other examples thereof are provided herein and below. The chemical moiety can be linked to the antibody through a linking group (maleimide), a cleavable linker, such as a cathepsin cleavable linkers (valine-citrulline), and in some embodiments, one or more spacers (e.g. para-aminobenzylcarbamate).

The antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ¹⁵⁷Mn, ⁵²Tr and ⁵⁶Fe.

The antibodies and antibody fragments may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules may also be conjugated to a cytotoxic factor such as diphtheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and neomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In some embodiments, antibodies (e.g. an anti-C1s antibody) are provided herein. In some embodiments, the antibody is a recombinant antibody that binds to C1s. In some embodiments, the antibody binds to the active form of C1s. In some embodiments, the antibody binds to active form preferentially over the inactive form of C1s. In some embodiments, the antibody binds to the active form with an affinity that is at least 10, 20, 30, 40, 50, 60, 70, 80, 90, 100, or 200% higher as compared to its affinity for the inactive form of C1s. In some embodiments, the C1s protein is a human C1s protein, such as the active form of C1s. In some embodiments, the antibody does not specifically bind to the inactive form of the C1s protein. As used herein, the term “recombinant antibody” refers to an antibody that is not naturally occurring. In some embodiments, the term “recombinant antibody” refers to an antibody that is not isolated from a human subject. In some embodiments, the antibody binds with at least 100 times more affinity to the active form of C1s as compared to proC1s.

In some embodiments, an antibody, or antigen binding fragment thereof is provided, wherein the antibody or antibody fragment comprises a peptide selected from the following table, which illustrates the CDRs based on Kabat numbering.

TABLE 2
Kabat CDRs

| Ab ID No | HCDR1 | HCDR2 | HCDR3 | LCDR1 | LCDR2 | LCDR3 |
|---|---|---|---|---|---|---|
| MAB1 | DYYMS | YISRSGSTKY | DQEDYALDY | QASQDISNY | DASNLET (SEQ ID | EQYEDYPLT |
| | (SEQ ID | YADSVKG | (SEQ ID NO: | LN (SEQ ID | NO: 65) | (SEQ ID NO: |
| | NO: 61) | (SEQ ID NO: | 63) | NO: 64) | | 66) |
| | | 62) | | | | |
| MAB2 | DYYMS | YISRSGSTKY | DETDYGWDY | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 67) | NO: 64) | | 68) |
| | | 62) | | | | |
| MAB3 | DYYMS | YISRSGSTKY | DETDYAFDE | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 69) | NO: 64) | | 68) |
| | | 62) | | | | |
| MAB4 | DYYMS | YISRSGSTKY | DESQYALDY | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 70) | NO: 64) | | 71) |
| | | 62) | | | | |
| MAB5 | DYYMS | YISRSGSTKY | DNTDYALDL | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 72) | NO: 64) | | 71) |
| | | 62) | | | | |
| MAB6 | DYYMS | YISRSGSTKY | DETDYAYDE | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 73) | NO: 64) | | 71) |
| | | 62) | | | | |
| MAB7 | DYYMS | YISRSGSTKY | DETDYAYDN | QASQDISNY | DASNLET (SEQ ID | QQYEDYPLV |
| | (SEQ ID | YADSVKG | (SEQ ID NO: | LN (SEQ ID | NO: 65) | (SEQ ID NO: |
| | NO: 61) | (SEQ ID NO: | 74) | NO: 64) | | 75) |
| | | 62) | | | | |
| MAB8 | DYYMS | YISRSGSTKY | DESDYAYDY | QASQDISNY | DASNLET (SEQ ID | QQHEDYPL |
| | (SEQ ID | YADSVKG | (SEQ ID NO: | LN (SEQ ID | NO: 65) | (SEQ ID NO: |
| | NO: 61) | (SEQ ID NO: | 76) | NO: 64) | | 77) |
| | | 62) | | | | |
| MAB9 | DYYMS | YISRSGSTKY | DENYALDW | QASQDISNY | DASNLET (SEQ ID | QHYEDYPL |
| | (SEQ ID | YADSVKG | (SEQ ID NO: | LN (SEQ ID | NO: 65) | (SEQ ID NO: |
| | NO: 61) | (SEQ ID NO: | 78) | NO: 64) | | 79) |
| | | 62) | | | | |
| MAB10 | DYYMS | YISRSGSTKY | DESDYALDF | QASQDISNY | DASNLET (SEQ ID | QQYEDLIPT |
| | (SEQ ID | YADSVKG | (SEQ ID NO: | LN (SEQ ID | NO: 65) | (SEQ ID NO: |
| | NO: 61) | (SEQ ID NO: | 80) | NO: 64) | | 81) |
| | | 62) | | | | |
| MAB11 | ELSMH | TFDPEEGETI | EGLAGVPFDL | RASQSISSW | KASSLES (SEQ ID | QSYNSYVWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 87) |
| | | 83) | | | | |
| MAB12 | ELSMH | TFDPEEGETI | EGLGGRPFDH | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 88) | NO: 85) | | 89) |
| | | 83 | | | | |
| MAB13 | ELSMH | TFDPEEGETI | EGLAGFPFDI | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 90) | NO: 85) | | 89) |
| | | 83 | | | | |
| MAB14 | ELSMH | TFDPEEGETI | EGLAWRPTDS | RASQSISSW | KASSLES (SEQ ID | QQYSSYAWT |
| | (SEQ ID | YAQKFQG | (SEQ ID NO: | LA (SEQ ID | NO: 86) | (SEQ ID NO: |
| | NO: 82) | (SEQ ID NO: | 91) | NO: 85) | | 92) |
| | | 83 | | | | |
| MAB15 | ELSMH | TFDPEEGETI | KGLAWLPYY | RASQSISSW | KASSLES (SEQ ID | QQARSYSWT |
| | (SEQ ID | YAQKFQG | S (SEQ ID NO: | LA (SEQ ID | NO: 86) | (SEQ ID NO: |
| | NO: 82) | (SEQ ID NO: | 93) | NO: 85) | | 94) |
| | | 83 | | | | |
| MAB16 | ELSMH | TFDPEEGETI | EGLAGVPFDL | RASQSISSW | KASSLES (SEQ ID | QQHNSYRWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 95) |
| | | 83 | | | | |
| MAB17 | ELSMH | TFDPEEGETI | EGEAGRPFDA | RASQSISSW | KASSLES (SEQ ID | VQYPSYSWT |
| | (SEQ ID | YAQKFQG | (SEQ ID NO: | LA (SEQ ID | NO: 86) | (SEQ ID NO: |
| | NO: 82) | (SEQ ID NO: | 96) | NO: 85) | | 97) |
| | | 83 | | | | |
| MAB18 | ELSMH | TFDPEEGETI | EGLAGRPYDV | RASQSISSW | KASSLES (SEQ ID | QQYNSYKLT |
| | (SEQ ID | YAQKFQG | (SEQ ID NO: | LA (SEQ ID | NO: 86) | (SEQ ID NO: |
| | NO: 82) | (SEQ ID NO: | 98) | NO: 85) | | 99) |
| | | 83 | | | | |
| MAB19 | ELSMH | TFDPEEGETI | EGLAGIPFDS | RASQSISSW | KASSLES (SEQ ID | QQYNSPSWL |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 101) |
| | | 83 | | | | |
| MAB20 | ELSMH | TFDPEEGETI | EGLAGIPFDS | RASQSISSW | KASSLES (SEQ ID | QQVNSLSWT |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 102) |
| | | 83 | | | | |
| MAB21 | DYGMS | GINWEGGST | DEQLGGTTYY | RASQGIRN | TASNLQS (SEQ ID | FQYNSYPLG |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 108) |
| | | 104) | 105) | | | |
| MAB22 | DYGMS | GINWEGGST | DEQLGGKYY | RASQGIRN | TASNLQS (SEQ ID | LQYRSHPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 110) |
| | | 104) | 109) | | | |
| MAB23 | DYGMS | GINWEGGST | DEQLGGQTY | RASQGIRN | TASNLQS (SEQ ID | LQYNQVPLT |
| | (SEQ ID | GYADSVKG | YYYYMDV | DLG (SEQ | NO: 107) | (SEQ ID NO: |
| | NO: 103) | (SEQ ID NO | (SEQ ID NO: | ID NO: 106) | | 112) |
| | | 104) | 111) | | | |
| MAB24 | DYGMS | GINWEGGST | DEQLGGTTYY | RASQGIRN | TASNLQS (SEQ ID | LOTNIYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 113) |
| | | 104) | 105) | | | |
| MAB25 | DYGMS | GINWEGGST | DEQLGGIKYY | RASQGIRN | TASNLQS (SEQ ID | LQYKQYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 115) |
| | | 104) | 114) | | | |
| MAB26 | DYGMS | GINWEGGST | DEQLGGLKY | RASQGIRN | TASNLQS (SEQ ID | LQTNIYPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 113) |
| | | 104) | 116) | | | |
| MAB27 | DYGMS | GINWEGGST | DEQLGGKVY | RASQGIRN | TASNLQS (SEQ ID | LQYNSYPLA |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 118) |
| | | 104) | 117) | | | |
| MAB28 | DYGMS | GINWEGGST | DEQLGGLKY | RASQGIRN | TASNLQS (SEQ ID | LQYHSYPLR |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 119) |
| | | 104) | 116) | | | |
| MAB29 | DYGMS | GINWEGGST | DEQLGGNHY | RASQGIRN | TASNLQS (SEQ ID | LQYASYPLK |
| | (SEQ ID | GYADSVKG | YYYYMDA | DLG (SEQ | NO: 107) | (SEQ ID NO: |
| | NO: 103) | (SEQ ID NO: | (SEQ ID NO: | ID NO: 106) | | 121) |
| | | 104) | 120) | | | |
| MAB30 | DYGMS | GINWEGGST | DEQLGGRHY | RASQGIRN | TASNLQS (SEQ ID | LQYNAYPLI |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 123) |
| | | 104) | 122) | | | |
| MAB31 | DYYMS | YISRSGSTKY | DQEDYALDY | QASQDISNY | DASNLET (SEQ ID | EQYEDYPLT |
| | (SEQ ID | YADSVKG | (SEQ ID NO: | LN (SEQ ID | NO: 65) | (SEQ ID NO: |
| | NO: 61) | (SEQ ID NO: | 63) | NO: 64) | | 66) |
| | | 62) | | | | |
| MAB32 | DYYMS | YISRSGSTKY | DETDYGWDY | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 67) | NO: 64) | | 68) |
| | | 62) | | | | |
| MAB33 | DYYMS | YISRSGSTKY | DETDYAFDE | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 69) | NO: 64) | | 68) |
| | | 62) | | | | |
| MAB34 | DYYMS | YISRSGSTKY | DESQYALDY | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 70) | NO: 64) | | 71) |
| | | 62) | | | | |
| MAB35 | DYYMS | YISRSGSTKY | DNTDYALDL | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 72) | NO: 64) | | 71) |
| | | 62) | | | | |
| MAB36 | DYYMS | YISRSGSTKY | DETDYAYDE | QASQDISNY | DASNLET (SEQ ID | HQYEDYPLT |
| | (SEQ ID | YADSVKG | (SEQ ID NO | LN (SEQ ID | NO: 65) | (SEQ ID NO: |
| | NO: 61) | (SEQ ID NO: | 73) | NO: 64) | | 71) |
| | | 62) | | | | |
| MAB37 | DYYMS | YISRSGSTKY | DETDYAYDN | QASQDISNY | DASNLET (SEQ ID | QQYEDYPLV |
| | (SEQ ID | YADSVKG | (SEQ ID NO: | LN (SEQ ID | NO: 65) | (SEQ ID NO: |
| | NO: 61) | (SEQ ID NO: | 74) | NO: 64) | | 75) |
| | | 62) | | | | |
| MAB38 | DYYMS | YISRSGSTKY | DESDYAYDY | QASQDISNY | DASNLET (SEQ ID | QQHEDYPL |
| | (SEQ ID | YADSVKG | (SEQ ID NO: | LN (SEQ ID | NO: 65) | (SEQ ID NO: |
| | NO: 61) | (SEQ ID NO: | 76) | NO: 64) | | 77) |
| | | 62) | | | | |
| MAB39 | DYYMS | YISRSGSTKY | DENYALDW | QASQDISNY | DASNLET (SEQ ID | QHYEDYPL |
| | (SEQ ID | YADSVKG | (SEQ ID NO: | LN (SEQ ID | NO: 65) | (SEQ ID NO: |
| | NO: 61) | (SEQ ID NO: | 78) | NO: 64) | | 79) |
| | | 62) | | | | |
| MAB40 | DYYMS | YISRSGSTKY | DESDYALDF | QASQDISNY | DASNLET (SEQ ID | QQYEDLIPT |
| | (SEQ ID | YADSVKG | (SEQ ID NO: | LN (SEQ ID | NO: 65) | (SEQ ID NO: |
| | NO: 61) | (SEQ ID NO: | 80) | NO: 64) | | 81) |
| | | 62) | | | | |
| MAB41 | ELSMH | TFDPEEGETI | EGLAGVPFDL | RASQSISSW | KASSLES (SEQ ID | QSYNSYVWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 87) |
| | | 83) | | | | |
| MAB42 | ELSMH | TFDPEEGETI | EGLGGRPFDH | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 88) | NO: 85) | | 89) |
| | | 83 | | | | |
| MAB43 | ELSMH | TFDPEEGETI | EGLAGFPFDI | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 90) | NO: 85) | | 89) |
| | | 83 | | | | |
| MAB44 | ELSMH | TFDPEEGETI | EGLAWRPTDS | RASQSISSW | KASSLES (SEQ ID | QQYSSYAWT |
| | (SEQ ID | YAQKFQG | (SEQ ID NO: | LA (SEQ ID | NO: 86) | (SEQ ID NO: |
| | NO: 82) | (SEQ ID NO: | 91) | NO: 85) | | 92) |
| | | 83 | | | | |
| MAB45 | ELSMH | TFDPEEGETI | KGLAWLPYY | RASQSISSW | KASSLES (SEQ ID | QQARSYSWT |
| | (SEQ ID | YAQKFQG | S (SEQ ID NO: | LA (SEQ ID | NO: 86) | (SEQ ID NO: |
| | NO: 82) | (SEQ ID NO: | 93) | NO: 85) | | 94) |
| | | 83 | | | | |
| MAB46 | ELSMH | TFDPEEGETI | EGLAGVPFDL | RASQSISSW | KASSLES (SEQ ID | QQHNSYRWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 95) |
| | | 83 | | | | |
| MAB47 | ELSMH | TFDPEEGETI | EGEAGRPFDA | RASQSISSW | KASSLES (SEQ ID | VQYPSYSWT |
| | (SEQ ID | YAQKFQG | (SEQ ID NO: | LA (SEQ ID | NO: 86) | (SEQ ID NO: |
| | NO: 82) | (SEQ ID NO: | 96) | NO: 85) | | 97) |
| | | 83 | | | | |
| MAB48 | ELSMH | TFDPEEGETI | EGLAGRPYDV | RASQSISSW | KASSLES (SEQ ID | QQYNSYKLT |
| | (SEQ ID | YAQKFQG | (SEQ ID NO: | LA (SEQ ID | NO: 86) | (SEQ ID NO: |
| | NO: 82) | (SEQ ID NO: | 98) | NO: 85) | | 99) |
| | | 83 | | | | |
| MAB49 | ELSMH | TFDPEEGETI | EGLAGIPFDS | RASQSISSW | KASSLES (SEQ ID | QQYNSPSWL |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 101) |
| | | 83 | | | | |
| MAB50 | ELSMH | TFDPEEGETI | EGLAGIPFDS | RASQSISSW | KASSLES (SEQ ID | QQVNSLSWT |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 102) |
| | | 83 | | | | |
| MAB51 | DYGMS | GINWEGGST | DEQLGGTTYY | RASQGIRN | TASNLQS (SEQ ID | FQYNSYPLG |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 108) |
| | | 104) | 105) | | | |
| MAB52 | DYGMS | GINWEGGST | DEQLGGKYY | RASQGIRN | TASNLQS (SEQ ID | LQYRSHPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 110) |
| | | 104) | 109) | | | |
| MAB53 | DYGMS | GINWEGGST | DEQLGGQTY | RASQGIRN | TASNLQS (SEQ ID | LQYNQVPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 112) |
| | | 104) | 111) | | | |
| MAB54 | DYGMS | GINWEGGST | DEQLGGTTYY | RASQGIRN | TASNLQS (SEQ ID | LQTNIYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 113) |
| | | 104) | 105) | | | |
| MAB55 | DYGMS | GINWEGGST | DEQLGGIKYY | RASQGIRN | TASNLQS (SEQ ID | LQYKQYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 115) |
| | | 104) | 114) | | | |
| MAB56 | DYGMS | GINWEGGST | DEQLGGLKY | RASQGIRN | TASNLQS (SEQ ID | LOTNIYPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 113) |
| | | 104) | 116) | | | |
| MAB57 | DYGMS | GINWEGGST | DEQLGGKVY | RASQGIRN | TASNLQS (SEQ ID | LQYNSYPLA |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 118) |
| | | 104) | 117) | | | |
| MAB58 | DYGMS | GINWEGGST | DEQLGGLKY | RASQGIRN | TASNLQS (SEQ ID | LQYHSYPLR |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 119) |
| | | 104) | 116) | | | |
| MAB59 | DYGMS | GINWEGGST | DEQLGGNHY | RASQGIRN | TASNLQS (SEQ ID | LQYASYPLK |
| | (SEQ ID | GYADSVKG | YYYYMDA | DLG (SEQ | NO: 107) | (SEQ ID NO: |
| | NO: 103) | (SEQ ID NO: | (SEQ ID NO: | ID NO: 106) | | 121) |
| | | 104) | 120) | | | |
| MAB60 | DYGMS | GINWEGGST | DEQLGGRHY | RASQGIRN | TASNLQS (SEQ ID | LQYNAYPLI |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 123) |
| | | 104) | 122) | | | |

The CDRs in the above-identified table can also be referred to according to Chothia CDRs or IMGT CDRs. These are illustrated in the following tables for the different antibodies.

TABLE 3
CHOTHIA CDRS

| Ab ID No | HCDR1 | HCDR2 | HCDR3 | LCDR1 | LCDR2 | LCDR3 |
|---|---|---|---|---|---|---|
| MAB1 | GFTFSDY | SRSGST | DQEDYALDY | QASQDISNY | DASNLET (SEQ ID | EQYEDYPLT |
| | (SEQ ID NO: | (SEQ ID | (SEQ ID NO: | LN (SEQ ID | NO: 65) | (SEQ ID NO: |
| | 124) | NO: 125) | 63) | NO: 64) | | 66) |
| MAB2 | GFTFSDY | SRSGST | DETDYGWDY | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 67) | NO: 64) | | 68) |
| MAB3 | GFTFSDY | SRSGST | DETDYAFDE | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 69) | NO: 64) | | 68) |
| MAB4 | GFTFSDY | SRSGST | DESQYALDY | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 70) | NO: 64) | | 71) |
| MAB5 | GFTFSDY | SRSGST | DNTDYALDL | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 72) | NO: 64) | | 71) |
| MAB6 | GFTFSDY | SRSGST | DETDYAYDE | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 73) | NO: 64) | | 71) |
| MAB7 | GFTFSDY | SRSGST | DETDYAYDN | QASQDISNY | DASNLET (SEQ ID | QQYEDYPLV |
| | (SEQ ID NO: | (SEQ ID | (SEQ ID NO: | LN (SEQ ID | NO: 65) | (SEQ ID NO: |
| | 124) | NO: 125) | 74) | NO: 64) | | 75) |
| MAB8 | GFTFSDY | SRSGST | DESDYAYDY | QASQDISNY | DASNLET (SEQ ID | QQHEDYPL |
| | (SEQ ID NO: | (SEQ ID | (SEQ ID NO: | LN (SEQ ID | NO: 65) | (SEQ ID NO: |
| | 124) | NO: 125) | 76) | NO: 64) | | 77) |
| MAB9 | GFTFSDY | SRSGST | DENYALDW | QASQDISNY | DASNLET (SEQ ID | QHYEDYPL |
| | (SEQ ID NO: | (SEQ ID | (SEQ ID NO: | LN (SEQ ID | NO: 65) | (SEQ ID NO: |
| | 124) | NO: 125) | 78) | NO: 64) | | 79) |
| MAB10 | GFTFSDY | SRSGST | DESDYALDF | QASQDISNY | DASNLET (SEQ ID | QQYEDLIPT |
| | (SEQ ID NO: | (SEQ ID | (SEQ ID NO: | LN (SEQ ID | NO: 65) | (SEQ ID NO: |
| | 124) | NO: 125) | 80) | NO: 64) | | 81) |
| MAB11 | GDTLTEL | DPEEGE | EGLAGVPFDL | RASQSISSW | KASSLES (SEQ ID | QSYNSYVWT |
| | (SEQ ID NO: | (SEQ ID | (SEQ ID NO: | LA (SEQ ID | NO: 86) | (SEQ ID NO: |
| | 126) | NO: 127) | 84) | NO: 85) | | 87) |
| MAB12 | GDTLTEL | DPEEGE | EGLGGRPFDH | RASQSISSW | KASSLES (SEQ ID | QQVNSYDWT |
| | (SEQ ID NO: | (SEQ ID | (SEQ ID NO: | LA (SEQ ID | NO: 86) | (SEQ ID NO |
| | 126) | NO: 127) | 88) | NO: 85) | | 89) |
| MAB13 | GDTLTEL | DPEEGE | EGLAGFPFDI | RASQSISSW | KASSLES (SEQ ID | QQVNSYDWT |
| | (SEQ ID NO: | (SEQ ID | (SEQ ID NO: | LA (SEQ ID | NO: 86) | (SEQ ID NO |
| | 126) | NO: 127) | 90) | NO: 85) | | 89) |
| MAB14 | GDTLTEL | DPEEGE | EGLAWRPTDS | RASQSISSW | KASSLES (SEQ ID | QQYSSYAWT |
| | (SEQ ID NO: | (SEQ ID | (SEQ ID NO: | LA (SEQ ID | NO: 86) | (SEQ ID NO: |
| | 126) | NO: 127) | 91) | NO: 85) | | 92) |
| MAB15 | GDTLTEL | DPEEGE | KGLAWLPYY | RASQSISSW | KASSLES (SEQ ID | QQARSYSWT |
| | (SEQ ID NO: | (SEQ ID | S (SEQ ID NO: | LA (SEQ ID | NO: 86) | (SEQ ID NO: |
| | 126) | NO: 127) | 93) | NO: 85) | | 94) |
| MAB16 | GDTLTEL | DPEEGE | EGLAGVPFDL | RASQSISSW | KASSLES (SEQ ID | QQHNSYRWT |
| | (SEQ ID NO: | (SEQ ID | (SEQ ID NO: | LA (SEQ ID | NO: 86) | (SEQ ID NO: |
| | 126) | NO: 127) | 84) | NO: 85) | | 95) |
| MAB17 | GDTLTEL | DPEEGE | EGEAGRPFDA | RASQSISSW | KASSLES (SEQ ID | VQYPSYSWT |
| | (SEQ ID NO: | (SEQ ID | (SEQ ID NO: | LA (SEQ ID | NO: 86) | (SEQ ID NO: |
| | 126) | NO: 127) | 96) | NO: 85) | | 97) |
| MAB18 | GDTLTEL | DPEEGE | EGLAGRPYDV | RASQSISSW | KASSLES (SEQ ID | QQYNSYKLT |
| | (SEQ ID NO: | (SEQ ID | (SEQ ID NO: | LA (SEQ ID | NO: 86) | (SEQ ID NO: |
| | 126) | NO: 127) | 98) | NO: 85) | | 99) |
| MAB19 | GDTLTEL | DPEEGE | EGLAGIPFDS | RASQSISSW | KASSLES (SEQ ID | QQYNSPSWL |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 101) |
| MAB20 | GDTLTEL | DPEEGE | EGLAGIPFDS | RASQSISSW | KASSLES (SEQ ID | QQVNSLSWT |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 102) |
| MAB21 | GFTFDDY | NWEGGS | DEQLGGTTYY | RASQGIRN | TASNLQS (SEQ ID | FQYNSYPLG |
| | (SEQ ID NO: | (SEQ II | YYYMDV | DLG (SEQ | NO: 107) | (SEQ ID NO: |
| | 128) | NO: 129) | (SEQ ID NO: | ID NO: 106) | | 108) |
| | | | 105) | | | |
| MAB22 | GFTFDDY | NWEGGS | DEQLGGKYY | RASQGIRN | TASNLQS (SEQ ID | LQYRSHPLT |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 110) |
| | | | 109) | | | |
| MAB23 | GFTFDDY | NWEGGS | DEQLGGQTY | RASQGIRN | TASNLQS (SEQ ID | LQYNQVPLT |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 112) |
| | | | 111) | | | |
| MAB24 | GFTFDDY | NWEGGS | DEQLGGTTYY | RASQGIRN | TASNLQS (SEQ ID | LOTNIYPLT |
| | (SEQ ID NO: | (SEQ ID | YYYMDV | DLG (SEQ | NO: 107) | (SEQ ID NO: |
| | 128) | NO: 129) | (SEQ ID NO: | ID NO: 106) | | 113) |
| | | | 105) | | | |
| MAB25 | GFTFDDY | NWEGGS | DEQLGGIKYY | RASQGIRN | TASNLQS (SEQ ID | LQYKQYPLT |
| | (SEQ ID NO: | (SEQ ID | YYYMDV | DLG (SEQ | NO: 107) | (SEQ ID NO: |
| | 128) | NO: 129) | (SEQ ID NO: | ID NO: 106) | | 115) |
| | | | 114) | | | |
| MAB26 | GFTFDDY | NWEGGS | DEQLGGLKY | RASQGIRN | TASNLQS (SEQ ID | LQTNIYPLT |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 113) |
| | | | 116) | | | |
| MAB27 | GFTFDDY | NWEGGS | DEQLGGKVY | RASQGIRN | TASNLQS (SEQ ID | LQYNSYPLA |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 118) |
| | | | 117) | | | |
| MAB28 | GFTFDDY | NWEGGS | DEQLGGLKY | RASQGIRN | TASNLQS (SEQ ID | LQYHSYPLR |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 119) |
| | | | 116) | | | |
| MAB29 | GFTFDDY | NWEGGS | DEQLGGNHY | RASQGIRN | TASNLQS (SEQ ID | LQYASYPLK |
| | (SEQ ID NO: | (SEQ ID | YYYYMDA | DLG (SEQ | NO: 107) | (SEQ ID NO: |
| | 128) | NO: 129) | (SEQ ID NO: | ID NO: 106) | | 121) |
| | | | 120) | | | |
| MAB30 | GFTFDDY | NWEGGS | DEQLGGRHY | RASQGIRN | TASNLQS (SEQ ID | LOYNAYPLI |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 123) |
| | | | 122) | | | |
| MAB31 | GFTFSDY | SRSGST | DQEDYALDY | QASQDISNY | DASNLET (SEQ ID | EQYEDYPLT |
| | (SEQ ID NO: | (SEQ ID | (SEQ ID NO: | LN (SEQ ID | NO: 65) | (SEQ ID NO: |
| | 124) | NO: 125) | 63) | NO: 64) | | 66) |
| MAB32 | GFTFSDY | SRSGST | DETDYGWDY | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 67) | NO: 64) | | 68) |
| MAB33 | GFTFSDY | SRSGST | DETDYAFDE | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 69) | NO: 64) | | 68) |
| MAB34 | GFTFSDY | SRSGST | DESQYALDY | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 70) | NO: 64) | | 71) |
| MAB35 | GFTFSDY | SRSGST | DNTDYALDL | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 72) | NO: 64) | | 71) |
| MAB36 | GFTFSDY | SRSGST | DETDYAYDE | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 73) | NO: 64) | | 71) |
| MAB37 | GFTFSDY | SRSGST | DETDYAYDN | QASQDISNY | DASNLET (SEQ ID | QQYEDYPLV |
| | (SEQ ID NO: | (SEQ ID | (SEQ ID NO: | LN (SEQ ID | NO: 65) | (SEQ ID NO: |
| | 124) | NO: 125) | 74) | NO: 64) | | 75) |
| MAB38 | GFTFSDY | SRSGST | DESDYAYDY | QASQDISNY | DASNLET (SEQ ID | QQHEDYPL |
| | (SEQ ID NO: | (SEQ ID | (SEQ ID NO: | LN (SEQ ID | NO: 65) | (SEQ ID NO: |
| | 124) | NO: 125) | 76) | NO: 64) | | 77) |
| MAB39 | GFTFSDY | SRSGST | DENYALDW | QASQDISNY | DASNLET (SEQ ID | QHYEDYPL |
| | (SEQ ID NO: | (SEQ ID | (SEQ ID NO: | LN (SEQ ID | NO: 65) | (SEQ ID NO: |
| | 124) | NO: 125) | 78) | NO: 64) | | 79) |
| MAB40 | GFTFSDY | SRSGST | DESDYALDF | QASQDISNY | DASNLET (SEQ ID | QQYEDLIPT |
| | (SEQ ID NO: | (SEQ ID | (SEQ ID NO: | LN (SEQ ID | NO: 65) | (SEQ ID NO: |
| | 124) | NO: 125) | 80) | NO: 64) | | 81) |
| MAB41 | GDTLTEL | DPEEGE | EGLAGVPFDL | RASQSISSW | KASSLES (SEQ ID | QSYNSYVWT |
| | (SEQ ID NO: | (SEQ ID | (SEQ ID NO: | LA (SEQ ID | NO: 86) | (SEQ ID NO: |
| | 126) | NO: 127) | 84) | NO: 85) | | 87) |
| MAB42 | GDTLTEL | DPEEGE | EGLGGRPFDH | RASQSISSW | KASSLES (SEQ ID | QQVNSYDWT |
| | (SEQ ID NO: | (SEQ ID | (SEQ ID NO | LA (SEQ ID | NO: 86) | (SEQ ID NO |
| | 126) | NO: 127) | 88) | NO: 85) | | 89) |
| MAB43 | GDTLTEL | DPEEGE | EGLAGFPFDI | RASQSISSW | KASSLES (SEQ ID | QQVNSYDWT |
| | (SEQ ID NO: | (SEQ ID | (SEQ ID NO | LA (SEQ ID | NO: 86) | (SEQ ID NO |
| | 126) | NO: 127) | 90) | NO: 85) | | 89) |
| MAB44 | GDTLTEL | DPEEGE | EGLAWRPTDS | RASQSISSW | KASSLES (SEQ ID | QQYSSYAWT |
| | (SEQ ID NO: | (SEQ ID | (SEQ ID NO: | LA (SEQ ID | NO: 86) | (SEQ ID NO: |
| | 126) | NO: 127) | 91) | NO: 85) | | 92) |
| MAB45 | GDTLTEL | DPEEGE | KGLAWLPYY | RASQSISSW | KASSLES (SEQ ID | QQARSYSWT |
| | (SEQ ID NO: | (SEQ ID | S (SEQ ID NO: | LA (SEQ ID | NO: 86) | (SEQ ID NO: |
| | 126) | NO: 127) | 93) | NO: 85) | | 94) |
| MAB46 | GDTLTEL | DPEEGE | EGLAGVPFDL | RASQSISSW | KASSLES (SEQ ID | QQHNSYRWT |
| | (SEQ ID NO: | (SEQ ID | (SEQ ID NO | LA (SEQ ID | NO: 86) | (SEQ ID NO: |
| | 126) | NO: 127) | 84) | NO: 85) | | 95) |
| MAB47 | GDTLTEL | DPEEGE | EGEAGRPFDA | RASQSISSW | KASSLES (SEQ ID | VQYPSYSWT |
| | (SEQ ID NO: | (SEQ ID | (SEQ ID NO: | LA (SEQ ID | NO: 86) | (SEQ ID NO |
| | 126) | NO: 127) | 96) | NO: 85) | | 97) |
| MAB48 | GDTLTEL | DPEEGE | EGLAGRPYDV | RASQSISSW | KASSLES (SEQ ID | QQYNSYKLT |
| | (SEQ ID NO: | (SEQ ID | (SEQ ID NO: | LA (SEQ ID | NO: 86) | (SEQ ID NO: |
| | 126) | NO: 127) | 98) | NO: 85) | | 99) |
| MAB49 | GDTLTEL | DPEEGE | EGLAGIPFDS | RASQSISSW | KASSLES (SEQ ID | QQYNSPSWL |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 101) |
| MAB50 | GDTLTEL | DPEEGE | EGLAGIPFDS | RASQSISSW | KASSLES (SEQ ID | QQVNSLSWT |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 102) |
| MAB51 | GFTFDDY | NWEGGS | DEQLGGTTYY | RASQGIRN | TASNLQS (SEQ ID | FQYNSYPLG |
| | (SEQ ID NO: | (SEQ ID | YYYMDV | DLG (SEQ | NO: 107) | (SEQ ID NO: |
| | 128) | NO: 129) | (SEQ ID NO: | ID NO: 106) | | 108) |
| | | | 105) | | | |
| MAB52 | GFTFDDY | NWEGGS | DEQLGGKYY | RASQGIRN | TASNLQS (SEQ ID | LQYRSHPLT |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 110) |
| | | | 109) | | | |
| MAB53 | GFTFDDY | NWEGGS | DEQLGGQTY | RASQGIRN | TASNLQS (SEQ ID | LQYNQVPLT |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 112) |
| | | | 111) | | | |
| MAB54 | GFTFDDY | NWEGGS | DEQLGGTTYY | RASQGIRN | TASNLQS (SEQ ID | LQTNIYPLT |
| | (SEQ ID NO: | (SEQ ID | YYYMDV | DLG (SEQ | NO: 107) | (SEQ ID NO: |
| | 128) | NO: 129) | (SEQ ID NO: | ID NO: 106) | | 113) |
| | | | 105) | | | |
| MAB55 | GFTFDDY | NWEGGS | DEQLGGIKYY | RASQGIRN | TASNLQS (SEQ ID | LQYKQYPLT |
| | (SEQ ID NO: | (SEQ ID | YYYMDV | DLG (SEQ | NO: 107) | (SEQ ID NO: |
| | 128) | NO: 129) | (SEQ ID NO: | ID NO: 106) | | 115) |
| | | | 114) | | | |
| MAB56 | GFTFDDY | NWEGGS | DEQLGGLKY | RASQGIRN | TASNLQS (SEQ ID | LQTNIYPLT |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 113) |
| | | | 116) | | | |
| MAB57 | GFTFDDY | NWEGGS | DEQLGGKVY | RASQGIRN | TASNLQS (SEQ ID | LQYNSYPLA |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 118) |
| | | | 117) | | | |
| MAB58 | GFTFDDY | NWEGGS | DEQLGGLKY | RASQGIRN | TASNLQS (SEQ ID | LQYHSYPLR |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 119) |
| | | | 116) | | | |
| MAB59 | GFTFDDY | NWEGGS | DEQLGGNHY | RASQGIRN | TASNLQS (SEQ ID | LQYASYPLK |
| | (SEQ ID NO: | (SEQ ID | YYYYMDA | DLG (SEQ | NO: 107) | (SEQ ID NO: |
| | 128) | NO: 129) | (SEQ ID NO: | ID NO: 106) | | 121) |
| | | | 120) | | | |
| MAB60 | GFTFDDY | NWEGGS | DEQLGGRHY | RASQGIRN | TASNLQS (SEQ ID | LQYNAYPLI |
| | (SEQ ID NO: | (SEQ ID | YYYYMDV | DLG (SEQ | NO: 107) | (SEQ ID NO: |
| | 128) | NO: 129) | (SEQ ID NO: | ID NO: 106) | | 123) |
| | | | 122) | | | |

TABLE 4
IMGT CDRS

| Ab ID No | HCDR1 | HCDR2 | HCDR3 | LCDR1 | LCDR2 | LCDR3 |
|---|---|---|---|---|---|---|
| MAB1 | GFTFSDY | ISRSGSTK | ARDQEDYAL | QDISNY | DAS (SEQ ID NO: | EQYEDYPLT |
| | Y (SEQ ID | (SEQ ID NO: | DY (SEQ ID | (SEQ ID NO: | 134) | (SEQ ID NO: |
| | NO: 130) | 131) | NO: 132) | 133) | | 66) |
| MAB2 | GFTFSDY | ISRSGSTK | ARDETDYGW | QDISNY | DAS (SEQ ID NO: | KQYEDYPLT |
| | Y (SEQ ID | (SEQ ID NO: | DY (SEQ ID | (SEQ ID NO: | 134) | (SEQ ID NO: |
| | NO: 130) | 131) | NO: 135) | 133) | | 68) |
| MAB3 | GFTFSDY | ISRSGSTK | ARDETDYAFD | QDISNY | DAS (SEQ ID NO: | KQYEDYPLT |
| | Y (SEQ ID | (SEQ ID NO: | E (SEQ ID NO: | (SEQ ID NO: | 134) | (SEQ ID NO: |
| | NO: 130) | 131) | 136) | 133) | | 68) |
| MAB4 | GFTFSDY | ISRSGSTK | ARDESQYALD | QDISNY | DAS (SEQ ID NO: | HQYEDYPLT |
| | Y (SEQ ID | (SEQ ID NO: | Y (SEQ ID NO: | (SEQ ID NO: | 134) | (SEQ ID NO: |
| | NO: 130) | 131) | 137) | 133) | | 71) |
| MAB5 | GFTFSDY | ISRSGSTK | ARDNTDYAL | QDISNY | DAS (SEQ ID NO: | HQYEDYPLT |
| | Y (SEQ ID | (SEQ ID NO: | DL (SEQ ID | (SEQ ID NO: | 134) | (SEQ ID NO: |
| | NO: 130) | 131) | NO: 138) | 133) | | 71) |
| MAB6 | GFTFSDY | ISRSGSTK | ARDETDYAY | QDISNY | DAS (SEQ ID NO: | HQYEDYPLT |
| | Y (SEQ ID | (SEQ ID NO: | DE (SEQ ID | (SEQ ID NO: | 134) | (SEQ ID NO: |
| | NO: 130) | 131) | NO: 139) | 133) | | 71) |
| MAB7 | GFTFSDY | ISRSGSTK | ARDETDYAY | QDISNY | DAS (SEQ ID NO: | QQYEDYPLV |
| | Y (SEQ ID | (SEQ ID NO: | DN (SEQ ID | (SEQ ID NO: | 134) | (SEQ ID NO: |
| | NO: 130) | 131) | NO: 140) | 133) | | 75) |
| MAB8 | GFTFSDY | ISRSGSTK | ARDESDYAY | QDISNY | DAS (SEQ ID NO: | QQHEDYPL |
| | Y (SEQ ID | (SEQ ID NO: | DY (SEQ ID | (SEQ ID NO: | 134) | (SEQ ID NO: |
| | NO: 130) | 131) | NO: 141) | 133) | | 77) |
| MAB9 | GFTFSDY | ISRSGSTK | ARDENYALD | QDISNY | DAS (SEQ ID NO: | QHYEDYPL |
| | | (SEQ ID NO: | W (SEQ ID NO: | (SEQ ID NO: | | (SEQ ID NO: |
| | Y (SEQ ID | 131) | 142) | 133) | 134) | 79) |
| | NO: 130) | | | | | |
| MAB10 | GFTFSDY | ISRSGSTK | ARDESDYALD | QDISNY | DAS (SEQ ID NO: | QQYEDLIPT |
| | Y (SEQ ID | (SEQ ID NO: | F (SEQ ID NO: | (SEQ ID NO: | 134) | (SEQ ID NO: |
| | NO: 130) | 131) | 143) | 133) | | 81) |
| MAB11 | GDTLTEL | FDPEEGET | VTEGLAGVPF | QSISSW | KAS (SEQ ID NO: | QSYNSYVWT |
| | S (SEQ ID | (SEQ ID NO: | DL (SEQ ID | (SEQ ID NO: | | (SEQ ID NO: |
| | NO: 144) | 145) | NO: 146) | 147) | 148) | 87) |
| MAB12 | GDTLTEL | FDPEEGET | VTEGLGGRPF | QSISSW | KAS (SEQ ID NO: | QQVNSYDWT |
| | S (SEQ ID | (SEQ ID NO: | DH (SEQ ID | (SEQ ID NO: | | (SEQ ID NO |
| | NO: 144) | 145) | NO: 149) | 147) | 148) | 89) |
| MAB13 | GDTLTEL | FDPEEGET | VTEGLAGFPF | QSISSW | KAS (SEQ ID NO: | QQVNSYDWT |
| | S (SEQ ID | (SEQ ID NO: | DI (SEQ ID | (SEQ ID NO: | 148) | (SEQ ID NO |
| | NO: 144) | 145) | NO: 150) | 147) | | 89) |
| MAB14 | GDTLTEL | FDPEEGET | VTEGLAWRPT | QSISSW | KAS (SEQ ID NO: | QQYSSYAWT |
| | S (SEQ ID | (SEQ ID NO: | DS (SEQ ID | (SEQ ID NO: | | (SEQ ID NO |
| | NO: 144) | 145) | NO: 151) | 147) | 148) | 92) |
| MAB15 | GDTLTEL | FDPEEGET | VTKGLAWLP | QSISSW | KAS (SEQ ID NO: | QQARSYSWT |
| | S (SEQ ID | (SEQ ID NO: | YYS (SEQ ID | (SEQ ID NO: | | (SEQ ID NO: |
| | NO: 144) | 145) | NO: 152) | 147) | 148) | 94) |
| MAB16 | GDTLTEL | FDPEEGET | VTEGLAGVPF | QSISSW | KAS (SEQ ID NO: | QQHNSYRWT |
| | S (SEQ ID | (SEQ ID NO: | DL (SEQ ID | (SEQ ID NO: | 148) | (SEQ ID NO: |
| | NO: 144) | 145) | NO: 146) | 147) | | 95) |
| MAB17 | GDTLTEL | FDPEEGET | VTEGEAGRPF | QSISSW | KAS (SEQ ID NO: | VQYPSYSWT |
| | S (SEQ ID | (SEQ ID NO: | DA (SEQ ID | (SEQ ID NO: | 148) | (SEQ ID NO: |
| | NO: 144) | 145) | NO: 153) | 147) | | 97) |
| MAB18 | GDTLTEL | FDPEEGET | VTEGLAGRPY | QSISSW | KAS (SEQ ID NO: | QQYNSYKLT |
| | S (SEQ ID | (SEQ ID NO: | DV (SEQ ID | (SEQ ID NO: | | (SEQ ID NO: |
| | NO: 144) | 145) | NO: 154) | 147) | 148) | 99) |
| MAB19 | GDTLTEL | FDPEEGET | VTEGLAGIPF | QSISSW | KAS (SEQ ID NO: | QQYNSPSWL |
| | S (SEQ ID | (SEQ ID NO: | DSW (SEQ ID | (SEQ ID NO: | 148) | (SEQ ID NO: |
| | NO: 144) | 145) | NO: 155) | 147) | | 101) |
| MAB20 | GDTLTEL | FDPEEGET | VTEGLAGIPF | QSISSW | KAS (SEQ ID NO: | QQVNSLSWT |
| | S (SEQ ID | (SEQ ID NO: | DSW (SEQ ID | (SEQ ID NO: | | (SEQ ID NO: |
| | NO: 144) | 145) | NO: 155) | 147) | 148) | 102) |
| MAB21 | GFTFDDY | INWEGGST | ARDEQLGGTT | QGIRND | TAS (SEQ ID NO: 160) | FQYNSYPLG |
| | G (SEQ ID | (SEQ ID NO: | YYYYYMDV | (SEQ ID NO: | | (SEQ ID NO: |
| | NO: 156) | 157) | (SEQ ID NO: | 159) | | 108) |
| | | | 158) | | | |
| MAB22 | GFTFDDY | INWEGGST | ARDEQLGGK | QGIRND | TAS (SEQ ID NO: 160) | LQYRSHPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 110) |
| | | | 161) | | | |
| MAB23 | GFTFDDY | INWEGGST | ARDEQLGGQ | QGIRND | TAS (SEQ ID NO: 160) | LQYNQVPLT |
| | G (SEQ ID | (SEQ ID NO: | TYYYYYMDV | (SEQ ID NO: | | (SEQ ID NO: |
| | NO: 156) | 157) | (SEQ ID NO: | 159) | | 112) |
| | | | 162) | | | |
| MAB24 | GFTFDDY | INWEGGST | ARDEQLGGTT | QGIRND | TAS (SEQ ID NO: 160) | LOTNIYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 113) |
| | | | 158) | | | |
| MAB25 | GFTFDDY | INWEGGST | ARDEQLGGIK | QGIRND | TAS (SEQ ID NO: 160) | LQYKQYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 115) |
| | | | 163) | | | |
| MAB26 | GFTFDDY | INWEGGST | ARDEQLGGL | QGIRND | TAS (SEQ ID NO: 160) | LQTNIYPLT |
| | G (SEQ ID | (SEQ ID NO: | KYYYYYMDV | (SEQ ID NO: | | (SEQ ID NO: |
| | NO: 156) | 157) | (SEQ ID NO: | 159) | | 113) |
| | | | 164) | | | |
| MAB27 | GFTFDDY | INWEGGST | ARDEQLGGK | QGIRND | TAS (SEQ ID NO: 160) | LQYNSYPLA |
| | G (SEQ ID | (SEQ ID NO: | VYYYYYMDV | (SEQ ID NO: | | (SEQ ID NO: |
| | NO: 156) | 157) | (SEQ ID NO: | 159) | | 118) |
| | | | 165) | | | |
| MAB28 | GFTFDDY | INWEGGST | ARDEQLGGL | QGIRND | TAS (SEQ ID NO: 160) | LQYHSYPLR |
| | G (SEQ ID | (SEQ ID NO: | KYYYYYMDV | (SEQ ID NO: | | (SEQ ID NO: |
| | NO: 156) | 157) | (SEQ ID NO: | 159) | | 119) |
| | | | 164) | | | |
| MAB29 | GFTFDDY | INWEGGST | ARDEQLGGN | QGIRND | TAS (SEQ ID NO: 160) | LQYASYPLK |
| | G (SEQ ID | (SEQ ID NO: | HYYYYYMDA | (SEQ ID NO: | | (SEQ ID NO: |
| | NO: 156) | 157) | (SEQ ID NO: | 159) | | 121) |
| | | | 166) | | | |
| MAB30 | GFTFDDY | INWEGGST | ARDEQLGGR | QGIRND | TAS (SEQ ID NO: 160) | LQYNAYPLI |
| | G (SEQ ID | (SEQ ID NO: | HYYYYYMDV | (SEQ ID NO: | | (SEQ ID NO: |
| | NO: 156) | 157) | (SEQ ID NO | 159) | | 123) |
| | | | 167) | | | |
| MAB31 | GFTFSDY | ISRSGSTK | ARDQEDYAL | QDISNY | DAS (SEQ ID NO: | EQYEDYPLT |
| | Y (SEQ ID | (SEQ ID NO: | DY (SEQ ID | (SEQ ID NO: | | (SEQ ID NO: |
| | NO: 130) | 131) | NO: 132) | 133) | 134) | 66) |
| MAB32 | GFTFSDY | ISRSGSTK | ARDETDYGW | QDISNY | DAS (SEQ ID NO: | KQYEDYPLT |
| | Y (SEQ ID | (SEQ ID NO: | DY (SEQ ID | (SEQ ID NO: | 134) | (SEQ ID NO: |
| | NO: 130) | 131) | NO: 135) | 133) | | 68) |
| MAB33 | GFTFSDY | ISRSGSTK | ARDETDYAFD | QDISNY | DAS (SEQ ID NO: | KQYEDYPLT |
| | Y (SEQ ID | (SEQ ID NO: | E (SEQ ID NO: | (SEQ ID NO: | 134) | (SEQ ID NO: |
| | NO: 130) | 131) | 136) | 133) | | 68) |
| MAB34 | GFTFSDY | ISRSGSTK | ARDESQYALD | QDISNY | DAS (SEQ ID NO: | HQYEDYPLT |
| | Y (SEQ ID | (SEQ ID NO: | Y (SEQ ID NO: | (SEQ ID NO: | 134) | (SEQ ID NO: |
| | NO: 130) | 131) | 137) | 133) | | 71) |
| MAB35 | GFTFSDY | ISRSGSTK | ARDNTDYAL | QDISNY | DAS (SEQ ID NO: | HQYEDYPLT |
| | Y (SEQ ID | (SEQ ID NO: | DL (SEQ ID | (SEQ ID NO: | 134) | (SEQ ID NO: |
| | NO: 130) | 131) | NO: 138) | 133) | | 71) |
| MAB36 | GFTFSDY | ISRSGSTK | ARDETDYAY | QDISNY | DAS (SEQ ID NO: | HQYEDYPLT |
| | Y (SEQ ID | (SEQ ID NO: | DE (SEQ ID | (SEQ ID NO: | | (SEQ ID NO: |
| | NO: 130) | 131) | NO: 139) | 133) | 134) | 71) |
| MAB37 | GFTFSDY | ISRSGSTK | ARDETDYAY | QDISNY | DAS (SEQ ID NO: | QQYEDYPLV |
| | Y (SEQ ID | (SEQ ID NO: | DN (SEQ ID | (SEQ ID NO: | 134) | (SEQ ID NO: |
| | NO: 130) | 131) | NO: 140) | 133) | | 75) |
| MAB38 | GFTFSDY | ISRSGSTK | ARDESDYAY | QDISNY | DAS (SEQ ID NO: | QQHEDYPL |
| | Y (SEQ ID | (SEQ ID NO: | DY (SEQ ID | (SEQ ID NO: | 134) | (SEQ ID NO: |
| | NO: 130) | 131) | NO: 141) | 133) | | 77) |
| MAB39 | GFTFSDY | ISRSGSTK | ARDENYALD | QDISNY | DAS (SEQ ID NO: | QHYEDYPL |
| | | (SEQ ID NO: | W (SEQ ID NO: | (SEQ ID NO: | | (SEQ ID NO: |
| | Y (SEQ ID | 131) | 142) | 133) | 134) | 79) |
| | NO: 130) | | | | | |
| MAB40 | GFTFSDY | ISRSGSTK | ARDESDYALD | QDISNY | DAS (SEQ ID NO: | QQYEDLIPT |
| | Y (SEQ ID | (SEQ ID NO: | F (SEQ ID NO: | (SEQ ID NO: | 134) | (SEQ ID NO: |
| | NO: 130) | 131) | 143) | 133) | | 81) |
| MAB41 | GDTLTEL | FDPEEGET | VTEGLAGVPF | QSISSW | KAS (SEQ ID NO: | QSYNSYVWT |
| | S (SEQ ID | (SEQ ID NO: | DL (SEQ ID | (SEQ ID NO: | 148) | (SEQ ID NO: |
| | NO: 144) | 145) | NO: 146) | 147) | | 87) |
| MAB42 | GDTLTEL | FDPEEGET | VTEGLGGRPF | QSISSW | KAS (SEQ ID NO: | QQVNSYDWT |
| | S (SEQ ID | (SEQ ID NO: | DH (SEQ ID | (SEQ ID NO: | | (SEQ ID NO |
| | NO: 144) | 145) | NO: 149) | 147) | 148) | 89) |
| MAB43 | GDTLTEL | FDPEEGET | VTEGLAGFPF | QSISSW | KAS (SEQ ID NO: | QQVNSYDWT |
| | S (SEQ ID | (SEQ ID NO: | DI (SEQ ID | (SEQ ID NO: | 148) | (SEQ ID NO |
| | NO: 144) | 145) | NO: 150) | 147) | | 89) |
| MAB44 | GDTLTEL | FDPEEGET | VTEGLAWRPT | QSISSW | KAS (SEQ ID NO: | QQYSSYAWT |
| | | (SEQ ID NO: | DS (SEQ ID | (SEQ ID NO: | | (SEQ ID NO: |
| | S (SEQ ID | 145) | NO: 151) | 147) | 148) | 92) |
| | NO: 144) | | | | | |
| MAB45 | GDTLTEL | FDPEEGET | VTKGLAWLP | QSISSW | KAS (SEQ ID NO: | QQARSYSWT |
| | S (SEQ ID | (SEQ ID NO: | YYS (SEQ ID | (SEQ ID NO: | 148) | (SEQ ID NO: |
| | NO: 144) | 145) | NO: 152) | 147) | | 94) |
| MAB46 | GDTLTEL | FDPEEGET | VTEGLAGVPF | QSISSW | KAS (SEQ ID NO: | QQHNSYRWT |
| | | (SEQ ID NO: | DL (SEQ ID | (SEQ ID NO: | | (SEQ ID NO: |
| | S (SEQ ID | 145) | NO: 146) | 147) | 148) | 95) |
| | NO: 144) | | | | | |
| MAB47 | GDTLTEL | FDPEEGET | VTEGEAGRPF | QSISSW | KAS (SEQ ID NO: | VQYPSYSWT |
| | S (SEQ ID | (SEQ ID NO: | DA (SEQ ID | (SEQ ID NO: | 148) | (SEQ ID NO: |
| | NO: 144) | 145) | NO: 153) | 147) | | 97) |
| MAB48 | GDTLTEL | FDPEEGET | VTEGLAGRPY | QSISSW | KAS (SEQ ID NO: | QQYNSYKLT |
| | S (SEQ ID | (SEQ ID NO: | DV (SEQ ID | (SEQ ID NO: | 148) | (SEQ ID NO: |
| | NO: 144) | 145) | NO: 154) | 147) | | 99) |
| MAB49 | GDTLTEL | FDPEEGET | VTEGLAGIPF | QSISSW | KAS (SEQ ID NO: | QQYNSPSWL |
| | S (SEQ ID | (SEQ ID NO: | DSW (SEQ ID | (SEQ ID NO: | 148) | (SEQ ID NO: |
| | NO: 144) | 145) | NO: 155) | 147) | | 101) |
| MAB50 | GDTLTEL | FDPEEGET | VTEGLAGIPF | QSISSW | KAS (SEQ ID NO: | QQVNSLSWT |
| | S (SEQ ID | (SEQ ID NO: | DSW (SEQ ID | (SEQ ID NO: | 148) | (SEQ ID NO: |
| | NO: 144) | 145) | NO: 155) | 147) | | 102) |
| MAB51 | GFTFDDY | INWEGGST | ARDEQLGGTT | QGIRND | TAS (SEQ ID NO: 160) | FQYNSYPLG |
| | G (SEQ ID | (SEQ ID NO | YYYYYMDV | (SEQ ID NO: | | (SEQ ID NO: |
| | NO: 156) | 157) | (SEQ ID NO: | 159) | | 108) |
| | | | 158) | | | |
| MAB52 | GFTFDDY | INWEGGST | ARDEQLGGK | QGIRND | TAS (SEQ ID NO: 160) | LQYRSHPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 110) |
| | | | 161) | | | |
| MAB53 | GFTFDDY | INWEGGST | ARDEQLGGQ | QGIRND | TAS (SEQ ID NO: 160) | LQYNQVPLT |
| | G (SEQ ID | (SEQ ID NO: | TYYYYYMDV | (SEQ ID NO: | | (SEQ ID NO: |
| | NO: 156) | 157) | (SEQ ID NO: | 159) | | 112) |
| | | | 162) | | | |
| MAB54 | GFTFDDY | INWEGGST | ARDEQLGGTT | QGIRND | TAS (SEQ ID NO: 160) | LQTNIYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 113) |
| | | | 158) | | | |
| MAB55 | GFTFDDY | INWEGGST | ARDEQLGGIK | QGIRND | TAS (SEQ ID NO: 160) | LQYKQYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 115) |
| | | | 163) | | | |
| MAB56 | GFTFDDY | INWEGGST | ARDEQLGGL | QGIRND | TAS (SEQ ID NO: 160) | LQTNIYPLT |
| | G (SEQ ID | (SEQ ID NO: | KYYYYYMDV | (SEQ ID NO: | | (SEQ ID NO: |
| | NO: 156) | 157) | (SEQ ID NO: | 159) | | 113) |
| | | | 164) | | | |
| MAB57 | GFTFDDY | INWEGGST | ARDEQLGGK | QGIRND | TAS (SEQ ID NO: 160) | LQYNSYPLA |
| | G (SEQ ID | (SEQ ID NO: | VYYYYYMDV | (SEQ ID NO: | | (SEQ ID NO: |
| | NO: 156) | 157) | (SEQ ID NO: | 159) | | 118) |
| | | | 165) | | | |
| MAB58 | GFTFDDY | INWEGGST | ARDEQLGGL | QGIRND | TAS (SEQ ID NO: 160) | LQYHSYPLR |
| | G (SEQ ID | (SEQ ID NO: | KYYYYYMDV | (SEQ ID NO: | | (SEQ ID NO: |
| | NO: 156) | 157) | (SEQ ID NO: | 159) | | 119) |
| | | | 164) | | | |
| MAB59 | GFTFDDY | INWEGGST | ARDEQLGGN | QGIRND | TAS (SEQ ID NO: 160) | LQYASYPLK |
| | G (SEQ ID | (SEQ ID NO: | HYYYYYMDA | (SEQ ID NO: | | (SEQ ID NO: |
| | NO: 156) | 157) | (SEQ ID NO: | 159) | | 121) |
| | | | 166) | | | |
| MAB60 | GFTFDDY | INWEGGST | ARDEQLGGR | QGIRND | TAS (SEQ ID NO: 160) | LQYNAYPLI |
| | G (SEQ ID | (SEQ ID NO: | HYYYYYMDV | (SEQ ID NO: | | (SEQ ID NO: |
| | NO: 156) | 157) | (SEQ ID NO | 159) | | 123) |
| | | | 167) | | | |

In some embodiments, the antibody comprises a variable heavy chain comprising a HCDR1 of SEQ ID NO: 128, a HCDR2 of SEQ ID NO: 129, and a HCDR3 of DEQLGGX₁X₂YYYYYMDX₃ (SEQ ID NO: 333), wherein:
X₁ is N, T, I, Q, L, K, or R;
X₂ is Y, T, K, H, or V; and
X₃ is V or A.

In some embodiments, X₁ is N, T, I, Q, L, K, or R, X₂ is Y, and X₃ is V. In some embodiments, X₁ is N, T, I, Q, L, K, or R, X₂ is Y, and X₃ is A. In some embodiments, X₁ is N, T, I, Q, L, K, or R, X₂ is T, and X₃ is V. In some embodiments, X₁ is N, T, I, Q, L, K, or R, X₂ is T, and X₃ is A. In some embodiments, X₁ is N, T, I, Q, L, K, or R, X₂ is K, and X₃ is V. In some embodiments, X₁ is N, T, I, Q, L, K, or R, X₂ is K, and X₃ is A.

In some embodiments, X₁ is N, T, I, Q, L, K, or R, X₂ is H, and X₃ is V. In some embodiments, X₁ is N, T, I, Q, L, K, or R, X₂ is H, and X₃ is A. In some embodiments, X₁ is N, T, I, Q, L, K, or R, X₂ is V, and X₃ is V. In some embodiments, X₁ is N, T, I, Q, L, K, or R, X₂ is V, and X₃ is A. In some embodiments, X₁ is N, X₂ is Y, and X₃ is A. In some embodiments, X₁ is N, T, I, Q, L, K, or R, X₂ is T, and X₃ is V. In some embodiments, X₁ is N, T, I, Q, L, K, or R, X₂ is T, and X₃ is A. In some embodiments, X₁ is N, T, I, Q, L, K, or R, X₂ is K, and X₃ is V. In some embodiments, X₁ is N, T, I, Q, L, K, or R, X₂ is K, and X₃ is A.

In some embodiments, X₁ is N, T, I, Q, L, K, or R, X₂ is H, and X₃ is V. In some embodiments, X₁ is N, T, I, Q, L, K, or R, X₂ is H, and X₃ is A. In some embodiments, X₁ is N, T, I, Q, L, K, or R, X₂ is V, and X₃ is V. In some embodiments, X₁ is N, T, I, Q, L, K, or R, X₂ is V, and X₃ is A. In some embodiments, X₁ is not N. In some embodiments, X₁ is not T. In some embodiments, X₁ is not I. In some embodiments, X₁ is not Q. In some embodiments, X₁ is not L. In some embodiments, X₁ is not K. In some embodiments, X₁ is not R.

In some embodiments, an antibody is provided comprising a variable light chain LCDR1 of SEQ ID NO: 106, a LCDR2 of SEQ ID NO: 107, and a LCDR3 comprising an amino acid sequence of:
X₄QX₅X₆X₇X₈PLX₉ (SEQ ID NO: 332), wherein:
X₄ is L or F;
X₅ is Y or T;
X₆ is N, K, A, H, or R;
X₇ is S, Q, I, or A;
X₈ is Y, V, or H; and
X₉ is T, G, K, R, I or A.

In some embodiments, X₄ is L; X₅ is Y or T; X₆ is N, K, A, H, or R; X₇ is S, Q, I, or A; X₈ is Y, V, or H; and X₉ is T, G, K, R, I or A. In some embodiments, X₄ is F; X₅ is Y or T; X₆ is N, K, A, H, or R; X₇ is S, Q, I, or A; X₈ is Y, V, or H; and X₉ is T, G, K, R, I or A. In some embodiments, X₄ is L or F; X₅ is Y; X₆ is N, K, A, H, or R; X₇ is S, Q, I, or A; X₈ is Y, V, or H; and X₉ is T, G, K, R, I or A. In some embodiments, X₄ is L or F; X₅ is T; X₆ is N, K, A, H, or R; X₇ is S, Q, I, or A; X₈ is Y, V, or H; and X₉ is T, G, K, R, I or A. In some embodiments, X₄ is L or F; X₅ is Y or T; X₆ is N; X₇ is S, Q, I, or A; X₈ is Y, V, or H; and X₉ is T, G, K, R, I or A. In some embodiments, X₄ is L or F; X₅ is Y or T; X₆ is K; X₇ is S, Q, I, or A; X₈ is Y, V, or H; and X₉ is T, G, K, R, I or A. In some embodiments, X₄ is L or F; X₅ is Y or T; X₆ is A; X₇ is S, Q, I, or A; X₈ is Y, V, or H; and X₉ is T, G, K, R, I or A. In some embodiments, X₄ is L or F; X₅ is Y or T; X₆ is H; X₇ is S, Q, I, or A; X₈ is Y, V, or H; and X₉ is T, G, K, R, I or A. In some embodiments, X₄ is L or F; X₅ is Y or T; X₆ is R; X₇ is S, Q, I, or A; X₈ is Y, V, or H; and X₉ is T, G, K, R, I or A. In some embodiments, X₄ is L or F; X₅ is Y or T; X₆ is N, K, A, H, or R; X₇ is S; X₈ is Y, V, or H; and X₉ is T, G, K, R, I or A. In some embodiments, X₄ is L or F; X₅ is Y or T; X₆ is N, K, A, H, or R; X₇ is Q; X₈ is Y, V, or H; and X₉ is T, G, K, R, I or A. In some embodiments, X₄ is L or F; X₅ is Y or T; X₆ is N, K, A, H, or R; X₇ is I; X₈ is Y, V, or H; and X₉ is T, G, K, R, I or A. In some embodiments, X₄ is L or F; X₅ is Y or T; X₆ is N, K, A, H, or R; X₇ is A; X₈ is Y, V, or H; and X₉ is T, G, K, R, I or A. In some embodiments, X₄ is L or F; X₅ is Y or T; X₆ is N, K, A, H, or R; X₇ is S, Q, I, or A; X₈ is Y; and X₉ is T, G, K, R, I or A. In some embodiments, X₄ is L or F; X₅ is Y or T; X₆ is N, K, A, H, or R; X₇ is S, Q, I, or A; X₈ is V; and X₉ is T, G, K, R, I or A. In some embodiments, X₄ is L or F; X₅ is Y or T; X₆ is N, K, A, H, or R; X₇ is S, Q, I, or A; X₈ is H; and X₉ is T, G, K, R, I or A. In some embodiments, X₄ is L or F; X₅ is Y or T; X₆ is N, K, A, H, or R; X₇ is S, Q, I, or A; X₈ is Y, V, or H; and X₉ is T. In some embodiments, X₄ is L or F; X₅ is Y or T; X₆ is N, K, A, H, or R; X₇ is S, Q, I, or A; X₈ is Y, V, or H; and X₉ is G. In some embodiments, X₄ is L or F; X₅ is Y or T; X₆ is N, K, A, H, or R; X₇ is S, Q, I, or A; X₈ is Y, V, or H; and X₉ is K. In some embodiments, X₄ is L or F; X₅ is Y or T; X₆ is N, K, A, H, or R; X₇ is S, Q, I, or A; X₈ is Y, V, or H; and X₉ is R. In some embodiments, X₄ is L or F; X₅ is Y or T; X₆ is N, K, A, H, or R; X₇ is S, Q, I, or A; X₈ is Y, V, or H; and X₉ is I. In some embodiments, X₄ is L or F; X₅ is Y or T; X₆ is N, K, A, H, or R; X₇ is S, Q, I, or A; X₈ is Y, V, or H; and X₉ is A.

In some embodiments, an antibody is provided comprising a variable heavy chain comprising a variable heavy chain comprising a HCDR1 of SEQ ID NO: 128, a HCDR2 of SEQ ID NO: 129, and a HCDR3 of DEQLGGX₁X₂YYYYYMDX₃ (SEQ ID NO: 333), wherein X₁ is N, T, I, Q, L, K, or R; X₂ is Y, T, K, H, or V; and X₃ is V or A; and a variable light chain LCDR1 of SEQ ID NO: 106, a LCDR2 of SEQ ID NO: 107, and a LCDR3 comprising an amino acid sequence of X₄QX₅X₆X₇X₈PLX₉ (SEQ ID NO: 332), wherein X₄ is L or F; X₅ is Y or T; X₆ is N, K, A, H, or R; X₇ is S, Q, I, or A; X₈ is Y, V, or H; and X₉ is T, G, K, R, I or A. In some embodiments, the variables of the HCDR3 and the LCDR3 are as set forth above.

In some embodiments, the antibody comprises a variable light chain and a heavy chain as set forth for MAB21, MAB22, MAB23, MAB24, MAB25, MAB26, MAB27, MAB28, MAB29, MAB30, MAB51, MAB52, MAB53, MAB54, MAB55, MAB56, MAB57, MAB58, MAB59, or MAB60, provided that the HCDR3 comprises an amino acid sequence of SEQ ID NO: 333 and the LCDR3 comprises as an amino acid sequence of SEQ ID NO: 332 as set forth herein. In some embodiments, the antibody comprises a variable light chain and a heavy chain that are each, independently, at least 85%, 86%, 87%, 88%, 89%, 90%, 91%, 92%, 93%, 94%, 95%, 96%, 97%, 98% or 99% as set forth for the VL and VH of MAB21, MAB22, MAB23, MAB24, MAB25, MAB26, MAB27, MAB28, MAB29, MAB30, MAB51, MAB52, MAB53, MAB54, MAB55, MAB56, MAB57, MAB58, MAB59, or MAB60, provided that the HCDR3 comprises an amino acid sequence of SEQ ID NO: 333 and the LCDR3 comprises as an amino acid sequence of SEQ ID NO: 332 as set forth herein.

In some embodiments, the antibody does not comprise a HCDR3 of EQLGGNYYYYYYMDV and/or a LCDR3 of LQYNSYPLT. In some embodiments, when an antibody comprises a HCDR3 of EQLGGNYYYYYYMDV, the antibody does not comprise a LCDR3 of LQYNSYPLT.

In some embodiments, the antibody comprises a variable heavy chain comprising a HCDR1 of SEQ ID NO: 61, a HCDR2 of SEQ ID NO: 62, and a HCDR3 of DX₁₀X₁₁X₁₂YX₁₃X₁₄DX₁₅ (SEQ ID NO: 329), wherein: X₁₀ is E, Q, or N; X₁₁ is T, E, S, or N; X₁₂ is D or Q; X₁₃ is A or G; X₁₄ is L, W, F, or Y; and X₁₅ is Y, E, L, N, F, or W. In some embodiments, X₁₀ is E. In some embodiments, X₁₀ is Q. In some embodiments, X₁₀ is N. In some embodiments, X₁₁ is T. In some embodiments, X₁₁ is E. In some embodiments, X₁₁ is S. In some embodiments, X₁₁ is N. In some embodiments, X₁₂ is D. In some embodiments, X₁₂ is D. In some embodiments, X₁₃ is A. In some embodiments, X₁₃ is G. In some embodiments, X₁₄ is L. In some embodiments, X₁₄ is W. In some embodiments, X₁₄ is F. In some embodiments, X₁₄ is or Y. In some embodiments, X₁₅ is Y. In some embodiments, X₁₅ is E. In some embodiments, X₁₅ is L. In some embodiments, X₁₅ is N. In some embodiments, X₁₅ is F. In some embodiments, X₁₅ is W.

In some embodiments, the antibody comprises a variable light chain comprising a LCDR1 of SEQ ID NO: 64, a LCDR2 of SEQ ID NO: 65, and a LCDR3 comprising an amino acid sequence of X₁₆X₁₇X₁₈EDX₁₉X₂₀X₂₁X₂₂ (SEQ ID NO: 328), wherein X₁₆ is Q, E, K, or H; X₁₇ is Q or H; X₁₈ is Y or H, X₁₉ is L or Y; X₂₀ is P or I; X₂₁ is L or P, and X₂₂ is T or V. In some embodiments, X₁₆ is Q. In some embodiments, X₁₆ is E. In some embodiments, X₁₆ is K. In some embodiments, X₁₆ is H. In some embodiments, X₁₇ is Q. In some embodiments, X₁₇ is H. In some embodiments, X₁₈ is Y. In some embodiments, X₁₈ is H. In some embodiments, X₁₉ is L. In some embodiments, X₁₉ is Y. In some embodiments, X₂₀ is P. In some embodiments, X₂₀ is I. In some embodiments, X₂₁ is L. In some embodiments, X₂₁ is P. In some embodiments, X₂₂ is T. In some embodiments, X₂₂ is V.

In some embodiments, an antibody is provided comprising a variable heavy chain comprising a variable heavy chain comprising a HCDR1 of SEQ ID NO: 61, a HCDR2 of SEQ ID NO: 62, and a HCDR3 of SEQ ID NO: 329 as set forth herein and a variable light chain LCDR1 of SEQ ID NO: 63, a LCDR2 of SEQ ID NO: 64, and a LCDR3 comprising an amino acid sequence of SEQ ID NO: 328 as set forth herein.

In some embodiments, the antibody comprises a variable light chain and a heavy chain as set forth for MAB1, MAB2, MAB3, MAB4, MAB5, MAB6, MAB7, MAB8, MAB9, MAB10, MAB31, MAB32, MAB33, MAB34, MAB35, MAB36, MAB38, MAB39, or MAB40, provided that the HCDR3 comprises an amino acid sequence of SEQ ID NO: 329 and the LCDR3 comprises as an amino acid sequence of SEQ ID NO: 328 as set forth herein. In some embodiments, the antibody comprises a variable light chain and a heavy chain that are each, independently, at least 85%, 86%, 87%, 88%, 89%, 90%, 91%, 92%, 93%, 94%, 95%, 96%, 97%, 98% or 99% as set forth for the VL and VH of MAB1, MAB2, MAB3, MAB4, MAB5, MAB6, MAB7, MAB8, MAB9, MAB10, MAB31, MAB32, MAB33, MAB34, MAB35, MAB36, MAB38, MAB39, or MAB40, provided that the HCDR3 comprises an amino acid sequence of SEQ ID NO: 329 and the LCDR3 comprises as an amino acid sequence of SEQ ID NO: 328 as set forth herein.

In some embodiments, a VH and VL is provided, provided that when the HCDR3 is DETDYALDY, the LCDR3 is not QQYEDLPLT. In some embodiments, SEQ ID NO: 329 is not DETDYALDY. In some embodiments, SEQ ID NO: 328 is not QQYEDLPLT.

In some embodiments, the antibody comprises a variable heavy chain comprising a HCDR1 of SEQ ID NO: 82, a HCDR2 of SEQ ID NO: 83, and a HCDR3 of X₂₃GX₂₄X₂₅X₂₆X₂₇PX₂₈X₂₉X₃₀ (SEQ ID NO: 330), wherein: X23 is E or K, X24 is L or E; X25 is A or G, X26 is G or W, X27 is R, V, L, I, or F, X28 is F, Y, or T, X29 is D or Y, and X30 is A, V, L, S, H, or I. In some embodiments, X23 is E. In some embodiments X24 is L. In some embodiments, X25 is A. In some embodiments X26 is G. In some embodiments X27 is R. In some embodiments, X27 is V. In some embodiments, X27 is L. In some embodiments, X27 is I. In some embodiments, X27 is F. In some embodiments, X28 is F. In some embodiments, X28 is Y. In some embodiments, X29 is D. In some embodiments, X30 is S or L.

In some embodiments, the antibody comprises a variable light chain comprising a LCDR1 of SEQ ID NO: 85, a LCDR2 of SEQ ID NO: 86, and a LCDR3 of X₃₁QX₃₂X₃₃SX₃₄X₃₅X₃₆X₃₇ (SEQ ID NO: 331), wherein X31 is Q or V; X32 is Y, A, V, or H; X33 is N, P, S, or R; X34 is Y, L, or P; X35 is S, K, V, A, D, or R; X36 is W or L; and X37 is T or L. In some embodiments, X31 is Q. In some embodiments, X31 is V. In some embodiments, X32 is Y. In some embodiments, X32 is A. In some embodiments, X32 is V. In some embodiments, X32 is H. In some embodiments, X33 is N. In some embodiments, X33 is P. In some embodiments, X33 is S. In some embodiments, X33 is R. In some embodiments, X34 is Y. In some embodiments, X34 is L. In some embodiments, X34 is P. In some embodiments, X35 is S. In some embodiments, X35 is K. In some embodiments, X35 is V. In some embodiments, X35 is A. In some embodiments, X35 is D. In some embodiments, X35 is R. In some embodiments, X36 is W. In some embodiments, X36 is L. In some embodiments, X37 is T. In some embodiments, X37 is L.

In some embodiments, an antibody is provided comprising a variable heavy chain comprising a variable heavy chain comprising a HCDR1 of SEQ ID NO: 82, a HCDR2 of SEQ ID NO: 83, and a HCDR3 of SEQ ID NO: 330 as set forth herein and a variable light chain comprising a LCDR1 of SEQ ID NO: 84, a LCDR2 of SEQ ID NO: 85, and a LCDR3 comprising an amino acid sequence of SEQ ID NO: 331 as set forth herein.

In some embodiments, the antibody comprises a variable light chain and a heavy chain as set forth for MAB11, MAB12, MAB13, MAB14, MAB15, MAB16, MAB17, MAB18, MAB19, MAB20, MAB41, MAB42, MAB43, MAB44, MAB45, MAB46, MAB47, MAB48, MAB49, or MAB50, provided that the HCDR3 comprises an amino acid sequence of SEQ ID NO: 330 and the LCDR3 comprises as an amino acid sequence of SEQ ID NO: 331 as set forth herein. In some embodiments, the antibody comprises a variable light chain and a heavy chain that are each, independently, at least 85%, 86%, 87%, 88%, 89%, 90%, 91%, 92%, 93%, 94%, 95%, 96%, 97%, 98% or 99% as set forth for the VL and VH of MAB11, MAB12, MAB13, MAB14, MAB15, MAB16, MAB17, MAB18, MAB19, MAB20, MAB41, MAB42, MAB43, MAB44, MAB45, MAB46, MAB47, MAB48, MAB49, or MAB50, provided that the HCDR3 comprises an amino acid sequence of SEQ ID NO: 330 and the LCDR3 comprises as an amino acid sequence of SEQ ID NO: 331 as set forth herein.

In some embodiments, a VH and VL is provided, provided that when the HCDR3 is EGLAGRPFDS, the LCDR3 is not QQYNSYSWTY. In some embodiments, SEQ ID NO: 330 is not EGLAGRPFDS. In some embodiments, SEQ ID NO: 331 is not QQYNSYSWTY.

In some embodiments, an antibody, or antibody binding fragment thereof, comprises a heavy or light chain CDR as provided in the tables above. In some embodiments, an antibody, or antibody binding fragment thereof, comprises a light chain LCDR having a sequence of SEQ ID NO: 64, 65, 66, 68, 71, 75, 77, 79, 81, 85, 86, 87, 89, 92, 94, 95, 97, 99, 101, 102, 106, 107, 108, 110, 112, 113, 115, 118, 119, 121, 123, 133, 134, 147, 148, 159, 160, or a combination thereof. In some embodiments, an antibody, or antibody binding fragment thereof, comprises a heavy chain HCDR having a sequence of SEQ ID NO: 61, 62, 63, 67, 69, 70, 72, 73, 74, 76, 78, 80, 82, 83, 84, 88, 90, 91, 93, 96, 98, 100, 103, 104, 105, 109, 111, 114, 116, 117, 120, 122, 124, 125, 126, 127, 128, 129, 130, 131, 132, 135, 136, 137, 138, 139, 140, 141, 142, 143, 144, 145, 146, 149, 150, 151, 152, 153, 154, 155, 156, 157, 158, 161, 162, 163, 164, 165, 166, 167, or a combination thereof.

In some embodiments, the heavy chain HCDR comprises a sequence that is a combination of one or more of SEQ ID NO: 61, 62, 63, 67, 69, 70, 72, 73, 74, 76, 78, 80, 82, 83, 84, 88, 90, 91, 93, 96, 98, 100, 103, 104, 105, 109, 111, 114, 116, 117, 120,122, 124, 125, 126, 127, 128, 129, 130, 131, 132, 135, 136, 137, 138, 139, 140, 141, 142, 143, 144, 145, 146, 149, 150, 151, 152, 153, 154, 155, 156, 157, 158, 161, 162, 163, 164, 165, 166, and 167.

It is to be understood that the CDRs referenced in the embodiments throughout the present specification can be interchanged with the CDRs that are characterized by different formats, such as Chothia and IMGT, which are illustrated in the tables above.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 66.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 68.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 71.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 75.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 77.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 79.

In some embodiments, an antibody, or antibody binding fragment thereof, comprises a light chain variable region having a LCDR1, a LCDR2, and a LCDR3, wherein the LCDR1 has a sequence of SEQ ID NO: 64, the LCDR2 has a sequence of SEQ ID NO: 65, and the LCDR3 has a sequence of SEQ ID NO: 81.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 87.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 89.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 92.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 94.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 95.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 97.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 99.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 101.

In some embodiments, an antibody, or antibody binding fragment thereof, comprises a light chain variable region having a LCDR1, a LCDR2, and a LCDR3, wherein the LCDR1 has a sequence of SEQ ID NO: 85, the LCDR2 has a sequence of SEQ ID NO: 86, and the LCDR3 has a sequence of SEQ ID NO: 102.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 108.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 110.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 112.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 113.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 115.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 118.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 119.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 121.

In some embodiments, an antibody, or antibody binding fragment thereof, comprises a light chain variable region having a LCDR1, a LCDR2, and a LCDR3, wherein the LCDR1 has a sequence of SEQ ID NO: 106, the LCDR2 has a sequence of SEQ ID NO: 107, and the LCDR3 has a sequence of SEQ ID NO: 123.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 66.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 68.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 71.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 75.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 77.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 79.

In some embodiments, an antibody, or antibody binding fragment thereof, comprises a light chain variable region having a LCDR1, a LCDR2, and a LCDR3, wherein the LCDR1 has a sequence of SEQ ID NO: 133, the LCDR2 has a sequence of SEQ ID NO: 134, and the LCDR3 has a sequence of SEQ ID NO: 81.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 87.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 89.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 92.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 94.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 95.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 97.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 99.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 101.

In some embodiments, an antibody, or antibody binding fragment thereof, comprises a light chain variable region having a LCDR1, a LCDR2, and a LCDR3, wherein the LCDR1 has a sequence of SEQ ID NO: 147, the LCDR2 has a sequence of SEQ ID NO: 148, and the LCDR3 has a sequence of SEQ ID NO: 102.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 108.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 110.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 112.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 113.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 115.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 118.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 119.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 112.

In some embodiments, an antibody, or antibody binding fragment thereof, comprises a light chain variable region having a LCDR1, a LCDR2, and a LCDR3, wherein the LCDR1 has a sequence of SEQ ID NO: 159, the LCDR2 has a sequence of SEQ ID NO: 160, and the LCDR3 has a sequence of SEQ ID NO: 123.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 63.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 67.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 69.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 70.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 72.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 73.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 74.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 76.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 78.

In some embodiments, an antibody, or antibody binding fragment thereof, comprises a heavy chain variable region having a HCDR1, a HCDR2, and a HCDR3, wherein the HCDR1 has a sequence of SEQ ID NO: 61, the HCDR2 has a sequence of SEQ ID NO: 62, and the HCDR3 has a sequence of SEQ ID NO: 80.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 84.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 88.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 90.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 91.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 93.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 96.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 98.

In some embodiments, an antibody, or antibody binding fragment thereof, comprises a heavy chain variable region having a HCDR1, a HCDR2, and a HCDR3, wherein the HCDR1 has a sequence of SEQ ID NO: 82, the HCDR2 has a sequence of SEQ ID NO: 83, and the HCDR3 has a sequence of SEQ ID NO: 100.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 105.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 109.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 111.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 114.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 116.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 117.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 120.

In some embodiments, an antibody, or antibody binding fragment thereof, comprises a heavy chain variable region having a HCDR1, a HCDR2, and a HCDR3, wherein the HCDR1 has a sequence of SEQ ID NO: 103, the HCDR2 has a sequence of SEQ ID NO: 104, and the HCDR3 has a sequence of SEQ ID NO: 122.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 63.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 67.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 69.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 70.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 72.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 73.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 74.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 76.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 78.

In some embodiments, an antibody, or antibody binding fragment thereof, comprises a heavy chain variable region having a HCDR1, a HCDR2, and a HCDR3, wherein the HCDR1 has a sequence of SEQ ID NO: 124, the HCDR2 has a sequence of SEQ ID NO: 125, and the HCDR3 has a sequence of SEQ ID NO: 80.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 84.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 88.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 90.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 91.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 93.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 96.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 98.

In some embodiments, an antibody, or antibody binding fragment thereof, comprises a heavy chain variable region having a HCDR1, a HCDR2, and a HCDR3, wherein the HCDR1 has a sequence of SEQ ID NO: 126, the HCDR2 has a sequence of SEQ ID NO: 127, and the HCDR3 has a sequence of SEQ ID NO: 100.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 105.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 109.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 111.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 114.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 116.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 117.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 120.

In some embodiments, an antibody, or antibody binding fragment thereof, comprises a heavy chain variable region having a HCDR1, a HCDR2, and a HCDR3, wherein the HCDR1 has a sequence of SEQ ID NO: 128, the HCDR2 has a sequence of SEQ ID NO: 129, and the HCDR3 has a sequence of SEQ ID NO: 122.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 132.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 135.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 136.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 137.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 138.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 139.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 140.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 141.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 142.

In some embodiments, an antibody, or antibody binding fragment thereof, comprises a heavy chain variable region having a HCDR1, a HCDR2, and a HCDR3, wherein the HCDR1 has a sequence of SEQ ID NO: 130, the HCDR2 has a sequence of SEQ ID NO: 131, and the HCDR3 has a sequence of SEQ ID NO: 143.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 146.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 149.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 150.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 151.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 152.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 153.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 154.

In some embodiments, an antibody, or antibody binding fragment thereof, comprises a heavy chain variable region having a HCDR1, a HCDR2, and a HCDR3, wherein the HCDR1 has a sequence of SEQ ID NO: 144, the HCDR2 has a sequence of SEQ ID NO: 145, and the HCDR3 has a sequence of SEQ ID NO: 155.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 158.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 161.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 162.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 163.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 164.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 165.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 166.

In some embodiments, an antibody, or antibody binding fragment thereof, comprises a heavy chain variable region having a HCDR1, a HCDR2, and a HCDR3, wherein the HCDR1 has a sequence of SEQ ID NO: 156, the HCDR2 has a sequence of SEQ ID NO: 157, and the HCDR3 has a sequence of SEQ ID NO: 167.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 66 and (ii) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62 and the heavy chain HCDR3 sequence has the amino acid sequence of SEQ ID NO: 63; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 68 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 67; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 68 and (ii) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62 and the heavy chain HCDR3 sequence has the amino acid sequence of SEQ ID NO: 69; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 70; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 71 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 72; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 71 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 73; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 75 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 74; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 77 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 76; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 79 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 78; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 81 and (ii) a heavy chain variable region comprising heavy chain HCDR1, HCDR2, and HCDR3 sequences, wherein the HCDR1 sequence has the amino acid sequence of SEQ ID NO: 61; the HCDR2 has the amino acid sequence of SEQ ID NO: 62 and the HCDR3 sequence has the amino acid sequence of SEQ ID NO: 80; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 87 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 84; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 89 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 88; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 89 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 90; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 92 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 91; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 94 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 93; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 95 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 84; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 97 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 96; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 99 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 98; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 101 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 100; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 85; the light chain LCDR2 sequence has the amino acid sequence of SEQ ID NO: 86; and the light chain LCDR3 sequence has the amino acid sequence of SEQ ID NO: 102 and (ii) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83 and the heavy chain HCDR3 sequence has the amino acid sequence of SEQ ID NO: 100; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 108 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 105; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 110 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 109; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 112 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 111; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 113 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 105; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 115 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 114; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 113 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 116; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 118 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 117; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 119 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 116; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 121 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 120; or variants or convention equivalents of any of the foregoing.

In some embodiments, an antibody, or antigen binding fragment thereof, comprises: (i) a light chain variable region comprising light chain LCDR1, LCDR2, and LCDR3 sequences, wherein the light chain LCDR1 sequence has the amino acid sequence SEQ ID NO: 106; the light chain LCDR2 sequence has the amino acid sequence of SEQ ID NO: 107; and the light chain LCDR3 sequence has the amino acid sequence of SEQ ID NO: 123 and (ii) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104 and the heavy chain HCDR3 sequence has the amino acid sequence of SEQ ID NO: 122; or variants or convention equivalents of any of the foregoing.

Although the preceding paragraphs may make reference to CDRs under the Kabat system the equivalent CDR sequences can be used from the IMGT and CHOTHIA designations.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 63, respectively, from SEQ ID NO: 124, SEQ ID NO: 125, and SEQ ID NO: 63, respectively, or from SEQ ID NO: 130, SEQ ID NO: 131, and SEQ ID NO: 132, respectively, and the LCDR1, LCDR2, and LCDR3 are selected from SEQ ID NO: 64, SEQ ID NO: 65, and SEQ ID NO: 66, respectively, from SEQ ID NO: 64, SEQ ID NO: 65, and SEQ ID NO: 66, respectively, or from SEQ ID NO: 133, SEQ ID NO: 134, and SEQ ID NO: 66 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 67, respectively, from SEQ ID NO: 124, SEQ ID NO: 125, and SEQ ID NO: 67, respectively, or from SEQ ID NO: 130, SEQ ID NO: 131, and SEQ ID NO: 135, respectively, and the LCDR1, LCDR2, and LCDR3 are selected from SEQ ID NO: 64, SEQ ID NO: 65, and SEQ ID NO: 68, respectively, from SEQ ID NO: 64, SEQ ID NO: 65, and SEQ ID NO: 68, respectively, or from SEQ ID NO: 133, SEQ ID NO: 134, and SEQ ID NO: 68 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 69, respectively, from SEQ ID NO: 124, SEQ ID NO: 125, and SEQ ID NO: 69, respectively, or from SEQ ID NO: 130, SEQ ID NO: 131, and SEQ ID NO: 136, respectively, and the LCDR1, LCDR2, and LCDR3 are selected from SEQ ID NO: 64, SEQ ID NO: 65, and SEQ ID NO: 68, respectively, from SEQ ID NO: 64, SEQ ID NO: 65, and SEQ ID NO: 68, respectively, or from SEQ ID NO: 133, SEQ ID NO: 134, and SEQ ID NO: 68 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 70, respectively, from SEQ ID NO: 124, SEQ ID NO: 125, and SEQ ID NO: 70, respectively, or from SEQ ID NO: 130, SEQ ID NO: 131, and SEQ ID NO: 137, respectively, and the LCDR1, LCDR2, and LCDR3 are selected from SEQ ID NO: 64, SEQ ID NO: 65, and SEQ ID NO: 71, respectively, from SEQ ID NO: 64, SEQ ID NO: 65, and SEQ ID NO: 71, respectively, or from SEQ ID NO: 133, SEQ ID NO: 134, and SEQ ID NO: 71 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 72, respectively, from SEQ ID NO: 124, SEQ ID NO: 125, and SEQ ID NO: 72, respectively, or from SEQ ID NO: 130, SEQ ID NO: 131, and SEQ ID NO: 138, respectively, and the LCDR1, LCDR2, and LCDR3 are selected from SEQ ID NO: 64, SEQ ID NO: 65, and SEQ ID NO: 71, respectively, from SEQ ID NO: 64, SEQ ID NO: 65, and SEQ ID NO: 71, respectively, or from SEQ ID NO: 133, SEQ ID NO: 134, and SEQ ID NO: 71 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 73, respectively, from SEQ ID NO: 124, SEQ ID NO: 125, and SEQ ID NO: 73, respectively, or from SEQ ID NO: 130, SEQ ID NO: 131, and SEQ ID NO: 139, respectively, and the LCDR1, LCDR2, and LCDR3 are selected from SEQ ID NO: 64, SEQ ID NO: 65, and SEQ ID NO: 71, respectively, from SEQ ID NO: 64, SEQ ID NO: 65, and SEQ ID NO: 71, respectively, or from SEQ ID NO: 133, SEQ ID NO: 134, and SEQ ID NO: 71 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 74, respectively, from SEQ ID NO: 124, SEQ ID NO: 125, and SEQ ID NO: 74, respectively, or from SEQ ID NO: 130, SEQ ID NO: 131, and SEQ ID NO: 140, respectively, and the LCDR1, LCDR2, and LCDR3 are selected from SEQ ID NO: 64, SEQ ID NO: 65, and SEQ ID NO: 75, respectively, from SEQ ID NO: 64, SEQ ID NO: 65, and SEQ ID NO: 75, respectively, or from SEQ ID NO: 133, SEQ ID NO: 134, and SEQ ID NO: 75 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 76, respectively, from SEQ ID NO: 124, SEQ ID NO: 125, and SEQ ID NO: 76, respectively, or from SEQ ID NO: 130, SEQ ID NO: 131, and SEQ ID NO: 141, respectively, and the LCDR1, LCDR2, and LCDR3 are selected from SEQ ID NO: 64, SEQ ID NO: 65, and SEQ ID NO: 77, respectively, from SEQ ID NO: 64, SEQ ID NO: 65, and SEQ ID NO: 77, respectively, or from SEQ ID NO: 133, SEQ ID NO: 134, and SEQ ID NO: 77 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 78, respectively, from SEQ ID NO: 124, SEQ ID NO: 125, and SEQ ID NO: 78, respectively, or from SEQ ID NO: 130, SEQ ID NO: 131, and SEQ ID NO: 142, respectively, and the LCDR1, LCDR2, and LCDR3 are selected from SEQ ID NO: 64, SEQ ID NO: 65, and SEQ ID NO: 79, respectively, from SEQ ID NO: 64, SEQ ID NO: 65, and SEQ ID NO: 79, respectively, or from SEQ ID NO: 133, SEQ ID NO: 134, and SEQ ID NO: 79 respectively.

In some embodiments, an antibody or antigen-binding fragment thereof is provided, wherein the HCDR1, HCDR2, and HCDR3 are selected from SEQ ID NO: 61, SEQ ID NO: 62, and SEQ ID NO: 80, respectively, from SEQ ID NO: 124, SEQ ID NO: 125, and SEQ ID NO: 80, respectively, or from SEQ ID NO: 130, SEQ ID NO: 131, and SEQ ID NO: 143, respectively, and the LCDR1, LCDR2, and LCDR3 are selected from SEQ ID NO: 64, SEQ ID NO: 65, and SEQ ID NO: 81, respectively, from SEQ ID NO: 64, SEQ ID NO: 65, and SEQ ID NO: 81, respectively, or from SEQ ID NO: 133, SEQ ID NO: 134, and SEQ ID NO: 81 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 84, respectively, from SEQ ID NO: 126, SEQ ID NO: 127, and SEQ ID NO: 84, respectively, or from SEQ ID NO: 144, SEQ ID NO: 145, and SEQ ID NO: 146, respectively, and the LCDR1, LCDR2, and LCDR3 are selected from SEQ ID NO: 85, SEQ ID NO: 86, and SEQ ID NO: 87, respectively, from SEQ ID NO: 85, SEQ ID NO: 86, and SEQ ID NO: 87, respectively, or from SEQ ID NO: 147, SEQ ID NO: 148, and SEQ ID NO: 87 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 88, respectively, from SEQ ID NO: 126, SEQ ID NO: 127, and SEQ ID NO: 88, respectively, or from SEQ ID NO: 144, SEQ ID NO: 145, and SEQ ID NO: 149, respectively, and the LCDR1, LCDR2, and LCDR3 are selected from SEQ ID NO: 85, SEQ ID NO: 86, and SEQ ID NO: 89, respectively, from SEQ ID NO: 85, SEQ ID NO: 86, and SEQ ID NO: 89, respectively, or from SEQ ID NO: 147, SEQ ID NO: 148, and SEQ ID NO: 89 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 90, respectively, from SEQ ID NO: 126, SEQ ID NO: 127, and SEQ ID NO: 90, respectively, or from SEQ ID NO: 144, SEQ ID NO: 145, and SEQ ID NO: 150, respectively, and the LCDR1, LCDR2, and LCDR3 are selected from SEQ ID NO: 85, SEQ ID NO: 86, and SEQ ID NO: 89, respectively, from SEQ ID NO: 85, SEQ ID NO: 86, and SEQ ID NO: 89, respectively, or from SEQ ID NO: 147, SEQ ID NO: 148, and SEQ ID NO: 89 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 91, respectively, from SEQ ID NO: 126, SEQ ID NO: 127, and SEQ ID NO: 91, respectively, or from SEQ ID NO: 144, SEQ ID NO: 145, and SEQ ID NO: 151, respectively, and the LCDR1, LCDR2, and LCDR3 are selected from SEQ ID NO: 85, SEQ ID NO: 86, and SEQ ID NO: 92, respectively, from SEQ ID NO: 85, SEQ ID NO: 86, and SEQ ID NO: 92, respectively, or from SEQ ID NO: 147, SEQ ID NO: 148, and SEQ ID NO: 92 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 93, respectively, from SEQ ID NO: 126, SEQ ID NO: 127, and SEQ ID NO: 93, respectively, or from SEQ ID NO: 144, SEQ ID NO: 145, and SEQ ID NO: 152, respectively, and the LCDR1, LCDR2, and LCDR3 are selected from SEQ ID NO: 85, SEQ ID NO: 86, and SEQ ID NO: 94, respectively, from SEQ ID NO: 85, SEQ ID NO: 86, and SEQ ID NO: 94, respectively, or from SEQ ID NO: 147, SEQ ID NO: 148, and SEQ ID NO: 94 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 84, respectively, from SEQ ID NO: 126, SEQ ID NO: 127, and SEQ ID NO: 84, respectively, or from SEQ ID NO: 144, SEQ ID NO: 145, and SEQ ID NO: 146, respectively, and the LCDR1, LCDR2, and LCDR3 are selected from SEQ ID NO: 85, SEQ ID NO: 86, and SEQ ID NO: 95, respectively, from SEQ ID NO: 85, SEQ ID NO: 86, and SEQ ID NO: 95, respectively, or from SEQ ID NO: 147, SEQ ID NO: 148, and SEQ ID NO: 95 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 96, respectively, from SEQ ID NO: 126, SEQ ID NO: 127, and SEQ ID NO: 96, respectively, or from SEQ ID NO: 144, SEQ ID NO: 145, and SEQ ID NO: 153, respectively, and the LCDR1, LCDR2, and LCDR3 are selected from SEQ ID NO: 85, SEQ ID NO: 86, and SEQ ID NO: 97, respectively, from SEQ ID NO: 85, SEQ ID NO: 86, and SEQ ID NO: 97, respectively, or from SEQ ID NO: 147, SEQ ID NO: 148, and SEQ ID NO: 97 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 98, respectively, from SEQ ID NO: 126, SEQ ID NO: 127, and SEQ ID NO: 98, respectively, or from SEQ ID NO: 144, SEQ ID NO: 145, and SEQ ID NO: 154, respectively, and the LCDR1, LCDR2, and LCDR3 are selected from SEQ ID NO: 85, SEQ ID NO: 86, and SEQ ID NO: 99, respectively, from SEQ ID NO: 85, SEQ ID NO: 86, and SEQ ID NO: 99, respectively, or from SEQ ID NO: 147, SEQ ID NO: 148, and SEQ ID NO: 99 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 100, respectively, from SEQ ID NO: 126, SEQ ID NO: 127, and SEQ ID NO: 100, respectively, or from SEQ ID NO: 144, SEQ ID NO: 145, and SEQ ID NO: 155, respectively, and the LCDR1, LCDR2, and LCDR3 are selected from SEQ ID NO: 85, SEQ ID NO: 86, and SEQ ID NO: 101, respectively, from SEQ ID NO: 85, SEQ ID NO: 86, and SEQ ID NO: 101, respectively, or from SEQ ID NO: 147, SEQ ID NO: 148, and SEQ ID NO: 101 respectively.

In some embodiments, an antibody or antigen-binding fragment thereof is provided, wherein the HCDR1, HCDR2, and HCDR3 are selected from SEQ ID NO: 82, SEQ ID NO: 83, and SEQ ID NO: 100, respectively, from SEQ ID NO: 126, SEQ ID NO: 127, and SEQ ID NO: 100, respectively, or from SEQ ID NO: 144, SEQ ID NO: 145, and SEQ ID NO: 155, respectively, and the LCDR1, LCDR2, and LCDR3 are selected from SEQ ID NO: 85, SEQ ID NO: 86, and SEQ ID NO: 102, respectively, from SEQ ID NO: 85, SEQ ID NO: 86, and SEQ ID NO: 102, respectively, or from SEQ ID NO: 147, SEQ ID NO: 148, and SEQ ID NO: 102 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 105, respectively, from SEQ ID NO: 128, SEQ ID NO: 129, and SEQ ID NO: 105, respectively, or from SEQ ID NO: 156, SEQ ID NO: 157, and SEQ ID NO: 158, respectively, and the LCDR1, LCDR2, and LCDR3 are selected from SEQ ID NO: 106, SEQ ID NO: 107, and SEQ ID NO: 108, respectively, from SEQ ID NO: 106, SEQ ID NO: 107, and SEQ ID NO: 108, respectively, or from SEQ ID NO: 159, SEQ ID NO: 160, and SEQ ID NO: 108 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 109, respectively, from SEQ ID NO: 128, SEQ ID NO: 129, and SEQ ID NO: 109, respectively, or from SEQ ID NO: 156, SEQ ID NO: 157, and SEQ ID NO: 161, respectively, and the LCDR1, LCDR2, and LCDR3 are selected from SEQ ID NO: 106, SEQ ID NO: 107, and SEQ ID NO: 110, respectively, from SEQ ID NO: 106, SEQ ID NO: 107, and SEQ ID NO: 110, respectively, or from SEQ ID NO: 159, SEQ ID NO: 160, and SEQ ID NO: 110 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 111, respectively, from SEQ ID NO: 128, SEQ ID NO: 129, and SEQ ID NO: 111, respectively, or from SEQ ID NO: 156, SEQ ID NO: 157, and SEQ ID NO: 162, respectively, and the LCDR1, LCDR2, and LCDR3 are selected from SEQ ID NO: 106, SEQ ID NO: 107, and SEQ ID NO: 112, respectively, from SEQ ID NO: 106, SEQ ID NO: 107, and SEQ ID NO: 112, respectively, or from SEQ ID NO: 159, SEQ ID NO: 160, and SEQ ID NO: 112 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 105, respectively, from SEQ ID NO: 128, SEQ ID NO: 129, and SEQ ID NO: 105, respectively, or from SEQ ID NO: 156, SEQ ID NO: 157, and SEQ ID NO: 158, respectively, and the LCDR1, LCDR2, and LCDR3 are selected from SEQ ID NO: 106, SEQ ID NO: 107, and SEQ ID NO: 113, respectively, from SEQ ID NO: 106, SEQ ID NO: 107, and SEQ ID NO: 113, respectively, or from SEQ ID NO: 159, SEQ ID NO: 160, and SEQ ID NO: 113 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 114, respectively, from SEQ ID NO: 128, SEQ ID NO: 129, and SEQ ID NO: 114, respectively, or from SEQ ID NO: 156, SEQ ID NO: 157, and SEQ ID NO: 163, respectively, and the LCDR1, LCDR2, and LCDR3 are selected from SEQ ID NO: 106, SEQ ID NO: 107, and SEQ ID NO: 115, respectively, from SEQ ID NO: 106, SEQ ID NO: 107, and SEQ ID NO: 115, respectively, or from SEQ ID NO: 159, SEQ ID NO: 160, and SEQ ID NO: 115 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 116, respectively, from SEQ ID NO: 128, SEQ ID NO: 129, and SEQ ID NO: 116, respectively, or from SEQ ID NO: 156, SEQ ID NO: 157, and SEQ ID NO: 164, respectively, and the LCDR1, LCDR2, and LCDR3 are selected from SEQ ID NO: 106, SEQ ID NO: 107, and SEQ ID NO: 113, respectively, from SEQ ID NO: 106, SEQ ID NO: 107, and SEQ ID NO: 113, respectively, or from SEQ ID NO: 159, SEQ ID NO: 160, and SEQ ID NO: 113 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 117, respectively, from SEQ ID NO: 128, SEQ ID NO: 129, and SEQ ID NO: 117, respectively, or from SEQ ID NO: 156, SEQ ID NO: 157, and SEQ ID NO: 165, respectively, and the LCDR1, LCDR2, and LCDR3 are selected from SEQ ID NO: 106, SEQ ID NO: 107, and SEQ ID NO: 118, respectively, from SEQ ID NO: 106, SEQ ID NO: 107, and SEQ ID NO: 118, respectively, or from SEQ ID NO: 159, SEQ ID NO: 160, and SEQ ID NO: 118 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 116, respectively, from SEQ ID NO: 128, SEQ ID NO: 129, and SEQ ID NO: 116, respectively, or from SEQ ID NO: 156, SEQ ID NO: 157, and SEQ ID NO: 164, respectively, and the LCDR1, LCDR2, and LCDR3 are selected from SEQ ID NO: 106, SEQ ID NO: 107, and SEQ ID NO: 119, respectively, from SEQ ID NO: 106, SEQ ID NO: 107, and SEQ ID NO: 119, respectively, or from SEQ ID NO: 159, SEQ ID NO: 160, and SEQ ID NO: 119 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 120, respectively, from SEQ ID NO: 128, SEQ ID NO: 129, and SEQ ID NO: 120, respectively, or from SEQ ID NO: 156, SEQ ID NO: 157, and SEQ ID NO: 166, respectively, and the LCDR1, LCDR2, and LCDR3 are selected from SEQ ID NO: 106, SEQ ID NO: 107, and SEQ ID NO: 121, respectively, from SEQ ID NO: 106, SEQ ID NO: 107, and SEQ ID NO: 121, respectively, or from SEQ ID NO: 159, SEQ ID NO: 160, and SEQ ID NO: 121 respectively.

In some embodiments, an antibody or antigen-binding fragment thereof is provided, wherein the HCDR1, HCDR2, and HCDR3 are selected from SEQ ID NO: 103, SEQ ID NO: 104, and SEQ ID NO: 122, respectively, from SEQ ID NO: 128, SEQ ID NO: 129, and SEQ ID NO: 122, respectively, or from SEQ ID NO: 156, SEQ ID NO: 157, and SEQ ID NO: 167, respectively, and the LCDR1, LCDR2, and LCDR3 are selected from SEQ ID NO: 106, SEQ ID NO: 107, and SEQ ID NO: 123, respectively, from SEQ ID NO: 106, SEQ ID NO: 107, and SEQ ID NO: 123, respectively, or from SEQ ID NO: 159, SEQ ID NO: 160, and SEQ ID NO: 123 respectively.

In some embodiments, the antibody comprises a heavy chain variable region peptide having one of the following sequences, or a variant thereof:

TABLE 5

| SEQ | AB | |
|---|---|---|
| ID NO: | ID NO. | VH Sequence |
| 1 | MAB1 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDQEDYALDYWGQGTLVTVSS |
| 3 | MAB2 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYGWDYWGQGTLVTVSS |
| 5 | MAB3 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAFDEWGQGTLVTVSS |
| 7 | MAB4 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESQYALDYWGQGTLVTVSS |
| 9 | MAB5 | QVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDNTDYALDLWGQGTLVTVSS |
| 11 | MAB6 | QVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDETDYAYDEWGQGTLVTVSS |
| 13 | MAB7 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAYDNWGQGTLVTVSS |
| 15 | MAB8 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYAYDYWGQGTLVTVSS |
| 17 | MAB9 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDENYALDWWGQGTLVTVSS |
| 19 | MAB10 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYALDFWGQGTLVTVSS |
| 21 | MAB11 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGVPFDLWGQGTLVTVSS |
| 23 | MAB12 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLGGRPFDHWGQGTLVTVSS |
| 25 | MAB13 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGFPFDIWGQGTLVTVSS |
| 27 | MAB14 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAWRPTDSWGQGTLVTVSS |
| 29 | MAB15 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTKGLAWLPYYSWGQGTLVTVSS |
| 31 | MAB16 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGVPFDLWGQGTLVTVSS |
| 33 | MAB17 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGEAGRPFDAWGQGTLVTVSS |
| 35 | MAB18 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGRPYDVWGQGTLVTVSS |
| 37 | MAB19 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSYLRSEDTAVYYCVTEGLAGIPFDSWGQGTLVTVSS |
| 39 | MAB20 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSYLRSEDTAVYYCVTEGLAGIPFDSWGQGTLVTVSS |
| 41 | MAB21 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 43 | MAB22 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKYYYYYYMDVWGKGT |
| | | TVTVSS |
| 45 | MAB23 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGQTYYYYYMDVWGKGT |
| | | TVTVSS |
| 47 | MAB24 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 49 | MAB25 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGIKYYYYYMDVWGKGTT |
| | | VTVSS |
| 51 | MAB26 | EVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 53 | MAB27 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKVYYYYYMDVWGKGT |
| | | TVTVSS |
| 55 | MAB28 | EVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 57 | MAB29 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGNHYYYYYMDAWGKGT |
| | | TVTVSS |
| 59 | MAB30 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGRHYYYYYMDVWGKGT |
| | | TVTVSS |
| 334 | MAB31 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDQEDYALDYWGQGTLVTVSS |
| 335 | MAB32 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYGWDYWGQGTLVTVSS |
| 336 | MAB33 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAFDEWGQGTLVTVSS |
| 337 | MAB34 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESQYALDYWGQGTLVTVSS |
| 338 | MAB35 | EVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDNTDYALDLWGQGTLVTVSS |
| 339 | MAB36 | EVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDETDYAYDEWGQGTLVTVSS |
| 340 | MAB37 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAYDNWGQGTLVTVSS |
| 341 | MAB38 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYAYDYWGQGTLVTVSS |
| 342 | MAB39 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDENYALDWWGQGTLVTVSS |
| 343 | MAB40 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYALDFWGQGTLVTVSS |
| 344 | MAB41 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGVPFDLWGQGTLVTVSS |
| 345 | MAB42 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLGGRPFDHWGQGTLVTVSS |
| 346 | MAB43 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGFPFDIWGQGTLVTVSS |
| 347 | MAB44 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAWRPTDSWGQGTLVTVSS |
| 348 | MAB45 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTKGLAWLPYYSWGQGTLVTVSS |
| 349 | MAB46 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGVPFDLWGQGTLVTVSS |
| 350 | MAB47 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGEAGRPFDAWGQGTLVTVSS |
| 351 | MAB48 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGRPYDVWGQGTLVTVSS |
| 352 | MAB49 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSYLRSEDTAVYYCVTEGLAGIPFDSWGQGTLVTVSS |
| 353 | MAB50 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSYLRSEDTAVYYCVTEGLAGIPFDSWGQGTLVTVSS |
| 354 | MAB51 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 355 | MAB52 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKYYYYYYMDVWGKGT |
| | | TVTVSS |
| 356 | MAB53 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGQTYYYYYMDVWGKGT |
| | | TVTVSS |
| 357 | MAB54 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 358 | MAB55 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGIKYYYYYMDVWGKGTT |
| | | VTVSS |
| 359 | MAB56 | QVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 360 | MAB57 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKVYYYYYMDVWGKGT |
| | | TVTVSS |
| 361 | MAB58 | QVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 362 | MAB59 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGNHYYYYYMDAWGKGT |
| | | TVTVSS |
| 363 | MAB60 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGRHYYYYYMDVWGKGT |
| | | TVTVSS |

In some embodiments, the antibody comprises the heavy chain variable chain sequence of: VQLVESGGGLVKPGGSLRLSCAASGFTFSDYYMSWIRQAPGKGLEWVSYISRSGSTKYY ADSVKGRFTISRDNAKNSLYLQMNSLRAEDTAVYYCARDENYALDWWGQGTLVTVSS (SEQ ID NO: 454). A heavy variable chain comprising this amino acid sequence can be paired with for example, the variable light chain comprising the amino acid sequence of SEQ ID NO 18 or the light chain of SEQ ID NO: 236. A variable heavy chain (VH) comprising the amino acid sequence of SEQ ID NO: 454 can be fused to a constant domain comprising a YTE or LS mutations as exemplified herein. For example, in some embodiments, the constant domain comprises the amino acid sequence of SEQ ID NO: 262 or SEQ ID NO: 261.

In some embodiments, the antibody comprises a heavy chain sequence of:

(SEQ ID NO: 455)
VQLVESGGGLVKPGGSLRLSCAASGFTFSDYYMSWIRQAPGKGLEWVSYI
SRSGSTKYYADSVKGRFTISRDNAKNSLYLQMNSLRAEDTAVYYCARDEN
YALDWWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFEGGPSVFLFPPKPKDTLYIT
REPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLG.

In some embodiments, the antibody comprises a light chain variable region peptide having one of the following sequences, or a variant thereof:

TABLE 6

| SEQ | AB | |
|---|---|---|
| ID NO: | ID NO. | VL Sequence |
| 2 | MAB1/MAB31 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCEQYEDYPLTFGGGTKVEIK |
| 4 | MAB2/MAB32 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCKQYEDYPLTFGGGTKVEIK |
| 6 | MAB3/MAB 33 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCKQYEDYPLTFGGGTKVEIK |
| 8 | MAB4/MAB 34 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCHQYEDYPLTFGGGTKVEIK |
| 10 | MAB5/MAB 35 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCHQYEDYPLTFGGGTKVEIK |
| 12 | MAB6/MAB 36 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCHQYEDYPLTFGGGTKVEIK |
| 14 | MAB7/MAB 37 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQQYEDYPLVFGGGTKVEIK |
| 16 | MAB8/MAB 38 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQQHEDYPLTFGGGTKVEIK |
| 18 | MAB9/MAB 39 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQHYEDYPLTFGGGTKVEIK |
| 20 | MAB10/MAB 40 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQQYEDLIPTFGGGTKVEIK |
| 22 | MAB11/MAB 41 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQSYNSYVWTFGQGTKVEIK |
| 24 | MAB12/MAB 42 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQVNSYDWTFGQGTKVEIK |
| 26 | MAB13/MAB 43 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQVNSYDWTFGQGTKVEIK |
| 28 | MAB14/MAB 44 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQYSSYAWTFGQGTKVEIK |
| 30 | MAB15/MAB 45 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQARSYSWTFGQGTKVEIK |
| 32 | MAB16/MAB 46 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQHNSYRWTFGQGTKVEIK |
| 34 | MAB17/MAB 47 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCVQYPSYSWTFGQGTKVEIK |
| 36 | MAB18/MAB 48 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQYNSYKLTFGQGTKVEIK |
| 38 | MAB19/MAB 49 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQYNSPSWLFGQGTKVEIK |
| 40 | MAB20/MAB 50 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQVNSLSWTFGQGTKVEIK |
| 42 | MAB21/MAB 51 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCFQYNSYPLGFGGGTKVEIK |
| 44 | MAB22/MAB 52 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYRSHPLTFGGGTKVEIK |
| 46 | MAB23/MAB 53 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYNQVPLTFGGGTKVEIK |
| 48 | MAB24/MAB 54 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQTNIYPLTFGGGTKVEIK |
| 50 | MAB25/MAB 55 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYKQYPLTFGGGTKVEIK |
| 52 | MAB26/MAB 56 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQTNIYPLTFGGGTKVEIK |
| 54 | MAB27/MAB 57 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYNSYPLAFGGGTKVEIK |
| 56 | MAB28/MAB 58 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYHSYPLRFGGGTKVEIK |
| 58 | MAB29/MAB 59 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYASYPLKFGGGTKVEIK |
| 60 | MAB30/MAB 60 | DIQMTQSPSSLSASVGDRVTITCRASQGIRNDLGWYQQKPG |
| | | KAPKRLIYTASNLQSGVPSRFSGSGSGTEFTLTISSLOPEDFAT |
| | | YYCLQYNAYPLIFGGGTKVEIK |

In some embodiments, an antibody, or antigen binding fragment thereof, comprises a sequence selected from one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, or any variants thereof.

In some embodiments, an antibody, or antigen binding fragment thereof, comprises a VH peptide of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 35358, 359, 360, 361, 362, or 363, or any variant thereof.

In some embodiments, an antibody, or antigen binding fragment thereof, comprises a VH peptide having at least 85%, 86%, 87%, 88%, 89%, 90%, 91%, 92%, 93%, 94%, 95%, 96%, 97%, 98%, or 99% identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 35358, 359, 360, 361, 362, or 363.

In some embodiments, an antibody, or antigen binding fragment thereof, comprises a VH peptide of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 35358, 359, 360, 361, 362, or 363.

In some embodiments, an antibody, or antigen binding fragment thereof, comprises a VH peptide of SEQ ID NO: 1. In some embodiments, an antibody, or antigen binding fragment thereof, comprises a V_{H} peptide having at least 85% identity to SEQ ID NO: 1. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 1.

In some embodiments, an antibody, or antigen binding fragment thereof, comprises a VH peptide of SEQ ID NO: 3. In some embodiments, an antibody, or antigen binding fragment thereof, comprises a V_{H} peptide having at least 85% identity to SEQ ID NO: 3.

In some embodiments, an antibody, or antigen binding fragment thereof, comprises a VH peptide having at least 85%, 86%, 87%, 88%, 89%, 90%, 91%, 92%, 93%, 94%, 95%, 96%, 97%, 98%, or 99% identity to SEQ ID NO: 3.

In some embodiments, an antibody, or antigen binding fragment thereof, comprises a VH peptide of SEQ ID NO: 5. In some embodiments, an antibody, or antigen binding fragment thereof, comprises a V_{H} peptide having at least 85% identity to SEQ ID NO: 5. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 5.

In some embodiments, an antibody, or antigen binding fragment thereof, comprises a VH peptide of SEQ ID NO: 7. In some embodiments, an antibody, or antigen binding fragment thereof, comprises a V_{H} peptide having at least 85% identity to SEQ ID NO: 7. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 7.

In some embodiments, an antibody, or antigen binding fragment thereof, comprises a VH peptide of SEQ ID NO: 9. In some embodiments, an antibody, or antigen binding fragment thereof, comprises a V_{H} peptide having at least 85% identity to SEQ ID NO: 9. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 9.

In some embodiments, an antibody, or antigen binding fragment thereof, comprises a V_{H} peptide of SEQ ID NO: 11. In some embodiments, an antibody, or antigen binding fragment thereof, comprises a V_{H} peptide having at least 85% identity to SEQ ID NO: 11. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 11.

In some embodiments, an antibody, or antigen binding fragment thereof, comprises a V_{H} peptide of SEQ ID NO: 13. In some embodiments, an antibody, or antigen binding fragment thereof, comprises a V_{H} peptide having at least 85% identity to SEQ ID NO: 13. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 13.

In some embodiments, an antibody, or antigen binding fragment thereof, comprises a V_{H} peptide of SEQ ID NO: 15. In some embodiments, an antibody, or antigen binding fragment thereof, comprises a V_{H} peptide having at least 85% identity to SEQ ID NO: 15. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 15.

In some embodiments, an antibody, or antigen binding fragment thereof, comprises a V_{H} peptide of SEQ ID NO: 17. In some embodiments, an antibody, or antigen binding fragment thereof, comprises a V_{H} peptide having at least 85% identity to SEQ ID NO: 17. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 17.

In some embodiments, an antibody, or antigen binding fragment thereof, comprises a V_{H} peptide of SEQ ID NO: 19. In some embodiments, an antibody, or antigen binding fragment thereof, comprises a V_{H} peptide having at least 85% identity to SEQ ID NO: 19. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 19.

In some embodiments, an antibody, or antigen binding fragment thereof, comprises a V_{H} peptide of SEQ ID NO: 21. In some embodiments, an antibody, or antigen binding fragment thereof, comprises a V_{H} peptide having at least 85% identity to SEQ ID NO: 21. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 21.

In some embodiments, an antibody, or antigen binding fragment thereof, comprises a V_{H} peptide of SEQ ID NO: 23. In some embodiments, an antibody, or antigen binding fragment thereof, comprises a V_{H} peptide having at least 85% identity to SEQ ID NO: 23. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 23.

In some embodiments, an antibody, or antigen binding fragment thereof, comprises a V_{H} peptide of SEQ ID NO: 25. In some embodiments, an antibody, or antigen binding fragment thereof, comprises a V_{H} peptide having at least 85% identity to SEQ ID NO: 25. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 25.

In some embodiments, an antibody, or antigen binding fragment thereof, comprises a V_{H} peptide of SEQ ID NO: 27. In some embodiments, an antibody, or antigen binding fragment thereof, comprises a V_{H} peptide having at least 85% identity to SEQ ID NO: 27. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 27.

In some embodiments, an antibody, or antigen binding fragment thereof, comprises a V_{H} peptide of SEQ ID NO: 29. In some embodiments, an antibody, or antigen binding fragment thereof, comprises a V_{H} peptide having at least 85% identity to SEQ ID NO: 29. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 29.

In some embodiments, an antibody, or antigen binding fragment thereof, comprises a V_{H} peptide of SEQ ID NO: 31. In some embodiments, an antibody, or antigen binding fragment thereof, comprises a V_{H} peptide having at least 85% identity to SEQ ID NO: 31. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 31.

In some embodiments, an antibody, or antigen binding fragment thereof, comprises a V_{H} peptide of SEQ ID NO: 33. In some embodiments, an antibody, or antigen binding fragment thereof, comprises a V_{H} peptide having at least 85% identity to SEQ ID NO: 33. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 33.

In some embodiments, an antibody, or antigen binding fragment thereof, comprises a V_{H} peptide of SEQ ID NO: 35. In some embodiments, an antibody, or antigen binding fragment thereof, comprises a V_{H} peptide having at least 85% identity to SEQ ID NO: 35. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 35.

In some embodiments, an antibody, or antigen binding fragment thereof, comprises a V_{H} peptide of SEQ ID NO: 37. In some embodiments, an antibody, or antigen binding fragment thereof, comprises a V_{H} peptide having at least 85% identity to SEQ ID NO: 37. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 37.

In some embodiments, an antibody, or antigen binding fragment thereof, comprises a V_{H} peptide of SEQ ID NO: 39. In some embodiments, an antibody, or antigen binding fragment thereof, comprises a V_{H} peptide having at least 85% identity to SEQ ID NO: 39. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 39.

In some embodiments, an antibody, or antigen binding fragment thereof, comprises a V_{H} peptide of SEQ ID NO: 41. In some embodiments, an antibody, or antigen binding fragment thereof, comprises a V_{H} peptide having at least 85% identity to SEQ ID NO: 41. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 41.

In some embodiments, an antibody, or antigen binding fragment thereof, comprises a V_{H} peptide of SEQ ID NO: 43. In some embodiments, an antibody, or antigen binding fragment thereof, comprises a V_{H} peptide having at least 85% identity to SEQ ID NO: 43. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 43.

In some embodiments, an antibody, or antigen binding fragment thereof, comprises a V_{H} peptide of SEQ ID NO: 45. In some embodiments, an antibody, or antigen binding fragment thereof, comprises a V_{H} peptide having at least 85% identity to SEQ ID NO: 45. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 45.

In some embodiments, an antibody, or antigen binding fragment thereof, comprises a V_{H} peptide of SEQ ID NO: 47. In some embodiments, an antibody, or antigen binding fragment thereof, comprises a V_{H} peptide having at least 85% identity to SEQ ID NO: 47. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 47.

In some embodiments, an antibody, or antigen binding fragment thereof, comprises a V_{H} peptide of SEQ ID NO: 49. In some embodiments, an antibody, or antigen binding fragment thereof, comprises a V_{H} peptide having at least 85% identity to SEQ ID NO: 49. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 49.

In some embodiments, an antibody, or antigen binding fragment thereof, comprises a V_{H} peptide of SEQ ID NO: 51. In some embodiments, an antibody, or antigen binding fragment thereof, comprises a V_{H} peptide having at least 85% identity to SEQ ID NO: 51. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 51.

In some embodiments, an antibody, or antigen binding fragment thereof, comprises a V_{H} peptide of SEQ ID NO: 53. In some embodiments, an antibody, or antigen binding fragment thereof, comprises a V_{H} peptide having at least 85% identity to SEQ ID NO: 53. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 53.

In some embodiments, an antibody, or antigen binding fragment thereof, comprises a V_{H} peptide of SEQ ID NO: 55. In some embodiments, an antibody, or antigen binding fragment thereof, comprises a V_{H} peptide having at least 85% identity to SEQ ID NO: 55. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 55.

In some embodiments, an antibody, or antigen binding fragment thereof, comprises a V_{H} peptide of SEQ ID NO: 57. In some embodiments, an antibody, or antigen binding fragment thereof, comprises a V_{H} peptide having at least 85% identity to SEQ ID NO: 57. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 57.

In some embodiments, an antibody, or antigen binding fragment thereof, comprises a V_{H} peptide of SEQ ID NO: 59. In some embodiments, an antibody, or antigen binding fragment thereof, comprises a V_{H} peptide having at least 85% identity to SEQ ID NO: 59. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 59.

In some embodiments, an antibody, or antigen binding fragment thereof, comprises a V_{H} peptide of SEQ ID NO: 334. In some embodiments, an antibody, or antigen binding fragment thereof, comprises a V_{H} peptide having at least 85% identity to SEQ ID NO: 334. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 334.

In some embodiments, an antibody, or antigen binding fragment thereof, comprises a V_{H} peptide of SEQ ID NO: 335. In some embodiments, an antibody, or antigen binding fragment thereof, comprises a V_{H} peptide having at least 85% identity to SEQ ID NO: 335. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 335.

In some embodiments, an antibody, or antigen binding fragment thereof, comprises a V_{H} peptide of SEQ ID NO: 336. In some embodiments, an antibody, or antigen binding fragment thereof, comprises a V_{H} peptide having at least 85% identity to SEQ ID NO: 336. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 336.

In some embodiments, an antibody, or antigen binding fragment thereof, comprises a V_{H} peptide of SEQ ID NO: 337. In some embodiments, an antibody, or antigen binding fragment thereof, comprises a V_{H} peptide having at least 85% identity to SEQ ID NO: 337. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 337.

In some embodiments, an antibody, or antigen binding fragment thereof, comprises a V_{H} peptide of SEQ ID NO: 338. In some embodiments, an antibody, or antigen binding fragment thereof, comprises a V_{H} peptide having at least 85% identity to SEQ ID NO: 338. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 338.

In some embodiments, an antibody, or antigen binding fragment thereof, comprises a V_{H} peptide of SEQ ID NO: 339. In some embodiments, an antibody, or antigen binding fragment thereof, comprises a V_{H} peptide having at least 85% identity to SEQ ID NO: 339. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 339.

In some embodiments, an antibody, or antigen binding fragment thereof, comprises a V_{H} peptide of SEQ ID NO: 340. In some embodiments, an antibody, or antigen binding fragment thereof, comprises a V_{H} peptide having at least 85% identity to SEQ ID NO: 340. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 340.

In some embodiments, an antibody, or antigen binding fragment thereof, comprises a V_{H} peptide of SEQ ID NO: 341. In some embodiments, an antibody, or antigen binding fragment thereof, comprises a V_{H} peptide having at least 85% identity to SEQ ID NO: 341. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 341.

In some embodiments, an antibody, or antigen binding fragment thereof, comprises a V_{H} peptide of SEQ ID NO: 342. In some embodiments, an antibody, or antigen binding fragment thereof, comprises a V_{H} peptide having at least 85% identity to SEQ ID NO: 342. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 342.

In some embodiments, an antibody, or antigen binding fragment thereof, comprises a V_{H} peptide of SEQ ID NO: 343. In some embodiments, an antibody, or antigen binding fragment thereof, comprises a V_{H} peptide having at least 85% identity to SEQ ID NO: 343. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 343.

In some embodiments, an antibody, or antigen binding fragment thereof, comprises a V_{H} peptide of SEQ ID NO: 344. In some embodiments, an antibody, or antigen binding fragment thereof, comprises a V_{H} peptide having at least 85% identity to SEQ ID NO: 344. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 344.

In some embodiments, an antibody, or antigen binding fragment thereof, comprises a V_{H} peptide of SEQ ID NO: 345. In some embodiments, an antibody, or antigen binding fragment thereof, comprises a V_{H} peptide having at least 85% identity to SEQ ID NO: 345. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 345.

In some embodiments, an antibody, or antigen binding fragment thereof, comprises a V_{H} peptide of SEQ ID NO: 346. In some embodiments, an antibody, or antigen binding fragment thereof, comprises a V_{H} peptide having at least 85% identity to SEQ ID NO: 346. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 346.

In some embodiments, an antibody, or antigen binding fragment thereof, comprises a V_{H} peptide of SEQ ID NO: 347. In some embodiments, an antibody, or antigen binding fragment thereof, comprises a V_{H} peptide having at least 85% identity to SEQ ID NO: 347. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 347.

In some embodiments, an antibody, or antigen binding fragment thereof, comprises a V_{H} peptide of SEQ ID NO: 348. In some embodiments, an antibody, or antigen binding fragment thereof, comprises a V_{H} peptide having at least 85% identity to SEQ ID NO: 348. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 348.

In some embodiments, an antibody, or antigen binding fragment thereof, comprises a V_{H} peptide of SEQ ID NO: 349. In some embodiments, an antibody, or antigen binding fragment thereof, comprises a V_{H} peptide having at least 85% identity to SEQ ID NO: 349. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 349.

In some embodiments, an antibody, or antigen binding fragment thereof, comprises a V_{H} peptide of SEQ ID NO: 350. In some embodiments, an antibody, or antigen binding fragment thereof, comprises a V_{H} peptide having at least 85% identity to SEQ ID NO: 350. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 350.

In some embodiments, an antibody, or antigen binding fragment thereof, comprises a V_{H} peptide of SEQ ID NO: 351. In some embodiments, an antibody, or antigen binding fragment thereof, comprises a V_{H} peptide having at least 85% identity to SEQ ID NO: 351. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 351.

In some embodiments, an antibody, or antigen binding fragment thereof, comprises a V_{H} peptide of SEQ ID NO: 352. In some embodiments, an antibody, or antigen binding fragment thereof, comprises a V_{H} peptide having at least 85% identity to SEQ ID NO: 352. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 352.

In some embodiments, an antibody, or antigen binding fragment thereof, comprises a V_{H} peptide of SEQ ID NO: 353. In some embodiments, an antibody, or antigen binding fragment thereof, comprises a V_{H} peptide having at least 85% identity to SEQ ID NO: 353. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 353.

In some embodiments, an antibody, or antigen binding fragment thereof, comprises a V_{H} peptide of SEQ ID NO: 354. In some embodiments, an antibody, or antigen binding fragment thereof, comprises a V_{H} peptide having at least 85% identity to SEQ ID NO: 354. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 354.

In some embodiments, an antibody, or antigen binding fragment thereof, comprises a V_{H} peptide of SEQ ID NO: 355. In some embodiments, an antibody, or antigen binding fragment thereof, comprises a V_{H} peptide having at least 85% identity to SEQ ID NO: 355. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 355.

In some embodiments, an antibody, or antigen binding fragment thereof, comprises a V_{H} peptide of SEQ ID NO: 356. In some embodiments, an antibody, or antigen binding fragment thereof, comprises a V_{H} peptide having at least 85% identity to SEQ ID NO: 356. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 356.

In some embodiments, an antibody, or antigen binding fragment thereof, comprises a V_{H} peptide of SEQ ID NO: 357. In some embodiments, an antibody, or antigen binding fragment thereof, comprises a V_{H} peptide having at least 85% identity to SEQ ID NO: 357. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 357.

In some embodiments, an antibody, or antigen binding fragment thereof, comprises a V_{H} peptide of SEQ ID NO: 358. In some embodiments, an antibody, or antigen binding fragment thereof, comprises a V_{H} peptide having at least 85% identity to SEQ ID NO: 358. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 358.

In some embodiments, an antibody, or antigen binding fragment thereof, comprises a V_{H} peptide of SEQ ID NO: 359. In some embodiments, an antibody, or antigen binding fragment thereof, comprises a V_{H} peptide having at least 85% identity to SEQ ID NO: 359. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 359.

In some embodiments, an antibody, or antigen binding fragment thereof, comprises a V_{H} peptide of SEQ ID NO: 360. In some embodiments, an antibody, or antigen binding fragment thereof, comprises a V_{H} peptide having at least 85% identity to SEQ ID NO: 360. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 360.

In some embodiments, an antibody, or antigen binding fragment thereof, comprises a V_{H} peptide of SEQ ID NO: 361. In some embodiments, an antibody, or antigen binding fragment thereof, comprises a V_{H} peptide having at least 85% identity to SEQ ID NO: 361. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 361.

In some embodiments, an antibody, or antigen binding fragment thereof, comprises a V_{H} peptide of SEQ ID NO: 362. In some embodiments, an antibody, or antigen binding fragment thereof, comprises a V_{H} peptide having at least 85% identity to SEQ ID NO: 362. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 362.

In some embodiments, an antibody, or antigen binding fragment thereof, comprises a V_{H} peptide of SEQ ID NO: 363. In some embodiments, an antibody, or antigen binding fragment thereof, comprises a V_{H} peptide having at least 85% identity to SEQ ID NO: 363. In some embodiments, an antibody, or antigen binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 363.

In some embodiments, an antibody, or antigen binding fragment thereof, comprises a VL peptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60, or any variant thereof. In some embodiments, an antibody, or antigen binding fragment thereof, comprises a V_{L} peptide having at least 85% identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60. In some embodiments, an antibody, or antigen binding fragment thereof, comprises a V_{L} peptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60.

In some embodiments, an antibody, or antigen binding fragment thereof, comprises a VL peptide of SEQ ID NO: 2. In some embodiments, an antibody, or antigen binding fragment thereof, comprises a V_{L} peptide having at least 85% identity to SEQ ID NO: 2. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 2.

In some embodiments, an antibody, or antigen binding fragment thereof, comprises a VL peptide of SEQ ID NO: 4. In some embodiments, an antibody, or antigen binding fragment thereof, comprises a V_{L} peptide having at least 85% identity to SEQ ID NO: 4. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 4.

In some embodiments, an antibody, or antigen binding fragment thereof, comprises a VL peptide of SEQ ID NO: 6. In some embodiments, an antibody, or antigen binding fragment thereof, comprises a V_{L} peptide having at least 85% identity to SEQ ID NO: 6. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 6.

In some embodiments, an antibody, or antigen binding fragment thereof, comprises a VL peptide of SEQ ID NO: 8. In some embodiments, an antibody, or antigen binding fragment thereof, comprises a V_{L} peptide having at least 85% identity to SEQ ID NO: 8. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 8.

In some embodiments, an antibody, or antigen binding fragment thereof, comprises a V_{L} peptide of SEQ ID NO: 10. In some embodiments, an antibody, or antigen binding fragment thereof, comprises a V_{L} peptide having at least 85% identity to SEQ ID NO: 10. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 10.

In some embodiments, an antibody, or antigen binding fragment thereof, comprises a V_{L} peptide of SEQ ID NO: 12. In some embodiments, an antibody, or antigen binding fragment thereof, comprises a V_{L} peptide having at least 85% identity to SEQ ID NO: 12. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 12.

In some embodiments, an antibody, or antigen binding fragment thereof, comprises a V_{L} peptide of SEQ ID NO: 14. In some embodiments, an antibody, or antigen binding fragment thereof, comprises a V_{L} peptide having at least 85% identity to SEQ ID NO: 14. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 14.

In some embodiments, an antibody, or antigen binding fragment thereof, comprises a V_{L} peptide of SEQ ID NO: 16. In some embodiments, an antibody, or antigen binding fragment thereof, comprises a V_{L} peptide having at least 85% identity to SEQ ID NO: 16. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 16.

In some embodiments, an antibody, or antigen binding fragment thereof, comprises a V_{L} peptide of SEQ ID NO: 18. In some embodiments, an antibody, or antigen binding fragment thereof, comprises a V_{L} peptide having at least 85% identity to SEQ ID NO: 18. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 18.

In some embodiments, an antibody, or antigen binding fragment thereof, comprises a V_{L} peptide of SEQ ID NO: 20. In some embodiments, an antibody, or antigen binding fragment thereof, comprises a V_{L} peptide having at least 85% identity to SEQ ID NO: 20. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 20.

In some embodiments, an antibody, or antigen binding fragment thereof, comprises a V_{L} peptide of SEQ ID NO: 22. In some embodiments, an antibody, or antigen binding fragment thereof, comprises a V_{L} peptide having at least 85% identity to SEQ ID NO: 22. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 22.

In some embodiments, an antibody, or antigen binding fragment thereof, comprises a V_{L} peptide of SEQ ID NO: 24. In some embodiments, an antibody, or antigen binding fragment thereof, comprises a V_{L} peptide having at least 85% identity to SEQ ID NO: 24. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 24.

In some embodiments, an antibody, or antigen binding fragment thereof, comprises a V_{L} peptide of SEQ ID NO: 26. In some embodiments, an antibody, or antigen binding fragment thereof, comprises a V_{L} peptide having at least 85% identity to SEQ ID NO: 26. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 26.

In some embodiments, an antibody, or antigen binding fragment thereof, comprises a V_{L} peptide of SEQ ID NO: 28. In some embodiments, an antibody, or antigen binding fragment thereof, comprises a V_{L} peptide having at least 85% identity to SEQ ID NO: 28. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 28.

In some embodiments, an antibody, or antigen binding fragment thereof, comprises a V_{L} peptide of SEQ ID NO: 30. In some embodiments, an antibody, or antigen binding fragment thereof, comprises a V_{L} peptide having at least 85% identity to SEQ ID NO: 30. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 30.

In some embodiments, an antibody, or antigen binding fragment thereof, comprises a V_{L} peptide of SEQ ID NO: 32. In some embodiments, an antibody, or antigen binding fragment thereof, comprises a V_{L} peptide having at least 85% identity to SEQ ID NO: 32. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 32.

In some embodiments, an antibody, or antigen binding fragment thereof, comprises a V_{L} peptide of SEQ ID NO: 34. In some embodiments, an antibody, or antigen binding fragment thereof, comprises a V_{L} peptide having at least 85% identity to SEQ ID NO: 34. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 34.

In some embodiments, an antibody, or antigen binding fragment thereof, comprises a V_{L} peptide of SEQ ID NO: 36. In some embodiments, an antibody, or antigen binding fragment thereof, comprises a V_{L} peptide having at least 85% identity to SEQ ID NO: 36. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 36.

In some embodiments, an antibody, or antigen binding fragment thereof, comprises a V_{L} peptide of SEQ ID NO: 38. In some embodiments, an antibody, or antigen binding fragment thereof, comprises a V_{L} peptide having at least 85% identity to SEQ ID NO: 38. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 38.

In some embodiments, an antibody, or antigen binding fragment thereof, comprises a V_{L} peptide of SEQ ID NO: 40. In some embodiments, an antibody, or antigen binding fragment thereof, comprises a V_{L} peptide having at least 85% identity to SEQ ID NO: 40. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 40.

In some embodiments, an antibody, or antigen binding fragment thereof, comprises a V_{L} peptide of SEQ ID NO: 42. In some embodiments, an antibody, or antigen binding fragment thereof, comprises a V_{L} peptide having at least 85% identity to SEQ ID NO: 42. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 42.

In some embodiments, an antibody, or antigen binding fragment thereof, comprises a V_{L} peptide of SEQ ID NO: 44. In some embodiments, an antibody, or antigen binding fragment thereof, comprises a V_{L} peptide having at least 85% identity to SEQ ID NO: 44. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 44.

In some embodiments, an antibody, or antigen binding fragment thereof, comprises a V_{L} peptide of SEQ ID NO: 46. In some embodiments, an antibody, or antigen binding fragment thereof, comprises a V_{L} peptide having at least 85% identity to SEQ ID NO: 46. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 46.

In some embodiments, an antibody, or antigen binding fragment thereof, comprises a V_{L} peptide of SEQ ID NO: 48. In some embodiments, an antibody, or antigen binding fragment thereof, comprises a V_{L} peptide having at least 85% identity to SEQ ID NO: 48. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 48.

In some embodiments, an antibody, or antigen binding fragment thereof, comprises a V_{L} peptide of SEQ ID NO: 50. In some embodiments, an antibody, or antigen binding fragment thereof, comprises a V_{L} peptide having at least 85% identity to SEQ ID NO: 50. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 50.

In some embodiments, an antibody, or antigen binding fragment thereof, comprises a V_{L} peptide of SEQ ID NO: 52. In some embodiments, an antibody, or antigen binding fragment thereof, comprises a V_{L} peptide having at least 85% identity to SEQ ID NO: 52. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 52.

In some embodiments, an antibody, or antigen binding fragment thereof, comprises a V_{L} peptide of SEQ ID NO: 54. In some embodiments, an antibody, or antigen binding fragment thereof, comprises a V_{L} peptide having at least 85% identity to SEQ ID NO: 54. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 54.

In some embodiments, an antibody, or antigen binding fragment thereof, comprises a V_{L} peptide of SEQ ID NO: 56. In some embodiments, an antibody, or antigen binding fragment thereof, comprises a V_{L} peptide having at least 85% identity to SEQ ID NO: 56. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 56.

In some embodiments, an antibody, or antigen binding fragment thereof, comprises a V_{L} peptide of SEQ ID NO: 58. In some embodiments, an antibody, or antigen binding fragment thereof, comprises a V_{L} peptide having at least 85% identity to SEQ ID NO: 58. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 58.

In some embodiments, an antibody, or antigen binding fragment thereof, comprises a V_{L} peptide of SEQ ID NO: 60. In some embodiments, an antibody, or antigen binding fragment thereof, comprises a V_{L} peptide having at least 85% identity to SEQ ID NO: 60. In some embodiments, an antibody, or antigen binding fragment thereof, comprises a V_{L} peptide having at least 85%, 86%, 87%, 88%, 89%, 90%, 91%, 92%, 93%, 94%, 95%, 96%, 97%, 98%, or 99% identity to SEQ ID NO: 60.

In some embodiments, an antibody, or antigen binding fragment thereof, comprises a V_{H} peptide and a V_{L} peptide, wherein the wherein the V_{H} peptide comprises a sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 35358, 359, 360, 361, 362, or 363, or a variant thereof, and the VL peptide comprises a sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60, or a variant thereof. In some embodiments, an antibody, or antigen binding fragment thereof, comprises a V_{H} peptide and a V_{L} peptide, wherein the wherein the V_{H} peptide has at least 85%, 86%, 87%, 88%, 89%, 90%, 91%, 92%, 93%, 94%, 95%, 96%, 97%, 98%, or 99% identity to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 35358, 359, 360, 361, 362, or 363; and the V_{L} peptide has at least 85%, 86%, 87%, 88%, 89%, 90%, 91%, 92%, 93%, 94%, 95%, 96%, 97%, 98%, or 99% identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60. In some of these embodiments, the V_{H} peptide comprises a sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 35358, 359, 360, 361, 362, or 363; and the V_{L} peptide comprises a sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 1, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 3, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 1339, maintains a LCDR2 sequence of SEQ ID NO: 65, or SEQ ID NO: 134, and maintains a LCDR3 sequence SEQ ID NO: 68.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 5, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 7, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 9, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 11, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 13, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 15, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 17, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 19, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 21, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 23, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 25, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 27, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 29, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 31, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 33, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 35, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 37, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 39, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 41, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 43, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 45, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 47, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 49, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 51, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 53, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 55, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 57, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 59, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 334, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 335, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 1339, maintains a LCDR2 sequence of SEQ ID NO: 65, or SEQ ID NO: 134, and maintains a LCDR3 sequence SEQ ID NO: 68.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 336, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 337, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 338, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 339, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 340, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 341, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 342, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 343, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 344, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 345, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 346, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 347, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 348, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 349, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 350, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 351, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 352, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 353, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 354, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 355, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 356, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 357, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 358, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 359, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 360, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 361, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 362, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the heavy chain of the antibody, or antigen binding fragment thereof, comprises a heavy chain variable region having at least 85%, 86%, 87%, 88%, 89%, 90%, 91%, 92%, 93%, 94%, 95%, 96%, 97%, 98%, 99%, or 100% identity to SEQ ID NO: 363, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 2, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 4, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 1339, maintains a LCDR2 sequence of SEQ ID NO: 65, or SEQ ID NO: 134, and maintains a LCDR3 sequence SEQ ID NO: 68.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 6, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 8, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 10, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 12, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 14, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 16, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 18, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 20, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 22, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 24, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 26, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 28, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 30, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 32, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 34, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 36, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 38, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 40, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 42, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 44, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 46, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 48, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 50, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 52, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 54, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 56, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 58, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the light chain of the antibody, or antigen binding fragment thereof, comprises a light chain variable region having at least 85%, 86%, 87%, 88%, 89%, 90%, 91%, 92%, 93%, 94%, 95%, 96%, 97%, 98%, 99%, or 100% identity to SEQ ID NO: 60, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

The VH and the VL sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 266; (GGGGA)ₙ (SEQ ID NO: 267, or any combination thereof, wherein each n is independently 1-5. In some embodiments, each n is, independently, 1. In some embodiments, each n is, independently, 2. In some embodiments, each n is, independently, 3. In some embodiments, each n is, independently, 4. In some embodiments, each n is, independently, 5. In some embodiments, the variable regions are not linked with a peptide linker.

In some embodiments, the antibody comprises SEQ ID NO: 1 and SEQ ID NO: 2. In some embodiments, the antibody comprises SEQ ID NO: 3 and SEQ ID NO: 4. In some embodiments, the antibody comprises SEQ ID NO: 5 and SEQ ID NO: 6. In some embodiments, the antibody comprises SEQ ID NO: 7 and SEQ ID NO: 8. In some embodiments, the antibody comprises SEQ ID NO: 9 and SEQ ID NO: 10. In some embodiments, the antibody comprises SEQ ID NO: 11 and SEQ ID NO: 12. In some embodiments, the antibody comprises SEQ ID NO: 13 and SEQ ID NO: 14. In some embodiments, the antibody comprises SEQ ID NO: 15 and SEQ ID NO: 16. In some embodiments, the antibody comprises SEQ ID NO: 17 and SEQ ID NO: 18. In some embodiments, the antibody comprises SEQ ID NO: 19 and SEQ ID NO: 20. In some embodiments, the antibody comprises SEQ ID NO: 21 and SEQ ID NO: 22. In some embodiments, the antibody comprises SEQ ID NO: 23 and SEQ ID NO: 24. In some embodiments, the antibody comprises SEQ ID NO: 25 and SEQ ID NO: 26. In some embodiments, the antibody comprises SEQ ID NO: 27 and SEQ ID NO: 28. In some embodiments, the antibody comprises SEQ ID NO: 29 and SEQ ID NO: 30. In some embodiments, the antibody comprises SEQ ID NO: 31 and SEQ ID NO: 32. In some embodiments, the antibody comprises SEQ ID NO: 33 and SEQ ID NO: 34. In some embodiments, the antibody comprises SEQ ID NO: 35 and SEQ ID NO: 36. In some embodiments, the antibody comprises SEQ ID NO: 37 and SEQ ID NO: 38. In some embodiments, the antibody comprises SEQ ID NO: 39 and SEQ ID NO: 40. In some embodiments, the antibody comprises SEQ ID NO: 41 and SEQ ID NO: 42. In some embodiments, the antibody comprises SEQ ID NO: 43 and SEQ ID NO: 44. In some embodiments, the antibody comprises SEQ ID NO: 45 and SEQ ID NO: 46. In some embodiments, the antibody comprises SEQ ID NO: 47 and SEQ ID NO: 48. In some embodiments, the antibody comprises SEQ ID NO: 49 and SEQ ID NO: 50. In some embodiments, the antibody comprises SEQ ID NO: 51 and SEQ ID NO: 52. In some embodiments, the antibody comprises SEQ ID NO: 53 and SEQ ID NO: 54. In some embodiments, the antibody comprises SEQ ID NO: 55 and SEQ ID NO: 56. In some embodiments, the antibody comprises SEQ ID NO: 57 and SEQ ID NO: 58. In some embodiments, the antibody comprises SEQ ID NO: 59 and SEQ ID NO: 60. In some embodiments, the antibody comprises SEQ ID NO: 334 and SEQ ID NO: 2. In some embodiments, the antibody comprises SEQ ID NO: 335 and SEQ ID NO: 4. In some embodiments, the antibody comprises SEQ ID NO: 336 and SEQ ID NO: 6. In some embodiments, the antibody comprises SEQ ID NO: 337 and SEQ ID NO: 8. In some embodiments, the antibody comprises SEQ ID NO: 338 and SEQ ID NO: 10. In some embodiments, the antibody comprises SEQ ID NO: 339 and SEQ ID NO: 12. In some embodiments, the antibody comprises SEQ ID NO: 340 and SEQ ID NO: 14. In some embodiments, the antibody comprises SEQ ID NO: 341 and SEQ ID NO: 16. In some embodiments, the antibody comprises SEQ ID NO: 342 and SEQ ID NO: 18. In some embodiments, the antibody comprises SEQ ID NO: 343 and SEQ ID NO: 20. In some embodiments, the antibody comprises SEQ ID NO: 344 and SEQ ID NO: 22. In some embodiments, the antibody comprises SEQ ID NO: 345 and SEQ ID NO: 24. In some embodiments, the antibody comprises SEQ ID NO: 346 and SEQ ID NO: 26. In some embodiments, the antibody comprises SEQ ID NO: 347 and SEQ ID NO: 28. In some embodiments, the antibody comprises SEQ ID NO: 348 and SEQ ID NO: 30. In some embodiments, the antibody comprises SEQ ID NO: 349 and SEQ ID NO: 32. In some embodiments, the antibody comprises SEQ ID NO: 350 and SEQ ID NO: 34. In some embodiments, the antibody comprises SEQ ID NO: 351 and SEQ ID NO: 36. In some embodiments, the antibody comprises SEQ ID NO: 352 and SEQ ID NO: 38. In some embodiments, the antibody comprises SEQ ID NO: 353 and SEQ ID NO: 40. In some embodiments, the antibody comprises SEQ ID NO: 354 and SEQ ID NO: 42. In some embodiments, the antibody comprises SEQ ID NO: 355 and SEQ ID NO: 44. In some embodiments, the antibody comprises SEQ ID NO: 356 and SEQ ID NO: 46. In some embodiments, the antibody comprises SEQ ID NO: 357 and SEQ ID NO: 48. In some embodiments, the antibody comprises SEQ ID NO: 358 and SEQ ID NO: 50. In some embodiments, the antibody comprises SEQ ID NO: 359 and SEQ ID NO: 52. In some embodiments, the antibody comprises SEQ ID NO: 360 and SEQ ID NO: 54. In some embodiments, the antibody comprises SEQ ID NO: 361 and SEQ ID NO: 56. In some embodiments, the antibody comprises SEQ ID NO: 362 and SEQ ID NO: 58. In some embodiments, the antibody comprises SEQ ID NO: 363 and SEQ ID NO: 60.

As provided for herein, the different peptides (V_{H} or V_{L}) described herein can be linked with a peptide linker or not linked with a peptide linker and instead form a contiguous sequence. In some embodiments, the heavy chain variable region and the light chain variable region are not linked by a linker. In some embodiments, the heavy chain variable region and the light chain variable region are linked via a peptide linker. In some embodiments, the peptide linker comprises a sequence of (GGGGS)ₙ (SEQ ID NO: 266); (GGGGA)ₙ (SEQ ID NO: 267), or any combination thereof, wherein each n is independently 1-5. In some embodiments, each n is, independently, 1. In some embodiments, each n is, independently, 2. In some embodiments, each n is, independently, 3. In some embodiments, each n is, independently, 4. In some embodiments, each n is, independently, 5. The linked peptide format can be represented by a formula of V_{H}-Z-V_{L} or V_{L}-Z-V_{H}, wherein Z is the peptide linker. In some embodiments, Z is (GGGGS)ₙ (SEQ ID NO: 266); (GGGGA)ₙ (SEQ ID NO: 267), or any combination thereof, wherein each n is independently 1-5. In some embodiments, each n is, independently, 1. In some embodiments, each n is, independently, 2. In some embodiments, each n is, independently, 3. In some embodiments, each n is, independently, 4. In some embodiments, each n is, independently, 5.

In some embodiments, the VH and VL polypeptides are linked to a Fc region, such as “IgG4 S228P L235E LS” or “IgG4 S228P L235E YTE”. In some embodiments, the Fc region is as provided for herein. In some embodiments, the Fc region is “IgG4 S228P L235E LS”. In some embodiments, the Fc region is “IgG4 S228 L235E YTE”.

In some embodiments, an antibody, or antigen binding fragment thereof, comprises a VH peptide and a VL peptide as provided for herein. In some embodiments, the VH and VL each, independently, comprise 1, 2, 3, 4, or 5 conservative amino acid substitutions. In some embodiments, the CDRs of the VH and VL comprise no more than 1 amino acid conservative amino acid substitution. Therefore, in some embodiments the HCDR1, HCDR2, or HCDR3 could comprise one or no conservative amino acid substitutions. Additionally, in some embodiments the LCDR1, LCDR2, or LCDR3 could comprise one or no conservative amino acid substitutions.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 2, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 1 and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 4, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 3, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 1339, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 6, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 5, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 8, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 7, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 10, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 9, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 12, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 11, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 14, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 13, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 16, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 15, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 18, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 17, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 20, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 19, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 22, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 21, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 24, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 23, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 26, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 25, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 28, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 27, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 30, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 29, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 32, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 31, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 34, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 33, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 36, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 35, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 38, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 37, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 40, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 39, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 44, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 41, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 44, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 43, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 46, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 45, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 48, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 47, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 50, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 49, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 52, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 51, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 54, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 55, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 56, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 55, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 58, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 57, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 60, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 59, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 2, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 334 and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 4, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 335, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 1339, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 6, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 336, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 8, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 337, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 10, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 338, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 12, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 1339, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 14, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 340, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 16, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 341, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 18, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 342, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 20, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 343, and wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 22, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 344, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 24, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 345, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 26, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 346, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 28, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 347, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 30, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 348, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 32, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 349, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 34, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 350, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 36, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 351, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 38, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 352, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 40, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 353, and wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 44, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 354, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 44, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 355, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 46, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 356, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 48, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 357, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 50, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 358, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 52, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 359, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 54, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 360, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 56, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 361, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 58, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 362, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the antibody or antigen-binding fragment thereof comprises a light chain variable region and a heavy chain variable region, wherein the light chain variable region of the light chain has at least 85%, 86%, 87%, 88%, 89%, 90%, 91%, 92%, 93%, 94%, 95%, 96%, 97%, 98%, 99%, or 100% identity to SEQ ID NO: 60, the heavy chain variable region of the heavy chain has at least 85%, 86%, 87%, 88%, 89%, 90%, 91%, 92%, 93%, 94%, 95%, 96%, 97%, 98%, 99%, or 100% identity to SEQ ID NO: 363, and wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

In some embodiments, an antibody, or antigen binding fragment thereof, comprises a VH peptide and a VL peptide, wherein the V_{H} peptide comprises a sequence of SEQ ID NO: 1 and the V_{L} peptide comprises a sequence of SEQ ID NO: 2.

In some embodiments, an antibody, or antigen binding fragment thereof, comprises a VH peptide and a VL peptide, wherein the V_{H} peptide comprises a sequence of SEQ ID NO: 3 and the V_{L} peptide comprises a sequence of SEQ ID NO: 4.

In some embodiments, an antibody, or antigen binding fragment thereof, comprises a VH peptide and a VL peptide, wherein the V_{H} peptide comprises a sequence of SEQ ID NO: 5 and the V_{L} peptide comprises a sequence of SEQ ID NO: 6.

In some embodiments, an antibody, or antigen binding fragment thereof, comprises a VH peptide and a VL peptide, wherein the VH peptide comprises a sequence of SEQ ID NO: 7 and the VL peptide comprises a sequence of SEQ ID NO: 8.

In some embodiments, an antibody, or antigen binding fragment thereof, comprises a VH peptide and a VL peptide, wherein the VH peptide comprises a sequence of SEQ ID NO: 9 and the VL peptide comprises a sequence of SEQ ID NO: 10.

In some embodiments, an antibody, or antigen binding fragment thereof, comprises a VH peptide and a VL peptide, wherein the VH peptide comprises a sequence of SEQ ID NO: 11 and the VL peptide comprises a sequence of SEQ ID NO: 12.

In some embodiments, an antibody, or antigen binding fragment thereof, comprises a VH peptide and a VL peptide, wherein the VH peptide comprises a sequence of SEQ ID NO: 13 and the VL peptide comprises a sequence of SEQ ID NO: 14.

In some embodiments, an antibody, or antigen binding fragment thereof, comprises a VH peptide and a VL peptide, wherein the VH peptide comprises a sequence of SEQ ID NO: 15 and the VL peptide comprises a sequence of SEQ ID NO: 16.

In some embodiments, an antibody, or antigen binding fragment thereof, comprises a VH peptide and a VL peptide, wherein the VH peptide comprises a sequence of SEQ ID NO: 17 and the VL peptide comprises a sequence of SEQ ID NO: 18.

In some embodiments, an antibody, or antigen binding fragment thereof, comprises a VH peptide and a VL peptide, wherein the VH peptide comprises a sequence of SEQ ID NO: 19 and the VL peptide comprises a sequence of SEQ ID NO: 20.

In some embodiments, an antibody, or antigen binding fragment thereof, comprises a VH peptide and a VL peptide, wherein the VH peptide comprises a sequence of SEQ ID NO: 21 and the VL peptide comprises a sequence of SEQ ID NO: 22.

In some embodiments, an antibody, or antigen binding fragment thereof, comprises a VH peptide and a VL peptide, wherein the VH peptide comprises a sequence of SEQ ID NO: 23 and the VL peptide comprises a sequence of SEQ ID NO: 24.

In some embodiments, an antibody, or antigen binding fragment thereof, comprises a VH peptide and a VL peptide, wherein the VH peptide comprises a sequence of SEQ ID NO: 25 and the VL peptide comprises a sequence of SEQ ID NO: 26.

In some embodiments, an antibody, or antigen binding fragment thereof, comprises a VH peptide and a VL peptide, wherein the VH peptide comprises a sequence of SEQ ID NO: 27 and the VL peptide comprises a sequence of SEQ ID NO: 28.

In some embodiments, an antibody, or antigen binding fragment thereof, comprises a VH peptide and a VL peptide, wherein the VH peptide comprises a sequence of SEQ ID NO: 29 and the VL peptide comprises a sequence of SEQ ID NO: 30.

In some embodiments, an antibody, or antigen binding fragment thereof, comprises a VH peptide and a VL peptide, wherein the VH peptide comprises a sequence of SEQ ID NO: 31 and the VL peptide comprises a sequence of SEQ ID NO: 32.

In some embodiments, an antibody, or antigen binding fragment thereof, comprises a VH peptide and a VL peptide, wherein the VH peptide comprises a sequence of SEQ ID NO: 33 and the VL peptide comprises a sequence of SEQ ID NO: 34.

In some embodiments, an antibody, or antigen binding fragment thereof, comprises a VH peptide and a VL peptide, wherein the VH peptide comprises a sequence of SEQ ID NO: 35 and the VL peptide comprises a sequence of SEQ ID NO: 36.

In some embodiments, an antibody, or antigen binding fragment thereof, comprises a VH peptide and a VL peptide, wherein the VH peptide comprises a sequence of SEQ ID NO: 37 and the VL peptide comprises a sequence of SEQ ID NO: 38.

In some embodiments, an antibody, or antigen binding fragment thereof, comprises a VH peptide and a VL peptide, wherein the VH peptide comprises a sequence of SEQ ID NO: 39 and the VL peptide comprises a sequence of SEQ ID NO: 40.

In some embodiments, an antibody, or antigen binding fragment thereof, comprises a VH peptide and a VL peptide, wherein the VH peptide comprises a sequence of SEQ ID NO: 41 and the VL peptide comprises a sequence of SEQ ID NO: 42.

In some embodiments, an antibody, or antigen binding fragment thereof, comprises a VH peptide and a VL peptide, wherein the VH peptide comprises a sequence of SEQ ID NO: 43 and the VL peptide comprises a sequence of SEQ ID NO: 44.

In some embodiments, an antibody, or antigen binding fragment thereof, comprises a VH peptide and a VL peptide, wherein the VH peptide comprises a sequence of SEQ ID NO: 45 and the VL peptide comprises a sequence of SEQ ID NO: 46.

In some embodiments, an antibody, or antigen binding fragment thereof, comprises a VH peptide and a VL peptide, wherein the VH peptide comprises a sequence of SEQ ID NO: 47 and the VL peptide comprises a sequence of SEQ ID NO: 48.

In some embodiments, an antibody, or antigen binding fragment thereof, comprises a VH peptide and a VL peptide, wherein the VH peptide comprises a sequence of SEQ ID NO: 49 and the VL peptide comprises a sequence of SEQ ID NO: 50.

In some embodiments, an antibody, or antigen binding fragment thereof, comprises a VH peptide and a VL peptide, wherein the VH peptide comprises a sequence of SEQ ID NO: 51 and the VL peptide comprises a sequence of SEQ ID NO: 52.

In some embodiments, an antibody, or antigen binding fragment thereof, comprises a VH peptide and a VL peptide, wherein the VH peptide comprises a sequence of SEQ ID NO: 53 and the VL peptide comprises a sequence of SEQ ID NO: 54.

In some embodiments, an antibody, or antigen binding fragment thereof, comprises a VH peptide and a VL peptide, wherein the VH peptide comprises a sequence of SEQ ID NO: 55 and the VL peptide comprises a sequence of SEQ ID NO: 56.

In some embodiments, an antibody, or antigen binding fragment thereof, comprises a VH peptide and a VL peptide, wherein the VH peptide comprises a sequence of SEQ ID NO: 57 and the VL peptide comprises a sequence of SEQ ID NO: 58.

In some embodiments, an antibody, or antigen binding fragment thereof, comprises a VH peptide and a VL peptide, wherein the VH peptide comprises a sequence of SEQ ID NO: 59 and the VL peptide comprises a sequence of SEQ ID NO: 60.

In some embodiments, an antibody, or antigen binding fragment thereof, comprises a VH peptide and a VL peptide, wherein the V_{H} peptide comprises a sequence of SEQ ID NO: 334 and the V_{L} peptide comprises a sequence of SEQ ID NO: 2.

In some embodiments, an antibody, or antigen binding fragment thereof, comprises a VH peptide and a VL peptide, wherein the V_{H} peptide comprises a sequence of SEQ ID NO: 335 and the V_{L} peptide comprises a sequence of SEQ ID NO: 4.

In some embodiments, an antibody, or antigen binding fragment thereof, comprises a VH peptide and a VL peptide, wherein the V_{H} peptide comprises a sequence of SEQ ID NO: 336 and the V_{L} peptide comprises a sequence of SEQ ID NO: 6.

In some embodiments, an antibody, or antigen binding fragment thereof, comprises a VH peptide and a VL peptide, wherein the VH peptide comprises a sequence of SEQ ID NO: 337 and the VL peptide comprises a sequence of SEQ ID NO: 8.

In some embodiments, an antibody, or antigen binding fragment thereof, comprises a VH peptide and a VL peptide, wherein the VH peptide comprises a sequence of SEQ ID NO: 338 and the VL peptide comprises a sequence of SEQ ID NO: 10.

In some embodiments, an antibody, or antigen binding fragment thereof, comprises a VH peptide and a VL peptide, wherein the VH peptide comprises a sequence of SEQ ID NO: 339 and the VL peptide comprises a sequence of SEQ ID NO: 12.

In some embodiments, an antibody, or antigen binding fragment thereof, comprises a VH peptide and a VL peptide, wherein the VH peptide comprises a sequence of SEQ ID NO: 340 and the VL peptide comprises a sequence of SEQ ID NO: 14.

In some embodiments, an antibody, or antigen binding fragment thereof, comprises a VH peptide and a VL peptide, wherein the VH peptide comprises a sequence of SEQ ID NO: 341 and the VL peptide comprises a sequence of SEQ ID NO: 16.

In some embodiments, an antibody, or antigen binding fragment thereof, comprises a VH peptide and a VL peptide, wherein the VH peptide comprises a sequence of SEQ ID NO: 342 and the VL peptide comprises a sequence of SEQ ID NO: 18.

In some embodiments, an antibody, or antigen binding fragment thereof, comprises a VH peptide and a VL peptide, wherein the VH peptide comprises a sequence of SEQ ID NO: 343 and the VL peptide comprises a sequence of SEQ ID NO: 20.

In some embodiments, an antibody, or antigen binding fragment thereof, comprises a VH peptide and a VL peptide, wherein the VH peptide comprises a sequence of SEQ ID NO: 344 and the VL peptide comprises a sequence of SEQ ID NO: 22.

In some embodiments, an antibody, or antigen binding fragment thereof, comprises a VH peptide and a VL peptide, wherein the VH peptide comprises a sequence of SEQ ID NO: 345 and the VL peptide comprises a sequence of SEQ ID NO: 24.

In some embodiments, an antibody, or antigen binding fragment thereof, comprises a VH peptide and a VL peptide, wherein the VH peptide comprises a sequence of SEQ ID NO: 346 and the VL peptide comprises a sequence of SEQ ID NO: 26.

In some embodiments, an antibody, or antigen binding fragment thereof, comprises a VH peptide and a VL peptide, wherein the VH peptide comprises a sequence of SEQ ID NO: 347 and the VL peptide comprises a sequence of SEQ ID NO: 28.

In some embodiments, an antibody, or antigen binding fragment thereof, comprises a VH peptide and a VL peptide, wherein the VH peptide comprises a sequence of SEQ ID NO: 348 and the VL peptide comprises a sequence of SEQ ID NO: 30.

In some embodiments, an antibody, or antigen binding fragment thereof, comprises a VH peptide and a VL peptide, wherein the VH peptide comprises a sequence of SEQ ID NO: 349 and the VL peptide comprises a sequence of SEQ ID NO: 32.

In some embodiments, an antibody, or antigen binding fragment thereof, comprises a VH peptide and a VL peptide, wherein the VH peptide comprises a sequence of SEQ ID NO: 350 and the VL peptide comprises a sequence of SEQ ID NO: 34.

In some embodiments, an antibody, or antigen binding fragment thereof, comprises a VH peptide and a VL peptide, wherein the VH peptide comprises a sequence of SEQ ID NO: 351 and the VL peptide comprises a sequence of SEQ ID NO: 36.

In some embodiments, an antibody, or antigen binding fragment thereof, comprises a VH peptide and a VL peptide, wherein the VH peptide comprises a sequence of SEQ ID NO: 352 and the VL peptide comprises a sequence of SEQ ID NO: 38.

In some embodiments, an antibody, or antigen binding fragment thereof, comprises a VH peptide and a VL peptide, wherein the VH peptide comprises a sequence of SEQ ID NO: 353 and the VL peptide comprises a sequence of SEQ ID NO: 40.

In some embodiments, an antibody, or antigen binding fragment thereof, comprises a VH peptide and a VL peptide, wherein the VH peptide comprises a sequence of SEQ ID NO: 354 and the VL peptide comprises a sequence of SEQ ID NO: 42.

In some embodiments, an antibody, or antigen binding fragment thereof, comprises a VH peptide and a VL peptide, wherein the VH peptide comprises a sequence of SEQ ID NO: 355 and the VL peptide comprises a sequence of SEQ ID NO: 44.

In some embodiments, an antibody, or antigen binding fragment thereof, comprises a VH peptide and a VL peptide, wherein the VH peptide comprises a sequence of SEQ ID NO: 356 and the VL peptide comprises a sequence of SEQ ID NO: 46.

In some embodiments, an antibody, or antigen binding fragment thereof, comprises a VH peptide and a VL peptide, wherein the VH peptide comprises a sequence of SEQ ID NO: 357 and the VL peptide comprises a sequence of SEQ ID NO: 48.

In some embodiments, an antibody, or antigen binding fragment thereof, comprises a VH peptide and a VL peptide, wherein the VH peptide comprises a sequence of SEQ ID NO: 358 and the VL peptide comprises a sequence of SEQ ID NO: 50.

In some embodiments, an antibody, or antigen binding fragment thereof, comprises a VH peptide and a VL peptide, wherein the VH peptide comprises a sequence of SEQ ID NO: 359 and the VL peptide comprises a sequence of SEQ ID NO: 52.

In some embodiments, an antibody, or antigen binding fragment thereof, comprises a VH peptide and a VL peptide, wherein the VH peptide comprises a sequence of SEQ ID NO: 360 and the VL peptide comprises a sequence of SEQ ID NO: 54.

In some embodiments, an antibody, or antigen binding fragment thereof, comprises a VH peptide and a VL peptide, wherein the VH peptide comprises a sequence of SEQ ID NO: 361 and the VL peptide comprises a sequence of SEQ ID NO: 56.

In some embodiments, an antibody, or antigen binding fragment thereof, comprises a VH peptide and a VL peptide, wherein the VH peptide comprises a sequence of SEQ ID NO: 362 and the VL peptide comprises a sequence of SEQ ID NO: 58.

In some embodiments, an antibody, or antigen binding fragment thereof, comprises a VH peptide and a VL peptide, wherein the VH peptide comprises a sequence of SEQ ID NO: 363 and the VL peptide comprises a sequence of SEQ ID NO: 60.

In some embodiments, the antibody comprises a heavy chain as set forth below, which includes the variable region and the constant domain:

TABLE 7

| SEQ ID NO: | AB ID NO. | Heavy Chain Sequence |
|---|---|---|
| 168 | MAB1 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDQEDYALDYWGQGTLVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFEGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVLHEALHSHYTQKSLSLSLG |
| 169 | MAB1 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDQEDYALDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 170 | MAB2 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDETDYGWDYWGQGTLVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFEGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVLHEALHSHYTQKSLSLSLG |
| 171 | MAB2 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDETDYGWDYWGQGTLVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFEGGPSVFLFPPKPKD |
| | | TLYITREPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| 172 | MAB3 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDETDYAFDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 173 | MAB3 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDETDYAFDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 174 | MAB4 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDESQYALDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 175 | MAB4 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDESQYALDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 176 | MAB5 with constant | QVQLVESGGGLVKPGGSLRLSCAASGFTFSDYYMSWIRQAP |
| | domain PE and LS | GKGLEWVSYISRSGSTKYYAHSVKGRFTISRDNAKNSLYLQ |
| | mutations | MNSLRAEDTAVYYCARDNTDYALDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 177 | MAB5 with constant | QVQLVESGGGLVKPGGSLRLSCAASGFTFSDYYMSWIRQAP |
| | domain PE and YTE | GKGLEWVSYISRSGSTKYYAHSVKGRFTISRDNAKNSLYLQ |
| | mutations | MNSLRAEDTAVYYCARDNTDYALDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 178 | MAB6 with constant | QVQLVESGGGLVKPGGSLRLSCAASGFTFSDYYMSWIRQAP |
| | domain PE and LS | GKGLEWVSYISRSGSTKYYAHSVKGRFTISRDNAKNSLYLQ |
| | mutations | MNSLRAEDTAVYYCARDETDYAYDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 179 | MAB6 with constant | QVQLVESGGGLVKPGGSLRLSCAASGFTFSDYYMSWIRQAP |
| | domain PE and YTE | GKGLEWVSYISRSGSTKYYAHSVKGRFTISRDNAKNSLYLQ |
| | mutations | MNSLRAEDTAVYYCARDETDYAYDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 180 | MAB7 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDETDYAYDNWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 181 | MAB7 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDETDYAYDNWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 182 | MAB8 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDESDYAYDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 183 | MAB8 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDESDYAYDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 184 | MAB9 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDENYALDWWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 185 | MAB9 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDENYALDWWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 186 | MAB 10 with constant | QVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDESDYALDFWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 187 | MAB 10 with constant | QVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDESDYALDFWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 188 | MAB11 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 189 | MAB11 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 190 | MAB12 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGLGGRPFDHWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 191 | MAB12 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGLGGRPFDHWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 192 | MAB13 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGLAGFPFDIWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 193 | MAB13 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGLAGFPFDIWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 194 | MAB14 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGLAWRPTDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 195 | MAB14 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGLAWRPTDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 196 | MAB15 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTKGLAWLPYYSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 197 | MAB15 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTKGLAWLPYYSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 198 | MAB 16 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 199 | MAB16 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 200 | MAB17 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGEAGRPFDAWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 201 | MAB17 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGEAGRPFDAWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 202 | MAB18 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSSLRSEDTAVYYCVTEGLAGRPYDVWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 203 | MAB18 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSSLRSEDTAVYYCVTEGLAGRPYDVWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 204 | MAB 19 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 205 | MAB 19 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 206 | MAB20 with constant | QVQLVQSGAEVKKPGASVKVSCKVSGDTLTELSMHWVRQ |
| | domain PE and LS | APGKGLEWMGTFDPEEGETIYAQKFQGRVTMTEDTSTDTA |
| | mutations | YMELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 207 | MAB20 with constant | QVQLVQSGAEVKKPGASVKVSCKVSGDTLTELSMHWVRQ |
| | domain PE and YTE | APGKGLEWMGTFDPEEGETIYAQKFQGRVTMTEDTSTDTA |
| | mutations | YMELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 208 | MAB21 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVLHEALHSHYTQKSLSLSLG |
| 209 | MAB21 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVMHEALHNHYTQKSLSLSLG |
| 210 | MAB22 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGKYYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 211 | MAB22 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGKYYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 212 | MAB23 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGQTYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 213 | MAB23 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGQTYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 214 | MAB24 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVLHEALHSHYTQKSLSLSLG |
| 215 | MAB24 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVMHEALHNHYTQKSLSLSLG |
| 216 | MAB25 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGIKYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVLHEALHSHYTQKSLSLSLG |
| 217 | MAB25 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGIKYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVMHEALHNHYTQKSLSLSLG |
| 218 | MAB26 with constant | EVQLVESGGGVVRPGGSLRLSCAASGFTFDDYGMSWVRQA |
| | domain PE and LS | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 219 | MAB26 with constant | EVQLVESGGGVVRPGGSLRLSCAASGFTFDDYGMSWVRQA |
| | domain PE and YTE | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 220 | MAB27 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGKVYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 221 | MAB27 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGKVYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 222 | MAB28 with constant | EVQLVESGGGVVRPGGSLRLSCAASGFTFDDYGMSWVRQA |
| | domain PE and LS | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 223 | MAB28 with constant | EVQLVESGGGVVRPGGSLRLSCAASGFTFDDYGMSWVRQA |
| | domain PE and YTE | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 224 | MAB29 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGNHYYYYYMDAWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 225 | MAB29 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGNHYYYYYMDAWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 226 | MAB30 with constant | EVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGRHYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 227 | MAB30 with constant | EVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGRHYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 364 | MAB31 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDQEDYALDYWGQGTLVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFEGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVLHEALHSHYTQKSLSLSLG |
| 365 | MAB31 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDQEDYALDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 366 | MAB32 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDETDYGWDYWGQGTLVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFEGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVLHEALHSHYTQKSLSLSLG |
| 367 | MAB32 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDETDYGWDYWGQGTLVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFEGGPSVFLFPPKPKD |
| | | TLYITREPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| 368 | MAB33 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDETDYAFDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 369 | MAB33 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDETDYAFDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 370 | MAB34 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDESQYALDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 371 | MAB34 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDESQYALDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 372 | MAB35 with constant | EVQLVESGGGLVKPGGSLRLSCAASGFTFSDYYMSWIRQAP |
| | domain PE and LS | GKGLEWVSYISRSGSTKYYAHSVKGRFTISRDNAKNSLYLQ |
| | mutations | MNSLRAEDTAVYYCARDNTDYALDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 373 | MAB35 with constant | EVQLVESGGGLVKPGGSLRLSCAASGFTFSDYYMSWIRQAP |
| | domain PE and YTE | GKGLEWVSYISRSGSTKYYAHSVKGRFTISRDNAKNSLYLQ |
| | mutations | MNSLRAEDTAVYYCARDNTDYALDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 374 | MAB36 with constant | EVQLVESGGGLVKPGGSLRLSCAASGFTFSDYYMSWIRQAP |
| | domain PE and LS | GKGLEWVSYISRSGSTKYYAHSVKGRFTISRDNAKNSLYLQ |
| | mutations | MNSLRAEDTAVYYCARDETDYAYDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 375 | MAB36 with constant | EVQLVESGGGLVKPGGSLRLSCAASGFTFSDYYMSWIRQAP |
| | domain PE and YTE | GKGLEWVSYISRSGSTKYYAHSVKGRFTISRDNAKNSLYLQ |
| | mutations | MNSLRAEDTAVYYCARDETDYAYDEWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 376 | MAB37 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDETDYAYDNWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 377 | MAB37 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDETDYAYDNWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 378 | MAB38 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDESDYAYDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 379 | MAB38 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDESDYAYDYWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 380 | MAB39 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDENYALDWWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFEGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLTCLVKGFYP |
| | | SDIAVEWESNGQPENNYKTTPPVLDSDGSFFLYSRLTVDKSR |
| | | WQEGNVFSCSVLHEALHSHYTQKSLSLSLG |
| 381 | MAB39 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDENYALDWWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFEGGPSVFLFPPKPKDTL |
| | | YITREPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLTCLVKGFYP |
| | | SDIAVEWESNGQPENNYKTTPPVLDSDGSFFLYSRLTVDKSR |
| | | WQEGNVFSCSVMHEALHNHYTQKSLSLSLG |
| 382 | MAB40 with constant | EVQLVESGGGLVKPGGSLRLSCAASGFTFSDYYMSWIRQAP |
| | domain PE and LS | GKGLEWVSYISRSGSTKYYADSVKGRFTISRDNAKNSLYLQ |
| | mutations | MNSLRAEDTAVYYCARDESDYALDFWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 383 | MAB40 with constant | EVQLVESGGGLVKPGGSLRLSCAASGFTFSDYYMSWIRQAP |
| | domain PE and YTE | GKGLEWVSYISRSGSTKYYADSVKGRFTISRDNAKNSLYLQ |
| | mutations | MNSLRAEDTAVYYCARDESDYALDFWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 384 | MAB41 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 385 | MAB41 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 386 | MAB42 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGLGGRPFDHWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 387 | MAB42 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGLGGRPFDHWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 388 | MAB43 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGLAGFPFDIWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 389 | MAB43 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGLAGFPFDIWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 390 | MAB44 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGLAWRPTDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 391 | MAB44 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGLAWRPTDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 392 | MAB45 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTKGLAWLPYYSWGQGTLVTVSSA |
| | | STKGPSVFPLAPCSRSTSESTAALGCLVKDYFPEPVTVSWNS |
| | | GALTSGVHTFPAVLQSSGLYSLSSVVTVPSSSLGTKTYTCNV |
| | | DHKPSNTKVDKRVESKYGPPCPPCPAPEFEGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVLHEALHSHYTQKSLSLSLG |
| 393 | MAB45 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTKGLAWLPYYSWGQGTLVTVSSA |
| | | STKGPSVFPLAPCSRSTSESTAALGCLVKDYFPEPVTVSWNS |
| | | GALTSGVHTFPAVLQSSGLYSLSSVVTVPSSSLGTKTYTCNV |
| | | DHKPSNTKVDKRVESKYGPPCPPCPAPEFEGGPSVFLFPPKP |
| | | KDTLYITREPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVMHEALHNHYTQKSLSLSLG |
| 394 | MAB46 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 395 | MAB46 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGLAGVPFDLWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 396 | MAB47 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGEAGRPFDAWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 397 | MAB47 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGEAGRPFDAWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 398 | MAB48 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSSLRSEDTAVYYCVTEGLAGRPYDVWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 399 | MAB48 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSSLRSEDTAVYYCVTEGLAGRPYDVWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 400 | MAB49 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 401 | MAB49 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 402 | MAB50 with constant | EVQLVQSGAEVKKPGASVKVSCKVSGDTLTELSMHWVRQA |
| | domain PE and LS | PGKGLEWMGTFDPEEGETIYAQKFQGRVTMTEDTSTDTAY |
| | mutations | MELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLMISRTPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVLHEALHSHYTQKSLSLSLG |
| 403 | MAB50 with constant | EVQLVQSGAEVKKPGASVKVSCKVSGDTLTELSMHWVRQA |
| | domain PE and YTE | PGKGLEWMGTFDPEEGETIYAQKFQGRVTMTEDTSTDTAY |
| | mutations | MELSYLRSEDTAVYYCVTEGLAGIPFDSWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFEGGPSVFLFPPK |
| | | PKDTLYITREPEVTCVVVDVSQEDPEVQFNWYVDGVEVHN |
| | | AKTKPREEQFNSTYRVVSVLTVLHQDWLNGKEYKCKVSNK |
| | | GLPSSIEKTISKAKGQPREPQVYTLPPSQEEMTKNQVSLTCLV |
| | | KGFYPSDIAVEWESNGQPENNYKTTPPVLDSDGSFFLYSRLT |
| | | VDKSRWQEGNVFSCSVMHEALHNHYTQKSLSLSLG |
| 404 | MAB51 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVLHEALHSHYTQKSLSLSLG |
| 405 | MAB51 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVMHEALHNHYTQKSLSLSLG |
| 406 | MAB52 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGKYYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 407 | MAB52 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGKYYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 408 | MAB53 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGQTYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 409 | MAB53 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGQTYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 410 | MAB54 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVLHEALHSHYTQKSLSLSLG |
| 411 | MAB54 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGTTYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVMHEALHNHYTQKSLSLSLG |
| 412 | MAB55 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGIKYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVLHEALHSHYTQKSLSLSLG |
| 413 | MAB55 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGIKYYYYYMDVWGKGTT |
| | | VTVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEFEGGPSVFL |
| | | FPPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFFLYS |
| | | RLTVDKSRWQEGNVFSCSVMHEALHNHYTQKSLSLSLG |
| 414 | MAB56 with constant | QVQLVESGGGVVRPGGSLRLSCAASGFTFDDYGMSWVRQA |
| | domain PE and LS | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 415 | MAB56 with constant | QVQLVESGGGVVRPGGSLRLSCAASGFTFDDYGMSWVRQA |
| | domain PE and YTE | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 416 | MAB57 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGKVYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 417 | MAB57 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGKVYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 418 | MAB58 with constant | QVQLVESGGGVVRPGGSLRLSCAASGFTFDDYGMSWVRQA |
| | domain PE and LS | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 419 | MAB58 with constant | QVQLVESGGGVVRPGGSLRLSCAASGFTFDDYGMSWVRQA |
| | domain PE and YTE | PGKGLEWVSGINWEGGSTGYADSVKGRFSISRDNAKNSLYL |
| | mutations | QMNSLRAEDTALYYCARDEQLGGLKYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 420 | MAB59 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGNHYYYYYMDAWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 421 | MAB59 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGNHYYYYYMDAWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |
| 422 | MAB60 with constant | QVQLVESGGGVVRPGGSLRLSCAASGFTFDDYGMSWVRQA |
| | domain PE and LS | PGKGLEWVSGINWEGGSTGYADSVKGRFTISRDNAKNSLYL |
| | mutations | QMNSLRAEDTALYYCARDEQLGGRHYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVLHEALHSHYTQKSLSLSLG |
| 423 | MAB60 with constant | QVQLVESGGGVVRPGGSLRLSCAASGFTFDDYGMSWVRQA |
| | domain PE and YTE | PGKGLEWVSGINWEGGSTGYADSVKGRFTISRDNAKNSLYL |
| | mutations | QMNSLRAEDTALYYCARDEQLGGRHYYYYYMDVWGKGT |
| | | TVTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFEGGPSVF |
| | | LFPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFFL |
| | | YSRLTVDKSRWQEGNVFSCSVMHEALHNHYTQKSLSLSLG |

In some embodiments, the antibody comprises a light chain as set forth below, which includes the variable region and a constant domain, such as the human kappa constant domain:

TABLE 8

| SEQ | | |
|---|---|---|
| ID | | |
| NO: | AB ID NO. | Light Chain Sequence |
| 228 | MAB1/MAB31 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCEQYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 229 | MAB2/MAB32 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCKQYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 230 | MAB3/MAB33 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCKQYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 231 | MAB4/MAB34 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCHQYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 232 | MAB5/MAB35 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCHQYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 233 | MAB6//MAB36 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCHQYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 234 | MAB7//MAB37 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCQQYEDYPLVFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 235 | MAB8//MAB38 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCQQHEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 236 | MAB9/MAB39 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCQHYEDYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 237 | MAB10/MAB40 plus | DIQMTQSPSSLSASVGDRVTITCQASQDISNYLNWYQQKPG |
| | kappa constant | KAPKLLIYDASNLETGVPSRFSGSGSGTDFTFTISSLQPEDIAT |
| | domain | YYCQQYEDLIPTFGGGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 238 | MAB11//MAB41 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQSYNSYVWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 239 | MAB12/MAB42 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQVNSYDWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 240 | MAB13//MAB43 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQVNSYDWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 241 | MAB14/MAB44 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQYSSYAWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 242 | MAB15/MAB45 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQARSYSWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 243 | MAB16/MAB46 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQHNSYRWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 244 | MAB17/MAB47 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCVQYPSYSWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 245 | MAB18/MAB48 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQYNSYKLTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 246 | MAB19//MAB49 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQYNSPSWLFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 247 | MAB20/MAB50 plus | DIQMTQSPSTLSASVGDRVTITCRASQSISSWLAWYQQKPGK |
| | kappa constant | APKLLIYKASSLESGVPSRFSGSGSGTEFTLTISSLQPDDFATY |
| | domain | YCQQVNSLSWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 248 | MAB21/MAB51 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCFQYNSYPLGFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 249 | MAB22/MAB52 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYRSHPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 250 | MAB23/MAB53 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYNQVPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 251 | MAB24/MAB54 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQTNIYPLTFGGGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 252 | MAB25/MAB55 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYKQYPLTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 253 | MAB26/MAB56 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQTNIYPLTFGGGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 254 | MAB27/MAB57 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYNSYPLAFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 255 | MAB28/MAB58 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYHSYPLRFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 256 | MAB29/MAB59 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYASYPLKFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 257 | MAB30/MAB60 plus | DIQMTQSPSSLSASVGDRVTITCRASQGIRNDLGWYQQKPG |
| | kappa constant | KAPKRLIYTASNLQSGVPSRFSGSGSGTEFTLTISSLQPEDFAT |
| | domain | YYCLQYNAYPLIFGGGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |

In some embodiments, the antibody, or antigen binding fragment thereof, of any of the embodiments as provided for herein may comprise any constant domain known, such as but not limited to an IgG constant domain. In some embodiments, the constant domain is as provided for herein. In some non-limiting embodiments, the constant domain is selected from the group including, but not limited to, SEQ ID NO: 261, 262, 263, 264, 265, or a variant thereof as provided for herein. In some embodiments, the constant domain has at least 90, 91, 92, 93, 94, 95, 96, 97, 98, or 99% sequence identity to a sequence selected from the group including, but not limited to, SEQ ID NO: 261, 262, 263, 264, 265, or a variant thereof as provided for herein. In some embodiments, the constant domain has at least 90, 91, 92, 93, 94, 95, 96, 97, 98, or 99% sequence identity to a sequence selected from the group including, but not limited to, SEQ ID NO: 261, 262, 263, 264, 265, provided that the constant domain comprises the PE, YTE, and/or LS mutations as described herein.

In some embodiments, the antibody or antigen-binding fragment thereof, comprises a light chain that has at least 90, 91, 92, 93, 94, 95, 96, 97, 98, 99% sequence identity to SEQ ID NO: 228, and a heavy chain that has at least 90, 91, 92, 93, 94, 95, 96, 97, 98, or 99% sequence identity to SEQ ID NO: 168 or SEQ ID NO: 169 (HC of MAB1), wherein the antibody, or antigen binding fragment thereof, maintains the sequences of the heavy chain and light chain CDRs of MAB1. In some embodiments, the antibody or antigen-binding fragment thereof, comprises a light chain that has at least 90, 91, 92, 93, 94, 95, 96, 97, 98, or 99% sequence identity to SEQ ID NO: 228, and a heavy chain that has at least 90, 91, 92, 93, 94, 95, 96, 97, 98, or 99% sequence identity to SEQ ID NO: 168 or SEQ ID NO: 169, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66.

In some embodiments, the antibody or antigen-binding fragment thereof, comprises a light chain that has at least 90, 91, 92, 93, 94, 95, 96, 97, 98, or 99% sequence identity to SEQ ID NO: 229, and a heavy chain that has at least 90, 91, 92, 93, 94, 95, 96, 97, 98, or 99% sequence identity to SEQ ID NO: 170 or SEQ ID NO: 171 (HC of MAB2), wherein the antibody, or antigen binding fragment thereof, maintains the sequences of the heavy chain and light chain CDRs of MAB2. In some embodiments, the antibody or antigen-binding fragment thereof, comprises a light chain that has at least 90, 91, 92, 93, 94, 95, 96, 97, 98, or 99% sequence identity to SEQ ID NO: 229, and a heavy chain that has at least 90, 91, 92, 93, 94, 95, 96, 97, 98, or 99% sequence identity to SEQ ID NO: 170 or SEQ ID NO: 171, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof, comprises a light chain that has at least 90, 91, 92, 93, 94, 95, 96, 97, 98, or 99% sequence identity to SEQ ID NO: 230, and a heavy chain that has at least 90, 91, 92, 93, 94, 95, 96, 97, 98, or 99% sequence identity to SEQ ID NO: 172 or SEQ ID NO: 173 (HC of MAB3), wherein the antibody, or antigen binding fragment thereof, maintains the sequences of the heavy chain and light chain CDRs of MAB3. In some embodiments, the antibody or antigen-binding fragment thereof, comprises a light chain that has at least 90, 91, 92, 93, 94, 95, 96, 97, 98, or 99% sequence identity to SEQ ID NO: 230, and a heavy chain that has at least 90, 91, 92, 93, 94, 95, 96, 97, 98, or 99% sequence identity to SEQ ID NO: 172 or SEQ ID NO: 173, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof, comprises a light chain that has at least 90, 91, 92, 93, 94, 95, 96, 97, 98, or 99% sequence identity to SEQ ID NO: 231, and a heavy chain that has at least 90, 91, 92, 93, 94, 95, 96, 97, 98, or 99% sequence identity to SEQ ID NO: 174 or SEQ ID NO: 175 (HC of MAB4), wherein the antibody, or antigen binding fragment thereof, maintains the sequences of the heavy chain and light chain CDRs of MAB4. In some embodiments, the antibody or antigen-binding fragment thereof, comprises a light chain that has at least 90, 91, 92, 93, 94, 95, 96, 97, 98, or 99% sequence identity to SEQ ID NO: 231, and a heavy chain that has at least 90, 91, 92, 93, 94, 95, 96, 97, 98, or 99% sequence identity to SEQ ID NO: 174 or SEQ ID NO: 175, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof, comprises a light chain that has at least 90, 91, 92, 93, 94, 95, 96, 97, 98, or 99% sequence identity to SEQ ID NO: 232, and a heavy chain that has at least 90, 91, 92, 93, 94, 95, 96, 97, 98, or 99% sequence identity to SEQ ID NO: 176 or SEQ ID NO: 177 (HC of MAB5), wherein the antibody, or antigen binding fragment thereof, maintains the sequences of the heavy chain and light chain CDRs of MAB5. In some embodiments, the antibody or antigen-binding fragment thereof, comprises a light chain that has at least 90, 91, 92, 93, 94, 95, 96, 97, 98, or 99% sequence identity to SEQ ID NO: 232, and a heavy chain that has at least 90, 91, 92, 93, 94, 95, 96, 97, 98, or 99% sequence identity to SEQ ID NO: 176 or SEQ ID NO: 177, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof, comprises a light chain that has at least 90, 91, 92, 93, 94, 95, 96, 97, 98, or 99% sequence identity to SEQ ID NO: 233, and a heavy chain that has at least 90, 91, 92, 93, 94, 95, 96, 97, 98, or 99% sequence identity to SEQ ID NO: 178 or SEQ ID NO: 179 (HC of MAB6), wherein the antibody, or antigen binding fragment thereof, maintains the sequences of the heavy chain and light chain CDRs of MAB6. In some embodiments, the antibody or antigen-binding fragment thereof, comprises a light chain that has at least 90, 91, 92, 93, 94, 95, 96, 97, 98, or 99% sequence identity to SEQ ID NO: 233, and a heavy chain that has at least 90, 91, 92, 93, 94, 95, 96, 97, 98, or 99% sequence identity to SEQ ID NO: 178 or SEQ ID NO: 179, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof, comprises a light chain that has at least 90, 91, 92, 93, 94, 95, 96, 97, 98, or 99% sequence identity to SEQ ID NO: 234, and a heavy chain that has at least 90, 91, 92, 93, 94, 95, 96, 97, 98, or 99% sequence identity to SEQ ID NO: 180 or SEQ ID NO: 181 (HC of MAB7), wherein the antibody, or antigen binding fragment thereof, maintains the sequences of the heavy chain and light chain CDRs of MAB7. In some embodiments, the antibody or antigen-binding fragment thereof, comprises a light chain that has at least 90, 91, 92, 93, 94, 95, 96, 97, 98, or 99% sequence identity to SEQ ID NO: 234, and a heavy chain that has at least 90, 91, 92, 93, 94, 95, 96, 97, 98, or 99% sequence identity to SEQ ID NO: 180 or SEQ ID NO: 181, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the antibody or antigen-binding fragment thereof, comprises a light chain that has at least 90, 91, 92, 93, 94, 95, 96, 97, 98, or 99% sequence identity to SEQ ID NO: 235, and a heavy chain that has at least 90, 91, 92, 93, 94, 95, 96, 97, 98, or 99% sequence identity to SEQ ID NO: 182 or SEQ ID NO: 183 (HC of MAB8), wherein the antibody, or antigen binding fragment thereof, maintains the sequences of the heavy chain and light chain CDRs of MAB8. In some embodiments, the antibody or antigen-binding fragment thereof, comprises a light chain that has at least 90, 91, 92, 93, 94, 95, 96, 97, 98, or 99% sequence identity to SEQ ID NO: 235, and a heavy chain that has at least 90, 91, 92, 93, 94, 95, 96, 97, 98, or 99% sequence identity to SEQ ID NO: 182 or SEQ ID NO: 183, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the antibody or antigen-binding fragment thereof, comprises a light chain that has at least 90, 91, 92, 93, 94, 95, 96, 97, 98, or 99% sequence identity to SEQ ID NO: 236, and a heavy chain that has at least 90, 91, 92, 93, 94, 95, 96, 97, 98, or 99% sequence identity to SEQ ID NO: 184 or SEQ ID NO: 185 (HC of MAB9), wherein the antibody, or antigen binding fragment thereof, maintains the sequences of the heavy chain and light chain CDRs of MAB9. In some embodiments, the antibody or antigen-binding fragment thereof, comprises a light chain that has at least 90, 91, 92, 93, 94, 95, 96, 97, 98, or 99% sequence identity to SEQ ID NO: 236, and a heavy chain that has at least 90, 91, 92, 93, 94, 95, 96, 97, 98, or 99% sequence identity to SEQ ID NO: 184 or SEQ ID NO: 185, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the antibody or antigen-binding fragment thereof, comprises a light chain that has at least 90, 91, 92, 93, 94, 95, 96, 97, 98, or 99% sequence identity to SEQ ID NO: 237, and a heavy chain that has at least 90, 91, 92, 93, 94, 95, 96, 97, 98, or 99% sequence identity to SEQ ID NO: 186 or SEQ ID NO: 187 (HC of MAB10), wherein the antibody, or antigen binding fragment thereof, maintains the sequences of the heavy chain and light chain CDRs of MAB10. In some embodiments, the antibody or antigen-binding fragment thereof, comprises a light chain that has at least 90, 91, 92, 93, 94, 95, 96, 97, 98, or 99% sequence identity to SEQ ID NO: 237, and a heavy chain that has at least 90, 91, 92, 93, 94, 95, 96, 97, 98, or 99% sequence identity to SEQ ID NO: 186 or SEQ ID NO: 187, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the antibody or antigen-binding fragment thereof, comprises a light chain that has at least 90, 91, 92, 93, 94, 95, 96, 97, 98, or 99% sequence identity to SEQ ID NO: 238, and a heavy chain that has at least 90, 91, 92, 93, 94, 95, 96, 97, 98, or 99% sequence identity to SEQ ID NO: 188 or SEQ ID NO: 189 (HC of MAB11), wherein the antibody, or antigen binding fragment thereof, maintains the sequences of the heavy chain and light chain CDRs of MAB11. In some embodiments, the antibody or antigen-binding fragment thereof, comprises a light chain that has at least 90, 91, 92, 93, 94, 95, 96, 97, 98, or 99% sequence identity to SEQ ID NO: 238, and a heavy chain that has at least 90, 91, 92, 93, 94, 95, 96, 97, 98, or 99% sequence identity to SEQ ID NO: 188 or SEQ ID NO: 189, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the antibody or antigen-binding fragment thereof, comprises a light chain that has at least 90, 91, 92, 93, 94, 95, 96, 97, 98, or 99% sequence identity to SEQ ID NO: 239, and a heavy chain that has at least 90, 91, 92, 93, 94, 95, 96, 97, 98, or 99% sequence identity to SEQ ID NO: 190 or SEQ ID NO: 191 (HC of MAB12), wherein the antibody, or antigen binding fragment thereof, maintains the sequences of the heavy chain and light chain CDRs of MAB12. In some embodiments, the antibody or antigen-binding fragment thereof, comprises a light chain that has at least 90, 91, 92, 93, 94, 95, 96, 97, 98, or 99% sequence identity to SEQ ID NO: 239, and a heavy chain that has at least 90, 91, 92, 93, 94, 95, 96, 97, 98, or 99% sequence identity to SEQ ID NO: 190 or SEQ ID NO: 191, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof, comprises a light chain that has at least 90, 91, 92, 93, 94, 95, 96, 97, 98, or 99% sequence identity to SEQ ID NO: 240, and a heavy chain that has at least 90, 91, 92, 93, 94, 95, 96, 97, 98, or 99% sequence identity to SEQ ID NO: 192 or SEQ ID NO: 193 (HC of MAB13), wherein the antibody, or antigen binding fragment thereof, maintains the sequences of the heavy chain and light chain CDRs of MAB13. In some embodiments, the antibody or antigen-binding fragment thereof, comprises a light chain that has at least 90, 91, 92, 93, 94, 95, 96, 97, 98, or 99% sequence identity to SEQ ID NO: 240, and a heavy chain that has at least 90, 91, 92, 93, 94, 95, 96, 97, 98, or 99% sequence identity to SEQ ID NO: 192 or SEQ ID NO: 193, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof, comprises a light chain that has at least 90, 91, 92, 93, 94, 95, 96, 97, 98, or 99% sequence identity to SEQ ID NO: 241, and a heavy chain that has at least 90, 91, 92, 93, 94, 95, 96, 97, 98, or 99% sequence identity to SEQ ID NO: 194 or SEQ ID NO: 195 (HC of MAB14), wherein the antibody, or antigen binding fragment thereof, maintains the sequences of the heavy chain and light chain CDRs of MAB14. In some embodiments, the antibody or antigen-binding fragment thereof, comprises a light chain that has at least 90, 91, 92, 93, 94, 95, 96, 97, 98, or 99% sequence identity to SEQ ID NO: 241, and a heavy chain that has at least 90, 91, 92, 93, 94, 95, 96, 97, 98, or 99% sequence identity to SEQ ID NO: 194 or SEQ ID NO: 195, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the antibody or antigen-binding fragment thereof, comprises a light chain that has at least 90, 91, 92, 93, 94, 95, 96, 97, 98, or 99% sequence identity to SEQ ID NO: 242, and a heavy chain that has at least 90, 91, 92, 93, 94, 95, 96, 97, 98, or 99% sequence identity to SEQ ID NO: 196 or SEQ ID NO: 197 (HC of MAB15), wherein the antibody, or antigen binding fragment thereof, maintains the sequences of the heavy chain and light chain CDRs of MAB15. In some embodiments, the antibody or antigen-binding fragment thereof, comprises a light chain that has at least 90, 91, 92, 93, 94, 95, 96, 97, 98, or 99% sequence identity to SEQ ID NO: 242, and a heavy chain that has at least 90, 91, 92, 93, 94, 95, 96, 97, 98, or 99% sequence identity to SEQ ID NO: 196 or SEQ ID NO: 197, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the antibody or antigen-binding fragment thereof, comprises a light chain that has at least 90, 91, 92, 93, 94, 95, 96, 97, 98, or 99% sequence identity to SEQ ID NO: 243, and a heavy chain that has at least 90, 91, 92, 93, 94, 95, 96, 97, 98, or 99% sequence identity to SEQ ID NO: 198 or SEQ ID NO: 199 (HC of MAB16), wherein the antibody, or antigen binding fragment thereof, maintains the sequences of the heavy chain and light chain CDRs of MAB16. In some embodiments, the antibody or antigen-binding fragment thereof, comprises a light chain that has at least 90, 91, 92, 93, 94, 95, 96, 97, 98, or 99% sequence identity to SEQ ID NO: 243, and a heavy chain that has at least 90, 91, 92, 93, 94, 95, 96, 97, 98, or 99% sequence identity to SEQ ID NO: 198 or SEQ ID NO: 199, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the antibody or antigen-binding fragment thereof, comprises a light chain that has at least 90, 91, 92, 93, 94, 95, 96, 97, 98, or 99% sequence identity to SEQ ID NO: 244, and a heavy chain that has at least 90, 91, 92, 93, 94, 95, 96, 97, 98, or 99% sequence identity to SEQ ID NO: 200 or SEQ ID NO: 201 (HC of MAB17), wherein the antibody, or antigen binding fragment thereof, maintains the sequences of the heavy chain and light chain CDRs of MAB17. In some embodiments, the antibody or antigen-binding fragment thereof, comprises a light chain that has at least 90, 91, 92, 93, 94, 95, 96, 97, 98, or 99% sequence identity to SEQ ID NO: 244, and a heavy chain that has at least 90, 91, 92, 93, 94, 95, 96, 97, 98, or 99% sequence identity to SEQ ID NO: 200 or SEQ ID NO: 201, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the antibody or antigen-binding fragment thereof, comprises a light chain that has at least 90, 91, 92, 93, 94, 95, 96, 97, 98, or 99% sequence identity to SEQ ID NO: 245, and a heavy chain that has at least 90, 91, 92, 93, 94, 95, 96, 97, 98, or 99% sequence identity to SEQ ID NO: 202 or SEQ ID NO: 203 (HC of MAB18), wherein the antibody, or antigen binding fragment thereof, maintains the sequences of the heavy chain and light chain CDRs of MAB18. In some embodiments, the antibody or antigen-binding fragment thereof, comprises a light chain that has at least 90, 91, 92, 93, 94, 95, 96, 97, 98, or 99% sequence identity to SEQ ID NO: 245, and a heavy chain that has at least 90, 91, 92, 93, 94, 95, 96, 97, 98, or 99% sequence identity to SEQ ID NO: 202 or SEQ ID NO: 203, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the antibody or antigen-binding fragment thereof, comprises a light chain that has at least 90, 91, 92, 93, 94, 95, 96, 97, 98, or 99% sequence identity to SEQ ID NO: 246, and a heavy chain that has at least 90, 91, 92, 93, 94, 95, 96, 97, 98, or 99% sequence identity to SEQ ID NO: 204 or SEQ ID NO: 205 (HC of MAB19), wherein the antibody, or antigen binding fragment thereof, maintains the sequences of the heavy chain and light chain CDRs of MAB19. In some embodiments, the antibody or antigen-binding fragment thereof, comprises a light chain that has at least 90, 91, 92, 93, 94, 95, 96, 97, 98, or 99% sequence identity to SEQ ID NO: 246, and a heavy chain that has at least 90, 91, 92, 93, 94, 95, 96, 97, 98, or 99% sequence identity to SEQ ID NO: 204 or SEQ ID NO: 205, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the antibody or antigen-binding fragment thereof, comprises a light chain that has at least 90, 91, 92, 93, 94, 95, 96, 97, 98, or 99% sequence identity to SEQ ID NO: 247, and a heavy chain that has at least 90, 91, 92, 93, 94, 95, 96, 97, 98, or 99% sequence identity to SEQ ID NO: 206 or SEQ ID NO: 207 (HC of MAB20), wherein the antibody, or antigen binding fragment thereof, maintains the sequences of the heavy chain and light chain CDRs of MAB20. In some embodiments, the antibody or antigen-binding fragment thereof, comprises a light chain that has at least 90, 91, 92, 93, 94, 95, 96, 97, 98, or 99% sequence identity to SEQ ID NO: 247, and a heavy chain that has at least 90, 91, 92, 93, 94, 95, 96, 97, 98, or 99% sequence identity to SEQ ID NO: 206 or SEQ ID NO: 207, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the antibody or antigen-binding fragment thereof, comprises a light chain that has at least 90, 91, 92, 93, 94, 95, 96, 97, 98, or 99% sequence identity to SEQ ID NO: 248, and a heavy chain that has at least 90, 91, 92, 93, 94, 95, 96, 97, 98, or 99% sequence identity to SEQ ID NO: 208 or SEQ ID NO: 209 (HC of MAB21), wherein the antibody, or antigen binding fragment thereof, maintains the sequences of the heavy chain and light chain CDRs of MAB21. In some embodiments, the antibody or antigen-binding fragment thereof, comprises a light chain that has at least 90, 91, 92, 93, 94, 95, 96, 97, 98, or 99% sequence identity to SEQ ID NO: 248, and a heavy chain that has at least 90, 91, 92, 93, 94, 95, 96, 97, 98, or 99% sequence identity to SEQ ID NO: 208 or SEQ ID NO: 209, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the antibody or antigen-binding fragment thereof, comprises a light chain that has at least 90, 91, 92, 93, 94, 95, 96, 97, 98, or 99% sequence identity to SEQ ID NO: 249, and a heavy chain that has at least 90, 91, 92, 93, 94, 95, 96, 97, 98, or 99% sequence identity to SEQ ID NO: 210 or SEQ ID NO: 211 (HC of MAB22), wherein the antibody, or antigen binding fragment thereof, maintains the sequences of the heavy chain and light chain CDRs of MAB22. In some embodiments, the antibody or antigen-binding fragment thereof, comprises a light chain that has at least 90, 91, 92, 93, 94, 95, 96, 97, 98, or 99% sequence identity to SEQ ID NO: 249, and a heavy chain that has at least 90, 91, 92, 93, 94, 95, 96, 97, 98, or 99% sequence identity to SEQ ID NO: 210 or SEQ ID NO: 211, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the antibody or antigen-binding fragment thereof, comprises a light chain that has at least 90, 91, 92, 93, 94, 95, 96, 97, 98, or 99% sequence identity to SEQ ID NO: 250, and a heavy chain that has at least 90, 91, 92, 93, 94, 95, 96, 97, 98, or 99% sequence identity to SEQ ID NO: 212 or SEQ ID NO: 213 (HC of MAB23), wherein the antibody, or antigen binding fragment thereof, maintains the sequences of the heavy chain and light chain CDRs of MAB23. In some embodiments, the antibody or antigen-binding fragment thereof, comprises a light chain that has at least 90, 91, 92, 93, 94, 95, 96, 97, 98, or 99% sequence identity to SEQ ID NO: 250, and a heavy chain that has at least 90, 91, 92, 93, 94, 95, 96, 97, 98, or 99% sequence identity to SEQ ID NO: 212 or SEQ ID NO: 213, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the antibody or antigen-binding fragment thereof, comprises a light chain that has at least 90, 91, 92, 93, 94, 95, 96, 97, 98, or 99% sequence identity to SEQ ID NO: 251, and a heavy chain that has at least 90, 91, 92, 93, 94, 95, 96, 97, 98, or 99% sequence identity to SEQ ID NO: 214 or SEQ ID NO: 215 (HC of MAB24), wherein the antibody, or antigen binding fragment thereof, maintains the sequences of the heavy chain and light chain CDRs of MAB24. In some embodiments, the antibody or antigen-binding fragment thereof, comprises a light chain that has at least 90, 91, 92, 93, 94, 95, 96, 97, 98, or 99% sequence identity to SEQ ID NO: 251, and a heavy chain that has at least 90, 91, 92, 93, 94, 95, 96, 97, 98, or 99% sequence identity to SEQ ID NO: 214 or SEQ ID NO: 215, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof, comprises a light chain that has at least 90, 91, 92, 93, 94, 95, 96, 97, 98, or 99% sequence identity to SEQ ID NO: 252, and a heavy chain that has at least 90, 91, 92, 93, 94, 95, 96, 97, 98, or 99% sequence identity to SEQ ID NO: 216 or SEQ ID NO: 217 (HC of MAB25), wherein the antibody, or antigen binding fragment thereof, maintains the sequences of the heavy chain and light chain CDRs of MAB25. In some embodiments, the antibody or antigen-binding fragment thereof, comprises a light chain that has at least 90, 91, 92, 93, 94, 95, 96, 97, 98, or 99% sequence identity to SEQ ID NO: 252, and a heavy chain that has at least 90, 91, 92, 93, 94, 95, 96, 97, 98, or 99% sequence identity to SEQ ID NO: 216 or SEQ ID NO: 217, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the antibody or antigen-binding fragment thereof, comprises a light chain that has at least 90, 91, 92, 93, 94, 95, 96, 97, 98, or 99% sequence identity to SEQ ID NO: 253, and a heavy chain that has at least 90, 91, 92, 93, 94, 95, 96, 97, 98, or 99% sequence identity to SEQ ID NO: 218 or SEQ ID NO: 219 (HC of MAB26), wherein the antibody, or antigen binding fragment thereof, maintains the sequences of the heavy chain and light chain CDRs of MAB26. In some embodiments, the antibody or antigen-binding fragment thereof, comprises a light chain that has at least 90, 91, 92, 93, 94, 95, 96, 97, 98, or 99% sequence identity to SEQ ID NO: 253, and a heavy chain that has at least 90, 91, 92, 93, 94, 95, 96, 97, 98, or 99% sequence identity to SEQ ID NO: 218 or SEQ ID NO: 219, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof, comprises a light chain that has at least 90, 91, 92, 93, 94, 95, 96, 97, 98, or 99% sequence identity to SEQ ID NO: 254, and a heavy chain that has at least 90, 91, 92, 93, 94, 95, 96, 97, 98, or 99% sequence identity to SEQ ID NO: 220 or SEQ ID NO: 221 (HC of MAB27), wherein the antibody, or antigen binding fragment thereof, maintains the sequences of the heavy chain and light chain CDRs of MAB27. In some embodiments, the antibody or antigen-binding fragment thereof, comprises a light chain that has at least 90, 91, 92, 93, 94, 95, 96, 97, 98, or 99% sequence identity to SEQ ID NO: 254, and a heavy chain that has at least 90, 91, 92, 93, 94, 95, 96, 97, 98, or 99% sequence identity to SEQ ID NO: 220 or SEQ ID NO: 221, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the antibody or antigen-binding fragment thereof, comprises a light chain that has at least 90, 91, 92, 93, 94, 95, 96, 97, 98, or 99% sequence identity to SEQ ID NO: 255, and a heavy chain that has at least 90, 91, 92, 93, 94, 95, 96, 97, 98, or 99% sequence identity to SEQ ID NO: 222 or SEQ ID NO: 223 (HC of MAB28), wherein the antibody, or antigen binding fragment thereof, maintains the sequences of the heavy chain and light chain CDRs of MAB28. In some embodiments, the antibody or antigen-binding fragment thereof, comprises a light chain that has at least 90, 91, 92, 93, 94, 95, 96, 97, 98, or 99% sequence identity to SEQ ID NO: 255, and a heavy chain that has at least 90, 91, 92, 93, 94, 95, 96, 97, 98, or 99% sequence identity to SEQ ID NO: 222 or SEQ ID NO: 223, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the antibody or antigen-binding fragment thereof, comprises a light chain that has at least 90, 91, 92, 93, 94, 95, 96, 97, 98, or 99% sequence identity to SEQ ID NO: 256, and a heavy chain that has at least 90, 91, 92, 93, 94, 95, 96, 97, 98, or 99% sequence identity to SEQ ID NO: 224 or SEQ ID NO: 225 (HC of MAB29), wherein the antibody, or antigen binding fragment thereof, maintains the sequences of the heavy chain and light chain CDRs of MAB29. In some embodiments, the antibody or antigen-binding fragment thereof, comprises a light chain that has at least 90, 91, 92, 93, 94, 95, 96, 97, 98, or 99% sequence identity to SEQ ID NO: 256, and a heavy chain that has at least 90, 91, 92, 93, 94, 95, 96, 97, 98, or 99% sequence identity to SEQ ID NO: 224 or SEQ ID NO: 225, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the antibody or antigen-binding fragment thereof, comprises a light chain that has at least 90, 91, 92, 93, 94, 95, 96, 97, 98, or 99% sequence identity to SEQ ID NO: 257, and a heavy chain that has at least 90, 91, 92, 93, 94, 95, 96, 97, 98, or 99% sequence identity to SEQ ID NO: 226 or SEQ ID NO: 227 (HC of MAB30), wherein the antibody, or antigen binding fragment thereof, maintains the sequences of the heavy chain and light chain CDRs of MAB30. In some embodiments, the antibody or antigen-binding fragment thereof, comprises a light chain that has at least 90, 91, 92, 93, 94, 95, 96, 97, 98, or 99% sequence identity to SEQ ID NO: 257, and a heavy chain that has at least 90, 91, 92, 93, 94, 95, 96, 97, 98, or 99% sequence identity to SEQ ID NO: 226 or SEQ ID NO: 227, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

In some embodiments, the antibody or antigen-binding fragment thereof, comprises a light chain that has at least 90, 91, 92, 93, 94, 95, 96, 97, 98, 99% sequence identity to SEQ ID NO: 228, and a heavy chain that has at least 90, 91, 92, 93, 94, 95, 96, 97, 98, or 99% sequence identity to SEQ ID NO: 364 or SEQ ID NO: 365 (HC of MAB31), wherein the antibody, or antigen binding fragment thereof, maintains the sequences of the heavy chain and light chain CDRs of MAB31. In some embodiments, the antibody or antigen-binding fragment thereof, comprises a light chain that has at least 90, 91, 92, 93, 94, 95, 96, 97, 98, or 99% sequence identity to SEQ ID NO: 228, and a heavy chain that has at least 90, 91, 92, 93, 94, 95, 96, 97, 98, or 99% sequence identity to SEQ ID NO: 364 or SEQ ID NO: 365, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 63 or SEQ ID NO: 132, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence of SEQ ID NO: 66.

In some embodiments, the antibody or antigen-binding fragment thereof, comprises a light chain that has at least 90, 91, 92, 93, 94, 95, 96, 97, 98, or 99% sequence identity to SEQ ID NO: 229, and a heavy chain that has at least 90, 91, 92, 93, 94, 95, 96, 97, 98, or 99% sequence identity to SEQ ID NO: 366 or SEQ ID NO: 367 (HC of MAB32), wherein the antibody, or antigen binding fragment thereof, maintains the sequences of the heavy chain and light chain CDRs of MAB32. In some embodiments, the antibody or antigen-binding fragment thereof, comprises a light chain that has at least 90, 91, 92, 93, 94, 95, 96, 97, 98, or 99% sequence identity to SEQ ID NO: 229, and a heavy chain that has at least 90, 91, 92, 93, 94, 95, 96, 97, 98, or 99% sequence identity to SEQ ID NO: 366 or SEQ ID NO: 367, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 67 or SEQ ID NO: 135, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof, comprises a light chain that has at least 90, 91, 92, 93, 94, 95, 96, 97, 98, or 99% sequence identity to SEQ ID NO: 230, and a heavy chain that has at least 90, 91, 92, 93, 94, 95, 96, 97, 98, or 99% sequence identity to SEQ ID NO: 368 or SEQ ID NO: 369 (HC of MAB33), wherein the antibody, or antigen binding fragment thereof, maintains the sequences of the heavy chain and light chain CDRs of MAB33. In some embodiments, the antibody or antigen-binding fragment thereof, comprises a light chain that has at least 90, 91, 92, 93, 94, 95, 96, 97, 98, or 99% sequence identity to SEQ ID NO: 230, and a heavy chain that has at least 90, 91, 92, 93, 94, 95, 96, 97, 98, or 99% sequence identity to SEQ ID NO: 368 or SEQ ID NO: 369, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 69 or SEQ ID NO: 136, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 68.

In some embodiments, the antibody or antigen-binding fragment thereof, comprises a light chain that has at least 90, 91, 92, 93, 94, 95, 96, 97, 98, or 99% sequence identity to SEQ ID NO: 231, and a heavy chain that has at least 90, 91, 92, 93, 94, 95, 96, 97, 98, or 99% sequence identity to SEQ ID NO: 370 or SEQ ID NO: 371 (HC of MAB34), wherein the antibody, or antigen binding fragment thereof, maintains the sequences of the heavy chain and light chain CDRs of MAB34. In some embodiments, the antibody or antigen-binding fragment thereof, comprises a light chain that has at least 90, 91, 92, 93, 94, 95, 96, 97, 98, or 99% sequence identity to SEQ ID NO: 231, and a heavy chain that has at least 90, 91, 92, 93, 94, 95, 96, 97, 98, or 99% sequence identity to SEQ ID NO: 370 or SEQ ID NO: 371, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 70 or SEQ ID NO: 137, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof, comprises a light chain that has at least 90, 91, 92, 93, 94, 95, 96, 97, 98, or 99% sequence identity to SEQ ID NO: 232, and a heavy chain that has at least 90, 91, 92, 93, 94, 95, 96, 97, 98, or 99% sequence identity to SEQ ID NO: 372 or SEQ ID NO: 373 (HC of MAB35), wherein the antibody, or antigen binding fragment thereof, maintains the sequences of the heavy chain and light chain CDRs of MAB35. In some embodiments, the antibody or antigen-binding fragment thereof, comprises a light chain that has at least 90, 91, 92, 93, 94, 95, 96, 97, 98, or 99% sequence identity to SEQ ID NO: 232, and a heavy chain that has at least 90, 91, 92, 93, 94, 95, 96, 97, 98, or 99% sequence identity to SEQ ID NO: 372 or SEQ ID NO: 373, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 72 or SEQ ID NO: 138, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof, comprises a light chain that has at least 90, 91, 92, 93, 94, 95, 96, 97, 98, or 99% sequence identity to SEQ ID NO: 233, and a heavy chain that has at least 90, 91, 92, 93, 94, 95, 96, 97, 98, or 99% sequence identity to SEQ ID NO: 374 or SEQ ID NO: 375 (HC of MAB36), wherein the antibody, or antigen binding fragment thereof, maintains the sequences of the heavy chain and light chain CDRs of MAB36. In some embodiments, the antibody or antigen-binding fragment thereof, comprises a light chain that has at least 90, 91, 92, 93, 94, 95, 96, 97, 98, or 99% sequence identity to SEQ ID NO: 233, and a heavy chain that has at least 90, 91, 92, 93, 94, 95, 96, 97, 98, or 99% sequence identity to SEQ ID NO: 374 or SEQ ID NO: 375, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 73 or SEQ ID NO: 139, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 71.

In some embodiments, the antibody or antigen-binding fragment thereof, comprises a light chain that has at least 90, 91, 92, 93, 94, 95, 96, 97, 98, or 99% sequence identity to SEQ ID NO: 234, and a heavy chain that has at least 90, 91, 92, 93, 94, 95, 96, 97, 98, or 99% sequence identity to SEQ ID NO: 376 or SEQ ID NO: 377 (HC of MAB37), wherein the antibody, or antigen binding fragment thereof, maintains the sequences of the heavy chain and light chain CDRs of MAB37. In some embodiments, the antibody or antigen-binding fragment thereof, comprises a light chain that has at least 90, 91, 92, 93, 94, 95, 96, 97, 98, or 99% sequence identity to SEQ ID NO: 234, and a heavy chain that has at least 90, 91, 92, 93, 94, 95, 96, 97, 98, or 99% sequence identity to SEQ ID NO: 376 or SEQ ID NO: 377, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 74 or SEQ ID NO: 140, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 75.

In some embodiments, the antibody or antigen-binding fragment thereof, comprises a light chain that has at least 90, 91, 92, 93, 94, 95, 96, 97, 98, or 99% sequence identity to SEQ ID NO: 235, and a heavy chain that has at least 90, 91, 92, 93, 94, 95, 96, 97, 98, or 99% sequence identity to SEQ ID NO: 378 or SEQ ID NO: 379 (HC of MAB38), wherein the antibody, or antigen binding fragment thereof, maintains the sequences of the heavy chain and light chain CDRs of MAB38. In some embodiments, the antibody or antigen-binding fragment thereof, comprises a light chain that has at least 90, 91, 92, 93, 94, 95, 96, 97, 98, or 99% sequence identity to SEQ ID NO: 235, and a heavy chain that has at least 90, 91, 92, 93, 94, 95, 96, 97, 98, or 99% sequence identity to SEQ ID NO: 378 or SEQ ID NO: 379, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 76 or SEQ ID NO: 141, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 77.

In some embodiments, the antibody or antigen-binding fragment thereof, comprises a light chain that has at least 90, 91, 92, 93, 94, 95, 96, 97, 98, or 99% sequence identity to SEQ ID NO: 236, and a heavy chain that has at least 90, 91, 92, 93, 94, 95, 96, 97, 98, or 99% sequence identity to SEQ ID NO: 380 or SEQ ID NO: 381 (HC of MAB39), wherein the antibody, or antigen binding fragment thereof, maintains the sequences of the heavy chain and light chain CDRs of MAB39. In some embodiments, the antibody or antigen-binding fragment thereof, comprises a light chain that has at least 90, 91, 92, 93, 94, 95, 96, 97, 98, or 99% sequence identity to SEQ ID NO: 236, and a heavy chain that has at least 90, 91, 92, 93, 94, 95, 96, 97, 98, or 99% sequence identity to SEQ ID NO: 380 or SEQ ID NO: 381, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 78 or SEQ ID NO: 142, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 79.

In some embodiments, the antibody or antigen-binding fragment thereof, comprises a light chain that has at least 90, 91, 92, 93, 94, 95, 96, 97, 98, or 99% sequence identity to SEQ ID NO: 237, and a heavy chain that has at least 90, 91, 92, 93, 94, 95, 96, 97, 98, or 99% sequence identity to SEQ ID NO: 382 or SEQ ID NO: 383 (HC of MAB40), wherein the antibody, or antigen binding fragment thereof, maintains the sequences of the heavy chain and light chain CDRs of MAB40. In some embodiments, the antibody or antigen-binding fragment thereof, comprises a light chain that has at least 90, 91, 92, 93, 94, 95, 96, 97, 98, or 99% sequence identity to SEQ ID NO: 237, and a heavy chain that has at least 90, 91, 92, 93, 94, 95, 96, 97, 98, or 99% sequence identity to SEQ ID NO: 382 or SEQ ID NO: 383, wherein the antibody, or antigen binding fragment thereof, maintains a HCDR1 sequence of SEQ ID NO: 61, SEQ ID NO: 124, or SEQ ID NO: 130, maintains a HCDR2 sequence of SEQ ID NO: 62, SEQ ID NO: 125, or SEQ ID NO: 131, maintains a HCDR3 sequence of SEQ ID NO: 80 or SEQ ID NO: 143, maintains a LCDR1 sequence of SEQ ID NO: 64, or SEQ ID NO: 133, maintains a LCDR2 sequence of SEQ ID NO: 65, or SEQ ID NO: 134, and maintains a LCDR3 sequence SEQ ID NO: 81.

In some embodiments, the antibody or antigen-binding fragment thereof, comprises a light chain that has at least 90, 91, 92, 93, 94, 95, 96, 97, 98, or 99% sequence identity to SEQ ID NO: 238, and a heavy chain that has at least 90, 91, 92, 93, 94, 95, 96, 97, 98, or 99% sequence identity to SEQ ID NO: 384 or SEQ ID NO: 385 (HC of MAB41), wherein the antibody, or antigen binding fragment thereof, maintains the sequences of the heavy chain and light chain CDRs of MAB41. In some embodiments, the antibody or antigen-binding fragment thereof, comprises a light chain that has at least 90, 91, 92, 93, 94, 95, 96, 97, 98, or 99% sequence identity to SEQ ID NO: 238, and a heavy chain that has at least 90, 91, 92, 93, 94, 95, 96, 97, 98, or 99% sequence identity to SEQ ID NO: 384 or SEQ ID NO: 385, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 87.

In some embodiments, the antibody or antigen-binding fragment thereof, comprises a light chain that has at least 90, 91, 92, 93, 94, 95, 96, 97, 98, or 99% sequence identity to SEQ ID NO: 239, and a heavy chain that has at least 90, 91, 92, 93, 94, 95, 96, 97, 98, or 99% sequence identity to SEQ ID NO: 386 or SEQ ID NO: 387 (HC of MAB42), wherein the antibody, or antigen binding fragment thereof, maintains the sequences of the heavy chain and light chain CDRs of MAB42. In some embodiments, the antibody or antigen-binding fragment thereof, comprises a light chain that has at least 90, 91, 92, 93, 94, 95, 96, 97, 98, or 99% sequence identity to SEQ ID NO: 239, and a heavy chain that has at least 90, 91, 92, 93, 94, 95, 96, 97, 98, or 99% sequence identity to SEQ ID NO: 386 or SEQ ID NO: 387, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 88 or SEQ ID NO: 149, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof, comprises a light chain that has at least 90, 91, 92, 93, 94, 95, 96, 97, 98, or 99% sequence identity to SEQ ID NO: 240, and a heavy chain that has at least 90, 91, 92, 93, 94, 95, 96, 97, 98, or 99% sequence identity to SEQ ID NO: 388 or SEQ ID NO: 389 (HC of MAB43), wherein the antibody, or antigen binding fragment thereof, maintains the sequences of the heavy chain and light chain CDRs of MAB43. In some embodiments, the antibody or antigen-binding fragment thereof, comprises a light chain that has at least 90, 91, 92, 93, 94, 95, 96, 97, 98, or 99% sequence identity to SEQ ID NO: 240, and a heavy chain that has at least 90, 91, 92, 93, 94, 95, 96, 97, 98, or 99% sequence identity to SEQ ID NO: 388 or SEQ ID NO: 389, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 90 or SEQ ID NO: 150, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 89.

In some embodiments, the antibody or antigen-binding fragment thereof, comprises a light chain that has at least 90, 91, 92, 93, 94, 95, 96, 97, 98, or 99% sequence identity to SEQ ID NO: 241, and a heavy chain that has at least 90, 91, 92, 93, 94, 95, 96, 97, 98, or 99% sequence identity to SEQ ID NO: 390 or SEQ ID NO: 391 (HC of MAB44), wherein the antibody, or antigen binding fragment thereof, maintains the sequences of the heavy chain and light chain CDRs of MAB44. In some embodiments, the antibody or antigen-binding fragment thereof, comprises a light chain that has at least 90, 91, 92, 93, 94, 95, 96, 97, 98, or 99% sequence identity to SEQ ID NO: 241, and a heavy chain that has at least 90, 91, 92, 93, 94, 95, 96, 97, 98, or 99% sequence identity to SEQ ID NO: 390 or SEQ ID NO: 391, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 91 or SEQ ID NO: 151, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 92.

In some embodiments, the antibody or antigen-binding fragment thereof, comprises a light chain that has at least 90, 91, 92, 93, 94, 95, 96, 97, 98, or 99% sequence identity to SEQ ID NO: 242, and a heavy chain that has at least 90, 91, 92, 93, 94, 95, 96, 97, 98, or 99% sequence identity to SEQ ID NO: 392 or SEQ ID NO: 393 (HC of MAB45), wherein the antibody, or antigen binding fragment thereof, maintains the sequences of the heavy chain and light chain CDRs of MAB45. In some embodiments, the antibody or antigen-binding fragment thereof, comprises a light chain that has at least 90, 91, 92, 93, 94, 95, 96, 97, 98, or 99% sequence identity to SEQ ID NO: 242, and a heavy chain that has at least 90, 91, 92, 93, 94, 95, 96, 97, 98, or 99% sequence identity to SEQ ID NO: 392 or SEQ ID NO: 393, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 93 or SEQ ID NO: 152, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 94.

In some embodiments, the antibody or antigen-binding fragment thereof, comprises a light chain that has at least 90, 91, 92, 93, 94, 95, 96, 97, 98, or 99% sequence identity to SEQ ID NO: 243, and a heavy chain that has at least 90, 91, 92, 93, 94, 95, 96, 97, 98, or 99% sequence identity to SEQ ID NO: 394 or SEQ ID NO: 395 (HC of MAB46), wherein the antibody, or antigen binding fragment thereof, maintains the sequences of the heavy chain and light chain CDRs of MAB46. In some embodiments, the antibody or antigen-binding fragment thereof, comprises a light chain that has at least 90, 91, 92, 93, 94, 95, 96, 97, 98, or 99% sequence identity to SEQ ID NO: 243, and a heavy chain that has at least 90, 91, 92, 93, 94, 95, 96, 97, 98, or 99% sequence identity to SEQ ID NO: 394 or SEQ ID NO: 395, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 84 or SEQ ID NO: 146, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 95.

In some embodiments, the antibody or antigen-binding fragment thereof, comprises a light chain that has at least 90, 91, 92, 93, 94, 95, 96, 97, 98, or 99% sequence identity to SEQ ID NO: 244, and a heavy chain that has at least 90, 91, 92, 93, 94, 95, 96, 97, 98, or 99% sequence identity to SEQ ID NO: 396 or SEQ ID NO: 397 (HC of MAB47), wherein the antibody, or antigen binding fragment thereof, maintains the sequences of the heavy chain and light chain CDRs of MAB47. In some embodiments, the antibody or antigen-binding fragment thereof, comprises a light chain that has at least 90, 91, 92, 93, 94, 95, 96, 97, 98, or 99% sequence identity to SEQ ID NO: 244, and a heavy chain that has at least 90, 91, 92, 93, 94, 95, 96, 97, 98, or 99% sequence identity to SEQ ID NO: 396 or SEQ ID NO: 397, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 96 or SEQ ID NO: 153, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 97.

In some embodiments, the antibody or antigen-binding fragment thereof, comprises a light chain that has at least 90, 91, 92, 93, 94, 95, 96, 97, 98, or 99% sequence identity to SEQ ID NO: 245, and a heavy chain that has at least 90, 91, 92, 93, 94, 95, 96, 97, 98, or 99% sequence identity to SEQ ID NO: 398 or SEQ ID NO: 399 (HC of MAB48), wherein the antibody, or antigen binding fragment thereof, maintains the sequences of the heavy chain and light chain CDRs of MAB48. In some embodiments, the antibody or antigen-binding fragment thereof, comprises a light chain that has at least 90, 91, 92, 93, 94, 95, 96, 97, 98, or 99% sequence identity to SEQ ID NO: 245, and a heavy chain that has at least 90, 91, 92, 93, 94, 95, 96, 97, 98, or 99% sequence identity to SEQ ID NO: 398 or SEQ ID NO: 399, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 98 or SEQ ID NO: 154, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 99.

In some embodiments, the antibody or antigen-binding fragment thereof, comprises a light chain that has at least 90, 91, 92, 93, 94, 95, 96, 97, 98, or 99% sequence identity to SEQ ID NO: 246, and a heavy chain that has at least 90, 91, 92, 93, 94, 95, 96, 97, 98, or 99% sequence identity to SEQ ID NO: 400 or SEQ ID NO: 401 (HC of MAB49), wherein the antibody, or antigen binding fragment thereof, maintains the sequences of the heavy chain and light chain CDRs of MAB49. In some embodiments, the antibody or antigen-binding fragment thereof, comprises a light chain that has at least 90, 91, 92, 93, 94, 95, 96, 97, 98, or 99% sequence identity to SEQ ID NO: 246, and a heavy chain that has at least 90, 91, 92, 93, 94, 95, 96, 97, 98, or 99% sequence identity to SEQ ID NO: 400 or SEQ ID NO: 401, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 101.

In some embodiments, the antibody or antigen-binding fragment thereof, comprises a light chain that has at least 90, 91, 92, 93, 94, 95, 96, 97, 98, or 99% sequence identity to SEQ ID NO: 247, and a heavy chain that has at least 90, 91, 92, 93, 94, 95, 96, 97, 98, or 99% sequence identity to SEQ ID NO: 402 or SEQ ID NO: 403 (HC of MAB50), wherein the antibody, or antigen binding fragment thereof, maintains the sequences of the heavy chain and light chain CDRs of MAB50. In some embodiments, the antibody or antigen-binding fragment thereof, comprises a light chain that has at least 90, 91, 92, 93, 94, 95, 96, 97, 98, or 99% sequence identity to SEQ ID NO: 247, and a heavy chain that has at least 90, 91, 92, 93, 94, 95, 96, 97, 98, or 99% sequence identity to SEQ ID NO: 402 or SEQ ID NO: 403, wherein the antibody, or antigen binding fragment thereof, maintains a HCDR1 sequence of SEQ ID NO: 82, SEQ ID NO: 126, or SEQ ID NO: 144, maintains a HCDR2 sequence of SEQ ID NO: 83, SEQ ID NO: 127, or SEQ ID NO: 145, maintains a HCDR3 sequence of SEQ ID NO: 100 or SEQ ID NO: 155, maintains a LCDR1 sequence of SEQ ID NO: 85, or SEQ ID NO: 147, maintains a LCDR2 sequence of SEQ ID NO: 86, or SEQ ID NO: 148, and maintains a LCDR3 sequence SEQ ID NO: 102.

In some embodiments, the antibody or antigen-binding fragment thereof, comprises a light chain that has at least 90, 91, 92, 93, 94, 95, 96, 97, 98, or 99% sequence identity to SEQ ID NO: 248, and a heavy chain that has at least 90, 91, 92, 93, 94, 95, 96, 97, 98, or 99% sequence identity to SEQ ID NO: 404 or SEQ ID NO: 405 (HC of MAB51), wherein the antibody, or antigen binding fragment thereof, maintains the sequences of the heavy chain and light chain CDRs of MAB51. In some embodiments, the antibody or antigen-binding fragment thereof, comprises a light chain that has at least 90, 91, 92, 93, 94, 95, 96, 97, 98, or 99% sequence identity to SEQ ID NO: 248, and a heavy chain that has at least 90, 91, 92, 93, 94, 95, 96, 97, 98, or 99% sequence identity to SEQ ID NO: 404 or SEQ ID NO: 405, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 108.

In some embodiments, the antibody or antigen-binding fragment thereof, comprises a light chain that has at least 90, 91, 92, 93, 94, 95, 96, 97, 98, or 99% sequence identity to SEQ ID NO: 249, and a heavy chain that has at least 90, 91, 92, 93, 94, 95, 96, 97, 98, or 99% sequence identity to SEQ ID NO: 406 or SEQ ID NO: 407 (HC of MAB52), wherein the antibody, or antigen binding fragment thereof, maintains the sequences of the heavy chain and light chain CDRs of MAB52. In some embodiments, the antibody or antigen-binding fragment thereof, comprises a light chain that has at least 90, 91, 92, 93, 94, 95, 96, 97, 98, or 99% sequence identity to SEQ ID NO: 249, and a heavy chain that has at least 90, 91, 92, 93, 94, 95, 96, 97, 98, or 99% sequence identity to SEQ ID NO: 406 or SEQ ID NO: 407, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 109 or SEQ ID NO: 161, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 110.

In some embodiments, the antibody or antigen-binding fragment thereof, comprises a light chain that has at least 90, 91, 92, 93, 94, 95, 96, 97, 98, or 99% sequence identity to SEQ ID NO: 250, and a heavy chain that has at least 90, 91, 92, 93, 94, 95, 96, 97, 98, or 99% sequence identity to SEQ ID NO: 408 or SEQ ID NO: 409 (HC of MAB53), wherein the antibody, or antigen binding fragment thereof, maintains the sequences of the heavy chain and light chain CDRs of MAB53. In some embodiments, the antibody or antigen-binding fragment thereof, comprises a light chain that has at least 90, 91, 92, 93, 94, 95, 96, 97, 98, or 99% sequence identity to SEQ ID NO: 250, and a heavy chain that has at least 90, 91, 92, 93, 94, 95, 96, 97, 98, or 99% sequence identity to SEQ ID NO: 408 or SEQ ID NO: 409, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 111 or SEQ ID NO: 162, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 112.

In some embodiments, the antibody or antigen-binding fragment thereof, comprises a light chain that has at least 90, 91, 92, 93, 94, 95, 96, 97, 98, or 99% sequence identity to SEQ ID NO: 251, and a heavy chain that has at least 90, 91, 92, 93, 94, 95, 96, 97, 98, or 99% sequence identity to SEQ ID NO: 410 or SEQ ID NO: 411 (HC of MAB54), wherein the antibody, or antigen binding fragment thereof, maintains the sequences of the heavy chain and light chain CDRs of MAB54. In some embodiments, the antibody or antigen-binding fragment thereof, comprises a light chain that has at least 90, 91, 92, 93, 94, 95, 96, 97, 98, or 99% sequence identity to SEQ ID NO: 251, and a heavy chain that has at least 90, 91, 92, 93, 94, 95, 96, 97, 98, or 99% sequence identity to SEQ ID NO: 410 or SEQ ID NO: 411, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 105 or SEQ ID NO: 158, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof, comprises a light chain that has at least 90, 91, 92, 93, 94, 95, 96, 97, 98, or 99% sequence identity to SEQ ID NO: 252, and a heavy chain that has at least 90, 91, 92, 93, 94, 95, 96, 97, 98, or 99% sequence identity to SEQ ID NO: 412 or SEQ ID NO: 413 (HC of MAB55), wherein the antibody, or antigen binding fragment thereof, maintains the sequences of the heavy chain and light chain CDRs of MAB55. In some embodiments, the antibody or antigen-binding fragment thereof, comprises a light chain that has at least 90, 91, 92, 93, 94, 95, 96, 97, 98, or 99% sequence identity to SEQ ID NO: 252, and a heavy chain that has at least 90, 91, 92, 93, 94, 95, 96, 97, 98, or 99% sequence identity to SEQ ID NO: 412 or SEQ ID NO: 413, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 114 or SEQ ID NO: 163, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 115.

In some embodiments, the antibody or antigen-binding fragment thereof, comprises a light chain that has at least 90, 91, 92, 93, 94, 95, 96, 97, 98, or 99% sequence identity to SEQ ID NO: 253, and a heavy chain that has at least 90, 91, 92, 93, 94, 95, 96, 97, 98, or 99% sequence identity to SEQ ID NO: 414 or SEQ ID NO: 415 (HC of MAB56), wherein the antibody, or antigen binding fragment thereof, maintains the sequences of the heavy chain and light chain CDRs of MAB56. In some embodiments, the antibody or antigen-binding fragment thereof, comprises a light chain that has at least 90, 91, 92, 93, 94, 95, 96, 97, 98, or 99% sequence identity to SEQ ID NO: 253, and a heavy chain that has at least 90, 91, 92, 93, 94, 95, 96, 97, 98, or 99% sequence identity to SEQ ID NO: 414 or SEQ ID NO: 415, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 113.

In some embodiments, the antibody or antigen-binding fragment thereof, comprises a light chain that has at least 90, 91, 92, 93, 94, 95, 96, 97, 98, or 99% sequence identity to SEQ ID NO: 254, and a heavy chain that has at least 90, 91, 92, 93, 94, 95, 96, 97, 98, or 99% sequence identity to SEQ ID NO: 416 or SEQ ID NO: 417 (HC of MAB57), wherein the antibody, or antigen binding fragment thereof, maintains the sequences of the heavy chain and light chain CDRs of MAB57. In some embodiments, the antibody or antigen-binding fragment thereof, comprises a light chain that has at least 90, 91, 92, 93, 94, 95, 96, 97, 98, or 99% sequence identity to SEQ ID NO: 254, and a heavy chain that has at least 90, 91, 92, 93, 94, 95, 96, 97, 98, or 99% sequence identity to SEQ ID NO: 416 or SEQ ID NO: 417, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 117 or SEQ ID NO: 165, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 118.

In some embodiments, the antibody or antigen-binding fragment thereof, comprises a light chain that has at least 90, 91, 92, 93, 94, 95, 96, 97, 98, or 99% sequence identity to SEQ ID NO: 255, and a heavy chain that has at least 90, 91, 92, 93, 94, 95, 96, 97, 98, or 99% sequence identity to SEQ ID NO: 418 or SEQ ID NO: 419 (HC of MAB58), wherein the antibody, or antigen binding fragment thereof, maintains the sequences of the heavy chain and light chain CDRs of MAB58. In some embodiments, the antibody or antigen-binding fragment thereof, comprises a light chain that has at least 90, 91, 92, 93, 94, 95, 96, 97, 98, or 99% sequence identity to SEQ ID NO: 255, and a heavy chain that has at least 90, 91, 92, 93, 94, 95, 96, 97, 98, or 99% sequence identity to SEQ ID NO: 418 or SEQ ID NO: 419, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 116 or SEQ ID NO: 164, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 119.

In some embodiments, the antibody or antigen-binding fragment thereof, comprises a light chain that has at least 90, 91, 92, 93, 94, 95, 96, 97, 98, or 99% sequence identity to SEQ ID NO: 256, and a heavy chain that has at least 90, 91, 92, 93, 94, 95, 96, 97, 98, or 99% sequence identity to SEQ ID NO: 420 or SEQ ID NO: 421 (HC of MAB59), wherein the antibody, or antigen binding fragment thereof, maintains the sequences of the heavy chain and light chain CDRs of MAB59. In some embodiments, the antibody or antigen-binding fragment thereof, comprises a light chain that has at least 90, 91, 92, 93, 94, 95, 96, 97, 98, or 99% sequence identity to SEQ ID NO: 256, and a heavy chain that has at least 90, 91, 92, 93, 94, 95, 96, 97, 98, or 99% sequence identity to SEQ ID NO: 420 or SEQ ID NO: 421, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 120 or SEQ ID NO: 166, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 121.

In some embodiments, the antibody or antigen-binding fragment thereof, comprises a light chain that has at least 90, 91, 92, 93, 94, 95, 96, 97, 98, or 99% sequence identity to SEQ ID NO: 257, and a heavy chain that has at least 90, 91, 92, 93, 94, 95, 96, 97, 98, or 99% sequence identity to SEQ ID NO: 422 or SEQ ID NO: 423 (HC of MAB60), wherein the antibody, or antigen binding fragment thereof, maintains the sequences of the heavy chain and light chain CDRs of MAB60. In some embodiments, the antibody or antigen-binding fragment thereof, comprises a light chain that has at least 90, 91, 92, 93, 94, 95, 96, 97, 98, or 99% sequence identity to SEQ ID NO: 257, and a heavy chain that has at least 90, 91, 92, 93, 94, 95, 96, 97, 98, or 99% sequence identity to SEQ ID NO: 422 or SEQ ID NO: 423, wherein the antibody, or antigen binding fragment thereof, maintains a HCDR1 sequence of SEQ ID NO: 103, SEQ ID NO: 128, or SEQ ID NO: 156, maintains a HCDR2 sequence of SEQ ID NO: 104, SEQ ID NO: 129, or SEQ ID NO: 157, maintains a HCDR3 sequence of SEQ ID NO: 122 or SEQ ID NO: 167, maintains a LCDR1 sequence of SEQ ID NO: 106, or SEQ ID NO: 159, maintains a LCDR2 sequence of SEQ ID NO: 107, or SEQ ID NO: 160, and maintains a LCDR3 sequence SEQ ID NO: 123.

In some embodiments, the antibody, or antigen-binding fragment thereof, comprises a light chain having a sequence of SEQ ID NO: 228 and a heavy chain that comprises a sequence of SEQ ID NO: 168 or SEQ ID NO: 169.

In some embodiments, the antibody, or antigen-binding fragment thereof, comprises a light chain having a sequence of SEQ ID NO: 229 and a heavy chain that comprises a sequence of SEQ ID NO: 170 or SEQ ID NO: 171.

In some embodiments, the antibody, or antigen-binding fragment thereof, comprises a light chain having a sequence of SEQ ID NO: 230 and a heavy chain that comprises a sequence of SEQ ID NO: 172 or SEQ ID NO: 173.

In some embodiments, the antibody, or antigen-binding fragment thereof, comprises a light chain having a sequence of SEQ ID NO: 231 and a heavy chain that comprises a sequence of SEQ ID NO: 174 or SEQ ID NO: 175.

In some embodiments, the antibody, or antigen-binding fragment thereof, comprises a light chain having a sequence of SEQ ID NO: 232 and a heavy chain that comprises a sequence of SEQ ID NO: 176 or SEQ ID NO: 177.

In some embodiments, the antibody, or antigen-binding fragment thereof, comprises a light chain having a sequence of SEQ ID NO: 233 and a heavy chain that comprises a sequence of SEQ ID NO: 178 or SEQ ID NO: 179.

In some embodiments, the antibody, or antigen-binding fragment thereof, comprises a light chain having a sequence of SEQ ID NO: 234 and a heavy chain that comprises a sequence of SEQ ID NO: 180 or SEQ ID NO: 181.

In some embodiments, the antibody, or antigen-binding fragment thereof, comprises a light chain having a sequence of SEQ ID NO: 235 and a heavy chain that comprises a sequence of SEQ ID NO: 182 or SEQ ID NO: 183.

In some embodiments, the antibody, or antigen-binding fragment thereof, comprises a light chain having a sequence of SEQ ID NO: 236 and a heavy chain that comprises a sequence of SEQ ID NO: 184 or SEQ ID NO: 185.

In some embodiments, the antibody, or antigen-binding fragment thereof, comprises a light chain having a sequence of SEQ ID NO: 237 and a heavy chain that comprises a sequence of SEQ ID NO: 186 or SEQ ID NO: 187.

In some embodiments, the antibody, or antigen-binding fragment thereof, comprises a light chain having a sequence of SEQ ID NO: 238 and a heavy chain that comprises a sequence of SEQ ID NO: 188 or SEQ ID NO: 189.

In some embodiments, the antibody, or antigen-binding fragment thereof, comprises a light chain having a sequence of SEQ ID NO: 239 and a heavy chain that comprises a sequence of SEQ ID NO: 190 or SEQ ID NO: 191.

In some embodiments, the antibody, or antigen-binding fragment thereof, comprises a light chain having a sequence of SEQ ID NO: 240 and a heavy chain that comprises a sequence of SEQ ID NO: 192 or SEQ ID NO: 193.

In some embodiments, the antibody, or antigen-binding fragment thereof, comprises a light chain having a sequence of SEQ ID NO: 241 and a heavy chain that comprises a sequence of SEQ ID NO: 194 or SEQ ID NO: 195.

In some embodiments, the antibody, or antigen-binding fragment thereof, comprises a light chain having a sequence of SEQ ID NO: 242 and a heavy chain that comprises a sequence of SEQ ID NO: 196 or SEQ ID NO: 197.

In some embodiments, the antibody, or antigen-binding fragment thereof, comprises a light chain having a sequence of SEQ ID NO: 243 and a heavy chain that comprises a sequence of SEQ ID NO: 198 or SEQ ID NO: 199.

In some embodiments, the antibody, or antigen-binding fragment thereof, comprises a light chain having a sequence of SEQ ID NO: 244 and a heavy chain that comprises a sequence of SEQ ID NO: 200 or SEQ ID NO: 201.

In some embodiments, the antibody, or antigen-binding fragment thereof, comprises a light chain having a sequence of SEQ ID NO: 245 and a heavy chain that comprises a sequence of SEQ ID NO: 202 or SEQ ID NO: 203.

In some embodiments, the antibody, or antigen-binding fragment thereof, comprises a light chain having a sequence of SEQ ID NO: 246 and a heavy chain that comprises a sequence of SEQ ID NO: 204 or SEQ ID NO: 205.

In some embodiments, the antibody, or antigen-binding fragment thereof, comprises a light chain having a sequence of SEQ ID NO: 247 and a heavy chain that comprises a sequence of SEQ ID NO: 206 or SEQ ID NO: 207.

In some embodiments, the antibody, or antigen-binding fragment thereof, comprises a light chain having a sequence of SEQ ID NO: 248 and a heavy chain that comprises a sequence of SEQ ID NO: 208 or SEQ ID NO: 209.

In some embodiments, the antibody, or antigen-binding fragment thereof, comprises a light chain having a sequence of SEQ ID NO: 249 and a heavy chain that comprises a sequence of SEQ ID NO: 210 or SEQ ID NO: 211.

In some embodiments, the antibody, or antigen-binding fragment thereof, comprises a light chain having a sequence of SEQ ID NO: 250 and a heavy chain that comprises a sequence of SEQ ID NO: 212 or SEQ ID NO: 213.

In some embodiments, the antibody, or antigen-binding fragment thereof, comprises a light chain having a sequence of SEQ ID NO: 251 and a heavy chain that comprises a sequence of SEQ ID NO: 214 or SEQ ID NO: 215.

In some embodiments, the antibody, or antigen-binding fragment thereof, comprises a light chain having a sequence of SEQ ID NO: 252 and a heavy chain that comprises a sequence of SEQ ID NO: 216 or SEQ ID NO: 217.

In some embodiments, the antibody, or antigen-binding fragment thereof, comprises a light chain having a sequence of SEQ ID NO: 253 and a heavy chain that comprises a sequence of SEQ ID NO: 218 or SEQ ID NO: 219.

In some embodiments, the antibody, or antigen-binding fragment thereof, comprises a light chain having a sequence of SEQ ID NO: 254 and a heavy chain that comprises a sequence of SEQ ID NO: 220 or SEQ ID NO: 221.

In some embodiments, the antibody, or antigen-binding fragment thereof, comprises a light chain having a sequence of SEQ ID NO: 255 and a heavy chain that comprises a sequence of SEQ ID NO: 222 or SEQ ID NO: 223.

In some embodiments, the antibody, or antigen-binding fragment thereof, comprises a light chain having a sequence of SEQ ID NO: 256 and a heavy chain that comprises a sequence of SEQ ID NO: 224 or SEQ ID NO: 225.

In some embodiments, the antibody, or antigen-binding fragment thereof, comprises a light chain having a sequence of SEQ ID NO: 257 and a heavy chain that comprises a sequence of SEQ ID NO: 226 or SEQ ID NO: 227.

In some embodiments, the antibody, or antigen-binding fragment thereof, comprises a light chain having a sequence of SEQ ID NO: 228 and a heavy chain that comprises a sequence of SEQ ID NO: 364 or SEQ ID NO: 365.

In some embodiments, the antibody, or antigen-binding fragment thereof, comprises a light chain having a sequence of SEQ ID NO: 229 and a heavy chain that comprises a sequence of SEQ ID NO: 366 or SEQ ID NO: 367.

In some embodiments, the antibody, or antigen-binding fragment thereof, comprises a light chain having a sequence of SEQ ID NO: 230 and a heavy chain that comprises a sequence of SEQ ID NO: 368 or SEQ ID NO: 369.

In some embodiments, the antibody, or antigen-binding fragment thereof, comprises a light chain having a sequence of SEQ ID NO: 231 and a heavy chain that comprises a sequence of SEQ ID NO: 370 or SEQ ID NO: 371.

In some embodiments, the antibody, or antigen-binding fragment thereof, comprises a light chain having a sequence of SEQ ID NO: 232 and a heavy chain that comprises a sequence of SEQ ID NO: 372 or SEQ ID NO: 373.

In some embodiments, the antibody, or antigen-binding fragment thereof, comprises a light chain having a sequence of SEQ ID NO: 233 and a heavy chain that comprises a sequence of SEQ ID NO: 374 or SEQ ID NO: 375.

In some embodiments, the antibody, or antigen-binding fragment thereof, comprises a light chain having a sequence of SEQ ID NO: 234 and a heavy chain that comprises a sequence of SEQ ID NO: 376 or SEQ ID NO: 377.

In some embodiments, the antibody, or antigen-binding fragment thereof, comprises a light chain having a sequence of SEQ ID NO: 235 and a heavy chain that comprises a sequence of SEQ ID NO: 378 or SEQ ID NO: 379.

In some embodiments, the antibody, or antigen-binding fragment thereof, comprises a light chain having a sequence of SEQ ID NO: 236 and a heavy chain that comprises a sequence of SEQ ID NO: 380 or SEQ ID NO: 381.

In some embodiments, the antibody, or antigen-binding fragment thereof, comprises a light chain having a sequence of SEQ ID NO: 237 and a heavy chain that comprises a sequence of SEQ ID NO: 382 or SEQ ID NO: 383.

In some embodiments, the antibody, or antigen-binding fragment thereof, comprises a light chain having a sequence of SEQ ID NO: 238 and a heavy chain that comprises a sequence of SEQ ID NO: 384 or SEQ ID NO: 385.

In some embodiments, the antibody, or antigen-binding fragment thereof, comprises a light chain having a sequence of SEQ ID NO: 239 and a heavy chain that comprises a sequence of SEQ ID NO: 386 or SEQ ID NO: 387.

In some embodiments, the antibody, or antigen-binding fragment thereof, comprises a light chain having a sequence of SEQ ID NO: 240 and a heavy chain that comprises a sequence of SEQ ID NO: 388 or SEQ ID NO: 389.

In some embodiments, the antibody, or antigen-binding fragment thereof, comprises a light chain having a sequence of SEQ ID NO: 241 and a heavy chain that comprises a sequence of SEQ ID NO: 390 or SEQ ID NO: 391.

In some embodiments, the antibody, or antigen-binding fragment thereof, comprises a light chain having a sequence of SEQ ID NO: 242 and a heavy chain that comprises a sequence of SEQ ID NO: 392 or SEQ ID NO: 393.

In some embodiments, the antibody, or antigen-binding fragment thereof, comprises a light chain having a sequence of SEQ ID NO: 243 and a heavy chain that comprises a sequence of SEQ ID NO: 394 or SEQ ID NO: 395.

In some embodiments, the antibody, or antigen-binding fragment thereof, comprises a light chain having a sequence of SEQ ID NO: 244 and a heavy chain that comprises a sequence of SEQ ID NO: 396 or SEQ ID NO: 397.

In some embodiments, the antibody, or antigen-binding fragment thereof, comprises a light chain having a sequence of SEQ ID NO: 245 and a heavy chain that comprises a sequence of SEQ ID NO: 398 or SEQ ID NO: 399.

In some embodiments, the antibody, or antigen-binding fragment thereof, comprises a light chain having a sequence of SEQ ID NO: 246 and a heavy chain that comprises a sequence of SEQ ID NO: 400 or SEQ ID NO: 401.

In some embodiments, the antibody, or antigen-binding fragment thereof, comprises a light chain having a sequence of SEQ ID NO: 247 and a heavy chain that comprises a sequence of SEQ ID NO: 402 or SEQ ID NO: 403.

In some embodiments, the antibody, or antigen-binding fragment thereof, comprises a light chain having a sequence of SEQ ID NO: 248 and a heavy chain that comprises a sequence of SEQ ID NO: 404 or SEQ ID NO: 405.

In some embodiments, the antibody, or antigen-binding fragment thereof, comprises a light chain having a sequence of SEQ ID NO: 249 and a heavy chain that comprises a sequence of SEQ ID NO: 406 or SEQ ID NO: 407.

In some embodiments, the antibody, or antigen-binding fragment thereof, comprises a light chain having a sequence of SEQ ID NO: 250 and a heavy chain that comprises a sequence of SEQ ID NO: 408 or SEQ ID NO: 409.

In some embodiments, the antibody, or antigen-binding fragment thereof, comprises a light chain having a sequence of SEQ ID NO: 251 and a heavy chain that comprises a sequence of SEQ ID NO: 410 or SEQ ID NO: 411.

In some embodiments, the antibody, or antigen-binding fragment thereof, comprises a light chain having a sequence of SEQ ID NO: 252 and a heavy chain that comprises a sequence of SEQ ID NO: 412 or SEQ ID NO: 413.

In some embodiments, the antibody, or antigen-binding fragment thereof, comprises a light chain having a sequence of SEQ ID NO: 253 and a heavy chain that comprises a sequence of SEQ ID NO: 414 or SEQ ID NO: 415.

In some embodiments, the antibody, or antigen-binding fragment thereof, comprises a light chain having a sequence of SEQ ID NO: 254 and a heavy chain that comprises a sequence of SEQ ID NO: 416 or SEQ ID NO: 417.

In some embodiments, the antibody, or antigen-binding fragment thereof, comprises a light chain having a sequence of SEQ ID NO: 255 and a heavy chain that comprises a sequence of SEQ ID NO: 418 or SEQ ID NO: 419.

In some embodiments, the antibody, or antigen-binding fragment thereof, comprises a light chain having a sequence of SEQ ID NO: 256 and a heavy chain that comprises a sequence of SEQ ID NO: 420 or SEQ ID NO: 421.

In some embodiments, the antibody, or antigen-binding fragment thereof, comprises a light chain having a sequence of SEQ ID NO: 257 and a heavy chain that comprises a sequence of SEQ ID NO: 422 or SEQ ID NO: 423.

In some embodiments, an antibody is provided that binds (e.g., specifically binds) to the active form of C1s. In some embodiments, the antibody has a K_{D} of less than 7×10⁻⁹ M, 6×10⁻⁹ M, 5×10⁻⁹ M, 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M. In some embodiments, the K_{D} is determined by surface plasmon resonance (SPR). In some embodiments, the K_{D} is measured as provided for herein, such as in Example 3. Briefly, and without being bound to any particular SPR method, in an SPR method, the antibody can be immobilized on a sensor surface and a buffer containing the target antigen (e.g., active C1s or the precursor form to show specificity) is passed over the antibody at various concentrations and the binding is determined using the sensor. In some embodiments, the antibody that has a K_{D} of less than 7×10⁻⁹ M, 6×10⁻⁹ M, 5×10⁻⁹ M, 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, or 4×10⁻¹⁰ M is an antibody as provided for herein.

In some embodiments, an antibody is provided that specifically binds to active C1s and inhibits the Wieslab complement classical pathway with an IC₅₀ of about 1 to 5 nM, about 0.1 to 1 nM, about 0.5 to about 3 nM, about 1 to about 2 nM, less than 5 nM, less than 4.5 nM, less than 3 nM, or less than 1 nM. In some embodiments, the IC₅₀ is determined by binding IgM to a surface (e.g., plate) to activate the complement classical pathway when human serum is added to the wells leading to deposition of components of the membrane attach complex (MAC) in the wells of the plate. Activation can then be measured by detecting the MAC by, for example, an ELISA, with an alkaline phosphatase labelled antibody specific to the neoantigen expressed during MAC formation. To determine the IC₅₀ of an inhibitory antibody, such as those provided for herein, in this assay, the assay can be modified by preincubating aliquots of the human serum with varying amounts of the inhibitory anti-C1s antibody for 15 minutes before initiating the assay, which then allows for the IC₅₀ to be determined for inhibition of C5bC9 formation. An assay for such determination can be performed using a kit SVAR LifeSciences, Malmo, Sweden and purchased from Eagle Bioscience, Amherst, NH (catalog number COMPL CL310). In some embodiments, the assay is performed as provided for in Example 4. In some embodiments, the antibody that specifically binds to active C1s and inhibits the Wieslab complement classical pathway with an IC₅₀ of about 1 to 5 nM, about 0.1 to 1 nM, about 0.5 to about 3 nM, about 1 to about 2 nM, less than 5 nM, less than 4.5 nM, less than 3 nM, or less than 1 nM is an antibody as provided for herein.

In some embodiments, an antibody is provided that specifically binds to active C1s and inhibits human serum complement-mediated human RBC lysis with an IC₅₀ of less than 20 nM, less than 19 nM, less than 18 nM, less than 15 nM, less than 13 nM, less than 12 nM, less than 12.5 nM, about 11 to about 20 nM, about 11 to about 15 nM, about 11 to about 13 nM, about 11 to about 12 nM, about 12 to about 15 nM. In some embodiments, the IC₅₀ is less than less than 10 nM, about 2 to about 10 nM, about 3 to about 10 nM, about 5 to about 10 nM, less than 25 nM, about 15 to about 25 nM, about 15 to about 21 nM, or about 20 to about 25 nM. In some embodiments, the inhibition of human serum complement-mediated human RBC lysis is determined by sensitizing human red blood cells (hRBCs) to IgG in human serum (such as, but not limited to, rabbit IgG). The percent human serum can be, for example, 20-30% human serum, or 25% human serum. In some embodiments, the assay is performed by incubating the test antibody with aliquots of diluted human serum added to sensitized hRBCs to determine inhibition of lysis. The lysis can be measured by taking measurements, for example, at an absorbance of about 415 nm. In some embodiments, the assay is performed as set forth in Example 5. In some embodiments, the antibody that specifically binds to active C1s and inhibits human serum complement-mediated human RBC lysis with an IC₅₀ of less than 20 nM, less than 19 nM, less than 18 nM, less than 15 nM, less than 13 nM, less than 12 nM, less than 12.5 nM, about 11 to about 20 nM, about 11 to about 15 nM, about 11 to about 13 nM, about 11 to about 12 nM, about 12 to about 15 nM is as provided for herein. In some embodiments, the antibody as provided for herein has an IC₅₀ that is less than less than 10 nM, about 2 to about 10 nM, about 3 to about 10 nM, about 5 to about 10 nM, less than 25 nM, about 15 to about 25 nM, about 15 to about 21 nM, or about 20 to about 25 nM. In some embodiments, the antibody has an IC₅₀ that is less (more potent) than the antibody referred to at TNT020, which is described in WO2018071676A1, which is hereby incorporated by reference in its entirety.

In some embodiments, the antibody has a K_{D} of less than 7×10⁻⁹ M, 6×10⁻⁹ M, 5×10⁻⁹ M, 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, or 4×10⁻¹⁰ M and inhibits the Wieslab complement classical pathway with an IC₅₀ of about 1 to 5 nM, about 0.1 to 1 nM, about 0.5 to about 3 nM, about 1 to about 2 nM, less than 5 nM, less than 4.5 nM, less than 3 nM, or less than 1 nM.

In some embodiments, the antibody has a K_{D} of about 3×10⁻¹⁰ to about 2.5×10⁻⁹ and an IC₅₀ of inhibiting the Wieslab complement classical pathway of about 1 to 5 nM, about 0.1 to 1 nM, about 0.5 to about 3 nM, about 1 to about 2 nM, less than 5 nM, less than 4.5 nM, less than 3 nM, or less than 1 nM. In some embodiments, the IC₅₀ is about 4 to about 5 nM. In some embodiments, the IC₅₀ is about 2 to about 3 nM. In some embodiments, the IC₅₀ is about 0.5 to about 1 nM. In some embodiments, the K_{D} is about 2.0×10⁻⁹ to about 2.5×10⁻⁹. In some embodiments, the K_{D} is about 1.0×10⁻⁹ to about 1.5×10⁻⁹. In some embodiments, the K_{D} is about 3.0×10⁻¹⁰ to about 3.5×10⁻¹⁰. In some embodiments, the IC₅₀ or K_{D} is about equal to that of MAB4, MAB9 or MAB17. In some embodiments, the antibody is an IgG4 isotype.

As provided for herein, the antibodies, or antigen binding fragments thereof can be variants of the sequences. Accordingly, in some embodiments, a variant of an antibody, or antigen binding fragment thereof, provided for herein is provided. In some embodiments, the variant comprises mutations selected from substitutions, deletions, insertions, or a combination thereof. In some embodiments, the variant comprises between 1 and 20 mutations. In some embodiments, the variant comprises between 1 and 10 mutations. In some embodiments, the variant comprises between 1 and 5 mutations. In some embodiments, the variant comprises 1, 2, 3, 4, 5, 6, 7, 8, 9, 10, 11, 12, 13, 14, 15, 16, 17, 18, 19, or 20 mutations. In some embodiments, the variant comprises at least 1 mutation. Accordingly, in some embodiments, the variant comprises up to or more than 20 mutations. In some embodiments, the variant comprises 1-10 mutations, wherein the mutations are selected from substitutions, deletions, insertions, or a combination thereof. In some embodiments, the variant comprises 1-10 mutations, wherein the mutations are conservative substitutions. Examples of conservative substitutions are as provided for herein (Table 1). Further, one of skill in the art will recognize and understand the substitutions that are encompassed by the term “conservative substitutions”. Such substitutions are within the scope of the present disclosure.

The antibody or antigen binding fragment may comprise an antibody fragment as defined and provided for herein. In some embodiments, the antibody binding fragment is as provided for herein. In some embodiments, the antibody fragment is an scFv antibody, a Fab fragment, a Fab′ fragment, or a F(ab′)₂ fragment. In some embodiments, the antibody fragment is an scFv antibody. In some embodiments, the antibody fragment is a Fab fragment. In some embodiments, the antibody fragment is a Fab′ fragment. In some embodiments, the antibody fragment is a F(ab′)₂ fragment.

The sequences of the antibodies can be modified to yield human IgG antibodies. The conversion of the sequences provided herein can be modified to yield other types of antibodies. The CDRs can also be linked to other antibodies, proteins, or molecules to create antibody fragments that bind C1s. This can be in the form of an antibody drug conjugate (“ADC”), or a multi-specific molecule. The sequences can also be made into chimeric antibodies as described herein.

In some embodiments, the antibody comprises an amino acid sequence comprising a sequence provided for herein or a fragment thereof. In some embodiments, the antibody is a monoclonal antibody. In some embodiments, the antibody is a polyclonal antibody. In some embodiments, the antibody is a humanized antibody. In some embodiments, the antibody comprises one or more amino acid sequences as provided herein, an antigen binding fragments, thereof, or a human IgG variant thereof. “A human IgG variant thereof” refers to an antibody that has been modified to be a human IgG when the starting antibody is not a human IgG antibody.

As described herein the production of antibodies with a known sequence is routine and can be done by any method. Accordingly, in some embodiments, a nucleic acid molecule encoding an antibody or fragment thereof is provided. In some embodiments, the nucleic acid encodes a sequence provided for herein. The antibodies can also be modified to be chimeric antibodies or human antibodies. The antibodies can also be used in injectable pharmaceutical compositions. As also described herein, the antibodies can be isolated antibodies or engineered antibodies.

In some embodiments, an isolated nucleic acid molecule is provided. In some embodiments, the nucleic acid molecule encodes for an antibody, or antigen binding fragment thereof as provided for herein. In some embodiments, the nucleic acid molecule encodes for an antibody, or antigen binding fragment thereof comprising a heavy chain variable region, a light chain variable region, a heavy chain constant region, a light chain constant region or any combination thereof as provided for herein. In some embodiments, the nucleic acid molecule encoding the variable heavy chain is as set forth below:

TABLE 9

| SEQ | AB | |
|---|---|---|
| ID | ID | |
| NO: | NO. | Sequence |
| 268 | MAB1 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CCAGGAAGATTACGCTCTTGACTACTGGGGCCA |
| | | AGGTACGCTGGTTACGGTC |
| 270 | MAB2 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGGCTGGGACTACTGGGGCCA |
| | | AGGTACGCTGGTTACGGTC |
| 272 | MAB3 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGCTTTCGACGAATGGGGCCA |
| | | AGGTACGCTGGTTACGGTC |
| 274 | MAB4 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAGTCAGTACGCTCTTGACTACTGGGGCCA |
| | | AGGTACGCTGGTTACGGTC |
| 276 | MAB5 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCACATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CAACACCGATTACGCTCTTGACCTGTGGGGCCA |
| | | AGGTACGCTGGTTACGGTC |
| 278 | MAB6 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCACATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGCTTACGACGAATGGGGCCA |
| | | AGGTACGCTGGTTACGGTC |
| 280 | MAB7 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGCTTACGACAACTGGGGCCA |
| | | AGGTACGCTGGTTACGGTC |
| 282 | MAB8 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAGTGATTACGCTTACGACTACTGGGGCCA |
| | | AGGTACGCTGGTTACGGTC |
| 284 | MAB9 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAATTACGCTCTTGACTGGTGGGGCCAAGG |
| | | TACGCTGGTTACGGTCTCG |
| 286 | MAB10 | CAGGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAGTGATTACGCTCTTGACTTCTGGGGCCA |
| | | AGGTACGCTGGTTACGGTC |
| 288 | MAB11 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTTTGGCCGGGGTGCCGTTTGATCTGTGG |
| | | GGGCAGGGGACGCTGGTAACGGTCTCGAGT |
| 290 | MAB12 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTTTGGGCGGGCGCCCGTTTGATCACTGG |
| | | GGGCAGGGGACGCTGGTAACGGTCTCGAGT |
| 292 | MAB13 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTTTGGCCGGGTTCCCGTTTGATATCTGG |
| | | GGGCAGGGGACGCTGGTAACGGTCTCGAGT |
| 294 | MAB14 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTTTGGCCTGGCGCCCGACCGATTCATGG |
| | | GGGCAGGGGACGCTGGTAACGGTCTCGAGT |
| 296 | MAB15 | CAAGTTCAACTGGTTCAAAGCGGGGCCGAGGTA |
| | | AAGAAGTCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | AAGGGTTTGGCCGGGCTGCCGTACTACTCATGG |
| | | GGGCAGGGGACGCTGGTAACGGTCTCGAGT |
| 298 | MAB16 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTTTGGCCGGGGTGCCGTTTGATCTGTGG |
| | | GGGCAGGGGACGCTGGTAACGGTCTCGAGT |
| 300 | MAB17 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTGAGGCCGGGCGCCCGTTTGATGCCTGG |
| | | GGGCAGGGGACGCTGGTAACGGTCTCGAGT |
| 302 | MAB18 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTCCCTCAGA |
| | | AGTGAAGATACTGCCGTCTACTACTGTGTGACC |
| | | GAGGGTTTGGCCGGGCGCCCGTACGATGTGTGG |
| | | GGGCAGGGGACGCTGGTAACGGTCTCGAGT |
| 304 | MAB19 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTACCTCAGA |
| | | AGTGAAGATACTGCCGTCTACTACTGTGTGACC |
| | | GAGGGTTTGGCCGGGATCCCGTTTGATTCATGG |
| | | GGGCAGGGGACGCTGGTAACGGTCTCGAGT |
| 306 | MAB20 | CAAGTTCAACTGGTTCAAAGCGGGGCCGAGGTA |
| | | AAGAAGCCAGGCGCTTCTGTGAAAGTCAGTTGC |
| | | AAGGTGAGTGGGGATACATTGACCGAGCTGAG |
| | | TATGCACTGGGTCCGGCAAGCTCCTGGCAAGGG |
| | | TTTGGAATGGATGGGTACTTTTGACCCGGAGGA |
| | | GGGAGAGACCATCTACGCGCAAAAATTCCAAG |
| | | GTAGGGTGACCATGACGGAGGATACCAGCACG |
| | | GATACTGCTTACATGGAGCTCAGCTACCTCAGA |
| | | AGTGAAGATACTGCCGTCTACTACTGTGTGACC |
| | | GAGGGTTTGGCCGGGATCCCGTTTGATTCATGG |
| | | GGGCAGGGGACGCTGGTAACGGTCTCGAGT |
| 308 | MAB21 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGACTACTTATTATTACTA |
| | | TTACATGGACGTGTGGGGTAAAGGAACGACCGT |
| | | CACAGTC |
| 310 | MAB22 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGAAGTATTACTATTACTA |
| | | TTACATGGACGTGTGGGGTAAAGGAACGACCGT |
| | | CACAGTC |
| 312 | MAB23 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCAGACTTATTATTACTA |
| | | TTACATGGACGTGTGGGGTAAAGGAACGACCGT |
| | | CACAGTC |
| 314 | MAB24 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGACCACCTATTATTACTA |
| | | TTACATGGACGTGTGGGGTAAAGGAACGACCGT |
| | | CACAGTC |
| 316 | MAB25 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGATCAAGTATTATTACTA |
| | | TTACATGGACGTGTGGGGTAAAGGAACGACCGT |
| | | CACAGTC |
| 318 | MAB26 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACTTTCGATGACTACGGA |
| | | ATGTCATGGGTACGACAAGCTCCCGGCAAAGGC |
| | | CTTGAATGGGTTTCTGGCATCAACTGGGAGGGC |
| | | GGTTCCACTGGCTACGCGGACTCAGTTAAGGGT |
| | | AGGTTCAGCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCTGAAGTATTATTACTA |
| | | TTACATGGACGTGTGGGGTAAAGGAACGACCGT |
| | | CACAGTC |
| 320 | MAB27 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGAAGGTGTATTATTACTA |
| | | TTACATGGACGTGTGGGGTAAAGGAACGACCGT |
| | | CACAGTC |
| 322 | MAB28 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACTTTCGATGACTACGGA |
| | | ATGTCATGGGTACGACAAGCTCCCGGCAAAGGC |
| | | CTTGAATGGGTTTCTGGCATCAACTGGGAGGGC |
| | | GGTTCCACTGGCTACGCGGACTCAGTTAAGGGT |
| | | AGGTTCAGCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCTGAAGTATTATTACTA |
| | | TTACATGGACGTGTGGGGTAAAGGAACGACCGT |
| | | CACAGTC |
| 324 | MAB29 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGAATCACTATTATTACTA |
| | | TTACATGGACGCTTGGGGTAAAGGAACGACCGT |
| | | CACAGTC |
| 326 | MAB30 | GAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCGCCACTATTATTACTA |
| | | TTACATGGACGTGTGGGGTAAAGGAACGACCGT |
| | | CACAGTC |
| 424 | MAB31 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CCAGGAAGATTACGCTCTTGACTACTGGGGCCA |
| | | AGGTACGCTGGTTACGGTC |
| 425 | MAB32 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGGCTGGGACTACTGGGGCCA |
| | | AGGTACGCTGGTTACGGTC |
| 426 | MAB33 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGCTTTCGACGAATGGGGCCA |
| | | AGGTACGCTGGTTACGGTC |
| 427 | MAB34 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAGTCAGTACGCTCTTGACTACTGGGGCCA |
| | | AGGTACGCTGGTTACGGTC |
| 428 | MAB35 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCACATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CAACACCGATTACGCTCTTGACCTGTGGGGCCA |
| | | AGGTACGCTGGTTACGGTC |
| 429 | MAB36 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCACATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGCTTACGACGAATGGGGCCA |
| | | AGGTACGCTGGTTACGGTC |
| 430 | MAB37 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGACCGATTACGCTTACGACAACTGGGGCCA |
| | | AGGTACGCTGGTTACGGTC |
| 431 | MAB38 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAGTGATTACGCTTACGACTACTGGGGCCA |
| | | AGGTACGCTGGTTACGGTC |
| 432 | MAB39 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAATTACGCTCTTGACTGGTGGGGCCAAGG |
| | | TACGCTGGTTACGGTCTCG |
| 433 | MAB40 | GAAGTGCAGCTTGTTGAAAGTGGTGGGGGTTTG |
| | | GTTAAACCTGGCGGTTCCCTTCGACTTAGCTGC |
| | | GCGGCGTCAGGGTTCACATTCTCAGATTATTAC |
| | | ATGTCTTGGATTCGGCAAGCCCCTGGTAAGGGA |
| | | CTGGAGTGGGTAAGCTACATATCTCGGTCAGGA |
| | | AGTACAAAGTACTATGCAGATTCAGTGAAGGGC |
| | | AGGTTTACGATTAGCCGAGACAACGCAAAGAA |
| | | CTCTCTTTATCTGCAGATGAATTCACTGAGAGC |
| | | AGAAGATACCGCTGTCTATTATTGTGCCAGAGA |
| | | CGAGAGTGATTACGCTCTTGACTTCTGGGGCCA |
| | | AGGTACGCTGGTTACGGTC |
| 434 | MAB41 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTTTGGCCGGGGTGCCGTTTGATCTGTG |
| | | GGGGCAGGGGACGCTGGTAACGGTCTCGAGT |
| 435 | MAB42 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTTTGGGCGGGCGCCCGTTTGATCACTG |
| | | GGGGCAGGGGACGCTGGTAACGGTCTCGAGT |
| 436 | MAB43 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTTTGGCCGGGTTCCCGTTTGATATCTG |
| | | GGGGCAGGGGACGCTGGTAACGGTCTCGAGT |
| 437 | MAB44 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTTTGGCCTGGCGCCCGACCGATTCATG |
| | | GGGGCAGGGGACGCTGGTAACGGTCTCGAGT |
| 438 | MAB45 | GAAGTTCAACTGGTTCAAAGCGGGGCCGAGGT |
| | | AAAGAAGTCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CAAGGGTTTGGCCGGGCTGCCGTACTACTCATG |
| | | GGGGCAGGGGACGCTGGTAACGGTCTCGAGT |
| 439 | MAB46 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTTTGGCCGGGGTGCCGTTTGATCTGTG |
| | | GGGGCAGGGGACGCTGGTAACGGTCTCGAGT |
| 440 | MAB47 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTGAGGCCGGGCGCCCGTTTGATGCCTG |
| | | GGGGCAGGGGACGCTGGTAACGGTCTCGAGT |
| 441 | MAB48 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTCCCTCAG |
| | | AAGTGAAGATACTGCCGTCTACTACTGTGTGAC |
| | | CGAGGGTTTGGCCGGGCGCCCGTACGATGTGTG |
| | | GGGGCAGGGGACGCTGGTAACGGTCTCGAGT |
| 442 | MAB49 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTACCTCAG |
| | | AAGTGAAGATACTGCCGTCTACTACTGTGTGAC |
| | | CGAGGGTTTGGCCGGGATCCCGTTTGATTCATG |
| | | GGGGCAGGGGACGCTGGTAACGGTCTCGAGT |
| 443 | MAB50 | GAAGTTCAACTGGTTCAAAGCGGGGCCGAGGT |
| | | AAAGAAGCCAGGCGCTTCTGTGAAAGTCAGTTG |
| | | CAAGGTGAGTGGGGATACATTGACCGAGCTGA |
| | | GTATGCACTGGGTCCGGCAAGCTCCTGGCAAGG |
| | | GTTTGGAATGGATGGGTACTTTTGACCCGGAGG |
| | | AGGGAGAGACCATCTACGCGCAAAAATTCCAA |
| | | GGTAGGGTGACCATGACGGAGGATACCAGCAC |
| | | GGATACTGCTTACATGGAGCTCAGCTACCTCAG |
| | | AAGTGAAGATACTGCCGTCTACTACTGTGTGAC |
| | | CGAGGGTTTGGCCGGGATCCCGTTTGATTCATG |
| | | GGGGCAGGGGACGCTGGTAACGGTCTCGAGT |
| 444 | MAB51 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGACTACTTATTATTACTA |
| | | TTACATGGACGTGTGGGGTAAAGGAACGACCGT |
| | | CACAGTC |
| 445 | MAB52 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGAAGTATTACTATTACTA |
| | | TTACATGGACGTGTGGGGTAAAGGAACGACCGT |
| | | CACAGTC |
| 446 | MAB53 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCAGACTTATTATTACTA |
| | | TTACATGGACGTGTGGGGTAAAGGAACGACCGT |
| | | CACAGTC |
| 447 | MAB54 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGACCACCTATTATTACTA |
| | | TTACATGGACGTGTGGGGTAAAGGAACGACCGT |
| | | CACAGTC |
| 448 | MAB55 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGATCAAGTATTATTACTA |
| | | TTACATGGACGTGTGGGGTAAAGGAACGACCGT |
| | | CACAGTC |
| 449 | MAB56 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACTTTCGATGACTACGGA |
| | | ATGTCATGGGTACGACAAGCTCCCGGCAAAGGC |
| | | CTTGAATGGGTTTCTGGCATCAACTGGGAGGGC |
| | | GGTTCCACTGGCTACGCGGACTCAGTTAAGGGT |
| | | AGGTTCAGCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCTGAAGTATTATTACTA |
| | | TTACATGGACGTGTGGGGTAAAGGAACGACCGT |
| | | CACAGTC |
| 450 | MAB57 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGAAGGTGTATTATTACTA |
| | | TTACATGGACGTGTGGGGTAAAGGAACGACCGT |
| | | CACAGTC |
| 451 | MAB58 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACTTTCGATGACTACGGA |
| | | ATGTCATGGGTACGACAAGCTCCCGGCAAAGGC |
| | | CTTGAATGGGTTTCTGGCATCAACTGGGAGGGC |
| | | GGTTCCACTGGCTACGCGGACTCAGTTAAGGGT |
| | | AGGTTCAGCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCTGAAGTATTATTACTA |
| | | TTACATGGACGTGTGGGGTAAAGGAACGACCGT |
| | | CACAGTC |
| 452 | MAB59 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGAATCACTATTATTACTA |
| | | TTACATGGACGCTTGGGGTAAAGGAACGACCGT |
| | | CACAGTC |
| 453 | MAB60 | CAAGTCCAGCTTGTTGAGTCTGGCGGAGGGGTG |
| | | GTTAGGCCAGGAGGTTCCCTTAGGCTTTCCTGT |
| | | GCGGCATCAGGATTTACCTTCGATGACTACGGA |
| | | ATGTCATGGGTACGACAAGCTCCCGGCAAAGGC |
| | | CTCGAATGGGTTTCTGGCATCAACTGGGAGGGC |
| | | GGTTCCACTGGCTACGCGGACTCAGTTAAGGGT |
| | | AGGTTCACCATAAGCAGGGACAATGCGAAGAA |
| | | CTCACTGTATCTCCAAATGAACAGTCTCCGCGC |
| | | CGAGGATACAGCCCTTTACTATTGTGCACGAGA |
| | | TGAGCAATTGGGGGGGCGCCACTATTATTACTA |
| | | TTACATGGACGTGTGGGGTAAAGGAACGACCGT |
| | | CACAGTC |

In some embodiments, the nucleic acid molecule encoding the variable light chain is as set forth below:

TABLE 10

| SEQ | AB | |
|---|---|---|
| ID | ID | |
| NO: | NO. | Sequence |
| 269 | MAB1 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TGAACAGTATGAGGACTACCCTCTTACCTTCGG |
| | | CGGTGGAACGAAAGTTGAGATTAAGCGAACC |
| 271 | MAB2 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TAAGCAGTATGAGGACTACCCTCTTACCTTCGG |
| | | CGGTGGAACGAAAGTTGAGATTAAGCGAACC |
| 273 | MAB3 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TAAGCAGTATGAGGACTACCCTCTTACCTTCGG |
| | | CGGTGGAACGAAAGTTGAGATTAAGCGAACC |
| 275 | MAB4 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCACCAGTATGAGGACTACCCTCTTACCTTCGG |
| | | CGGTGGAACGAAAGTTGAGATTAAGCGAACC |
| 277 | MAB5 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCACCAGTATGAGGACTACCCTCTTACCTTCGG |
| | | CGGTGGAACGAAAGTTGAGATTAAGCGAACC |
| 279 | MAB6 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCACCAGTATGAGGACTACCCTCTTACCTTCGG |
| | | CGGTGGAACGAAAGTTGAGATTAAGCGAACC |
| 281 | MAB7 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCAGCAGTATGAGGACTACCCTCTTGTGTTCGG |
| | | CGGTGGAACGAAAGTTGAGATTAAGCGAACC |
| 283 | MAB8 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCAGCAGCACGAGGACTACCCTCTTACCTTCGG |
| | | CGGTGGAACGAAAGTTGAGATTAAGCGAACC |
| 285 | MAB9 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCAGCACTATGAGGACTACCCTCTTACCTTCGG |
| | | CGGTGGAACGAAAGTTGAGATTAAGCGAACC |
| 287 | MAB10 | GACATTCAGATGACGCAAAGCCCCTCTAGCTTG |
| | | TCCGCTAGTGTGGGTGACAGGGTCACCATTACC |
| | | TGCCAGGCTTCACAAGACATCAGTAATTACCTC |
| | | AACTGGTATCAGCAGAAACCTGGGAAGGCCCCT |
| | | AAGCTGTTGATTTACGATGCTAGTAACTTGGAG |
| | | ACCGGGGTACCCAGCAGATTTAGCGGGAGCGG |
| | | AAGTGGTACGGATTTTACGTTTACCATTAGCAG |
| | | CTTGCAGCCCGAGGACATCGCTACATATTACTG |
| | | TCAGCAGTATGAGGACCTCATCCCCACCTTCGG |
| | | CGGTGGAACGAAAGTTGAGATTAAGCGAACC |
| 289 | MAB11 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGTGGATCAGGA |
| | | TCTGGTACTGAATTCACTCTGACCATTTCAAGTC |
| | | TTCAGCCTGATGACTTTGCGACCTATTATTGCCA |
| | | GAGCTACAATAGCTACGTGTGGACGTTCGGGCA |
| | | GGGTACTAAAGTCGAGATTAAACGAACC |
| 291 | MAB12 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGTGGATCAGGA |
| | | TCTGGTACTGAATTCACTCTGACCATTTCAAGTC |
| | | TTCAGCCTGATGACTTTGCGACCTATTATTGCCA |
| | | GCAGGTGAATAGCTACGACTGGACGTTCGGGCA |
| | | GGGTACTAAAGTCGAGATTAAACGAACC |
| 293 | MAB13 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGTGGATCAGGA |
| | | TCTGGTACTGAATTCACTCTGACCATTTCAAGTC |
| | | TTCAGCCTGATGACTTTGCGACCTATTATTGCCA |
| | | GCAGGTGAATAGCTACGACTGGACGTTCGGGCA |
| | | GGGTACTAAAGTCGAGATTAAACGAACC |
| 295 | MAB14 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCCA |
| | | GCAGTACAGCAGCTACGCCTGGACGTTCGGGCA |
| | | GGGTACTAAAGTCGAGATTAAACGAACC |
| 297 | MAB15 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCCA |
| | | GCAGGCCCGCAGCTACTCATGGACGTTCGGGCA |
| | | GGGTACTAAAGTCGAGATTAAACGAACC |
| 299 | MAB16 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCCA |
| | | GCAGCACAATAGCTACCGCTGGACGTTCGGGCA |
| | | GGGTACTAAAGTCGAGATTAAACGAACC |
| 301 | MAB17 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCGT |
| | | GCAGTACCCCAGCTACTCATGGACGTTCGGGCA |
| | | GGGTACTAAAGTCGAGATTAAACGAACC |
| 303 | MAB18 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCCA |
| | | GCAGTACAATAGCTACAAGCTGACGTTCGGGCA |
| | | GGGTACTAAAGTCGAGATTAAACGAACC |
| 305 | MAB19 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCCA |
| | | GCAGTACAATAGCCCCTCATGGCTGTTCGGGCA |
| | | GGGTACTAAAGTCGAGATTAAACGAACC |
| 307 | MAB20 | GACATACAAATGACTCAATCACCCTCTACACTT |
| | | TCCGCCTCTGTCGGCGACAGGGTAACGATAACT |
| | | TGCCGGGCAAGTCAATCAATCAGCTCCTGGCTT |
| | | GCTTGGTATCAACAAAAACCAGGCAAGGCACCT |
| | | AAGCTCCTCATTTACAAGGCGTCATCACTTGAA |
| | | TCAGGGGTGCCTTCACGATTCAGCGGATCAGGA |
| | | TCTGGTACTGAATTCACTCTGACCATTTCAAGTC |
| | | TTCAGCCTGATGACTTTGCGACCTATTATTGCCA |
| | | GCAGGTGAATAGCCTGTCATGGACGTTCGGGCA |
| | | GGGTACTAAAGTCGAGATTAAACGAACC |
| 309 | MAB21 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TCCAGTATAATAGTTATCCTCTCGGCTTTGGCGG |
| | | TGGCACCAAAGTGGAGATTAAGCGAACC |
| 311 | MAB22 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTACCGCAGTCACTATCCTCTCACTTTTGG |
| | | CGGTGGCACCAAAGTGGAGATTAAGCGAACC |
| 313 | MAB23 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTATAATCAGGTGCCTCTCACTTTTGGCG |
| | | GTGGCACCAAAGTGGAGATTAAGCGAACC |
| 315 | MAB24 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGACCAATATCTATCCTCTCACTTTTGGCGG |
| | | TGGCACCAAAGTGGAGATTAAGCGAACC |
| 317 | MAB25 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTATAAGCAGTATCCTCTCACTTTTGGCG |
| | | GTGGCACCAAAGTGGAGATTAAGCGAACC |
| 319 | MAB26 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGACCAATATCTATCCTCTCACTTTTGGCGG |
| | | TGGCACCAAAGTGGAGATTAAGCGAACC |
| 321 | MAB27 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTATAATAGTTATCCTCTCGCCTTTGGCGG |
| | | TGGCACCAAAGTGGAGATTAAGCGAACC |
| 323 | MAB28 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTATCACAGTTATCCTCTCCGCTTTGGCGG |
| | | TGGCACCAAAGTGGAGATTAAGCGAACC |
| 325 | MAB29 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTATGCCAGTTATCCTCTCAAGTTTGGCG |
| | | GTGGCACCAAAGTGGAGATTAAGCGAACC |
| 327 | MAB30 | GACATACAGATGACTCAGTCCCCAAGTAGTCTG |
| | | AGCGCCTCTGTCGGCGACCGCGTTACCATTACT |
| | | TGCCGGGCGAGTCAAGGGATACGCAACGATTTG |
| | | GGTTGGTATCAGCAAAAGCCCGGAAAGGCCCC |
| | | AAAGCGGCTGATATACACCGCCTCAAACCTCCA |
| | | GTCTGGAGTTCCATCAAGGTTTAGTGGATCTGG |
| | | ATCTGGCACTGAGTTTACTCTCACTATAAGCTCC |
| | | CTTCAGCCTGAAGACTTCGCAACTTATTATTGCT |
| | | TGCAGTATAATGCCTATCCTCTCATCTTTGGCGG |
| | | TGGCACCAAAGTGGAGATTAAGCGAACC |

In some embodiments, the nucleic acid molecule comprises a sequence of SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435 SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, or SEQ ID NO: 453, as set forth in the table above. It is to be understood that the nucleotide sequences of are exemplary sequences and are not meant to be limiting in any way. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is an mRNA molecule. Accordingly, in some embodiments, the nucleic acid molecule comprises a sequence that is substantially similar to those provided for herein. In some embodiments, the nucleic acid molecule comprises a sequence that has at least 60%, 65%, 70%, 75%, 80%, 85%, 90%, 95%, or 100% identity to the sequences provided for herein, or any percent identity falling within any of the recited percent identities. In some embodiments, the nucleic acid molecule comprises a sequence that has at least 60%, 65%, 70%, 75%, 80%, 85%, 90%, 95%, or 100% identity to the nucleic acid molecules provided for herein, or any percent identity falling within any of the recited percent identities.

In some embodiments, an expression vector is provided. In some embodiments, the expression vector comprises a nucleic acid molecule encoding an antibody or antigen-binding fragment thereof as provided for herein. In some embodiments, the expression vector comprises a nucleic acid molecule as provided for herein. The vector may be any vector known in the art. In some embodiments the vector is as provided for herein. In some embodiments, the vector is a plasmid. In some embodiments the vector is a virus.

In some embodiments, “derivatives” of the antibodies, fragments, regions or derivatives thereof, which term includes those proteins encoded by truncated or modified genes to yield molecular species functionally resembling the immunoglobulin fragments are provided. The modifications include, but are not limited to, addition of genetic sequences coding for cytotoxic proteins such as plant and bacterial toxins. The modification can also include a reporter protein, such as a fluorescent or chemiluminescent tag. The fragments and derivatives can be produced in any manner.

The identification of these antibodies, or antigen binding fragments thereof, provide the information necessary to generate additional monoclonal antibodies with similar binding characteristics and therapeutic or diagnostic utility that parallel the embodiments of this application.

The antibodies can be generated according to the examples provided herein. Once the sequences are known, the antibodies can also be generated according to known methods. The antibodies can also be converted to different types, such as being converted to Human IgGs and the like. By converting the antibodies to a human antibody, a human subject should not identify the antibodies as foreign. The conversion of a non-human IgG antibody to a human IgG antibody is well known and can routinely be done once the native sequence is known. As discussed herein, the antibodies can be modified according to known methods. Such methods are described in, for example, Riechmann L, Clark M, Waldmann H, Winter G (1988). Reshaping human antibodies for therapy”. Nature 332 (6162): 332-323; Tsurushita N, Park M, Pakabunto K, Ong K, Avdalovic A, Fu H, Jia A, Visquez M, Kumar S. (2004), which is incorporated by reference in its entirety.

In some embodiments, a host cell is provided. In some embodiments, the host cell comprises an antibody or antigen-binding fragment thereof as provided for herein. In some embodiments, the host cell comprises a nucleic acid molecule encoding for antibody or antigen-binding fragment thereof as provided for herein. In some embodiments, the host cell comprises nucleic acid molecules as provided for herein, or any variants thereof as provided for herein, or any combination thereof. In some embodiments, the host cell comprises a vector, said vector comprising comprises a nucleic acid molecule encoding for antibody or antigen-binding fragment thereof as provided for herein. In some embodiments, the vector comprises nucleic acid molecules as provided for herein, any variants thereof as provided for herein, or any combination thereof.

In some embodiments, the host cell produces an antibody or antigen-binding fragment thereof as provided for herein. In some embodiments, an antibody or antigen-binding fragment thereof as provided for herein is provided, wherein the antibody or antigen-binding fragment thereof as provided for herein is produced by the host cell.

In some embodiments, a method of producing a polypeptide is provided. In some embodiments, the polypeptide comprises a heavy chain variable region as provided for herein, a light chain variable region as provided for herein, or a combination thereof. In some embodiments, the method comprises growing a host cell under conditions so that the host cell expresses the polypeptide comprising the heavy chain variable region, light chain variable region, or combination thereof, and purifying the polypeptide comprising the heavy chain variable region, the light chain variable region, or a combination thereof, thereby producing the polypeptide. In some embodiments, the host cell is a host cell as provided for herein.

In some embodiments, a method of producing an antibody, or antigen binding fragment thereof, is provided. In some embodiments, the antibody binds human C1s. In some embodiments, the antigen binding fragment binds human C1s. In some embodiments, the method comprises growing a host cell under conditions so that the host cell expresses a polypeptide or polypeptides comprising the immunoglobulin heavy chain variable region and/or the immunoglobulin light chain variable region, thereby producing the antibody or the antigen binding fragment of the antibody, and purifying the antibody or the antigen binding fragment of the antibody. In some embodiments, the polypeptide or polypeptides comprise the immunoglobulin heavy chain variable region. In some embodiments, the polypeptide or polypeptides comprise the immunoglobulin light chain variable region. In some embodiments, the polypeptide or polypeptides comprise the immunoglobulin heavy chain variable region and the immunoglobulin light chain variable region. In some embodiments, the host cell is a host cell as provided for herein. In some embodiments, the antibody, or antigen binding fragment thereof, is as provided for herein.

The polypeptide or antibody-producing cell contributing the nucleotide sequences encoding the antigen-binding region of the chimeric antibody can also be produced by transformation of a non-human, such as a primate, or a human cell. For example, a B lymphocyte which produces the antibody can be infected and transformed with a virus such as Epstein-Barr virus to yield an immortal antibody producing cell (Kozbor et al., Immunol. Today 4:72 79 (1983)). Alternatively, the B lymphocyte can be transformed by providing a transforming gene or transforming gene product, as is well-known in the art. See, e.g., Ausubel infra, Harlow infra, and Colligan infra, the contents of which references are incorporated entirely herein by reference. The cell fusions are accomplished by standard procedures well known to those skilled in the field of immunology, such as producing cells can also be a hybridoma which is generated by fusing a B-cell with an immortal myeloma cell. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

The nucleic acid sequence encoding an antibody described herein can be genomic DNA or cDNA, or RNA (e.g. mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize C1s can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe, et al., J. Molec. Biol. 183:1 12 (1985). Using the “codon usage rules” of Lathe, a single oligonucleotide, or a set of oligonucleotides, that contains a theoretical “most probable” nucleotide sequence capable of encoding an antibody variable or constant region sequences is identified.

The variable regions described herein can be combined with any type of constant region including a human constant region or murine constant region. Human genes which encode the constant (C) regions of the antibodies, fragments and regions can be derived from a human fetal liver library, by known methods. Human C region genes can be derived from any human cell including those which express and produce human immunoglobulins. The human C_{H} region can be derived from any of the known classes or isotypes of human H chains, including gamma, p, a, 6 or 8, and subtypes thereof, such as G1, G2, G3 and G4. Since the H chain isotype is responsible for the various effector functions of an antibody, as well as antibody recycling by interaction with FcRn, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (IgG3), gamma 4 (IgG4), or ρ (IgM). The human C_{L} region can be derived from either human L chain isotype, kappa or lambda.

Methods of purifying polypeptides or proteins (including antibodies or antigen binding fragments thereof) are known in the art, and any such method is within the scope of the present application. Further, the host cell is not limited by the examples recited above. Any suitable cell may be used to generate the polypeptides or proteins of the present disclosure.

### Pharmaceutical Compositions

In some embodiments, a pharmaceutical composition is provided. In some embodiments, the pharmaceutical composition comprises an antibody or antigen-binding fragment thereof as provided for herein.

In some embodiments, to prepare pharmaceutical or sterile compositions of the anti-C1s antibodies or other proteins provided herein, the antibody, or antigen binding fragment thereof, or other proteins provided herein are admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic or the antibodies provided herein may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments, the antibodies are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In some embodiments, a composition is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the composition is an injectable pharmaceutical composition. In some embodiments, the composition is formulated for intravenous or subcutaneous injection. In some embodiments, the composition is formulated for intravenous injection. In some embodiments, the composition is formulated for subcutaneous injection.

In some embodiments, the antibody, or antigen binding fragment thereof, can be administered by an invasive route such as by injection. In some embodiments, the antibodies or antigen binding fragment thereof, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present embodiments.

In some embodiments, the anti-C1s antibody, or antigen binding fragment thereof, is administered in combination with at least one additional therapeutic agent, such as, but not limited to any therapeutic used to treat the disorders provided for herein. In some embodiments, the antibody is administered in combination with another treatment for the disorders provided for herein.

Compositions can be administered with medical devices known in the art. For example, a pharmaceutical composition of the invention can be administered by injection with a hypodermic needle, including, e.g., a prefilled syringe or autoinjector.

The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose can be made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies may be desirable.

Antibodies or antigen binding fragments thereof can be provided by continuous infusion, or by doses administered, e.g., daily, 1-7 times per week, weekly, bi-weekly, monthly, bimonthly, or quarterly. In some embodiments, a total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang, et al. (2003) New Engl. J Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). Doses may also be provided to achieve a pre-determined target concentration of the antibody in the subject's serum, such as 0.1, 0.3, 1, 3, 10, 30, 100, 300 μg/ml or more. In other embodiments, a fully human antibody is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of the antibody, or antigen binding fragment thereof, that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. In some embodiments, an amount is a therapeutically effective amount if it is an amount that can be used to treat or ameliorate tumors or gastric tumors.

The term “subject” as used throughout includes any organism, such as an animal, including a mammal (e.g., rat, mouse, dog, cat, rabbit) and, for example, a human. In one embodiment, the subject is a human. A subject can also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein.

In some embodiments, the methods comprise administering a therapeutically or prophylactically effective amount of one or more antibodies or antigen binding fragments of the antibodies described herein to a susceptible subject or to one exhibiting a condition in which C1s is known to have caused the pathology observed. Any active form of the antibody can be administered, including, but not limited to scFv, Fab and F(ab′)2 fragments and other forms of antibodies provided for herein.

The present disclosure provides a method to treat an individual having a complement-mediated disease or disorder, the method comprising administering to the individual an anti-C1s antibody of any of the embodiments disclosed herein or a pharmaceutical composition thereof. In some embodiments, the individual is a mammal. In some embodiments, the individual is a human. In some embodiments, the administering is intravenous. In some embodiments, the administering is subcutaneous. In some embodiments, the administering is intrathecal. In some embodiments, the administering results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (l) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction in autoantibody mediated blister formation; (ad) a reduction in autoantibody induced pruritis; (ae) a reduction in autoantibody induced erythematosus; (af) a reduction in autoantibody mediated skin erosion; (ag) a reduction in red blood cell destruction due to transfusion reactions; (ah) a reduction in red blood cell lysis due to alloantibodies; (ai) a reduction in hemolysis due to transfusion reactions; (aj) a reduction in allo-antibody mediated platelet lysis; (ak) a reduction in platelet lysis due to transfusion reactions; (al) a reduction in mast cell activation; (am) a reduction in mast cell histamine release; (an) a reduction in vascular permeability; (ao) a reduction in edema; (ap) a reduction in complement deposition on transplant graft endothelium; (aq) a reduction of anaphylatoxin generation in transplant graft endothelium; (ar) a reduction in the separation of the dermal-epidermal junction; (as) a reduction in the generation of anaphylatoxins in the dermal-epidermal junction; (at) a reduction in alloantibody mediated complement activation in transplant graft endothelium; (au) a reduction in antibody mediated loss of the neuromuscular junction; (av) a reduction in complement activation at the neuromuscular junction; (aw) a reduction in anaphylatoxin generation at the neuromuscular junction; (ax) a reduction in complement deposition at the neuromuscular junction; (ay) a reduction in paralysis; (az) a reduction in numbness; (ba) increased bladder control; (bb) increased bowel control; (bc) a reduction in mortality associated with autoantibodies; and (bd) a reduction in morbidity associated with autoantibodies. In some embodiments, the reduction in glial cell activation comprises reduction in astrocyte activation or reduction in microglia activation.

In some embodiments, a method of treating a subject with a C1s mediated disorder is provided. In some embodiments, the method comprises administering to the subject an antibody or antigen-binding fragment thereof as provided for herein or a pharmaceutical composition comprising an antibody or antigen-binding fragment thereof as provided for herein, thereby treating the C1s mediated disorder. The antibody or antigen-binding fragment thereof of any of the embodiments provided for herein or the pharmaceutical compositions of any of the embodiments provided for herein inhibit complement C1s activity in an individual having a complement-mediated disease or disorder. In some embodiments, the individual is a mammal. In some embodiments, the individual is a human. In some embodiments, the administering is intravenous. In some embodiments, the administering is subcutaneous. In some embodiments, the administering is intrathecal. In some embodiments, the administering results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (l) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction in autoantibody mediated blister formation; (ad) a reduction in autoantibody induced pruritis; (ae) a reduction in autoantibody induced erythematosus; (af) a reduction in autoantibody mediated skin erosion; (ag) a reduction in red blood cell destruction due to transfusion reactions; (ah) a reduction in red blood cell lysis due to alloantibodies; (ai) a reduction in hemolysis due to transfusion reactions; (aj) a reduction in allo-antibody mediated platelet lysis; (ak) a reduction in platelet lysis due to transfusion reactions; (al) a reduction in mast cell activation; (am) a reduction in mast cell histamine release; (an) a reduction in vascular permeability; (ao) a reduction in edema; (ap) a reduction in complement deposition on transplant graft endothelium; (aq) a reduction of anaphylatoxin generation in transplant graft endothelium; (ar) a reduction in the separation of the dermal-epidermal junction; (as) a reduction in the generation of anaphylatoxins in the dermal-epidermal junction; (at) a reduction in alloantibody mediated complement activation in transplant graft endothelium; (au) a reduction in antibody mediated loss of the neuromuscular junction; (av) a reduction in complement activation at the neuromuscular junction; (aw) a reduction in anaphylatoxin generation at the neuromuscular junction; (ax) a reduction in complement deposition at the neuromuscular junction; (ay) a reduction in paralysis; (az) a reduction in numbness; (ba) increased bladder control; (bb) increased bowel control; (bc) a reduction in mortality associated with autoantibodies; and (bd) a reduction in morbidity associated with autoantibodies. In some embodiments, the reduction in glial cell activation comprises reduction in astrocyte activation or reduction in microglia activation. In some embodiments, the C1s mediated disorder is selected from the group including, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, these conditions can be caused by stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomysositis. In some embodiments, the C1s mediated disorder is Anti MAG neuropathy. In some embodiments, the C1s mediated disorder is due to stroke. In some embodiments, the C1s mediated disorder is due to spinal cord injury.

In some embodiments, the antibody or antigen-binding fragment thereof of any of the embodiments as provided for herein, or a pharmaceutical composition comprising the antibody or antigen-binding fragment thereof of any of the embodiments as provided for herein is for the use in the treatment of a complement-mediated disease or disorder. In some embodiments, the complement-mediated disorder is selected from the group including, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, these conditions can be due to stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to spinal cord injury. In some embodiments, the C1s mediated disorder is selected from the group including, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to Spinal Cord Injury.

The present disclosure provides use of an anti-C1s antibody of any of the embodiments in the manufacture of a medicament for the treatment of an individual having a complement-mediated disease or disorder. Accordingly, in some embodiments, an antibody, or antigen binding fragment thereof, or a pharmaceutical composition is provided for use as a medicament. In some embodiments, an antibody, or antigen binding fragment thereof, is provided for use as a medicament. In some embodiments, a pharmaceutical composition is provided for use as a medicament. In some embodiments, the antibody, or antigen binding fragment thereof, is an antibody or antigen binding fragment as provided for herein. In some embodiments, the pharmaceutical composition comprises an antibody or antigen binding fragment as provided for herein. In some embodiments, the pharmaceutical composition is as provided for herein. In some embodiments, the medicament is for use in treatment of a C1s mediated disease or disorder. In some embodiments, the complement-mediated disorder is, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the conditions can be due to stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to spinal cord injury.

In some embodiments, a use of an antibody or antigen binding fragment as provided for herein or a pharmaceutical composition as provided for herein is provided. In some embodiments, the use is for the treatment of a C1s mediated disorder. In some embodiments, a use of an antibody or antigen binding fragment as provided for herein is provided, the use for the treatment of a C1s mediated disorder. In some embodiments, a use of a pharmaceutical composition comprising an antibody or antigen binging fragment as provided for herein is provided, the use for the treatment of a C1s mediated disorder. In some embodiments, the pharmaceutical composition is as provided for herein. The antibody of any of the embodiments or pharmaceutical compositions thereof inhibit complement C1s activity in an individual having a complement-mediated disease or disorder. In some embodiments, the complement-mediated disorder is, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the conditions can be due to stroke, or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to spinal cord injury.

The present disclosure provides use of an anti-C1s antibody of any of the embodiments or a pharmaceutical composition thereof in the manufacture of a medicament for inhibiting complement C1s activity. In some embodiments, the present disclosure provides use of an anti-C1s antibody of any of the embodiments or a pharmaceutical composition thereof in the manufacture of a medicament for inhibiting complement C1s activity in an individual having a complement-mediated disease or disorder. In some embodiments, the complement-mediated disorder is, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments the conditions are caused by a stroke or due to spinal cord injury.

The present disclosure provides an anti-C1s antibody of any of the embodiments or a pharmaceutical composition thereof for use in medical therapy.

The present disclosure provides an anti-C1s antibody of any of the embodiments or a pharmaceutical composition thereof for treating an individual having a complement-mediated disease or disorder. In some embodiments, the complement-mediated disorder is, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the conditions are due to stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to Spinal Cord Injury.

The present disclosure provides an anti-C1s antibody of any of the embodiments or a pharmaceutical composition thereof for inhibiting complement C1s protein activity. The present disclosure provides an anti-C1s antibody of any of the embodiments or a pharmaceutical composition thereof for inhibiting complement C1s protein activity in an individual having a complement-mediated disease or disorder. In some embodiments, the complement-mediated disorder is, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the conditions are caused by a stroke or spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to spinal cord injury.

The present disclosure provides a method to diagnose a complement-mediated disease or disorder in an individual, the method comprising: (a) determining the amount of a complement C1s protein in a biological sample obtained from the individual, wherein the step of determining comprises: (i) contacting the biological sample with an anti-C1s antibody of any of the embodiments; and (ii) quantitating binding of the antibody to complement C1s protein present in the sample; and (b) comparing the amount of the complement C1s protein to a normal control value that indicates the amount of complement C1s protein in a normal control individual, wherein a significant difference between the amount of C1s protein in the biological sample and the normal control value indicates that the individual has a complement-mediated disease or disorder. In some embodiments, the biological sample is selected from the group consisting of blood, serum, plasma, urine, saliva, cerebrospinal fluid, interstitial fluid, ocular fluid, synovial fluid, solid tissue sample, tissue culture sample, and cellular sample.

The present disclosure provides a method to monitor progression of a complement-mediated disease or disorder in an individual, the method comprising: (a) determining a first amount of complement a C1s protein in a biological sample obtained from the individual at a first time point; (b) determining a second amount of complement a C1s protein in a biological sample obtained from the individual at a second time point; and (c) comparing the second amount of complement C1s protein with the first amount of complement C1s protein. The steps of determining comprise: (i) contacting the biological sample with an anti-C1s antibody of any of the embodiments; and (ii) quantitating binding of the antibody to complement C1s protein present in the sample. In some embodiments, the first time point is a time point before initiation of a treatment regimen, and the second time point is a time point after initiation of a treatment regimen. In some embodiments, the biological sample is selected from the group consisting of blood, serum, plasma, urine, saliva, cerebrospinal fluid, interstitial fluid, ocular fluid, synovial fluid, solid tissue sample, tissue culture sample, and cellular sample.

The present disclosure provides an in vitro method to detect complement C1s protein in a biological sample obtained from an individual, the method comprising: (a) contacting the biological sample with an anti-C1s antibody of any of the embodiments; and (b) detecting binding of the antibody to complement C1s protein present in the sample. In some embodiments, the biological sample is selected from the group consisting of blood, serum, plasma, urine, saliva, cerebrospinal fluid, interstitial fluid, ocular fluid, synovial fluid, solid tissue sample, tissue culture sample, and cellular sample. In some embodiments, the method is quantitative.

The present disclosure provides a method to detect complement C1s protein in a living individual in vivo, the method comprising: (a) administering to the individual an anti-C1s antibody of any of the embodiments; and (b) detecting binding of the antibody to complement C1s protein in the individual using an imaging method. In some embodiments, the binding is detected in the individual at a site altered by a complement-mediated disease or disorder. In some embodiments, the binding is detected in the brain of the individual. In some of the embodiments, the antibody comprises a contrast agent suitable for use in the imaging method. In some embodiments, the imaging method is selected from the group consisting of magnetic resonance imaging, positron emission tomography, and IVIS instrumentation. In some embodiments, the method is quantitative.

In some embodiments, the biological sample is selected from the group consisting of blood, serum, plasma, urine, saliva, cerebrospinal fluid, interstitial fluid, ocular fluid, synovial fluid, solid tissue sample, tissue culture sample, and a cellular sample.

In some embodiments, the methods of the present disclosure provide that the individual is suspected of having a complement-mediated disease or disorder, has been diagnosed as having a complement-mediated disease or disorder, or has a genetic predisposition to developing a complement-mediated disease or disorder.

The present disclosure provides a composition comprising: (a) an anti-C1s antibody of any of the embodiments; and (b) a solution comprising one or more agents that maintain an organ or a tissue intended for transplantation into a recipient individual. In some embodiments, the solution is an organ preservation solution or a tissue preservation solution. In some embodiments, the solution is an organ perfusion solution or a tissue perfusion solution. In some embodiments, the solution comprises: i) a salt; ii) an agent that reduces edema; iii) an oxygen free radical scavenger; and iii) an energy supply system component. In some embodiments, the composition comprises potassium lactobionate, KH₂PO₄, MgSO₄, raffinose, adenosine, glutathione, allopurinol, and/or hydroxyethyl starch.

The present disclosure provides an organ or tissue preservation solution comprising an anti-C1s antibody of any of the embodiments or a pharmaceutical composition thereof.

The present disclosure provides an organ or tissue perfusion solution comprising an anti-C1s antibody of any of the embodiments or a pharmaceutical composition thereof.

The present disclosure provides a method for maintaining an organ or tissue for transplant, the method comprising contacting the organ or the tissue with a composition comprising: (a) an anti-C1s antibody of any of the embodiments; and (b) an organ or tissue preservation solution of any of the embodiments or an organ or tissue perfusion solution of any of the embodiments.

The present disclosure provides an isolated organ or tissue maintained in a composition comprising: (a) an anti-C1s antibody of any of the embodiments; and (b) an organ or tissue preservation solution of any of the embodiments or an organ or tissue perfusion solution of any of the embodiments. In some embodiments, the organ is selected from the group consisting of an eye, a heart, an intestine, a kidney, a liver, a lung, a pancreas, a stomach, and a thymus. In some embodiments, the tissue is selected from the group consisting of bone, bone marrow, cornea, heart valve, islet of Langerhans, tendon, skin, and vein.

The present disclosure provides an in vitro method for inhibiting complement C1s activity in an organ or a tissue, the method comprising contacting the organ or the tissue with an anti-C1s antibody of any of the embodiments or a pharmaceutical composition thereof.

Treatment of individuals may comprise the administration of a therapeutically effective amount of the antibodies described herein. The antibodies can be provided in a kit, such as those provided herein. The antibodies can be used or administered alone or in admixture with another therapeutic, analgesic, or diagnostic agent, such as provided for herein. In providing a patient with an antibody, or fragment thereof, capable of binding to C1s, or an antibody capable of protecting against C1s in a recipient patient, the dosage of administered agent will vary depending upon such factors as the patient's age, weight, height, sex, general medical condition, previous medical history, etc.

An antibody, capable treating a condition associated with C1s activity or use to treat a C1s related pathology, is intended to be provided to subjects in an amount sufficient to affect a reduction, resolution, or amelioration in the C1s related symptom or pathology. Examples of such pathologies are provided for herein.

Accordingly, in some embodiments, methods of treating a subject with a C1s mediated disorder are provided. In some embodiments, the method comprises administering a pharmaceutical composition comprising an antibody, or antigen binding fragment thereof, as provided herein. In some embodiments, the disorder is as provided for herein.

As provided for herein, the antibodies, or antigen binding fragments thereof, can be administered with other therapeutics. These can be administered simultaneously or sequentially.

Kits are also provided which are useful for carrying out embodiments described herein. The present kits can comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

In some embodiments, antibodies that bind to a C1s protein are provided. In some embodiments, the antibodies are antibodies or antigen binding fragments as provided for herein. In some embodiments, the antibodies or antigen binding fragments comprise an amino acid sequence as provided for herein, or a variant thereof as provided for herein. In some embodiments, the antibody is isolated. In some embodiments, the antibody binds specifically to the active form of C1s.

In some embodiments, the antibody inhibits or neutralizes the function of an active form of C1s protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the function facilitated by C1s.

In some embodiments, methods are provided comprising administering an antibody provided for herein, or a pharmaceutical composition comprising the same, to a subject, wherein antibody inhibits the membrane attack complex (MAC) deposition in a subject in need thereof, such as a patient with Myasthenia Gravis. The MAC is a complex of proteins that can form from a complex of complement proteins (see, Bayly-Jones et al., 2017 The mystery behind membrane insertion: a review of the complement membrane attack complex Phil. Trans. R. Soc.; Vol. 372, 20160221, which is hereby incorporated by reference in its entirety). Thus, in some embodiments, the antibody can prevent or reduce the formation of the MAC deposition in patients.

In some embodiments, methods are provided comprising administering an antibody provided for herein, or a pharmaceutical composition comprising the same, to a subject, wherein antibody inhibits or prevents neurotransmission impairment in a subject in need thereof, such as a patient with Myasthenia Gravis. In some embodiments, the method comprises inhibiting neurotransmission impairment. In some embodiments, the method comprises preventing neurotransmission impairment.

In some embodiments, methods are provided comprising administering an antibody provided for herein, or a pharmaceutical composition comprising the same, to a subject, wherein antibody enhances muscle contraction in a subject thereof, such as a patient with Myasthenia Gravis.

In some embodiments, methods are provided comprising administering an antibody provided for herein, or a pharmaceutical composition comprising the same, to a subject, to ameliorate or alleviate muscle paralysis in a subject in need thereof, such as a patient with Myasthenia Gravis. In some embodiments, the method comprises ameliorating muscle paralysis. In some embodiments, the method comprises alleviating muscle paralysis.

In some embodiments, methods are provided comprising administering an antibody provided for herein, or a pharmaceutical composition comprising the same, to a subject, to inhibit the loss of muscle tone or muscle mass in a subject in need thereof, such as a patient with Myasthenia Gravis. In some embodiments, the method comprises inhibiting the loss of muscle tone. In some embodiments, the method comprises inhibiting the loss of muscle mass.

In some embodiments, methods are provided comprising administering an antibody provided for herein, or a pharmaceutical composition comprising the same, to a subject, to reduce the muscle fatigue index in a subject in need thereof, such as a patient with Myasthenia Gravis. In some embodiments, the muscle fatigue index is based on answers to Norwegian version of the FQ (Fatigue Questionnaire) (Loge J H, Ekeberg O, Kaasa S. Fatigue in the general Norwegian population: normative data and associations. J Psychosom Res. 1998; 45:53-65; Chalder T, Berelowitz G, Pawlikowska T, Watts L, Wessely S, Wright D, Wallace E P. Development of a fatigue scale. J Psychosom Res. 1993; 37:147-153, each of which of is hereby incorporated by reference in its entirety. Other examples include, Chalder Fatigue Questionnaire (CFQ) (Chalder T. et al. Development of a fatigue scale; J Psychosom Res. 1993; 37: 147-153); Fatigue Impact Scale (FIS) (Fisk J. D. et al, Measuring the functional impact of fatigue: initial validation of the fatigue impact scale. Clin Infect Dis. 1994; 1: S79-S83); Fatigue Scale for Motor and Cognitive function (FSMC) (Penner I., Raselli C., Stocklin M., Opwis K., Kappos L., Calabrese P. The fatigue scale for motor and cognitive functions (FSMC): validation of a new instrument to assess multiple sclerosis-related fatigue. 2009; 15(12):1509-17); Fatigue Severity Scale (FSS) (Krupp L. B. et al., The fatigue severity scale. Application to patients with multiple sclerosis and systemic lupus erythematosus, Arch Neurol. 1989; 46: 1121-1123); Fatigue Survey (FS) (Grohar-Murray M. E. et al, Self-care actions to manage fatigue among myasthenia gravis patients, J Neurosci Nursing: J Am Assoc Neurosci Nurses. 1998; 30: 191-199); Myasthenia Gravis Fatigue Scale (MGFS) (Grohar-Murray M. E. et al, Self-care actions to manage fatigue among myasthenia gravis patients. J Neurosci Nursing: J Am Assoc Neurosci Nurses. 1998; 30: 191-199); or Neuro-QoL-Fatigue short form (Neuro-QoL-F-SF) (Cella D. et al., Neuro-QOL. Brief Measur Health-relat Qual Life Clin Res Neurol. 2012; 78: 1860-1867), each of which are hereby incorporated by reference in its entirety.

### ENUMERATED EMBODIMENTS

In some embodiments, the following embodiments are provided.
1. An antibody, or an antigen binding fragment thereof, wherein the antibody or antibody fragment comprises:
   (a) a heavy chain variable region comprising heavy chain HCDR1, HCDR2, and HCDR3 sequences, wherein the heavy chain HCDR1 sequence has the amino acid sequence selected from SEQ ID NO: 61 SEQ ID NO: 124, or SEQ ID NO: 130; the heavy chain HCDR2 has the amino acid sequence of SEQ ID NO: 62, SEQ ID NO: 125, or SEQ ID NO: 131, and the heavy chain HCDR3 sequence has the amino acid sequence of SEQ ID NO: 63, or SEQ ID NO: 132, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65 or SEQ ID NO: 134; and the light chain LCDR3 sequence has the amino acid sequence of SEQ ID NO: 66; or variants or convention equivalents of any of the foregoing;
   (b) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 67, or SEQ ID NO: 135, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65 or SEQ ID NO: 134; and the light chain LCDR3 sequence has the amino acid sequence of SEQ ID NO: 68; or variants or convention equivalents of any of the foregoing;
   (c) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 69, or SEQ ID NO: 136, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 68; or variants or convention equivalents of any of the foregoing;
   (d) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 70, or SEQ ID NO: 137, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 71; or variants or convention equivalents of any of the foregoing;
   (e) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 72, or SEQ ID NO: 138, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 71; or variants or convention equivalents of any of the foregoing;
   (f) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 73, or SEQ ID NO: 139, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 71, or variants or convention equivalents of any of the foregoing;
   (g) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 74, or SEQ ID NO: 140, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 75; or variants or convention equivalents of any of the foregoing;
   (h) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 76, or SEQ ID NO: 141, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 77; or variants or convention equivalents of any of the foregoing;
   (i) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 78, or SEQ ID NO: 142, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 79; or variants or convention equivalents of any of the foregoing;
   (j) a heavy chain variable region comprising heavy chain HCDR1, HCDR2, and HCDR3 sequences, wherein the heavy chain HCDR1 sequence has the amino acid sequence of SEQ ID NO: 61, SEQ ID NO: 124, or SEQ ID NO: 130; the heavy chain HCDR2 has the amino acid sequence of SEQ ID NO: 62, SEQ ID NO: 125, or SEQ ID NO: 131; and the heavy chain HCDR3 sequence has the amino acid sequence of SEQ ID NO: 80, or SEQ ID NO: 143, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64, or SEQ ID NO: 133; the light chain LCDR2 sequence has the amino acid sequence of SEQ ID NO: 65, or SEQ ID NO: 134; and the light chain LCDR3 sequence has the amino acid sequence of SEQ ID NO: 81; or variants or convention equivalents of any of the foregoing;
   (k) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 84, or SEQ ID NO: 146, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 87; or variants or convention equivalents of any of the foregoing;
   (l) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 88, or SEQ ID NO: 149, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 89; or variants or convention equivalents of any of the foregoing;
   (m) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 90, or SEQ ID NO: 150, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 89; or variants or convention equivalents of any of the foregoing;
   (n) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 91, or SEQ ID NO: 151, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 92; or variants or convention equivalents of any of the foregoing;
   (o) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 93, or SEQ ID NO: 152, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 94; or variants or convention equivalents of any of the foregoing;
   (p) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 84, or SEQ ID NO: 146, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 95; or variants or convention equivalents of any of the foregoing;
   (q) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 96, or SEQ ID NO: 153, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 97; or variants or convention equivalents of any of the foregoing;
   (r) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 98, or SEQ ID NO: 154, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 99; or variants or convention equivalents of any of the foregoing;
   (s) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 100, or SEQ ID NO: 155, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 101; or variants or convention equivalents of any of the foregoing;
   (t) a heavy chain variable region comprising heavy chain HCDR1, HCDR2, and HCDR3 sequences, wherein the heavy chain HCDR1 sequence has the amino acid sequence of SEQ ID NO: 82, SEQ ID NO: 126, or SEQ ID NO: 144; the heavy chain HCDR2 has the amino acid sequence of SEQ ID NO: 83, SEQ ID NO: 127, or SEQ ID NO: 145; and the heavy chain HCDR3 sequence has the amino acid sequence of SEQ ID NO: 100, or SEQ ID NO: 155, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 85, or SEQ ID NO: 147; the light chain LCDR2 sequence has the amino acid sequence of SEQ ID NO: 86, or SEQ ID NO: 148; and the light chain LCDR3 sequence has the amino acid sequence of SEQ ID NO: 102; or variants or convention equivalents of any of the foregoing;
   (u) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 105, or SEQ ID NO: 158, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 108; or variants or convention equivalents of any of the foregoing;
   (v) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 109, or SEQ ID NO: 161, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 110; or variants or convention equivalents of any of the foregoing;
   (w) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 111, or SEQ ID NO: 162, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 112; or variants or convention equivalents of any of the foregoing;
   (x) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 105, or SEQ ID NO: 158, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 113; or variants or convention equivalents of any of the foregoing;
   (y) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 114, or SEQ ID NO: 163, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 115; or variants or convention equivalents of any of the foregoing;
   (z) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 116, or SEQ ID NO: 164, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 113; or variants or convention equivalents of any of the foregoing;
   (aa) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 117, or SEQ ID NO: 165, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 118; or variants or convention equivalents of any of the foregoing;
   (ab) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 116, or SEQ ID NO: 164, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 119; or variants or convention equivalents of any of the foregoing;
   (ac) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 120, or SEQ ID NO: 166, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 121; or variants or convention equivalents of any of the foregoing;
   (ad) a heavy chain variable region comprising heavy chain HCDR1, HCDR2, and HCDR3 sequences, wherein the heavy chain HCDR1 sequence has the amino acid sequence of SEQ ID NO: 103, SEQ ID NO: 128, or SEQ ID NO: 156; the heavy chain HCDR2 has the amino acid sequence of SEQ ID NO: 104, SEQ ID NO: 129, or SEQ ID NO: 157; and the heavy chain HCDR3 sequence has the amino acid sequence of SEQ ID NO: 122, or SEQ ID NO: 167, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 106, or SEQ ID NO: 159; the light chain LCDR2 sequence has the amino acid sequence of SEQ ID NO: 107, or SEQ ID NO: 160; and the light chain LCDR3 sequence has the amino acid sequence of SEQ ID NO: 123; or variants or convention equivalents of any of the foregoing.
2. The antibody or antigen-binding fragment thereof of embodiment 1, wherein the heavy chain comprises:
   (a) a heavy chain variable region having at least 90% sequence identity to SEQ ID NO: 1, wherein the antibody, or antigen binding fragment thereof, maintains the sequences of HCDR1, HCDR2, HCDR3, LCDR1, LCDR2, and LCDR3 set forth in subpart a of embodiment 1; or
   (b) a heavy chain variable region having at least 90% sequence identity to SEQ ID NO: 3, wherein the antibody, or antigen binding fragment thereof, maintains the sequences of HCDR1, HCDR2, HCDR3, LCDR1, LCDR2, and LCDR3 set forth in subpart b of embodiment 1; or
   (c) a heavy chain variable region having at least 90% sequence identity to SEQ ID NO: 5, wherein the antibody, or antigen binding fragment thereof, maintains the sequences of HCDR1, HCDR2, HCDR3, LCDR1, LCDR2, and LCDR3 set forth in subpart c of embodiment
   (d) a heavy chain variable region having at least 90% sequence identity to SEQ ID NO: 7, wherein the antibody, or antigen binding fragment thereof, maintains the sequences of HCDR1, HCDR2, HCDR3, LCDR1, LCDR2, and LCDR3 set forth in subpart d of embodiment 1; or
   (e) a heavy chain variable region having at least 90% sequence identity to SEQ ID NO: 9, wherein the antibody, or antigen binding fragment thereof, maintains the sequences of HCDR1, HCDR2, HCDR3, LCDR1, LCDR2, and LCDR3 set forth in subpart e of embodiment 1; or
   (f) a heavy chain variable region having at least 90% sequence identity to SEQ ID NO: 11, wherein the antibody, or antigen binding fragment thereof, maintains the sequences of HCDR1, HCDR2, HCDR3, LCDR1, LCDR2, and LCDR3 set forth in subpart f of embodiment 1; or
   (g) a heavy chain variable region having at least 90% sequence identity to SEQ ID NO: 13, wherein the antibody, or antigen binding fragment thereof, maintains the sequences of HCDR1, HCDR2, HCDR3, LCDR1, LCDR2, and LCDR3 set forth in subpart g of embodiment 1; or
   (h) a heavy chain variable region having at least 90% sequence identity to SEQ ID NO: 15, wherein the antibody, or antigen binding fragment thereof, maintains the sequences of HCDR1, HCDR2, HCDR3, LCDR1, LCDR2, and LCDR3 set forth in subpart h of embodiment 1; or
   (i) a heavy chain variable region having at least 90% sequence identity to SEQ ID NO: 17, wherein the antibody, or antigen binding fragment thereof, maintains the sequences of HCDR1, HCDR2, HCDR3, LCDR1, LCDR2, and LCDR3 set forth in subpart i of embodiment 1; or
   (j) a heavy chain variable region having at least 90% sequence identity to SEQ ID NO: 19, wherein the antibody, or antigen binding fragment thereof, maintains the sequences of HCDR1, HCDR2, HCDR3, LCDR1, LCDR2, and LCDR3 set forth in subpart j of embodiment 1; or
   (k) a heavy chain variable region having at least 90% sequence identity to SEQ ID NO: 21, wherein the antibody, or antigen binding fragment thereof, maintains the sequences of HCDR1, HCDR2, HCDR3, LCDR1, LCDR2, and LCDR3 set forth in subpart k of embodiment 1; or
   (l) a heavy chain variable region having at least 90% sequence identity to SEQ ID NO: 23, wherein the antibody, or antigen binding fragment thereof, maintains the sequences of HCDR1, HCDR2, HCDR3, LCDR1, LCDR2, and LCDR3 set forth in subpart 1 of embodiment 1; or
   (m) a heavy chain variable region having at least 90% sequence identity to SEQ ID NO: 25, wherein the antibody, or antigen binding fragment thereof, maintains the sequences of HCDR1, HCDR2, HCDR3, LCDR1, LCDR2, and LCDR3 set forth in subpart m of embodiment 1; or
   (n) a heavy chain variable region having at least 90% sequence identity to SEQ ID NO: 27, wherein the antibody, or antigen binding fragment thereof, maintains the sequences of HCDR1, HCDR2, HCDR3, LCDR1, LCDR2, and LCDR3 set forth in subpart n of embodiment 1; or
   (o) a heavy chain variable region having at least 90% sequence identity to SEQ ID NO: 29, wherein the antibody, or antigen binding fragment thereof, maintains the sequences of HCDR1, HCDR2, HCDR3, LCDR1, LCDR2, and LCDR3 set forth in subpart o of embodiment 1; or
   (p) a heavy chain variable region having at least 90% sequence identity to SEQ ID NO: 31, wherein the antibody, or antigen binding fragment thereof, maintains the sequences of HCDR1, HCDR2, HCDR3, LCDR1, LCDR2, and LCDR3 set forth in subpart p of embodiment 1; or
   (q) a heavy chain variable region having at least 90% sequence identity to SEQ ID NO: 33, wherein the antibody, or antigen binding fragment thereof, maintains the sequences of HCDR1, HCDR2, HCDR3, LCDR1, LCDR2, and LCDR3 set forth in subpart q of embodiment 1; or
   (r) a heavy chain variable region having at least 90% sequence identity to SEQ ID NO: 35, wherein the antibody, or antigen binding fragment thereof, maintains the sequences of HCDR1, HCDR2, HCDR3, LCDR1, LCDR2, and LCDR3 set forth in subpart r of embodiment
   (s) a heavy chain variable region having at least 90% sequence identity to SEQ ID NO: 37, wherein the antibody, or antigen binding fragment thereof, maintains the sequences of HCDR1, HCDR2, HCDR3, LCDR1, LCDR2, and LCDR3 set forth in subpart s of embodiment 1; or
   (t) a heavy chain variable region having at least 90% sequence identity to SEQ ID NO: 39, wherein the antibody, or antigen binding fragment thereof, maintains the sequences of HCDR1, HCDR2, HCDR3, LCDR1, LCDR2, and LCDR3 set forth in subpart t of embodiment 1; or
   (u) a heavy chain variable region having at least 90% sequence identity to SEQ ID NO: 41, wherein the antibody, or antigen binding fragment thereof, maintains the sequences of HCDR1, HCDR2, HCDR3, LCDR1, LCDR2, and LCDR3 set forth in subpart u of embodiment 1; or
   (v) a heavy chain variable region having at least 90% sequence identity to SEQ ID NO: 43, wherein the antibody, or antigen binding fragment thereof, maintains the sequences of HCDR1, HCDR2, HCDR3, LCDR1, LCDR2, and LCDR3 set forth in subpart v of embodiment 1; or
   (w) a heavy chain variable region having at least 90% sequence identity to SEQ ID NO: 45, wherein the antibody, or antigen binding fragment thereof, maintains the sequences of HCDR1, HCDR2, HCDR3, LCDR1, LCDR2, and LCDR3 set forth in subpart w of embodiment 1; or
   (x) a heavy chain variable region having at least 90% sequence identity to SEQ ID NO: 47, wherein the antibody, or antigen binding fragment thereof, maintains the sequences of HCDR1, HCDR2, HCDR3, LCDR1, LCDR2, and LCDR3 set forth in subpart x of embodiment 1; or
   (y) a heavy chain variable region having at least 90% sequence identity to SEQ ID NO: 49, wherein the antibody, or antigen binding fragment thereof, maintains the sequences of HCDR1, HCDR2, HCDR3, LCDR1, LCDR2, and LCDR3 set forth in subpart y of embodiment 1; or
   (z) a heavy chain variable region having at least 90% sequence identity to SEQ ID NO: 51, wherein the antibody, or antigen binding fragment thereof, maintains the sequences of HCDR1, HCDR2, HCDR3, LCDR1, LCDR2, and LCDR3 set forth in subpart z of embodiment 1; or
   (aa) a heavy chain variable region having at least 90% sequence identity to SEQ ID NO: 53, wherein the antibody, or antigen binding fragment thereof, maintains the sequences of HCDR1, HCDR2, HCDR3, LCDR1, LCDR2, and LCDR3 set forth in subpart ab of embodiment 1; or
   (ab) a heavy chain variable region having at least 90% sequence identity to SEQ ID NO: 55, wherein the antibody, or antigen binding fragment thereof, maintains the sequences of HCDR1, HCDR2, HCDR3, LCDR1, LCDR2, and LCDR3 set forth in subpart ac of embodiment 1; or
   (ac) a heavy chain variable region having at least 90% sequence identity to SEQ ID NO: 57, wherein the antibody, or antigen binding fragment thereof, maintains the sequences of HCDR1, HCDR2, HCDR3, LCDR1, LCDR2, and LCDR3 set forth in subpart ad of embodiment 1; or
   (ad) a heavy chain variable region having at least 90% sequence identity to SEQ ID NO: 59, wherein the antibody, or antigen binding fragment thereof, maintains the sequences of HCDR1, HCDR2, HCDR3, LCDR1, LCDR2, and LCDR3 set forth in subpart ae of embodiment 1; or
   (ae) a heavy chain variable region having at least 90% sequence identity to SEQ ID NO: 334, wherein the antibody, or antigen binding fragment thereof, maintains the sequences of HCDR1, HCDR2, HCDR3, LCDR1, LCDR2, and LCDR3 set forth in subpart a of embodiment 1; or
   (af) a heavy chain variable region having at least 90% sequence identity to SEQ ID NO: 335, wherein the antibody, or antigen binding fragment thereof, maintains the sequences of HCDR1, HCDR2, HCDR3, LCDR1, LCDR2, and LCDR3 set forth in subpart b of embodiment 1; or
   (ag) a heavy chain variable region having at least 90% sequence identity to SEQ ID NO: 336, wherein the antibody, or antigen binding fragment thereof, maintains the sequences of HCDR1, HCDR2, HCDR3, LCDR1, LCDR2, and LCDR3 set forth in subpart c of embodiment
   (ah) a heavy chain variable region having at least 90% sequence identity to SEQ ID NO: 337, wherein the antibody, or antigen binding fragment thereof, maintains the sequences of HCDR1, HCDR2, HCDR3, LCDR1, LCDR2, and LCDR3 set forth in subpart d of embodiment 1; or
   (ai) a heavy chain variable region having at least 90% sequence identity to SEQ ID NO: 338, wherein the antibody, or antigen binding fragment thereof, maintains the sequences of HCDR1, HCDR2, HCDR3, LCDR1, LCDR2, and LCDR3 set forth in subpart e of embodiment 1; or
   (aj) a heavy chain variable region having at least 90% sequence identity to SEQ ID NO: 339, wherein the antibody, or antigen binding fragment thereof, maintains the sequences of HCDR1, HCDR2, HCDR3, LCDR1, LCDR2, and LCDR3 set forth in subpart f of embodiment 1; or
   (ak) a heavy chain variable region having at least 90% sequence identity to SEQ ID NO: 340, wherein the antibody, or antigen binding fragment thereof, maintains the sequences of HCDR1, HCDR2, HCDR3, LCDR1, LCDR2, and LCDR3 set forth in subpart g of embodiment 1; or
   (al) a heavy chain variable region having at least 90% sequence identity to SEQ ID NO: 341, wherein the antibody, or antigen binding fragment thereof, maintains the sequences of HCDR1, HCDR2, HCDR3, LCDR1, LCDR2, and LCDR3 set forth in subpart h of embodiment 1; or
   (am) a heavy chain variable region having at least 90% sequence identity to SEQ ID NO: 342, wherein the antibody, or antigen binding fragment thereof, maintains the sequences of HCDR1, HCDR2, HCDR3, LCDR1, LCDR2, and LCDR3 set forth in subpart i of embodiment 1; or
   (an) a heavy chain variable region having at least 90% sequence identity to SEQ ID NO: 343, wherein the antibody, or antigen binding fragment thereof, maintains the sequences of HCDR1, HCDR2, HCDR3, LCDR1, LCDR2, and LCDR3 set forth in subpart j of embodiment 1; or
   (ao) a heavy chain variable region having at least 90% sequence identity to SEQ ID NO: 344, wherein the antibody, or antigen binding fragment thereof, maintains the sequences of HCDR1, HCDR2, HCDR3, LCDR1, LCDR2, and LCDR3 set forth in subpart k of embodiment 1; or
   (ap) a heavy chain variable region having at least 90% sequence identity to SEQ ID NO: 345, wherein the antibody, or antigen binding fragment thereof, maintains the sequences of HCDR1, HCDR2, HCDR3, LCDR1, LCDR2, and LCDR3 set forth in subpart 1 of embodiment 1; or
   (aq) a heavy chain variable region having at least 90% sequence identity to SEQ ID NO: 346, wherein the antibody, or antigen binding fragment thereof, maintains the sequences of HCDR1, HCDR2, HCDR3, LCDR1, LCDR2, and LCDR3 set forth in subpart m of embodiment 1; or
   (ar) a heavy chain variable region having at least 90% sequence identity to SEQ ID NO: 347, wherein the antibody, or antigen binding fragment thereof, maintains the sequences of HCDR1, HCDR2, HCDR3, LCDR1, LCDR2, and LCDR3 set forth in subpart n of embodiment 1; or
   (as) a heavy chain variable region having at least 90% sequence identity to SEQ ID NO: 348, wherein the antibody, or antigen binding fragment thereof, maintains the sequences of HCDR1, HCDR2, HCDR3, LCDR1, LCDR2, and LCDR3 set forth in subpart o of embodiment 1; or
   (at) a heavy chain variable region having at least 90% sequence identity to SEQ ID NO: 349, wherein the antibody, or antigen binding fragment thereof, maintains the sequences of HCDR1, HCDR2, HCDR3, LCDR1, LCDR2, and LCDR3 set forth in subpart p of embodiment 1; or
   (au) a heavy chain variable region having at least 90% sequence identity to SEQ ID NO: 350, wherein the antibody, or antigen binding fragment thereof, maintains the sequences of HCDR1, HCDR2, HCDR3, LCDR1, LCDR2, and LCDR3 set forth in subpart q of embodiment 1; or
   (av) a heavy chain variable region having at least 90% sequence identity to SEQ ID NO: 351, wherein the antibody, or antigen binding fragment thereof, maintains the sequences of HCDR1, HCDR2, HCDR3, LCDR1, LCDR2, and LCDR3 set forth in subpart r of embodiment (aw) a heavy chain variable region having at least 90% sequence identity to SEQ ID NO: 352, wherein the antibody, or antigen binding fragment thereof, maintains the sequences of HCDR1, HCDR2, HCDR3, LCDR1, LCDR2, and LCDR3 set forth in subpart s of embodiment 1; or
   (ax) a heavy chain variable region having at least 90% sequence identity to SEQ ID NO: 353, wherein the antibody, or antigen binding fragment thereof, maintains the sequences of HCDR1, HCDR2, HCDR3, LCDR1, LCDR2, and LCDR3 set forth in subpart t of embodiment 1; or
   (ay) a heavy chain variable region having at least 90% sequence identity to SEQ ID NO: 354, wherein the antibody, or antigen binding fragment thereof, maintains the sequences of HCDR1, HCDR2, HCDR3, LCDR1, LCDR2, and LCDR3 set forth in subpart u of embodiment 1; or
   (az) a heavy chain variable region having at least 90% sequence identity to SEQ ID NO: 355, wherein the antibody, or antigen binding fragment thereof, maintains the sequences of HCDR1, HCDR2, HCDR3, LCDR1, LCDR2, and LCDR3 set forth in subpart v of embodiment 1; or
   (ba) a heavy chain variable region having at least 90% sequence identity to SEQ ID NO: 356, wherein the antibody, or antigen binding fragment thereof, maintains the sequences of HCDR1, HCDR2, HCDR3, LCDR1, LCDR2, and LCDR3 set forth in subpart w of embodiment 1; or
   (bb) a heavy chain variable region having at least 90% sequence identity to SEQ ID NO: 357, wherein the antibody, or antigen binding fragment thereof, maintains the sequences of HCDR1, HCDR2, HCDR3, LCDR1, LCDR2, and LCDR3 set forth in subpart x of embodiment 1; or
   (bc) a heavy chain variable region having at least 90% sequence identity to SEQ ID NO: 358, wherein the antibody, or antigen binding fragment thereof, maintains the sequences of HCDR1, HCDR2, HCDR3, LCDR1, LCDR2, and LCDR3 set forth in subpart y of embodiment 1; or
   (bd) a heavy chain variable region having at least 90% sequence identity to SEQ ID NO: 359, wherein the antibody, or antigen binding fragment thereof, maintains the sequences of HCDR1, HCDR2, HCDR3, LCDR1, LCDR2, and LCDR3 set forth in subpart z of embodiment 1; or
   (be) a heavy chain variable region having at least 90% sequence identity to SEQ ID NO: 360, wherein the antibody, or antigen binding fragment thereof, maintains the sequences of HCDR1, HCDR2, HCDR3, LCDR1, LCDR2, and LCDR3 set forth in subpart ab of embodiment 1; or
   (bf) a heavy chain variable region having at least 90% sequence identity to SEQ ID NO: 361, wherein the antibody, or antigen binding fragment thereof, maintains the sequences of HCDR1, HCDR2, HCDR3, LCDR1, LCDR2, and LCDR3 set forth in subpart ac of embodiment 1; or
   (bg) a heavy chain variable region having at least 90% sequence identity to SEQ ID NO: 362, wherein the antibody, or antigen binding fragment thereof, maintains the sequences of HCDR1, HCDR2, HCDR3, LCDR1, LCDR2, and LCDR3 set forth in subpart ad of embodiment 1; or
   (bh) a heavy chain variable region having at least 90% sequence identity to SEQ ID NO: 363, wherein the antibody, or antigen binding fragment thereof, maintains the sequences of HCDR1, HCDR2, HCDR3, LCDR1, LCDR2, and LCDR3 set forth in subpart ae of embodiment 1.
3. The antibody or antigen-binding fragment thereof of embodiments 1 or 2, wherein the light chain comprises:
   (a) a light chain variable region having at least 90% sequence identity to SEQ ID NO: 2, wherein the antibody, or antigen binding fragment thereof, maintains the sequences of HCDR1, HCDR2, HCDR3, LCDR1, LCDR2, and LCDR3 set forth in subpart a of embodiment 1; or
   (b) a light chain variable region having at least 90% sequence identity to SEQ ID NO: 4, wherein the antibody, or antigen binding fragment thereof, maintains the sequences of HCDR1, HCDR2, HCDR3, LCDR1, LCDR2, and LCDR3 set forth in subpart b of embodiment 1; or
   (c) a light chain variable region having at least 90% sequence identity to SEQ ID NO: 6, wherein the antibody, or antigen binding fragment thereof, maintains the sequences of HCDR1, HCDR2, HCDR3, LCDR1, LCDR2, and LCDR3 set forth in subpart c of embodiment 1; or
   (d) a light chain variable region having at least 90% sequence identity to SEQ ID NO: 8, wherein the antibody, or antigen binding fragment thereof, maintains the sequences of HCDR1, HCDR2, HCDR3, LCDR1, LCDR2, and LCDR3 set forth in subpart d of embodiment 1; or
   (e) a light chain variable region having at least 90% sequence identity to SEQ ID NO: 10, wherein the antibody, or antigen binding fragment thereof, maintains the sequences of HCDR1, HCDR2, HCDR3, LCDR1, LCDR2, and LCDR3 set forth in subpart e of embodiment 1; or
   (f) a light chain variable region having at least 90% sequence identity to SEQ ID NO: 12, wherein the antibody, or antigen binding fragment thereof, maintains the sequences of HCDR1, HCDR2, HCDR3, LCDR1, LCDR2, and LCDR3 set forth in subpart f of embodiment 1; or
   (g) a light chain variable region having at least 90% sequence identity to SEQ ID NO: 14, wherein the antibody, or antigen binding fragment thereof, maintains the sequences of HCDR1, HCDR2, HCDR3, LCDR1, LCDR2, and LCDR3 set forth in subpart g of embodiment 1; or
   (h) a light chain variable region having at least 90% sequence identity to SEQ ID NO: 16, wherein the antibody, or antigen binding fragment thereof, maintains the sequences of HCDR1, HCDR2, HCDR3, LCDR1, LCDR2, and LCDR3 set forth in subpart h of embodiment 1; or
   (i) a light chain variable region having at least 90% sequence identity to SEQ ID NO: 18, wherein the antibody, or antigen binding fragment thereof, maintains the sequences of HCDR1, HCDR2, HCDR3, LCDR1, LCDR2, and LCDR3 set forth in subpart i of embodiment 1; or
   (j) a light chain variable region having at least 90% sequence identity to SEQ ID NO: 20, wherein the antibody, or antigen binding fragment thereof, maintains the sequences of HCDR1, HCDR2, HCDR3, LCDR1, LCDR2, and LCDR3 set forth in subpart j of embodiment
   (k) a light chain variable region having at least 90% sequence identity to SEQ ID NO: 22, wherein the antibody, or antigen binding fragment thereof, maintains the sequences of HCDR1, HCDR2, HCDR3, LCDR1, LCDR2, and LCDR3 set forth in subpart k of embodiment 1; or
   (l) a light chain variable region having at least 90% sequence identity to SEQ ID NO: 24, wherein the antibody, or antigen binding fragment thereof, maintains the sequences of HCDR1, HCDR2, HCDR3, LCDR1, LCDR2, and LCDR3 set forth in subpart 1 of embodiment 1; or
   (m) a light chain variable region having at least 90% sequence identity to SEQ ID NO: 26, wherein the antibody, or antigen binding fragment thereof, maintains the sequences of HCDR1, HCDR2, HCDR3, LCDR1, LCDR2, and LCDR3 set forth in subpart m of embodiment 1; or
   (n) a light chain variable region having at least 90% sequence identity to SEQ ID NO: 28, wherein the antibody, or antigen binding fragment thereof, maintains the sequences of HCDR1, HCDR2, HCDR3, LCDR1, LCDR2, and LCDR3 set forth in subpart n of embodiment 1; or
   (o) a light chain variable region having at least 90% sequence identity to SEQ ID NO: 30, wherein the antibody, or antigen binding fragment thereof, maintains the sequences of HCDR1, HCDR2, HCDR3, LCDR1, LCDR2, and LCDR3 set forth in subpart o of embodiment 1; or
   (p) a light chain variable region having at least 90% sequence identity to SEQ ID NO: 32, wherein the antibody, or antigen binding fragment thereof, maintains the sequences of HCDR1, HCDR2, HCDR3, LCDR1, LCDR2, and LCDR3 set forth in subpart p of embodiment 1; or
   (q) a light chain variable region having at least 90% sequence identity to SEQ ID NO: 34, wherein the antibody, or antigen binding fragment thereof, maintains the sequences of HCDR1, HCDR2, HCDR3, LCDR1, LCDR2, and LCDR3 set forth in subpart q of embodiment 1; or
   (r) a light chain variable region having at least 90% sequence identity to SEQ ID NO: 36, wherein the antibody, or antigen binding fragment thereof, maintains the sequences of HCDR1, HCDR2, HCDR3, LCDR1, LCDR2, and LCDR3 set forth in subpart r of embodiment 1; or
   (s) a light chain variable region having at least 90% sequence identity to SEQ ID NO: 38, wherein the antibody, or antigen binding fragment thereof, maintains the sequences of HCDR1, HCDR2, HCDR3, LCDR1, LCDR2, and LCDR3 set forth in subpart s of embodiment 1; or
   (t) a light chain variable region having at least 90% sequence identity to SEQ ID NO: 40, wherein the antibody, or antigen binding fragment thereof, maintains the sequences of HCDR1, HCDR2, HCDR3, LCDR1, LCDR2, and LCDR3 set forth in subpart t of embodiment 1; or
   (u) a light chain variable region having at least 90% sequence identity to SEQ ID NO: 42, wherein the antibody, or antigen binding fragment thereof, maintains the sequences of HCDR1, HCDR2, HCDR3, LCDR1, LCDR2, and LCDR3 set forth in subpart u of embodiment 1; or
   (v) a light chain variable region having at least 90% sequence identity to SEQ ID NO: 44, wherein the antibody, or antigen binding fragment thereof, maintains the sequences of HCDR1, HCDR2, HCDR3, LCDR1, LCDR2, and LCDR3 set forth in subpart v of embodiment 1; or
   (w) a light chain variable region having at least 90% sequence identity to SEQ ID NO: 46, wherein the antibody, or antigen binding fragment thereof, maintains the sequences of HCDR1, HCDR2, HCDR3, LCDR1, LCDR2, and LCDR3 set forth in subpart w of embodiment 1; or
   (x) a light chain variable region having at least 90% sequence identity to SEQ ID NO: 48, wherein the antibody, or antigen binding fragment thereof, maintains the sequences of HCDR1, HCDR2, HCDR3, LCDR1, LCDR2, and LCDR3 set forth in subpart x of embodiment 1; or
   (y) a light chain variable region having at least 90% sequence identity to SEQ ID NO: 50, wherein the antibody, or antigen binding fragment thereof, maintains the sequences of HCDR1, HCDR2, HCDR3, LCDR1, LCDR2, and LCDR3 set forth in subpart y of embodiment
   (z) a light chain variable region having at least 90% sequence identity to SEQ ID NO: 52, wherein the antibody, or antigen binding fragment thereof, maintains the sequences of HCDR1, HCDR2, HCDR3, LCDR1, LCDR2, and LCDR3 set forth in subpart z of embodiment 1; or
   (aa) a light chain variable region having at least 90% sequence identity to SEQ ID NO: 54, wherein the antibody, or antigen binding fragment thereof, maintains the sequences of HCDR1, HCDR2, HCDR3, LCDR1, LCDR2, and LCDR3 set forth in subpart ab of embodiment 1; or
   (ab) a light chain variable region having at least 90% sequence identity to SEQ ID NO: 56, wherein the antibody, or antigen binding fragment thereof, maintains the sequences of HCDR1, HCDR2, HCDR3, LCDR1, LCDR2, and LCDR3 set forth in subpart ac of embodiment 1; or
   (ac) a light chain variable region having at least 90% sequence identity to SEQ ID NO: 58, wherein the antibody, or antigen binding fragment thereof, maintains the sequences of HCDR1, HCDR2, HCDR3, LCDR1, LCDR2, and LCDR3 set forth in subpart ad of embodiment 1; or
   (ad) a light chain variable region having at least 90% sequence identity to SEQ ID NO: 60, wherein the antibody, or antigen binding fragment thereof, maintains the sequences of HCDR1, HCDR2, HCDR3, LCDR1, LCDR2, and LCDR3 set forth in subpart ae of embodiment 1.
4. The antibody or antigen-binding fragment thereof of any one of embodiments 1-3 wherein the antibody, or antigen binding fragment thereof, comprises:
   (a) a light chain variable region of the light chain having at least 90% sequence identity to SEQ ID NO: 2, and a heavy chain variable region of the heavy chain having at least 90% sequence identity to SEQ ID NO: 1, wherein the antibody, or antigen binding fragment thereof, maintains the sequences of HCDR1, HCDR2, HCDR3, LCDR1, LCDR2, and LCDR3 set forth in subpart a of embodiment 1; or
   (b) a light chain variable region of the light chain having at least 90% sequence identity to SEQ ID NO: 4, and a heavy chain variable region of the heavy chain having at least 90% sequence identity to SEQ ID NO: 3, wherein the antibody, or antigen binding fragment set forth in subpart b of embodiment 1; or
   (c) a light chain variable region of the light chain having at least 90% sequence identity to SEQ ID NO: 6, and a heavy chain variable region of the heavy chain having at least 90% sequence identity to SEQ ID NO: 5, wherein the antibody, or antigen binding fragment thereof, maintains the sequences of HCDR1, HCDR2, HCDR3, LCDR1, LCDR2, and LCDR3 set forth in subpart c of embodiment 1; or
   (d) a light chain variable region of the light chain having at least 90% sequence identity to SEQ ID NO: 8, and a heavy chain variable region of the heavy chain having at least 90% sequence identity to SEQ ID NO: 7, wherein the antibody, or antigen binding fragment thereof, maintains the sequences of HCDR1, HCDR2, HCDR3, LCDR1, LCDR2, and LCDR3 set forth in subpart d of embodiment 1; or
   (e) a light chain variable region of the light chain having at least 90% sequence identity to SEQ ID NO: 10, and a heavy chain variable region of the heavy chain having at least 90% sequence identity to SEQ ID NO: 9, wherein the antibody, or antigen binding fragment thereof, maintains the sequences of HCDR1, HCDR2, HCDR3, LCDR1, LCDR2, and LCDR3 set forth in subpart e of embodiment 1; or
   (f) a light chain variable region of the light chain having at least 90% sequence identity to SEQ ID NO: 12, and a heavy chain variable region of the heavy chain having at least 90% sequence identity to SEQ ID NO: 11, wherein the antibody, or antigen binding fragment thereof, maintains the sequences of HCDR1, HCDR2, HCDR3, LCDR1, LCDR2, and LCDR3 set forth in subpart f of embodiment 1; or
   (g) a light chain variable region of the light chain having at least 90% sequence identity to SEQ ID NO: 14, and a heavy chain variable region of the heavy chain having at least 90% sequence identity to SEQ ID NO: 13, wherein the antibody, or antigen binding fragment thereof, maintains the sequences of HCDR1, HCDR2, HCDR3, LCDR1, LCDR2, and LCDR3 set forth in subpart g of embodiment 1; or
   (h) a light chain variable region of the light chain having at least 90% sequence identity to SEQ ID NO: 16, and a heavy chain variable region of the heavy chain having at least 90% sequence identity to SEQ ID NO: 15, wherein the antibody, or antigen binding fragment set forth in subpart h of embodiment 1; or
   (i) a light chain variable region of the light chain having at least 90% sequence identity to SEQ ID NO: 18, and a heavy chain variable region of the heavy chain having at least 90% sequence identity to SEQ ID NO: 17, wherein the antibody, or antigen binding fragment thereof, maintains the sequences of HCDR1, HCDR2, HCDR3, LCDR1, LCDR2, and LCDR3 set forth in subpart i of embodiment 1; or
   (j) a light chain variable region of the light chain having at least 90% sequence identity to SEQ ID NO: 20, and a heavy chain variable region of the heavy chain having at least 90% sequence identity to SEQ ID NO: 19, wherein the antibody, or antigen binding fragment thereof, maintains the sequences of HCDR1, HCDR2, HCDR3, LCDR1, LCDR2, and LCDR3 set forth in subpart j of embodiment 1; or
   (k) a light chain variable region of the light chain having at least 90% sequence identity to SEQ ID NO: 22, and a heavy chain variable region of the heavy chain having at least 90% sequence identity to SEQ ID NO: 21, wherein the antibody, or antigen binding fragment thereof, maintains the sequences of HCDR1, HCDR2, HCDR3, LCDR1, LCDR2, and LCDR3 set forth in subpart k of embodiment 1; or
   (l) a light chain variable region of the light chain having at least 90% sequence identity to SEQ ID NO: 24, and a heavy chain variable region of the heavy chain having at least 90% sequence identity to SEQ ID NO: 23, wherein the antibody, or antigen binding fragment thereof, maintains the sequences of HCDR1, HCDR2, HCDR3, LCDR1, LCDR2, and LCDR3 set forth in subpart 1 of embodiment 1; or
   (m) a light chain variable region of the light chain having at least 90% sequence identity to SEQ ID NO: 26, and a heavy chain variable region of the heavy chain having at least 90% sequence identity to SEQ ID NO: 25, wherein the antibody, or antigen binding fragment thereof, maintains the sequences of HCDR1, HCDR2, HCDR3, LCDR1, LCDR2, and LCDR3 set forth in subpart m of embodiment 1; or
   (n) a light chain variable region of the light chain having at least 90% sequence identity to SEQ ID NO: 28, and a heavy chain variable region of the heavy chain having at least 90% sequence identity to SEQ ID NO: 27, wherein the antibody, or antigen binding fragment set forth in subpart n of embodiment 1; or
   (o) a light chain variable region of the light chain having at least 90% sequence identity to SEQ ID NO: 30, and a heavy chain variable region of the heavy chain having at least 90% sequence identity to SEQ ID NO: 29, wherein the antibody, or antigen binding fragment thereof, maintains the sequences of HCDR1, HCDR2, HCDR3, LCDR1, LCDR2, and LCDR3 set forth in subpart o of embodiment 1; or
   (p) a light chain variable region of the light chain having at least 90% sequence identity to SEQ ID NO: 32, and a heavy chain variable region of the heavy chain having at least 90% sequence identity to SEQ ID NO: 31, wherein the antibody, or antigen binding fragment thereof, maintains the sequences of HCDR1, HCDR2, HCDR3, LCDR1, LCDR2, and LCDR3 set forth in subpart p of embodiment 1; or
   (q) a light chain variable region of the light chain having at least 90% sequence identity to SEQ ID NO: 34, and a heavy chain variable region of the heavy chain having at least 90% sequence identity to SEQ ID NO: 33, wherein the antibody, or antigen binding fragment thereof, maintains the sequences of HCDR1, HCDR2, HCDR3, LCDR1, LCDR2, and LCDR3 set forth in subpart q of embodiment 1; or
   (r) a light chain variable region of the light chain having at least 90% sequence identity to SEQ ID NO: 36, and a heavy chain variable region of the heavy chain having at least 90% sequence identity to SEQ ID NO: 35, wherein the antibody, or antigen binding fragment thereof, maintains the sequences of HCDR1, HCDR2, HCDR3, LCDR1, LCDR2, and LCDR3 set forth in subpart r of embodiment 1; or
   (s) a light chain variable region of the light chain having at least 90% sequence identity to SEQ ID NO: 38, and a heavy chain variable region of the heavy chain having at least 90% sequence identity to SEQ ID NO: 37, wherein the antibody, or antigen binding fragment thereof, maintains the sequences of HCDR1, HCDR2, HCDR3, LCDR1, LCDR2, and LCDR3 set forth in subpart s of embodiment 1; or
   (t) a light chain variable region of the light chain having at least 90% sequence identity to SEQ ID NO: 40, and a heavy chain variable region of the heavy chain having at least 90% sequence identity to SEQ ID NO: 39, wherein the antibody, or antigen binding fragment set forth in subpart t of embodiment 1; or
   (u) a light chain variable region of the light chain having at least 90% sequence identity to SEQ ID NO: 42, and a heavy chain variable region of the heavy chain having at least 90% sequence identity to SEQ ID NO: 41, wherein the antibody, or antigen binding fragment thereof, maintains the sequences of HCDR1, HCDR2, HCDR3, LCDR1, LCDR2, and LCDR3 set forth in subpart u of embodiment 1; or
   (v) a light chain variable region of the light chain having at least 90% sequence identity to SEQ ID NO: 44, and a heavy chain variable region of the heavy chain having at least 90% sequence identity to SEQ ID NO: 43, wherein the antibody, or antigen binding fragment thereof, maintains the sequences of HCDR1, HCDR2, HCDR3, LCDR1, LCDR2, and LCDR3 set forth in subpart v of embodiment 1; or
   (w) a light chain variable region of the light chain having at least 90% sequence identity to SEQ ID NO: 46, and a heavy chain variable region of the heavy chain having at least 90% sequence identity to SEQ ID NO: 45, wherein the antibody, or antigen binding fragment thereof, maintains the sequences of HCDR1, HCDR2, HCDR3, LCDR1, LCDR2, and LCDR3 set forth in subpart w of embodiment 1; or
   (x) a light chain variable region of the light chain having at least 90% sequence identity to SEQ ID NO: 48, and a heavy chain variable region of the heavy chain having at least 90% sequence identity to SEQ ID NO: 47, wherein the antibody, or antigen binding fragment thereof, maintains the sequences of HCDR1, HCDR2, HCDR3, LCDR1, LCDR2, and LCDR3 set forth in subpart x of embodiment 1; or
   (y) a light chain variable region of the light chain having at least 90% sequence identity to SEQ ID NO: 50, and a heavy chain variable region of the heavy chain having at least 90% sequence identity to SEQ ID NO: 49, wherein the antibody, or antigen binding fragment thereof, maintains the sequences of HCDR1, HCDR2, HCDR3, LCDR1, LCDR2, and LCDR3 set forth in subpart y of embodiment 1; or
   (z) a light chain variable region of the light chain having at least 90% sequence identity to SEQ ID NO: 52, and a heavy chain variable region of the heavy chain having at least 90% sequence identity to SEQ ID NO: 51, wherein the antibody, or antigen binding fragment set forth in subpart z of embodiment 1; or
   (aa) a light chain variable region of the light chain having at least 90% sequence identity to SEQ ID NO: 54, and a heavy chain variable region of the heavy chain having at least 90% sequence identity to SEQ ID NO: 53, wherein the antibody, or antigen binding fragment thereof, maintains the sequences of HCDR1, HCDR2, HCDR3, LCDR1, LCDR2, and LCDR3 set forth in subpart ab of embodiment 1; or
   (ab) a light chain variable region of the light chain having at least 90% sequence identity to SEQ ID NO: 56, and a heavy chain variable region of the heavy chain having at least 90% sequence identity to SEQ ID NO: 55, wherein the antibody, or antigen binding fragment thereof, maintains the sequences of HCDR1, HCDR2, HCDR3, LCDR1, LCDR2, and LCDR3 set forth in subpart ac of embodiment 1; or
   (ac) a light chain variable region of the light chain having at least 90% sequence identity to SEQ ID NO: 58, and a heavy chain variable region of the heavy chain having at least 90% sequence identity to SEQ ID NO: 57, wherein the antibody, or antigen binding fragment thereof, maintains the sequences of HCDR1, HCDR2, HCDR3, LCDR1, LCDR2, and LCDR3 set forth in subpart ad of embodiment 1; or
   (ad) a light chain variable region of the light chain having at least 90% sequence identity to SEQ ID NO: 60, and a heavy chain variable region of the heavy chain having at least 90% sequence identity to SEQ ID NO: 59, wherein the antibody, or antigen binding fragment thereof, maintains the sequences of HCDR1, HCDR2, HCDR3, LCDR1, LCDR2, and LCDR3 set forth in subpart ae of embodiment 1; or
   (ae) a light chain variable region of the light chain having at least 90% sequence identity to SEQ ID NO: 2, and a heavy chain variable region of the heavy chain having at least 90% sequence identity to SEQ ID NO: 334, wherein the antibody, or antigen binding fragment thereof, maintains the sequences of HCDR1, HCDR2, HCDR3, LCDR1, LCDR2, and LCDR3 set forth in subpart a of embodiment 1; or
   (af) a light chain variable region of the light chain having at least 90% sequence identity to SEQ ID NO: 4, and a heavy chain variable region of the heavy chain having at least 90% sequence identity to SEQ ID NO: 335, wherein the antibody, or antigen binding fragment set forth in subpart b of embodiment 1; or
   (ag) a light chain variable region of the light chain having at least 90% sequence identity to SEQ ID NO: 6, and a heavy chain variable region of the heavy chain having at least 90% sequence identity to SEQ ID NO: 336, wherein the antibody, or antigen binding fragment thereof, maintains the sequences of HCDR1, HCDR2, HCDR3, LCDR1, LCDR2, and LCDR3 set forth in subpart c of embodiment 1; or
   (ah) a light chain variable region of the light chain having at least 90% sequence identity to SEQ ID NO: 8, and a heavy chain variable region of the heavy chain having at least 90% sequence identity to SEQ ID NO: 337, wherein the antibody, or antigen binding fragment thereof, maintains the sequences of HCDR1, HCDR2, HCDR3, LCDR1, LCDR2, and LCDR3 set forth in subpart d of embodiment 1; or
   (ai) a light chain variable region of the light chain having at least 90% sequence identity to SEQ ID NO: 10, and a heavy chain variable region of the heavy chain having at least 90% sequence identity to SEQ ID NO: 338, wherein the antibody, or antigen binding fragment thereof, maintains the sequences of HCDR1, HCDR2, HCDR3, LCDR1, LCDR2, and LCDR3 set forth in subpart e of embodiment 1; or
   (aj) a light chain variable region of the light chain having at least 90% sequence identity to SEQ ID NO: 12, and a heavy chain variable region of the heavy chain having at least 90% sequence identity to SEQ ID NO: 339, wherein the antibody, or antigen binding fragment thereof, maintains the sequences of HCDR1, HCDR2, HCDR3, LCDR1, LCDR2, and LCDR3 set forth in subpart f of embodiment 1; or
   (ak) a light chain variable region of the light chain having at least 90% sequence identity to SEQ ID NO: 14, and a heavy chain variable region of the heavy chain having at least 90% sequence identity to SEQ ID NO: 340, wherein the antibody, or antigen binding fragment thereof, maintains the sequences of HCDR1, HCDR2, HCDR3, LCDR1, LCDR2, and LCDR3 set forth in subpart g of embodiment 1; or
   (al) a light chain variable region of the light chain having at least 90% sequence identity to SEQ ID NO: 16, and a heavy chain variable region of the heavy chain having at least 90% sequence identity to SEQ ID NO: 341, wherein the antibody, or antigen binding fragment set forth in subpart h of embodiment 1; or
   (am) a light chain variable region of the light chain having at least 90% sequence identity to SEQ ID NO: 18, and a heavy chain variable region of the heavy chain having at least 90% sequence identity to SEQ ID NO: 342, wherein the antibody, or antigen binding fragment thereof, maintains the sequences of HCDR1, HCDR2, HCDR3, LCDR1, LCDR2, and LCDR3 set forth in subpart i of embodiment 1; or
   (an) a light chain variable region of the light chain having at least 90% sequence identity to SEQ ID NO: 20, and a heavy chain variable region of the heavy chain having at least 90% sequence identity to SEQ ID NO: 343, wherein the antibody, or antigen binding fragment thereof, maintains the sequences of HCDR1, HCDR2, HCDR3, LCDR1, LCDR2, and LCDR3 set forth in subpart j of embodiment 1; or
   (ao) a light chain variable region of the light chain having at least 90% sequence identity to SEQ ID NO: 22, and a heavy chain variable region of the heavy chain having at least 90% sequence identity to SEQ ID NO: 344, wherein the antibody, or antigen binding fragment thereof, maintains the sequences of HCDR1, HCDR2, HCDR3, LCDR1, LCDR2, and LCDR3 set forth in subpart k of embodiment 1; or
   (ap) a light chain variable region of the light chain having at least 90% sequence identity to SEQ ID NO: 24, and a heavy chain variable region of the heavy chain having at least 90% sequence identity to SEQ ID NO: 345, wherein the antibody, or antigen binding fragment thereof, maintains the sequences of HCDR1, HCDR2, HCDR3, LCDR1, LCDR2, and LCDR3 set forth in subpart 1 of embodiment 1; or
   (aq) a light chain variable region of the light chain having at least 90% sequence identity to SEQ ID NO: 26, and a heavy chain variable region of the heavy chain having at least 90% sequence identity to SEQ ID NO: 346, wherein the antibody, or antigen binding fragment thereof, maintains the sequences of HCDR1, HCDR2, HCDR3, LCDR1, LCDR2, and LCDR3 set forth in subpart m of embodiment 1; or
   (ar) a light chain variable region of the light chain having at least 90% sequence identity to SEQ ID NO: 28, and a heavy chain variable region of the heavy chain having at least 90% sequence identity to SEQ ID NO: 347, wherein the antibody, or antigen binding fragment set forth in subpart n of embodiment 1; or
   (as) a light chain variable region of the light chain having at least 90% sequence identity to SEQ ID NO: 30, and a heavy chain variable region of the heavy chain having at least 90% sequence identity to SEQ ID NO: 348, wherein the antibody, or antigen binding fragment thereof, maintains the sequences of HCDR1, HCDR2, HCDR3, LCDR1, LCDR2, and LCDR3 set forth in subpart o of embodiment 1; or
   (at) a light chain variable region of the light chain having at least 90% sequence identity to SEQ ID NO: 32, and a heavy chain variable region of the heavy chain having at least 90% sequence identity to SEQ ID NO: 349, wherein the antibody, or antigen binding fragment thereof, maintains the sequences of HCDR1, HCDR2, HCDR3, LCDR1, LCDR2, and LCDR3 set forth in subpart p of embodiment 1; or
   (au) a light chain variable region of the light chain having at least 90% sequence identity to SEQ ID NO: 34, and a heavy chain variable region of the heavy chain having at least 90% sequence identity to SEQ ID NO: 350, wherein the antibody, or antigen binding fragment thereof, maintains the sequences of HCDR1, HCDR2, HCDR3, LCDR1, LCDR2, and LCDR3 set forth in subpart q of embodiment 1; or
   (av) a light chain variable region of the light chain having at least 90% sequence identity to SEQ ID NO: 36, and a heavy chain variable region of the heavy chain having at least 90% sequence identity to SEQ ID NO: 351, wherein the antibody, or antigen binding fragment thereof, maintains the sequences of HCDR1, HCDR2, HCDR3, LCDR1, LCDR2, and LCDR3 set forth in subpart r of embodiment 1; or
   (aw) a light chain variable region of the light chain having at least 90% sequence identity to SEQ ID NO: 38, and a heavy chain variable region of the heavy chain having at least 90% sequence identity to SEQ ID NO: 352, wherein the antibody, or antigen binding fragment thereof, maintains the sequences of HCDR1, HCDR2, HCDR3, LCDR1, LCDR2, and LCDR3 set forth in subpart s of embodiment 1; or
   (ax) a light chain variable region of the light chain having at least 90% sequence identity to SEQ ID NO: 40, and a heavy chain variable region of the heavy chain having at least 90% sequence identity to SEQ ID NO: 353, wherein the antibody, or antigen binding fragment set forth in subpart t of embodiment 1; or
   (ay) a light chain variable region of the light chain having at least 90% sequence identity to SEQ ID NO: 42, and a heavy chain variable region of the heavy chain having at least 90% sequence identity to SEQ ID NO: 354, wherein the antibody, or antigen binding fragment thereof, maintains the sequences of HCDR1, HCDR2, HCDR3, LCDR1, LCDR2, and LCDR3 set forth in subpart u of embodiment 1; or
   (az) a light chain variable region of the light chain having at least 90% sequence identity to SEQ ID NO: 44, and a heavy chain variable region of the heavy chain having at least 90% sequence identity to SEQ ID NO: 355, wherein the antibody, or antigen binding fragment thereof, maintains the sequences of HCDR1, HCDR2, HCDR3, LCDR1, LCDR2, and LCDR3 set forth in subpart v of embodiment 1; or
   (ba) a light chain variable region of the light chain having at least 90% sequence identity to SEQ ID NO: 46, and a heavy chain variable region of the heavy chain having at least 90% sequence identity to SEQ ID NO: 356, wherein the antibody, or antigen binding fragment thereof, maintains the sequences of HCDR1, HCDR2, HCDR3, LCDR1, LCDR2, and LCDR3 set forth in subpart w of embodiment 1; or
   (bb) a light chain variable region of the light chain having at least 90% sequence identity to SEQ ID NO: 48, and a heavy chain variable region of the heavy chain having at least 90% sequence identity to SEQ ID NO: 357, wherein the antibody, or antigen binding fragment thereof, maintains the sequences of HCDR1, HCDR2, HCDR3, LCDR1, LCDR2, and LCDR3 set forth in subpart x of embodiment 1; or
   (bc) a light chain variable region of the light chain having at least 90% sequence identity to SEQ ID NO: 50, and a heavy chain variable region of the heavy chain having at least 90% sequence identity to SEQ ID NO: 358, wherein the antibody, or antigen binding fragment thereof, maintains the sequences of HCDR1, HCDR2, HCDR3, LCDR1, LCDR2, and LCDR3 set forth in subpart y of embodiment 1; or
   (bd) a light chain variable region of the light chain having at least 90% sequence identity to SEQ ID NO: 52, and a heavy chain variable region of the heavy chain having at least 90% sequence identity to SEQ ID NO: 359, wherein the antibody, or antigen binding fragment set forth in subpart z of embodiment 1; or
   (be) a light chain variable region of the light chain having at least 90% sequence identity to SEQ ID NO: 54, and a heavy chain variable region of the heavy chain having at least 90% sequence identity to SEQ ID NO: 360, wherein the antibody, or antigen binding fragment thereof, maintains the sequences of HCDR1, HCDR2, HCDR3, LCDR1, LCDR2, and LCDR3 set forth in subpart ab of embodiment 1; or
   (bf) a light chain variable region of the light chain having at least 90% sequence identity to SEQ ID NO: 56, and a heavy chain variable region of the heavy chain having at least 90% sequence identity to SEQ ID NO: 361, wherein the antibody, or antigen binding fragment thereof, maintains the sequences of HCDR1, HCDR2, HCDR3, LCDR1, LCDR2, and LCDR3 set forth in subpart ac of embodiment 1; or
   (bg) a light chain variable region of the light chain having at least 90% sequence identity to SEQ ID NO: 58, and a heavy chain variable region of the heavy chain having at least 90% sequence identity to SEQ ID NO: 362, wherein the antibody, or antigen binding fragment thereof, maintains the sequences of HCDR1, HCDR2, HCDR3, LCDR1, LCDR2, and LCDR3 set forth in subpart ad of embodiment 1; or
   (bh) a light chain variable region of the light chain having at least 90% sequence identity to SEQ ID NO: 60, and a heavy chain variable region of the heavy chain having at least 90% sequence identity to SEQ ID NO: 363, wherein the antibody, or antigen binding fragment thereof, maintains the sequences of HCDR1, HCDR2, HCDR3, LCDR1, LCDR2, and LCDR3 set forth in subpart ae of embodiment 1.
5. The antibody or antigen-binding fragment thereof of embodiment 4, wherein;
   (a) the light chain variable region comprises SEQ ID NO: 2, and the heavy chain variable region comprises SEQ ID NO: 1;
   (b) the light chain variable region comprises SEQ ID NO: 4, and the heavy chain variable region comprises SEQ ID NO: 3;
   (c) the light chain variable region comprises SEQ ID NO: 6, and the heavy chain variable region comprises SEQ ID NO: 5
   (d) the light chain variable region comprises SEQ ID NO: 8, and the heavy chain variable region comprises SEQ ID NO: 7
   (e) the light chain variable region comprises SEQ ID NO: 10, and the heavy chain variable region comprises SEQ ID NO: 9
   (f) the light chain variable region comprises SEQ ID NO: 12, and the heavy chain variable region comprises SEQ ID NO: 11;
   (g) the light chain variable region comprises SEQ ID NO: 14, and the heavy chain variable region comprises SEQ ID NO: 13;
   (h) the light chain variable region comprises SEQ ID NO: 16, and the heavy chain variable region comprises SEQ ID NO: 15;
   (i) the light chain variable region comprises SEQ ID NO: 18, and the heavy chain variable region comprises SEQ ID NO: 17;
   (j) the light chain variable region comprises SEQ ID NO: 20, and the heavy chain variable region comprises SEQ ID NO: 19;
   (k) the light chain variable region comprises SEQ ID NO: 22, and the heavy chain variable region comprises SEQ ID NO: 21;
   (l) the light chain variable region comprises SEQ ID NO: 24, and the heavy chain variable region comprises SEQ ID NO: 23;
   (m) the light chain variable region comprises SEQ ID NO: 26, and the heavy chain variable region comprises SEQ ID NO: 25;
   (n) the light chain variable region comprises SEQ ID NO: 28, and the heavy chain variable region comprises SEQ ID NO: 27;
   (o) the light chain variable region comprises SEQ ID NO: 30, and the heavy chain variable region comprises SEQ ID NO: 29;
   (p) the light chain variable region comprises SEQ ID NO: 32, and the heavy chain variable region comprises SEQ ID NO: 31;
   (q) the light chain variable region comprises SEQ ID NO: 34, and the heavy chain variable region comprises SEQ ID NO: 33;
   (r) the light chain variable region comprises SEQ ID NO: 36, and the heavy chain variable region comprises SEQ ID NO: 35;
   (s) the light chain variable region comprises SEQ ID NO: 38, and the heavy chain variable region comprises SEQ ID NO: 37;
   (t) the light chain variable region comprises SEQ ID NO: 40, and the heavy chain variable region comprises SEQ ID NO: 39;
   (u) the light chain variable region comprises SEQ ID NO: 42, and the heavy chain variable region comprises SEQ ID NO: 41;
   (v) the light chain variable region comprises SEQ ID NO: 44, and the heavy chain variable region comprises SEQ ID NO: 43;
   (w) the light chain variable region comprises SEQ ID NO: 46, and the heavy chain variable region comprises SEQ ID NO: 45;
   (x) the light chain variable region comprises SEQ ID NO: 48, and the heavy chain variable region comprises SEQ ID NO: 47;
   (y) the light chain variable region comprises SEQ ID NO: 50, and the heavy chain variable region comprises SEQ ID NO: 49;
   (z) the light chain variable region comprises SEQ ID NO: 52, and the heavy chain variable region comprises SEQ ID NO: 51;
   (aa) the light chain variable region comprises SEQ ID NO: 54, and the heavy chain variable region comprises SEQ ID NO: 53;
   (ab) the light chain variable region comprises SEQ ID NO: 56, and the heavy chain variable region comprises SEQ ID NO: 55;
   (ac) the light chain variable region comprises SEQ ID NO: 58, and the heavy chain variable region comprises SEQ ID NO: 57;
   (ad) the light chain variable region comprises SEQ ID NO: 60, and the heavy chain variable region comprises SEQ ID NO: 59; or
   (ae) the light chain variable region comprises SEQ ID NO: 2, and the heavy chain variable region comprises SEQ ID NO: 334;
   (af) the light chain variable region comprises SEQ ID NO: 4, and the heavy chain variable region comprises SEQ ID NO: 335;
   (ag) the light chain variable region comprises SEQ ID NO: 6, and the heavy chain variable region comprises SEQ ID NO: 336;
   (ah) the light chain variable region comprises SEQ ID NO: 8, and the heavy chain variable region comprises SEQ ID NO: 337;
   (ai) the light chain variable region comprises SEQ ID NO: 10, and the heavy chain variable region comprises SEQ ID NO: 338;
   (aj) the light chain variable region comprises SEQ ID NO: 12, and the heavy chain variable region comprises SEQ ID NO: 339;
   (ak) the light chain variable region comprises SEQ ID NO: 14, and the heavy chain variable region comprises SEQ ID NO: 340;
   (al) the light chain variable region comprises SEQ ID NO: 16, and the heavy chain variable region comprises SEQ ID NO: 341;
   (am) the light chain variable region comprises SEQ ID NO: 18, and the heavy chain variable region comprises SEQ ID NO: 342;
   (an) the light chain variable region comprises SEQ ID NO: 20, and the heavy chain variable region comprises SEQ ID NO: 343;
   (ao) the light chain variable region comprises SEQ ID NO: 22, and the heavy chain variable region comprises SEQ ID NO: 344;
   (ap) the light chain variable region comprises SEQ ID NO: 24, and the heavy chain variable region comprises SEQ ID NO: 345;
   (aq) the light chain variable region comprises SEQ ID NO: 26, and the heavy chain variable region comprises SEQ ID NO: 346;
   (ar) the light chain variable region comprises SEQ ID NO: 28, and the heavy chain variable region comprises SEQ ID NO: 347;
   (as) the light chain variable region comprises SEQ ID NO: 30, and the heavy chain variable region comprises SEQ ID NO: 348;
   (at) the light chain variable region comprises SEQ ID NO: 32, and the heavy chain variable region comprises SEQ ID NO: 349;
   (au) the light chain variable region comprises SEQ ID NO: 34, and the heavy chain variable region comprises SEQ ID NO: 350;
   (av) the light chain variable region comprises SEQ ID NO: 36, and the heavy chain variable region comprises SEQ ID NO: 351;
   (aw) the light chain variable region comprises SEQ ID NO: 38, and the heavy chain variable region comprises SEQ ID NO: 352;
   (ax) the light chain variable region comprises SEQ ID NO: 40, and the heavy chain variable region comprises SEQ ID NO: 353;
   (ay) the light chain variable region comprises SEQ ID NO: 42, and the heavy chain variable region comprises SEQ ID NO: 354;
   (az) the light chain variable region comprises SEQ ID NO: 44, and the heavy chain variable region comprises SEQ ID NO: 355;
   (ba) the light chain variable region comprises SEQ ID NO: 46, and the heavy chain variable region comprises SEQ ID NO: 356;
   (bb) the light chain variable region comprises SEQ ID NO: 48, and the heavy chain variable region comprises SEQ ID NO: 357;
   (bc) the light chain variable region comprises SEQ ID NO: 50, and the heavy chain variable region comprises SEQ ID NO: 358;
   (bd) the light chain variable region comprises SEQ ID NO: 52, and the heavy chain variable region comprises SEQ ID NO: 359;
   (be) the light chain variable region comprises SEQ ID NO: 54, and the heavy chain variable region comprises SEQ ID NO: 360;
   (bf) the light chain variable region comprises SEQ ID NO: 56, and the heavy chain variable region comprises SEQ ID NO: 361;
   (bg) the light chain variable region comprises SEQ ID NO: 58, and the heavy chain variable region comprises SEQ ID NO: 362; or
   (bh) the light chain variable region comprises SEQ ID NO: 60, and the heavy chain variable region comprises SEQ ID NO: 363.
6. The antibody or antigen-binding fragment thereof of any one of embodiments 1-5, wherein:
   (a) the heavy chain has at least 90% sequence identity to SEQ ID NO: 168 or SEQ ID NO: 169, wherein the antibody, or antigen binding fragment thereof, maintains the sequences of HCDR1, HCDR2, HCDR3, LCDR1, LCDR2, and LCDR3 set forth in subpart a of embodiment
   (b) the heavy chain has at least 90% sequence identity to SEQ ID NO: 170 or SEQ ID NO: 171, wherein the antibody, or antigen binding fragment thereof, maintains the sequences of HCDR1, HCDR2, HCDR3, LCDR1, LCDR2, and LCDR3 set forth in subpart b of embodiment 1; or
   (c) the heavy chain has at least 90% sequence identity to SEQ ID NO: 172 or SEQ ID NO: 173, wherein the antibody, or antigen binding fragment thereof, maintains the sequences of HCDR1, HCDR2, HCDR3, LCDR1, LCDR2, and LCDR3 set forth in subpart c of embodiment 1; or
   (d) the heavy chain has at least 90% sequence identity to SEQ ID NO: 174 or SEQ ID NO: 175, wherein the antibody, or antigen binding fragment thereof, maintains the sequences of HCDR1, HCDR2, HCDR3, LCDR1, LCDR2, and LCDR3 set forth in subpart d of embodiment 1; or
   (e) the heavy chain has at least 90% sequence identity to SEQ ID NO: 176 or SEQ ID NO: 177, wherein the antibody, or antigen binding fragment thereof, maintains the sequences of HCDR1, HCDR2, HCDR3, LCDR1, LCDR2, and LCDR3 set forth in subpart e of embodiment 1; or
   (f) the heavy chain has at least 90% sequence identity to SEQ ID NO: 178 or SEQ ID NO: 179, wherein the antibody, or antigen binding fragment thereof, maintains the sequences of HCDR1, HCDR2, HCDR3, LCDR1, LCDR2, and LCDR3 set forth in subpart f of embodiment 1; or
   (g) the heavy chain has at least 90% sequence identity to SEQ ID NO: 180 or SEQ ID NO: 181, wherein the antibody, or antigen binding fragment thereof, maintains the sequences of HCDR1, HCDR2, HCDR3, LCDR1, LCDR2, and LCDR3 set forth in subpart g of embodiment 1; or
   (h) the heavy chain has at least 90% sequence identity to SEQ ID NO: 182 or SEQ ID NO: 183, wherein the antibody, or antigen binding fragment thereof, maintains the sequences of HCDR1, HCDR2, HCDR3, LCDR1, LCDR2, and LCDR3 set forth in subpart h of embodiment 1; or
   (i) the heavy chain has at least 90% sequence identity to SEQ ID NO: 184 or SEQ ID NO: 185, wherein the antibody, or antigen binding fragment thereof, maintains the sequences of HCDR1, HCDR2, HCDR3, LCDR1, LCDR2, and LCDR3 set forth in subpart i of embodiment 1; or
   (j) the heavy chain has at least 90% sequence identity to SEQ ID NO: 186 or SEQ ID NO: 187, wherein the antibody, or antigen binding fragment thereof, maintains the sequences of HCDR1, HCDR2, HCDR3, LCDR1, LCDR2, and LCDR3 set forth in subpart j of embodiment 1; or
   (k) the heavy chain has at least 90% sequence identity to SEQ ID NO: 188 or SEQ ID NO: 189, wherein the antibody, or antigen binding fragment thereof, maintains the sequences of HCDR1, HCDR2, HCDR3, LCDR1, LCDR2, and LCDR3 set forth in subpart k of embodiment 1; or
   (l) the heavy chain has at least 90% sequence identity to SEQ ID NO: 190 or SEQ ID NO: 191, wherein the antibody, or antigen binding fragment thereof, maintains the sequences of HCDR1, HCDR2, HCDR3, LCDR1, LCDR2, and LCDR3 set forth in subpart 1 of embodiment 1; or
   (m) the heavy chain has at least 90% sequence identity to SEQ ID NO: 192 or SEQ ID NO: 193, wherein the antibody, or antigen binding fragment thereof, maintains the sequences of HCDR1, HCDR2, HCDR3, LCDR1, LCDR2, and LCDR3 set forth in subpart m of embodiment 1; or
   (n) the heavy chain has at least 90% sequence identity to SEQ ID NO: 194 or SEQ ID NO: 195, wherein the antibody, or antigen binding fragment thereof, maintains the sequences of HCDR1, HCDR2, HCDR3, LCDR1, LCDR2, and LCDR3 set forth in subpart n of embodiment 1; or
   (o) the heavy chain has at least 90% sequence identity to SEQ ID NO: 196 or SEQ ID NO: 197, wherein the antibody, or antigen binding fragment thereof, maintains the sequences of HCDR1, HCDR2, HCDR3, LCDR1, LCDR2, and LCDR3 set forth in subpart o of embodiment 1; or
   (p) the heavy chain has at least 90% sequence identity to SEQ ID NO: 198 or SEQ ID NO: 199, wherein the antibody, or antigen binding fragment thereof, maintains the sequences of HCDR1, HCDR2, HCDR3, LCDR1, LCDR2, and LCDR3 set forth in subpart p of embodiment
   (q) the heavy chain has at least 90% sequence identity to SEQ ID NO: 200 or SEQ ID NO: 201, wherein the antibody, or antigen binding fragment thereof, maintains the sequences of HCDR1, HCDR2, HCDR3, LCDR1, LCDR2, and LCDR3 set forth in subpart q of embodiment 1; or
   (r) the heavy chain has at least 90% sequence identity to SEQ ID NO: 202 or SEQ ID NO: 203, wherein the antibody, or antigen binding fragment thereof, maintains the sequences of HCDR1, HCDR2, HCDR3, LCDR1, LCDR2, and LCDR3 set forth in subpart r of embodiment 1; or
   (s) the heavy chain has at least 90% sequence identity to SEQ ID NO: 204 or SEQ ID NO: 205, wherein the antibody, or antigen binding fragment thereof, maintains the sequences of HCDR1, HCDR2, HCDR3, LCDR1, LCDR2, and LCDR3 set forth in subpart s of embodiment 1; or
   (t) the heavy chain has at least 90% sequence identity to SEQ ID NO: 206 or SEQ ID NO: 207, wherein the antibody, or antigen binding fragment thereof, maintains the sequences of HCDR1, HCDR2, HCDR3, LCDR1, LCDR2, and LCDR3 set forth in subpart t of embodiment 1; or
   (u) the heavy chain has at least 90% sequence identity to SEQ ID NO: 208 or SEQ ID NO: 209, wherein the antibody, or antigen binding fragment thereof, maintains the sequences of HCDR1, HCDR2, HCDR3, LCDR1, LCDR2, and LCDR3 set forth in subpart u of embodiment 1; or
   (v) the heavy chain has at least 90% sequence identity to SEQ ID NO: 210 or SEQ ID NO: 211, wherein the antibody, or antigen binding fragment thereof, maintains the sequences of HCDR1, HCDR2, HCDR3, LCDR1, LCDR2, and LCDR3 set forth in subpart v of embodiment 1; or
   (w) the heavy chain has at least 90% sequence identity to SEQ ID NO: 212 or SEQ ID NO: 213, wherein the antibody, or antigen binding fragment thereof, maintains the sequences of HCDR1, HCDR2, HCDR3, LCDR1, LCDR2, and LCDR3 set forth in subpart w of embodiment 1; or
   (x) the heavy chain has at least 90% sequence identity to SEQ ID NO: 214 or SEQ ID NO: 215, wherein the antibody, or antigen binding fragment thereof, maintains the sequences of HCDR1, HCDR2, HCDR3, LCDR1, LCDR2, and LCDR3 set forth in subpart x of embodiment 1; or
   (y) the heavy chain has at least 90% sequence identity to SEQ ID NO: 216 or SEQ ID NO: 217, wherein the antibody, or antigen binding fragment thereof, maintains the sequences of HCDR1, HCDR2, HCDR3, LCDR1, LCDR2, and LCDR3 set forth in subpart y of embodiment 1; or
   (z) the heavy chain has at least 90% sequence identity to SEQ ID NO: 218 or SEQ ID NO: 219, wherein the antibody, or antigen binding fragment thereof, maintains the sequences of HCDR1, HCDR2, HCDR3, LCDR1, LCDR2, and LCDR3 set forth in subpart z of embodiment 1; or
   (aa) the heavy chain has at least 90% sequence identity to SEQ ID NO: 220 or SEQ ID NO: 221, wherein the antibody, or antigen binding fragment thereof, maintains the sequences of HCDR1, HCDR2, HCDR3, LCDR1, LCDR2, and LCDR3 set forth in subpart ab of embodiment 1; or
   (ab) the heavy chain has at least 90% sequence identity to SEQ ID NO: 222 or SEQ ID NO: 223, wherein the antibody, or antigen binding fragment thereof, maintains the sequences of HCDR1, HCDR2, HCDR3, LCDR1, LCDR2, and LCDR3 set forth in subpart ac of embodiment 1; or
   (ac) the heavy chain has at least 90% sequence identity to SEQ ID NO: 224 or SEQ ID NO: 225, wherein the antibody, or antigen binding fragment thereof, maintains the sequences of HCDR1, HCDR2, HCDR3, LCDR1, LCDR2, and LCDR3 set forth in subpart ad of embodiment 1; or
   (ad) the heavy chain has at least 90% sequence identity to SEQ ID NO: 226 or SEQ ID NO: 227, wherein the antibody, or antigen binding fragment thereof, maintains the sequences of HCDR1, HCDR2, HCDR3, LCDR1, LCDR2, and LCDR3 set forth in subpart ae of embodiment 1; or
   (ae) the heavy chain has at least 90% sequence identity to SEQ ID NO: 364 or SEQ ID NO: 365, wherein the antibody, or antigen binding fragment thereof, maintains the sequences of HCDR1, HCDR2, HCDR3, LCDR1, LCDR2, and LCDR3 set forth in subpart a of embodiment
   (af) the heavy chain has at least 90% sequence identity to SEQ ID NO: 366 or SEQ ID NO: 367, wherein the antibody, or antigen binding fragment thereof, maintains the sequences of HCDR1, HCDR2, HCDR3, LCDR1, LCDR2, and LCDR3 set forth in subpart b of embodiment 1; or
   (ag) the heavy chain has at least 90% sequence identity to SEQ ID NO: 368 or SEQ ID NO: 369, wherein the antibody, or antigen binding fragment thereof, maintains the sequences of HCDR1, HCDR2, HCDR3, LCDR1, LCDR2, and LCDR3 set forth in subpart c of embodiment 1; or
   (ah) the heavy chain has at least 90% sequence identity to SEQ ID NO: 370 or SEQ ID NO: 371, wherein the antibody, or antigen binding fragment thereof, maintains the sequences of HCDR1, HCDR2, HCDR3, LCDR1, LCDR2, and LCDR3 set forth in subpart d of embodiment 1; or
   (ai) the heavy chain has at least 90% sequence identity to SEQ ID NO: 372 or SEQ ID NO: 373, wherein the antibody, or antigen binding fragment thereof, maintains the sequences of HCDR1, HCDR2, HCDR3, LCDR1, LCDR2, and LCDR3 set forth in subpart e of embodiment 1; or
   (aj) the heavy chain has at least 90% sequence identity to SEQ ID NO: 374 or SEQ ID NO: 375, wherein the antibody, or antigen binding fragment thereof, maintains the sequences of HCDR1, HCDR2, HCDR3, LCDR1, LCDR2, and LCDR3 set forth in subpart f of embodiment 1; or
   (ak) the heavy chain has at least 90% sequence identity to SEQ ID NO: 376 or SEQ ID NO: 377, wherein the antibody, or antigen binding fragment thereof, maintains the sequences of HCDR1, HCDR2, HCDR3, LCDR1, LCDR2, and LCDR3 set forth in subpart g of embodiment 1; or
   (al) the heavy chain has at least 90% sequence identity to SEQ ID NO: 378 or SEQ ID NO: 379, wherein the antibody, or antigen binding fragment thereof, maintains the sequences of HCDR1, HCDR2, HCDR3, LCDR1, LCDR2, and LCDR3 set forth in subpart h of embodiment 1; or
   (am) the heavy chain has at least 90% sequence identity to SEQ ID NO: 380 or SEQ ID NO: 381, wherein the antibody, or antigen binding fragment thereof, maintains the sequences of HCDR1, HCDR2, HCDR3, LCDR1, LCDR2, and LCDR3 set forth in subpart i of embodiment 1; or
   (an) the heavy chain has at least 90% sequence identity to SEQ ID NO: 382 or SEQ ID NO: 383, wherein the antibody, or antigen binding fragment thereof, maintains the sequences of HCDR1, HCDR2, HCDR3, LCDR1, LCDR2, and LCDR3 set forth in subpart j of embodiment 1; or
   (ao) the heavy chain has at least 90% sequence identity to SEQ ID NO: 384 or SEQ ID NO: 385, wherein the antibody, or antigen binding fragment thereof, maintains the sequences of HCDR1, HCDR2, HCDR3, LCDR1, LCDR2, and LCDR3 set forth in subpart k of embodiment 1; or
   (ap) the heavy chain has at least 90% sequence identity to SEQ ID NO: 386 or SEQ ID NO: 387, wherein the antibody, or antigen binding fragment thereof, maintains the sequences of HCDR1, HCDR2, HCDR3, LCDR1, LCDR2, and LCDR3 set forth in subpart 1 of embodiment 1; or
   (aq) the heavy chain has at least 90% sequence identity to SEQ ID NO: 388 or SEQ ID NO: 389, wherein the antibody, or antigen binding fragment thereof, maintains the sequences of HCDR1, HCDR2, HCDR3, LCDR1, LCDR2, and LCDR3 set forth in subpart m of embodiment 1; or
   (ar) the heavy chain has at least 90% sequence identity to SEQ ID NO: 390 or SEQ ID NO: 391, wherein the antibody, or antigen binding fragment thereof, maintains the sequences of HCDR1, HCDR2, HCDR3, LCDR1, LCDR2, and LCDR3 set forth in subpart n of embodiment 1; or
   (as) the heavy chain has at least 90% sequence identity to SEQ ID NO: 392 or SEQ ID NO: 393, wherein the antibody, or antigen binding fragment thereof, maintains the sequences of HCDR1, HCDR2, HCDR3, LCDR1, LCDR2, and LCDR3 set forth in subpart o of embodiment 1; or
   (at) the heavy chain has at least 90% sequence identity to SEQ ID NO: 394 or SEQ ID NO: 395, wherein the antibody, or antigen binding fragment thereof, maintains the sequences of HCDR1, HCDR2, HCDR3, LCDR1, LCDR2, and LCDR3 set forth in subpart p of embodiment
   (au) the heavy chain has at least 90% sequence identity to SEQ ID NO: 396 or SEQ ID NO: 397, wherein the antibody, or antigen binding fragment thereof, maintains the sequences of HCDR1, HCDR2, HCDR3, LCDR1, LCDR2, and LCDR3 set forth in subpart q of embodiment 1; or
   (av) the heavy chain has at least 90% sequence identity to SEQ ID NO: 398 or SEQ ID NO: 399, wherein the antibody, or antigen binding fragment thereof, maintains the sequences of HCDR1, HCDR2, HCDR3, LCDR1, LCDR2, and LCDR3 set forth in subpart r of embodiment 1; or
   (aw) the heavy chain has at least 90% sequence identity to SEQ ID NO: 400 or SEQ ID NO: 401, wherein the antibody, or antigen binding fragment thereof, maintains the sequences of HCDR1, HCDR2, HCDR3, LCDR1, LCDR2, and LCDR3 set forth in subpart s of embodiment 1; or
   (ax) the heavy chain has at least 90% sequence identity to SEQ ID NO: 402 or SEQ ID NO: 403, wherein the antibody, or antigen binding fragment thereof, maintains the sequences of HCDR1, HCDR2, HCDR3, LCDR1, LCDR2, and LCDR3 set forth in subpart t of embodiment 1; or
   (ay) the heavy chain has at least 90% sequence identity to SEQ ID NO: 404 or SEQ ID NO: 405, wherein the antibody, or antigen binding fragment thereof, maintains the sequences of HCDR1, HCDR2, HCDR3, LCDR1, LCDR2, and LCDR3 set forth in subpart u of embodiment 1; or
   (az) the heavy chain has at least 90% sequence identity to SEQ ID NO: 406 or SEQ ID NO: 407, wherein the antibody, or antigen binding fragment thereof, maintains the sequences of HCDR1, HCDR2, HCDR3, LCDR1, LCDR2, and LCDR3 set forth in subpart v of embodiment 1; or
   (ba) the heavy chain has at least 90% sequence identity to SEQ ID NO: 408 or SEQ ID NO: 409, wherein the antibody, or antigen binding fragment thereof, maintains the sequences of HCDR1, HCDR2, HCDR3, LCDR1, LCDR2, and LCDR3 set forth in subpart w of embodiment 1; or
   (bb) the heavy chain has at least 90% sequence identity to SEQ ID NO: 410 or SEQ ID NO: 411, wherein the antibody, or antigen binding fragment thereof, maintains the sequences of HCDR1, HCDR2, HCDR3, LCDR1, LCDR2, and LCDR3 set forth in subpart x of embodiment 1; or
   (bc) the heavy chain has at least 90% sequence identity to SEQ ID NO: 412 or SEQ ID NO: 413, wherein the antibody, or antigen binding fragment thereof, maintains the sequences of HCDR1, HCDR2, HCDR3, LCDR1, LCDR2, and LCDR3 set forth in subpart y of embodiment 1; or
   (bd) the heavy chain has at least 90% sequence identity to SEQ ID NO: 414 or SEQ ID NO: 415, wherein the antibody, or antigen binding fragment thereof, maintains the sequences of HCDR1, HCDR2, HCDR3, LCDR1, LCDR2, and LCDR3 set forth in subpart z of embodiment 1; or
   (be) the heavy chain has at least 90% sequence identity to SEQ ID NO: 416 or SEQ ID NO: 417, wherein the antibody, or antigen binding fragment thereof, maintains the sequences of HCDR1, HCDR2, HCDR3, LCDR1, LCDR2, and LCDR3 set forth in subpart ab of embodiment 1; or
   (bf) the heavy chain has at least 90% sequence identity to SEQ ID NO: 418 or SEQ ID NO: 419, wherein the antibody, or antigen binding fragment thereof, maintains the sequences of HCDR1, HCDR2, HCDR3, LCDR1, LCDR2, and LCDR3 set forth in subpart ac of embodiment 1; or
   (bg) the heavy chain has at least 90% sequence identity to SEQ ID NO: 420 or SEQ ID NO: 421, wherein the antibody, or antigen binding fragment thereof, maintains the sequences of HCDR1, HCDR2, HCDR3, LCDR1, LCDR2, and LCDR3 set forth in subpart ad of embodiment 1; or
   (bh) the heavy chain has at least 90% sequence identity to SEQ ID NO: 422 or SEQ ID NO: 423, wherein the antibody, or antigen binding fragment thereof, maintains the sequences of HCDR1, HCDR2, HCDR3, LCDR1, LCDR2, and LCDR3 set forth in subpart ae of embodiment 1.
7. The antibody or antigen-binding fragment thereof of any one of embodiments 1-6, wherein the light chain comprises:
   (a) the light chain has at least 90% sequence identity to SEQ ID NO: 228, wherein the antibody, or antigen binding fragment thereof, maintains the sequences of HCDR1, HCDR2, HCDR3, LCDR1, LCDR2, and LCDR3 set forth in subpart a of embodiment 1; or
   (b) the light chain has least 90% sequence identity to SEQ ID NO: 229, wherein the antibody, or antigen binding fragment thereof, maintains the sequences of HCDR1, HCDR2, HCDR3, LCDR1, LCDR2, and LCDR3 set forth in subpart b of embodiment 1; or
   (c) the light chain has at least 90% sequence identity to SEQ ID NO: 230, wherein the antibody, or antigen binding fragment thereof, maintains the sequences of HCDR1, HCDR2, HCDR3, LCDR1, LCDR2, and LCDR3 set forth in subpart c of embodiment 1; or
   (d) the light chain has at least 90% sequence identity to SEQ ID NO: 231, wherein the antibody, or antigen binding fragment thereof, maintains the sequences of HCDR1, HCDR2, HCDR3, LCDR1, LCDR2, and LCDR3 set forth in subpart d of embodiment 1; or
   (e) the light chain has at least 90% sequence identity to SEQ ID NO: 232, wherein the antibody, or antigen binding fragment thereof, maintains the sequences of HCDR1, HCDR2, HCDR3, LCDR1, LCDR2, and LCDR3 set forth in subpart e of embodiment 1; or
   (f) the light chain has at least 90% sequence identity to SEQ ID NO: 233, wherein the antibody, or antigen binding fragment thereof, maintains the sequences of HCDR1, HCDR2, HCDR3, LCDR1, LCDR2, and LCDR3 set forth in subpart f of embodiment 1; or
   (g) the light chain has at least 90% sequence identity to SEQ ID NO: 234, wherein the antibody, or antigen binding fragment thereof, maintains the sequences of HCDR1, HCDR2, HCDR3, LCDR1, LCDR2, and LCDR3 set forth in subpart g of embodiment 1; or
   (h) the light chain has at least 90% sequence identity to SEQ ID NO: 235, wherein the antibody, or antigen binding fragment thereof, maintains the sequences of HCDR1, HCDR2, HCDR3, LCDR1, LCDR2, and LCDR3 set forth in subpart h of embodiment 1; or
   (i) the light chain has at least 90% sequence identity to SEQ ID NO: 236, wherein the antibody, or antigen binding fragment thereof, maintains the sequences of HCDR1, HCDR2, HCDR3, LCDR1, LCDR2, and LCDR3 set forth in subpart i of embodiment 1; or
   (j) the light chain has at least 90% sequence identity to SEQ ID NO: 237, wherein the antibody, or antigen binding fragment thereof, maintains the sequences of HCDR1, HCDR2, HCDR3, LCDR1, LCDR2, and LCDR3 set forth in subpart j of embodiment 1; or
   (k) the light chain has at least 90% sequence identity to SEQ ID NO: 238, wherein the antibody, or antigen binding fragment thereof, maintains the sequences of HCDR1, HCDR2, HCDR3, LCDR1, LCDR2, and LCDR3 set forth in subpart k of embodiment 1; or
   (l) the light chain has at least 90% sequence identity to SEQ ID NO: 239, wherein the antibody, or antigen binding fragment thereof, maintains the sequences of HCDR1, HCDR2, HCDR3, LCDR1, LCDR2, and LCDR3 set forth in subpart 1 of embodiment 1; or
   (m) the light chain has at least 90% sequence identity to SEQ ID NO: 240, wherein the antibody, or antigen binding fragment thereof, maintains the sequences of HCDR1, HCDR2, HCDR3, LCDR1, LCDR2, and LCDR3 set forth in subpart m of embodiment 1; or
   (n) the light chain has at least 90% sequence identity to SEQ ID NO: 241, wherein the antibody, or antigen binding fragment thereof, maintains the sequences of HCDR1, HCDR2, HCDR3, LCDR1, LCDR2, and LCDR3 set forth in subpart n of embodiment 1; or
   (o) the light chain has at least 90% sequence identity to SEQ ID NO: 242, wherein the antibody, or antigen binding fragment thereof, maintains the sequences of HCDR1, HCDR2, HCDR3, LCDR1, LCDR2, and LCDR3 set forth in subpart o of embodiment 1; or
   (p) the light chain has at least 90% sequence identity to SEQ ID NO: 243, wherein the antibody, or antigen binding fragment thereof, maintains the sequences of HCDR1, HCDR2, HCDR3, LCDR1, LCDR2, and LCDR3 set forth in subpart p of embodiment 1; or
   (q) the light chain has at least 90% sequence identity to SEQ ID NO: 244, wherein the antibody, or antigen binding fragment thereof, maintains the sequences of HCDR1, HCDR2, HCDR3, LCDR1, LCDR2, and LCDR3 set forth in subpart q of embodiment 1; or
   (r) the light chain has at least 90% sequence identity to SEQ ID NO: 245, wherein the antibody, or antigen binding fragment thereof, maintains the sequences of HCDR1, HCDR2, HCDR3, LCDR1, LCDR2, and LCDR3 set forth in subpart r of embodiment 1; or
   (s) the light chain has at least 90% sequence identity to SEQ ID NO: 246, wherein the antibody, or antigen binding fragment thereof, maintains the sequences of HCDR1, HCDR2, HCDR3, LCDR1, LCDR2, and LCDR3 set forth in subpart s of embodiment 1; or
   (t) the light chain has at least 90% sequence identity to SEQ ID NO: 247, wherein the antibody, or antigen binding fragment thereof, maintains the sequences of HCDR1, HCDR2, HCDR3, LCDR1, LCDR2, and LCDR3 set forth in subpart t of embodiment 1; or
   (u) the light chain has at least 90% sequence identity to SEQ ID NO: 248, wherein the antibody, or antigen binding fragment thereof, maintains the sequences of HCDR1, HCDR2, HCDR3, LCDR1, LCDR2, and LCDR3 set forth in subpart u of embodiment 1; or
   (v) the light chain has at least 90% sequence identity to SEQ ID NO: 249, wherein the antibody, or antigen binding fragment thereof, maintains the sequences of HCDR1, HCDR2, HCDR3, LCDR1, LCDR2, and LCDR3 set forth in subpart v of embodiment 1; or
   (w) the light chain has at least 90% sequence identity to SEQ ID NO: 250, wherein the antibody, or antigen binding fragment thereof, maintains the sequences of HCDR1, HCDR2, HCDR3, LCDR1, LCDR2, and LCDR3 set forth in subpart w of embodiment 1; or
   (x) the light chain has at least 90% sequence identity to SEQ ID NO: 251, wherein the antibody, or antigen binding fragment thereof, maintains the sequences of HCDR1, HCDR2, HCDR3, LCDR1, LCDR2, and LCDR3 set forth in subpart x of embodiment 1; or
   (y) the light chain has at least 90% sequence identity to SEQ ID NO: 252, wherein the antibody, or antigen binding fragment thereof, maintains the sequences of HCDR1, HCDR2, HCDR3, LCDR1, LCDR2, and LCDR3 set forth in subpart y of embodiment 1; or
   (z) the light chain has at least 90% sequence identity to SEQ ID NO: 253, wherein the antibody, or antigen binding fragment thereof, maintains the sequences of HCDR1, HCDR2, HCDR3, LCDR1, LCDR2, and LCDR3 set forth in subpart z of embodiment 1; or
   (aa) the light chain has at least 90% sequence identity to SEQ ID NO: 254, wherein the antibody, or antigen binding fragment thereof, maintains the sequences of HCDR1, HCDR2, HCDR3, LCDR1, LCDR2, and LCDR3 set forth in subpart ab of embodiment 1; or
   (ab) the light chain has at least 90% sequence identity to SEQ ID NO: 255, wherein the antibody, or antigen binding fragment thereof, maintains the sequences of HCDR1, HCDR2, HCDR3, LCDR1, LCDR2, and LCDR3 set forth in subpart ac of embodiment 1; or
   (ac) the light chain has at least 90% sequence identity to SEQ ID NO: 256, wherein the antibody, or antigen binding fragment thereof, maintains the sequences of HCDR1, HCDR2, HCDR3, LCDR1, LCDR2, and LCDR3 set forth in subpart ad of embodiment 1; or
   (ad) the light chain has at least 90% sequence identity to SEQ ID NO: 257, wherein the antibody, or antigen binding fragment thereof, maintains the sequences of HCDR1, HCDR2, HCDR3, LCDR1, LCDR2, and LCDR3 set forth in subpart ae of embodiment 1.
8. The antibody, or antigen-binding fragment thereof, of any one of embodiments 1-7, wherein:
   (a) the light chain has at least 90% sequence identity to SEQ ID NO: 228, and the heavy chain has at least 90% sequence identity to SEQ ID NO: 168 or SEQ ID NO: 169, wherein the antibody, or antigen binding fragment thereof, maintains the sequences of HCDR1, HCDR2, HCDR3, LCDR1, LCDR2, and LCDR3 set forth in subpart a of embodiment 1; or
   (b) the light chain has at least 90% sequence identity to SEQ ID NO: 229, and the heavy chain has at least 90% sequence identity to SEQ ID NO: 170 or SEQ ID NO: 171, wherein the antibody, or antigen binding fragment thereof, maintains the sequences of HCDR1, HCDR2, HCDR3, LCDR1, LCDR2, and LCDR3 set forth in subpart b of embodiment 1; or
   (c) the light chain has at least 90% sequence identity to SEQ ID NO: 230, and the heavy chain has at least 90% sequence identity to SEQ ID NO: 172 or SEQ ID NO: 173, wherein the antibody, or antigen binding fragment thereof, maintains the sequences of HCDR1, HCDR2, HCDR3, LCDR1, LCDR2, and LCDR3 set forth in subpart c of embodiment 1; or
   (d) the light chain has at least 90% sequence identity to SEQ ID NO: 231, and the heavy chain has at least 90% sequence identity to SEQ ID NO: 174 or SEQ ID NO: 175, wherein the antibody, or antigen binding fragment thereof, maintains the sequences of HCDR1, HCDR2, HCDR3, LCDR1, LCDR2, and LCDR3 set forth in subpart d of embodiment 1; or
   (e) the light chain has at least 90% sequence identity to SEQ ID NO: 232, and the heavy chain has at least 90% sequence identity to SEQ ID NO: 176 or SEQ ID NO: 177, wherein the antibody, or antigen binding fragment thereof, maintains the sequences of HCDR1, HCDR2, HCDR3, LCDR1, LCDR2, and LCDR3 set forth in subpart e of embodiment 1; or
   (f) the light chain has at least 90% sequence identity to SEQ ID NO: 233, and the heavy chain has at least 90% sequence identity to SEQ ID NO: 178 or SEQ ID NO: 179, wherein the antibody, or antigen binding fragment thereof, maintains the sequences of HCDR1, HCDR2, HCDR3, LCDR1, LCDR2, and LCDR3 set forth in subpart f of embodiment 1; or
   (g) the light chain has at least 90% sequence identity to SEQ ID NO: 234, and the heavy chain has at least 90% sequence identity to SEQ ID NO: 180 or SEQ ID NO: 181, wherein the antibody, or antigen binding fragment thereof, maintains the sequences of HCDR1, HCDR2, HCDR3, LCDR1, LCDR2, and LCDR3 set forth in subpart g of embodiment 1; or
   (h) the light chain has at least 90% sequence identity to SEQ ID NO: 235, and the heavy chain has at least 90% sequence identity to SEQ ID NO: 182 or SEQ ID NO: 183, wherein the antibody, or antigen binding fragment thereof, maintains the sequences of HCDR1, HCDR2, HCDR3, LCDR1, LCDR2, and LCDR3 set forth in subpart h of embodiment 1; or
   (i) the light chain has at least 90% sequence identity to SEQ ID NO: 236, and the heavy chain has at least 90% sequence identity to SEQ ID NO: 184 or SEQ ID NO: 185, wherein the antibody, or antigen binding fragment thereof, maintains the sequences of HCDR1, HCDR2, HCDR3, LCDR1, LCDR2, and LCDR3 set forth in subpart i of embodiment 1; or
   (j) the light chain has at least 90% sequence identity to SEQ ID NO: 237, and the heavy chain has at least 90% sequence identity to SEQ ID NO: 186 or SEQ ID NO: 187, wherein the antibody, or antigen binding fragment thereof, maintains the sequences of HCDR1, HCDR2, HCDR3, LCDR1, LCDR2, and LCDR3 set forth in subpart j of embodiment 1; or
   (k) the light chain has at least 90% sequence identity to SEQ ID NO: 238, and the heavy chain has at least 90% sequence identity to SEQ ID NO: 188 or SEQ ID NO: 189, wherein the antibody, or antigen binding fragment thereof, maintains the sequences of HCDR1, HCDR2, HCDR3, LCDR1, LCDR2, and LCDR3 set forth in subpart k of embodiment 1; or
   (l) the light chain has at least 90% sequence identity to SEQ ID NO: 239, and the heavy chain has at least 90% sequence identity to SEQ ID NO: 190 or SEQ ID NO: 191, wherein the antibody, or antigen binding fragment thereof, maintains the sequences of HCDR1, HCDR2, HCDR3, LCDR1, LCDR2, and LCDR3 set forth in subpart 1 of embodiment 1; or
   (m) the light chain has at least 90% sequence identity to SEQ ID NO: 240, and the heavy chain has at least 90% sequence identity to SEQ ID NO: 192 or SEQ ID NO: 193, wherein the antibody, or antigen binding fragment thereof, maintains the sequences of HCDR1, HCDR2, HCDR3, LCDR1, LCDR2, and LCDR3 set forth in subpart m of embodiment 1; or
   (n) the light chain has at least 90% sequence identity to SEQ ID NO: 241, and the heavy chain has at least 90% sequence identity to SEQ ID NO: 194 or SEQ ID NO: 195, wherein the antibody, or antigen binding fragment thereof, maintains the sequences of HCDR1, HCDR2, HCDR3, LCDR1, LCDR2, and LCDR3 set forth in subpart n of embodiment 1; or
   (o) the light chain has at least 90% sequence identity to SEQ ID NO: 242, and the heavy chain has at least 90% sequence identity to SEQ ID NO: 196 or SEQ ID NO: 197, wherein the antibody, or antigen binding fragment thereof, maintains the sequences of HCDR1, HCDR2, HCDR3, LCDR1, LCDR2, and LCDR3 set forth in subpart o of embodiment 1; or
   (p) the light chain has at least 90% sequence identity to SEQ ID NO: 243, and the heavy chain has at least 90% sequence identity to SEQ ID NO: 198 or SEQ ID NO: 199, wherein the antibody, or antigen binding fragment thereof, maintains the sequences of HCDR1, HCDR2, HCDR3, LCDR1, LCDR2, and LCDR3 set forth in subpart p of embodiment 1; or
   (q) the light chain has at least 90% sequence identity to SEQ ID NO: 244, and the heavy chain has at least 90% sequence identity to SEQ ID NO: 200 or SEQ ID NO: 201, wherein the antibody, or antigen binding fragment thereof, maintains the sequences of HCDR1, HCDR2, HCDR3, LCDR1, LCDR2, and LCDR3 set forth in subpart q of embodiment 1; or
   (r) the light chain has at least 90% sequence identity to SEQ ID NO: 245, and the heavy chain has at least 90% sequence identity to SEQ ID NO: 202 or SEQ ID NO: 203, wherein the antibody, or antigen binding fragment thereof, maintains the sequences of HCDR1, HCDR2, HCDR3, LCDR1, LCDR2, and LCDR3 set forth in subpart r of embodiment 1; or
   (s) the light chain has at least 90% sequence identity to SEQ ID NO: 246, and the heavy chain has at least 90% sequence identity to SEQ ID NO: 204 or SEQ ID NO: 205, wherein the antibody, or antigen binding fragment thereof, maintains the sequences of HCDR1, HCDR2, HCDR3, LCDR1, LCDR2, and LCDR3 set forth in subpart s of embodiment 1; or
   (t) the light chain has at least 90% sequence identity to SEQ ID NO: 247, and the heavy chain has at least 90% sequence identity to SEQ ID NO: 206 or SEQ ID NO: 207, wherein the antibody, or antigen binding fragment thereof, maintains the sequences of HCDR1, HCDR2, HCDR3, LCDR1, LCDR2, and LCDR3 set forth in subpart t of embodiment 1; or
   (u) the light chain has at least 90% sequence identity to SEQ ID NO: 248, and the heavy chain has at least 90% sequence identity to SEQ ID NO: 208 or SEQ ID NO: 209, wherein the antibody, or antigen binding fragment thereof, maintains the sequences of HCDR1, HCDR2, HCDR3, LCDR1, LCDR2, and LCDR3 set forth in subpart u of embodiment 1; or
   (v) the light chain has at least 90% sequence identity to SEQ ID NO: 249, and the heavy chain has at least 90% sequence identity to SEQ ID NO: 210 or SEQ ID NO: 211, wherein the antibody, or antigen binding fragment thereof, maintains the sequences of HCDR1, HCDR2, HCDR3, LCDR1, LCDR2, and LCDR3 set forth in subpart v of embodiment 1; or
   (w) the light chain has at least 90% sequence identity to SEQ ID NO: 250, and the heavy chain has at least 90% sequence identity to SEQ ID NO: 212 or SEQ ID NO: 213, wherein the antibody, or antigen binding fragment thereof, maintains the sequences of HCDR1, HCDR2, HCDR3, LCDR1, LCDR2, and LCDR3 set forth in subpart w of embodiment 1; or
   (x) the light chain has at least 90% sequence identity to SEQ ID NO: 251, and the heavy chain has at least 90% sequence identity to SEQ ID NO: 214 or SEQ ID NO: 215, wherein the antibody, or antigen binding fragment thereof, maintains the sequences of HCDR1, HCDR2, HCDR3, LCDR1, LCDR2, and LCDR3 set forth in subpart x of embodiment 1; or
   (y) the light chain has at least 90% sequence identity to SEQ ID NO: 252, and the heavy chain has at least 90% sequence identity to SEQ ID NO: 216 or SEQ ID NO: 217, wherein the antibody, or antigen binding fragment thereof, maintains the sequences of HCDR1, HCDR2, HCDR3, LCDR1, LCDR2, and LCDR3 set forth in subpart y of embodiment 1; or
   (z) the light chain has at least 90% sequence identity to SEQ ID NO: 253, and the heavy chain has at least 90% sequence identity to SEQ ID NO: 218 or SEQ ID NO: 219, wherein the antibody, or antigen binding fragment thereof, maintains the sequences of HCDR1, HCDR2, HCDR3, LCDR1, LCDR2, and LCDR3 set forth in subpart z of embodiment 1; or
   (aa) the light chain has at least 90% sequence identity to SEQ ID NO: 254, and the heavy chain has at least 90% sequence identity to SEQ ID NO: 220 or SEQ ID NO: 221, wherein the antibody, or antigen binding fragment thereof, maintains the sequences of HCDR1, HCDR2, HCDR3, LCDR1, LCDR2, and LCDR3 set forth in subpart ab of embodiment 1; or
   (ab) the light chain has at least 90% sequence identity to SEQ ID NO: 255, and the heavy chain has at least 90% sequence identity to SEQ ID NO: 222 or SEQ ID NO: 223, wherein the antibody, or antigen binding fragment thereof, maintains the sequences of HCDR1, HCDR2, HCDR3, LCDR1, LCDR2, and LCDR3 set forth in subpart ac of embodiment 1; or
   (ac) the light chain has at least 90% sequence identity to SEQ ID NO: 256, and the heavy chain has at least 90% sequence identity to SEQ ID NO: 224 or SEQ ID NO: 225, wherein the antibody, or antigen binding fragment thereof, maintains the sequences of HCDR1, HCDR2, HCDR3, LCDR1, LCDR2, and LCDR3 set forth in subpart ad of embodiment 1; or
   (ad) the light chain has at least 90% sequence identity to SEQ ID NO: 257, and the heavy chain has at least 90% sequence identity to SEQ ID NO: 226 or SEQ ID NO: 227, wherein the antibody, or antigen binding fragment thereof, maintains the sequences of HCDR1, HCDR2, HCDR3, LCDR1, LCDR2, and LCDR3 set forth in subpart ae of embodiment 1; or
   (ae) the light chain has at least 90% sequence identity to SEQ ID NO: 228, and the heavy chain has at least 90% sequence identity to SEQ ID NO: 364 or SEQ ID NO: 365, wherein the antibody, or antigen binding fragment thereof, maintains the sequences of HCDR1, HCDR2, HCDR3, LCDR1, LCDR2, and LCDR3 set forth in subpart a of embodiment 1; or
   (af) the light chain has at least 90% sequence identity to SEQ ID NO: 229, and the heavy chain has at least 90% sequence identity to SEQ ID NO: 366 or SEQ ID NO: 367, wherein the antibody, or antigen binding fragment thereof, maintains the sequences of HCDR1, HCDR2, HCDR3, LCDR1, LCDR2, and LCDR3 set forth in subpart b of embodiment 1; or
   (ag) the light chain has at least 90% sequence identity to SEQ ID NO: 230, and the heavy chain has at least 90% sequence identity to SEQ ID NO: 368 or SEQ ID NO: 369, wherein the antibody, or antigen binding fragment thereof, maintains the sequences of HCDR1, HCDR2, HCDR3, LCDR1, LCDR2, and LCDR3 set forth in subpart c of embodiment 1; or
   (ah) the light chain has at least 90% sequence identity to SEQ ID NO: 231, and the heavy chain has at least 90% sequence identity to SEQ ID NO: 370 or SEQ ID NO: 371, wherein the antibody, or antigen binding fragment thereof, maintains the sequences of HCDR1, HCDR2, HCDR3, LCDR1, LCDR2, and LCDR3 set forth in subpart d of embodiment 1; or
   (ai) the light chain has at least 90% sequence identity to SEQ ID NO: 232, and the heavy chain has at least 90% sequence identity to SEQ ID NO: 372 or SEQ ID NO: 373, wherein the antibody, or antigen binding fragment thereof, maintains the sequences of HCDR1, HCDR2, HCDR3, LCDR1, LCDR2, and LCDR3 set forth in subpart e of embodiment 1; or
   (aj) the light chain has at least 90% sequence identity to SEQ ID NO: 233, and the heavy chain has at least 90% sequence identity to SEQ ID NO: 374 or SEQ ID NO: 375, wherein the antibody, or antigen binding fragment thereof, maintains the sequences of HCDR1, HCDR2, HCDR3, LCDR1, LCDR2, and LCDR3 set forth in subpart f of embodiment 1; or
   (ak) the light chain has at least 90% sequence identity to SEQ ID NO: 234, and the heavy chain has at least 90% sequence identity to SEQ ID NO: 376 or SEQ ID NO: 377, wherein the antibody, or antigen binding fragment thereof, maintains the sequences of HCDR1, HCDR2, HCDR3, LCDR1, LCDR2, and LCDR3 set forth in subpart g of embodiment 1; or
   (al) the light chain has at least 90% sequence identity to SEQ ID NO: 235, and the heavy chain has at least 90% sequence identity to SEQ ID NO: 378 or SEQ ID NO: 379, wherein the antibody, or antigen binding fragment thereof, maintains the sequences of HCDR1, HCDR2, HCDR3, LCDR1, LCDR2, and LCDR3 set forth in subpart h of embodiment 1; or
   (am) the light chain has at least 90% sequence identity to SEQ ID NO: 236, and the heavy chain has at least 90% sequence identity to SEQ ID NO: 380 or SEQ ID NO: 381, wherein the antibody, or antigen binding fragment thereof, maintains the sequences of HCDR1, HCDR2, HCDR3, LCDR1, LCDR2, and LCDR3 set forth in subpart i of embodiment 1; or
   (an) the light chain has at least 90% sequence identity to SEQ ID NO: 237, and the heavy chain has at least 90% sequence identity to SEQ ID NO: 382 or SEQ ID NO: 383, wherein the antibody, or antigen binding fragment thereof, maintains the sequences of HCDR1, HCDR2, HCDR3, LCDR1, LCDR2, and LCDR3 set forth in subpart j of embodiment 1; or
   (ao) the light chain has at least 90% sequence identity to SEQ ID NO: 238, and the heavy chain has at least 90% sequence identity to SEQ ID NO: 384 or SEQ ID NO: 385, wherein the antibody, or antigen binding fragment thereof, maintains the sequences of HCDR1, HCDR2, HCDR3, LCDR1, LCDR2, and LCDR3 set forth in subpart k of embodiment 1; or
   (ap) the light chain has at least 90% sequence identity to SEQ ID NO: 239, and the heavy chain has at least 90% sequence identity to SEQ ID NO: 386 or SEQ ID NO: 387, wherein the antibody, or antigen binding fragment thereof, maintains the sequences of HCDR1, HCDR2, HCDR3, LCDR1, LCDR2, and LCDR3 set forth in subpart 1 of embodiment 1; or
   (aq) the light chain has at least 90% sequence identity to SEQ ID NO: 240, and the heavy chain has at least 90% sequence identity to SEQ ID NO: 388 or SEQ ID NO: 389, wherein the antibody, or antigen binding fragment thereof, maintains the sequences of HCDR1, HCDR2, HCDR3, LCDR1, LCDR2, and LCDR3 set forth in subpart m of embodiment 1; or
   (ar) the light chain has at least 90% sequence identity to SEQ ID NO: 241, and the heavy chain has at least 90% sequence identity to SEQ ID NO: 390 or SEQ ID NO: 391, wherein the antibody, or antigen binding fragment thereof, maintains the sequences of HCDR1, HCDR2, HCDR3, LCDR1, LCDR2, and LCDR3 set forth in subpart n of embodiment 1; or
   (as) the light chain has at least 90% sequence identity to SEQ ID NO: 242, and the heavy chain has at least 90% sequence identity to SEQ ID NO: 392 or SEQ ID NO: 393, wherein the antibody, or antigen binding fragment thereof, maintains the sequences of HCDR1, HCDR2, HCDR3, LCDR1, LCDR2, and LCDR3 set forth in subpart o of embodiment 1; or
   (at) the light chain has at least 90% sequence identity to SEQ ID NO: 243, and the heavy chain has at least 90% sequence identity to SEQ ID NO: 394 or SEQ ID NO: 395, wherein the antibody, or antigen binding fragment thereof, maintains the sequences of HCDR1, HCDR2, HCDR3, LCDR1, LCDR2, and LCDR3 set forth in subpart p of embodiment 1; or
   (au) the light chain has at least 90% sequence identity to SEQ ID NO: 244, and the heavy chain has at least 90% sequence identity to SEQ ID NO: 396 or SEQ ID NO: 397, wherein the antibody, or antigen binding fragment thereof, maintains the sequences of HCDR1, HCDR2, HCDR3, LCDR1, LCDR2, and LCDR3 set forth in subpart q of embodiment 1; or
   (av) the light chain has at least 90% sequence identity to SEQ ID NO: 245, and the heavy chain has at least 90% sequence identity to SEQ ID NO: 398 or SEQ ID NO: 399, wherein the antibody, or antigen binding fragment thereof, maintains the sequences of HCDR1, HCDR2, HCDR3, LCDR1, LCDR2, and LCDR3 set forth in subpart r of embodiment 1; or
   (aw) the light chain has at least 90% sequence identity to SEQ ID NO: 246, and the heavy chain has at least 90% sequence identity to SEQ ID NO: 400 or SEQ ID NO: 401, wherein the antibody, or antigen binding fragment thereof, maintains the sequences of HCDR1, HCDR2, HCDR3, LCDR1, LCDR2, and LCDR3 set forth in subpart s of embodiment 1; or
   (ax) the light chain has at least 90% sequence identity to SEQ ID NO: 247, and the heavy chain has at least 90% sequence identity to SEQ ID NO: 402 or SEQ ID NO: 403, wherein the antibody, or antigen binding fragment thereof, maintains the sequences of HCDR1, HCDR2, HCDR3, LCDR1, LCDR2, and LCDR3 set forth in subpart t of embodiment 1; or
   (ay) the light chain has at least 90% sequence identity to SEQ ID NO: 248, and the heavy chain has at least 90% sequence identity to SEQ ID NO: 404 or SEQ ID NO: 405, wherein the antibody, or antigen binding fragment thereof, maintains the sequences of HCDR1, HCDR2, HCDR3, LCDR1, LCDR2, and LCDR3 set forth in subpart u of embodiment 1; or
   (az) the light chain has at least 90% sequence identity to SEQ ID NO: 249, and the heavy chain has at least 90% sequence identity to SEQ ID NO: 406 or SEQ ID NO: 407, wherein the antibody, or antigen binding fragment thereof, maintains the sequences of HCDR1, HCDR2, HCDR3, LCDR1, LCDR2, and LCDR3 set forth in subpart v of embodiment 1; or
   (ba) the light chain has at least 90% sequence identity to SEQ ID NO: 250, and the heavy chain has at least 90% sequence identity to SEQ ID NO: 408 or SEQ ID NO: 409, wherein the antibody, or antigen binding fragment thereof, maintains the sequences of HCDR1, HCDR2, HCDR3, LCDR1, LCDR2, and LCDR3 set forth in subpart w of embodiment 1; or
   (bb) the light chain has at least 90% sequence identity to SEQ ID NO: 251, and the heavy chain has at least 90% sequence identity to SEQ ID NO: 410 or SEQ ID NO: 411, wherein the antibody, or antigen binding fragment thereof, maintains the sequences of HCDR1, HCDR2, HCDR3, LCDR1, LCDR2, and LCDR3 set forth in subpart x of embodiment 1; or
   (bc) the light chain has at least 90% sequence identity to SEQ ID NO: 252, and the heavy chain has at least 90% sequence identity to SEQ ID NO: 412 or SEQ ID NO: 413, wherein the antibody, or antigen binding fragment thereof, maintains the sequences of HCDR1, HCDR2, HCDR3, LCDR1, LCDR2, and LCDR3 set forth in subpart y of embodiment 1; or
   (bd) the light chain has at least 90% sequence identity to SEQ ID NO: 253, and the heavy chain has at least 90% sequence identity to SEQ ID NO: 414 or SEQ ID NO: 415, wherein the antibody, or antigen binding fragment thereof, maintains the sequences of HCDR1, HCDR2, HCDR3, LCDR1, LCDR2, and LCDR3 set forth in subpart z of embodiment 1; or
   (be) the light chain has at least 90% sequence identity to SEQ ID NO: 254, and the heavy chain has at least 90% sequence identity to SEQ ID NO: 416 or SEQ ID NO: 417, wherein the antibody, or antigen binding fragment thereof, maintains the sequences of HCDR1, HCDR2, HCDR3, LCDR1, LCDR2, and LCDR3 set forth in subpart ab of embodiment 1; or
   (bf) the light chain has at least 90% sequence identity to SEQ ID NO: 255, and the heavy chain has at least 90% sequence identity to SEQ ID NO: 418 or SEQ ID NO: 419, wherein the antibody, or antigen binding fragment thereof, maintains the sequences of HCDR1, HCDR2, HCDR3, LCDR1, LCDR2, and LCDR3 set forth in subpart ac of embodiment 1; or
   (bg) the light chain has at least 90% sequence identity to SEQ ID NO: 256, and the heavy chain has at least 90% sequence identity to SEQ ID NO: 420 or SEQ ID NO: 421, wherein the antibody, or antigen binding fragment thereof, maintains the sequences of HCDR1, HCDR2, HCDR3, LCDR1, LCDR2, and LCDR3 set forth in subpart ad of embodiment 1; or
   (bh) the light chain has at least 90% sequence identity to SEQ ID NO: 257, and the heavy chain has at least 90% sequence identity to SEQ ID NO: 422 or SEQ ID NO: 423, wherein the antibody, or antigen binding fragment thereof, maintains the sequences of HCDR1, HCDR2, HCDR3, LCDR1, LCDR2, and LCDR3 set forth in subpart ae of embodiment 1.
9. The antibody, or antigen-binding fragment thereof, of embodiment 8, wherein:
   (a) the light chain comprises SEQ ID NO: 228, and the heavy chain comprises SEQ ID NO: 168 or SEQ ID NO: 169; or
   (b) the light chain comprises SEQ ID NO: 229, and the heavy chain comprises SEQ ID NO: 170 or SEQ ID NO: 171; or
   (c) the light chain comprises SEQ ID NO: 230, and the heavy chain comprises SEQ ID NO: 172 or SEQ ID NO: 173; or
   (d) the light chain comprises SEQ ID NO: 231, and the heavy chain comprises SEQ ID NO: 174 or SEQ ID NO: 175; or
   (e) the light chain comprises SEQ ID NO: 232, and the heavy chain comprises SEQ ID NO: 176 or SEQ ID NO: 177; or
   (f) the light chain comprises SEQ ID NO: 233, and the heavy chain comprises SEQ ID NO: 178 or SEQ ID NO: 179; or
   (g) the light chain comprises SEQ ID NO: 234, and the heavy chain comprises SEQ ID NO: 180 or SEQ ID NO: 181; or
   (h) the light chain comprises SEQ ID NO: 235, and the heavy chain comprises SEQ ID NO: 182 or SEQ ID NO: 183; or
   (i) the light chain comprises SEQ ID NO: 236, and the heavy chain comprises SEQ ID NO: 184 or SEQ ID NO: 185; or
   (j) the light chain comprises SEQ ID NO: 237, and the heavy chain comprises SEQ ID NO: 186 or SEQ ID NO: 187; or
   (k) the light chain comprises SEQ ID NO: 238, and the heavy chain comprises SEQ ID NO: 188 or SEQ ID NO: 189; or
   (l) the light chain comprises SEQ ID NO: 239, and the heavy chain comprises SEQ ID NO: 190 or SEQ ID NO: 191; or
   (m) the light chain comprises SEQ ID NO: 240, and the heavy chain comprises SEQ ID NO: 192 or SEQ ID NO: 193; or
   (n) the light chain comprises SEQ ID NO: 241, and the heavy chain comprises SEQ ID NO: 194 or SEQ ID NO: 195; or
   (o) the light chain comprises SEQ ID NO: 242, and the heavy chain comprises SEQ ID NO: 196 or SEQ ID NO: 197; or
   (p) the light chain comprises SEQ ID NO: 243, and the heavy chain comprises SEQ ID NO: 198 or SEQ ID NO: 199; or
   (q) the light chain comprises SEQ ID NO: 244, and the heavy chain comprises SEQ ID NO: 200 or SEQ ID NO: 201; or
   (r) the light chain comprises SEQ ID NO: 245, and the heavy chain comprises SEQ ID NO: 202 or SEQ ID NO: 203; or
   (s) the light chain comprises SEQ ID NO: 246, and the heavy chain comprises SEQ ID NO: 204 or SEQ ID NO: 205; or
   (t) the light chain comprises SEQ ID NO: 247, and the heavy chain comprises SEQ ID NO: 206 or SEQ ID NO: 207; or
   (u) the light chain comprises SEQ ID NO: 248, and the heavy chain comprises SEQ ID NO: 208 or SEQ ID NO: 209; or
   (v) the light chain comprises SEQ ID NO: 249, and the heavy chain comprises SEQ ID NO: 210 or SEQ ID NO: 211; or
   (w) the light chain comprises SEQ ID NO: 250, and the heavy chain comprises SEQ ID NO: 212 or SEQ ID NO: 213; or
   (x) the light chain comprises SEQ ID NO: 251, and the heavy chain comprises SEQ ID NO: 214 or SEQ ID NO: 215; or
   (y) the light chain comprises SEQ ID NO: 252, and the heavy chain comprises SEQ ID NO: 216 or SEQ ID NO: 217; or
   (z) the light chain comprises SEQ ID NO: 253, and the heavy chain comprises SEQ ID NO: 218 or SEQ ID NO: 219; or
   (aa) the light chain comprises SEQ ID NO: 254, and the heavy chain comprises SEQ ID NO: 220 or SEQ ID NO: 221; or
   (ab) the light chain comprises SEQ ID NO: 255, and the heavy chain comprises SEQ ID NO: 222 or SEQ ID NO: 223; or
   (ac) the light chain comprises SEQ ID NO: 256, and the heavy chain comprises SEQ ID NO: 224 or SEQ ID NO: 225; or
   (ad) the light chain comprises SEQ ID NO: 257, and the heavy chain comprises SEQ ID NO: 226 or SEQ ID NO: 227; or
   (ae) the light chain comprises SEQ ID NO: 228, and the heavy chain comprises SEQ ID NO: 364 or SEQ ID NO: 365; or
   (af) the light chain comprises SEQ ID NO: 229, and the heavy chain comprises SEQ ID NO: 366 or SEQ ID NO: 367; or
   (ag) the light chain comprises SEQ ID NO: 230, and the heavy chain comprises SEQ ID NO: 368 or SEQ ID NO: 369; or
   (ah) the light chain comprises SEQ ID NO: 231, and the heavy chain comprises SEQ ID NO: 370 or SEQ ID NO: 371; or
   (ai) the light chain comprises SEQ ID NO: 232, and the heavy chain comprises SEQ ID NO: 372 or SEQ ID NO: 373; or
   (aj) the light chain comprises SEQ ID NO: 233, and the heavy chain comprises SEQ ID NO: 374 or SEQ ID NO: 375; or
   (ak) the light chain comprises SEQ ID NO: 234, and the heavy chain comprises SEQ ID NO: 376 or SEQ ID NO: 377; or
   (al) the light chain comprises SEQ ID NO: 235, and the heavy chain comprises SEQ ID NO: 378 or SEQ ID NO: 379; or
   (am) the light chain comprises SEQ ID NO: 236, and the heavy chain comprises SEQ ID NO: 380 or SEQ ID NO: 381; or
   (an) the light chain comprises SEQ ID NO: 237, and the heavy chain comprises SEQ ID NO: 382 or SEQ ID NO: 383; or
   (ao) the light chain comprises SEQ ID NO: 238, and the heavy chain comprises SEQ ID NO: 384 or SEQ ID NO: 385; or
   (ap) the light chain comprises SEQ ID NO: 239, and the heavy chain comprises SEQ ID NO: 386 or SEQ ID NO: 387; or
   (aq) the light chain comprises SEQ ID NO: 240, and the heavy chain comprises SEQ ID NO: 388 or SEQ ID NO: 389; or
   (ar) the light chain comprises SEQ ID NO: 241, and the heavy chain comprises SEQ ID NO: 390 or SEQ ID NO: 391; or
   (as) the light chain comprises SEQ ID NO: 242, and the heavy chain comprises SEQ ID NO: 392 or SEQ ID NO: 393; or
   (at) the light chain comprises SEQ ID NO: 243, and the heavy chain comprises SEQ ID NO: 394 or SEQ ID NO: 395; or
   (au) the light chain comprises SEQ ID NO: 244, and the heavy chain comprises SEQ ID NO: 396 or SEQ ID NO: 397; or
   (av) the light chain comprises SEQ ID NO: 245, and the heavy chain comprises SEQ ID NO: 398 or SEQ ID NO: 399; or
   (aw) the light chain comprises SEQ ID NO: 246, and the heavy chain comprises SEQ ID NO: 400 or SEQ ID NO: 401; or
   (ax) the light chain comprises SEQ ID NO: 247, and the heavy chain comprises SEQ ID NO: 402 or SEQ ID NO: 403; or
   (ay) the light chain comprises SEQ ID NO: 248, and the heavy chain comprises SEQ ID NO: 404 or SEQ ID NO: 405; or
   (az) the light chain comprises SEQ ID NO: 249, and the heavy chain comprises SEQ ID NO: 406 or SEQ ID NO: 407; or
   (ba) the light chain comprises SEQ ID NO: 250, and the heavy chain comprises SEQ ID NO: 408 or SEQ ID NO: 409; or
   (bb) the light chain comprises SEQ ID NO: 251, and the heavy chain comprises SEQ ID NO: 410 or SEQ ID NO: 411; or
   (bc) the light chain comprises SEQ ID NO: 252, and the heavy chain comprises SEQ ID NO: 412 or SEQ ID NO: 413; or
   (bd) the light chain comprises SEQ ID NO: 253, and the heavy chain comprises SEQ ID NO: 414 or SEQ ID NO: 415; or
   (be) the light chain comprises SEQ ID NO: 254, and the heavy chain comprises SEQ ID NO: 416 or SEQ ID NO: 417; or
   (bf) the light chain comprises SEQ ID NO: 255, and the heavy chain comprises SEQ ID NO: 418 or SEQ ID NO: 419; or
   (bg) the light chain comprises SEQ ID NO: 256, and the heavy chain comprises SEQ ID NO: 420 or SEQ ID NO: 421; or
   (bh) the light chain comprises SEQ ID NO: 257, and the heavy chain comprises SEQ ID NO: 422 or SEQ ID NO: 423.
10. The antibody or antigen-binding fragment thereof of any one of the preceding embodiments, wherein the antibody binding fragment is a scFv antibody, a Fab fragment, a Fab′ fragment, or an F(ab′)₂ fragment.
11. The antibody, or antigen binding fragment thereof, of any one of the preceding embodiments, wherein the antibody is a monoclonal antibody.
12. The antibody, or antigen binding fragment thereof, of any one of the preceding embodiments, wherein the antibody is a humanized antibody.
13. The antibody, or antigen binding fragment thereof, of any one of the preceding embodiments, wherein the antibody, or antigen binding fragment thereof, comprises the sequence selected from one or more of the following sequences: SEQ ID NO: 1; SEQ ID NO: 2; SEQ ID NO: 3; SEQ ID NO: 4; SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, or a variant thereof.
14. The antibody, or antigen binding fragment thereof, of any one of the preceding embodiments, wherein the antibody, or antigen binding fragment thereof, comprises a V_{L} peptide of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60, or any variant thereof.
15. The antibody, or antigen binding fragment thereof, of any one of the preceding embodiments, wherein the antibody, or antigen binding fragment thereof, comprises a V_{H} peptide of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 334, 335, 336, 337, 338, 339, 340, 341, 342, 343, 344, 345, 346, 347, 348, 349, 350, 351, 352, 353, 354, 355, 356, 357, 358, 359, 360, 361, 362, or 363, or any variant thereof.
16. An antibody, or antigen binding fragment thereof, wherein the antibody comprises:
   a variable heavy chain comprising a HCDR1 of SEQ ID NO: 128, a HCDR2 of SEQ ID NO: 129, and a HCDR3 of DEQLGGX₁X₂YYYYYMDX₃ (SEQ ID NO: 333), wherein: X₁ is N, T, I, Q, L, K, or R; X₂ is Y, T, K, H, or V; and X₃ is V or A; and
   a variable light chain LCDR1 of SEQ ID NO: 106, a LCDR2 of SEQ ID NO: 107, and a LCDR3 comprising an amino acid sequence of: X₄QX₅X₆X₇X₈PLX₉ (SEQ ID NO: 332), wherein: X₄ is L or F; X₅ is Y or T; X₆ is N, K, A, H, or R; X₇ is S, Q, I, or A; X₈ is Y, V, or H; and X₉ is T, G, K, R, I or A.
17. An antibody, or antigen binding fragment thereof, wherein the antibody comprises:
   a variable heavy chain comprising a HCDR1 of SEQ ID NO: 61, a HCDR2 of SEQ ID NO: 62, and a HCDR3 of DX₁₀X₁₁X₁₂YX₁₃X₁₄DX₁₅ (SEQ ID NO: 329), wherein: X₁₀ is E, Q, or N; X₁₁ is T, E, S, or N; X₁₂ is D or Q; X₁₃ is A or G; X₁₄ is L, W, F, or Y; and X₁₅ is Y, E, L, N, F, or W; and
   a variable light chain comprising a LCDR1 of SEQ ID NO: 64, a LCDR2 of SEQ ID NO: 65, and a LCDR3 comprising an amino acid sequence of X₁₆X₁₇X₁₈EDX₁₉X₂₀X₂₁X₂₂ (SEQ ID NO: 328), wherein X₁₆ is Q, E, K, or H; X₁₇ is Q or H; X₁₈ is Y or H, X₁₉ is L or Y; X₂₀ is P or I;
   X₂₁ is L or P, and X₂₂ is T or V.
18. An antibody, or antigen binding fragment thereof, wherein the antibody comprises:
   a variable heavy chain comprising a HCDR1 of SEQ ID NO: 82, a HCDR2 of SEQ ID NO: 83, and a HCDR3 of X₂₃GX₂₄X₂₅X₂₆X₂₇PX₂₈X₂₉X₃₀ (SEQ ID NO: 330), wherein: X23 is E or K, X24 is L or E; X25 is A or G, X26 is G or W, X27 is R, V, L, I, or F, X28 is F, Y, or T, X29 is D or Y, X30 is A, V, L, S, H, or I; and
   a variable light chain comprising a LCDR1 of SEQ ID NO: 85, a LCDR2 of SEQ ID NO: 86, and a LCDR3 of X₃₁QX₃₂X₃₃SX₃₄X₃₅X₃₆X₃₇ (SEQ ID NO: 331), wherein X31 is Q or V; X32 is Y, A, V, or H; X33 is N, P, S, or R; X34 is Y, L, or P; X35 is S, K, V, A, D, or R; X36 is W or L; and X37 is T or L.
19. The antibody, or antigen binding fragment thereof, of any one of the preceding embodiments, wherein the antibody, or antigen binding fragment thereof, comprises a constant region as provided for herein.
20. The antibody, or antigen binding fragment thereof, of any one of the preceding embodiments, wherein the heavy chain variable region and the light chain variable region are not linked by a linker.
21. The antibody, or antigen binding fragment thereof, of any one of the preceding embodiments, wherein the heavy chain variable region and the light chain variable region are linked with a peptide linker.
22. The antibody, or antigen binding fragment thereof, of embodiment 21, wherein the peptide linker comprises a sequence of (GGGGS)ₙ (SEQ ID NO: 266); (GGGGA)ₙ (SEQ ID NO: 267), or any combination thereof, wherein each n is independently 1-5.
23. A variant of the antibody, or antigen binding fragment thereof, of any one of the preceding embodiments, wherein the variant has 1-10 substitutions, deletions, or insertions.
24. A variant of the antibody, or antigen binding fragment thereof, of any one of the preceding embodiments, wherein the variant has 1-10 conservative substitutions.
25. A recombinant antibody or an antigen binding fragment thereof that binds to a C1s, such as specifically binding to active C1s, wherein the antibody, or antigen binding fragment thereof, comprises an amino acid sequence, or a variant thereof, as provided for herein.
26. An antibody that specifically binds to active C1s, wherein the antibody has a K_{D} of less than 7×10⁻⁹ M, 6×10⁻⁹ M, 5×10⁻⁹ M, 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M.
27. The antibody of embodiment 26, wherein the K_{D} is measured as set forth in Example 3.
28. The antibody of embodiments 26 or 27, wherein the antibody is an antibody of any one of embodiments 1-25.
29. An antibody that specifically binds to active C1s and inhibits the Wieslab complement classical pathway with an IC₅₀ of about 1 to 5 nM, about 0.1 to 1 nM, about 0.5 to about 3 nM, about 1 to about 2 nM, less than 5 nM, less than 4.5 nM, less than 3 nM, or less than 1 nM.
30. The antibody of embodiment 29, wherein the IC₅₀ is determined as set forth in Example 4.
31. The antibody of embodiments 29 or 30, wherein the antibody is an antibody of any one of embodiments 1-25.
32. An antibody that specifically binds to active C1s and inhibits human serum complement-mediated human RBC lysis with an IC₅₀ of less than 20 nM, less than 19 nM, less than 18 nM, less than 15 nM, less than 13 nM, less than 12 nM, less than 12.5 nM, about 11 to about 20 nM, about 11 to about 15 nM, about 11 to about 13 nM, about 11 to about 12 nM, about 12 to about 15 nM.
33. The antibody of embodiment 32, wherein the IC₅₀ is determined as set forth in Example 5.
34. The antibody of embodiments 32 or 33, wherein the antibody is an antibody of any one of embodiments 1-25.
35. An isolated nucleic acid molecule encoding an antibody, or an antigen binding fragment thereof, a heavy chain variable region, a light chain variable region, heavy chain, or light chain, of any of the preceding embodiments.
36. The isolated nucleic acid sequence of embodiment 35 comprising a nucleic acid molecule as set forth herein, such as a sequence of SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, or SEQ ID NO: 453.
37. An expression vector comprising the nucleic acid molecule of embodiments 35 or 36.
38. A host cell comprising the nucleic acid molecule of embodiments 35 or 36 or the vector of embodiment 37.
39. An antibody or antigen-binding fragment produced by the host cell of embodiment 38.
40. A pharmaceutical composition comprising an antibody, or antigen binding fragment thereof, of any one of embodiments 1-34.
41. The pharmaceutical composition of embodiment 40, wherein the composition is formulated for intravenous or subcutaneous injection.
42. The pharmaceutical composition of embodiment 40, wherein the composition is an injectable pharmaceutical composition.
43. A method of producing a polypeptide comprising a heavy chain variable region or light chain variable region, the method comprising:
   (a) growing the host cell of embodiment 41 under conditions so that the host cell expresses the polypeptide comprising the heavy chain variable region or the light chain variable region; and
   (b) purifying the polypeptide comprising the heavy chain variable region or the light chain variable region.
44. A method of producing an antibody that binds human C1s, or an antigen binding fragment of the antibody, the method comprising:
   (a) growing the host cell of embodiment 41 under conditions so that the host cell expresses a polypeptide or polypeptides comprising the immunoglobulin heavy chain variable region and/or the immunoglobulin light chain variable region, thereby producing the antibody or the antigen-binding fragment of the antibody; and
   (b) purifying the antibody or the antigen-binding fragment of the antibody.
45. A method of treating a subject with C1s mediated disorder, the method comprising administering to the subject an antibody, or antigen binding fragment thereof, of any one of embodiments 1-34 or 39 or a pharmaceutical composition of any one of embodiments 40-42.
46. The method of embodiment 45, wherein the disorder is hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy.
47. The method of embodiment 45, wherein the C1s mediated disorder is Cold Agglutinin Disease or Myasthenia Gravis.
48. A method of inhibiting membrane attack complex (MAC) formation or deposition in a subject in need thereof (e.g. such as a patient with Myasthenia Gravis), the method comprising administering to the subject an antibody, or antigen binding fragment thereof, of any one of embodiments 1-34 or 39 or a pharmaceutical composition of any one of embodiments 40-42.
49. A method of inhibiting or preventing neurotransmission impairment in a subject in need thereof, (e.g. such as a patient with Myasthenia Gravis), the method comprising administering to the subject an antibody, or antigen binding fragment thereof, of any one of embodiments 1-34 or 39 or a pharmaceutical composition of any one of embodiments 40-42.
50. A method of enhancing, allowing, or permitting muscle contraction in a subject in need thereof, (e.g. such as a patient with Myasthenia Gravis), the method comprising administering to the subject an antibody, or antigen binding fragment thereof, of any one of embodiments 1-34 or 39 or a pharmaceutical composition of any one of embodiments 40-42.
51. A method of ameliorating or alleviating muscle paralysis in a subject in need thereof, (e.g. such as a patient with Myasthenia Gravis), the method comprising administering to the subject an antibody, or antigen binding fragment thereof, of any one of embodiments 1-34 or 39 or a pharmaceutical composition of any one of embodiments 40-42.
52. A method of inhibiting the loss of muscle tone or muscle mass in a subject in need thereof, (e.g. such as a patient with Myasthenia Gravis), the method comprising administering to the subject an antibody, or antigen binding fragment thereof, of any one of embodiments 1-34 or 39 or a pharmaceutical composition of any one of embodiments 40-42.
53. A method of reducing the muscle fatigue index in a subject in need thereof, (e.g. such as a patient with Myasthenia Gravis), the method comprising administering to the subject an antibody, or antigen binding fragment thereof, of any one of embodiments 1-34 or 39 or a pharmaceutical composition of any one of embodiments 40-42.
54. The method of any one of embodiments 45-53, wherein the antibody or pharmaceutical composition is administered intravenously.
55. The method of any one of embodiments 45-53, wherein the antibody or pharmaceutical composition is administered subcutaneously.
56. An antibody, or antigen binding fragment thereof, of any one of embodiments 1-34 or 39, or a pharmaceutical composition comprising the antibody or antigen binding fragment thereof, for the use in the treatment of a C1s mediated disorder, such as those provided for herein, such as hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy.
57. An antibody, or antigen binding fragment thereof, of any one of embodiments 1-34 or 39, or a pharmaceutical composition comprising the antibody or antigen binding fragment thereof, for the use in the treatment of a C1S mediated disorder, such as Cold Agglutinin Disease or Myasthenia Gravis.
58. An antibody, or antigen binding fragment thereof, of any one of embodiments 1-34 or 39 or a pharmaceutical composition comprising the antibody, or antigen binding fragment thereof, for use as a medicament.
59. The antibody or binding fragment thereof, or pharmaceutical composition of embodiment 58 for use in the treatment of a C1s mediated disorder, such as those provided for herein.
60. Use of the antibody, or antigen binding fragment thereof, of any one of embodiments 1-34 or 39, or a pharmaceutical composition comprising the antibody or antigen binding fragment thereof, for the treatment of a C1s mediated disorder, such as those provided for herein.

In some embodiments, the following embodiments are provided.
1. An antibody, or an antigen binding fragment thereof, wherein the antibody or antibody fragment comprises:
   a heavy chain variable region comprising heavy chain HCDR1, HCDR2, and HCDR3 sequences, wherein the HCDR1 has the amino acid sequence of SEQ ID NO: 61, the HCDR2 has the amino acid sequence of SEQ ID NO: 62, the HCDR3 has the amino acid sequence of SEQ ID NO: 78; and
   a light chain variable region comprising light chain LCDR1, LCDR3, and LCDR3 sequences, wherein the LCDR1 has the amino acid sequence of SEQ ID NO: 64, the LCDR2 has the amino acid sequence of SEQ ID NO: 65, the LCDR3 has the amino acid sequence of SEQ ID NO: 79.
2. The antibody of embodiment 1, or an antigen binding fragment thereof, wherein the heavy chain variable region comprises an amino acid sequence at least 90% identical to the amino acid sequence of SEQ ID NO: 342.
3. The antibody of embodiment 1, or an antigen binding fragment thereof, wherein the heavy chain variable region comprises an amino acid sequence at least 95% identical to the amino acid sequence of SEQ ID NO: 342.
4. The antibody of embodiment 1, or an antigen binding fragment thereof, wherein the heavy chain variable region comprises the amino acid sequence of SEQ ID NO: 342.
5. The antibody of embodiment 1, or an antigen binding fragment thereof, wherein the heavy chain variable region comprises an amino acid sequence at least 90% identical to the amino acid sequence of SEQ ID NO: 17.
6. The antibody of embodiment 1, or an antigen binding fragment thereof, wherein the heavy chain variable region comprises an amino acid sequence at least 95% identical to the amino acid sequence of SEQ ID NO: 17.
7. The antibody of embodiment 1, or an antigen binding fragment thereof, wherein the heavy chain variable region comprises the amino acid sequence of SEQ ID NO: 17.
8. The antibody of embodiment 1, or an antigen binding fragment thereof, wherein the heavy chain variable region comprises an amino acid sequence at least 90% identical to the amino acid sequence of SEQ ID NO: 454.
9. The antibody of embodiment 1, or an antigen binding fragment thereof, wherein the heavy chain variable region comprises an amino acid sequence at least 95% identical to the amino acid sequence of SEQ ID NO: 454.
10. The antibody of embodiment 1, or an antigen binding fragment thereof, wherein the heavy chain variable region comprises the amino acid sequence of SEQ ID NO: 454.
11. The antibody of embodiment 1, or an antigen binding fragment thereof, wherein the antibody comprises a heavy chain that is at least 90% identical to the amino acid sequence of SEQ ID NO: 381.
12. The antibody of embodiment 1, or an antigen binding fragment thereof, wherein the antibody comprises a heavy chain that is at least 95% identical to the amino acid sequence of SEQ ID NO: 381.
13. The antibody of embodiment 1, or an antigen binding fragment thereof, wherein the antibody comprises a heavy chain comprises the amino acid sequence of SEQ ID NO: 381.
14. The antibody of embodiment 1, or an antigen binding fragment thereof, wherein the antibody comprises a heavy chain that is at least 90% identical to the amino acid sequence of SEQ ID NO: 185.
15. The antibody of embodiment 1, or an antigen binding fragment thereof, wherein the antibody comprises a heavy chain that is at least 95% identical to the amino acid sequence of SEQ ID NO: 185.
16. The antibody of embodiment 1, or an antigen binding fragment thereof, wherein the antibody comprises a heavy chain comprises the amino acid sequence of SEQ ID NO: 185.
17. The antibody of any one of embodiments 1-16, or an antigen binding fragment thereof, wherein the light chain variable region comprises an amino acid sequence that is at least 90% identical to the amino acid sequence of SEQ ID NO: 18.
18. The antibody of any one of embodiments 1-16, or an antigen binding fragment thereof, wherein the light chain variable region comprises an amino acid sequence that is at least 95% identical to the amino acid sequence of SEQ ID NO: 18.
19. The antibody of any one of embodiments 1-16, or an antigen binding fragment thereof, wherein the light chain variable region comprises the amino acid sequence of SEQ ID NO: 18.
20. The antibody of any one of embodiments 1-16, or an antigen binding fragment thereof, wherein the antibody comprises a light chain that is at least 90% identical to the amino acid sequence of SEQ ID NO: 236.
21. The antibody of any one of embodiments 1-16, or an antigen binding fragment thereof, wherein the antibody comprises a light chain that is at least 95% identical to the amino acid sequence of SEQ ID NO: 236.
22. The antibody of any one of embodiments 1-16, or an antigen binding fragment thereof, wherein the antibody comprises a heavy chain comprises the amino acid sequence of SEQ ID NO: 236.
23. The antibody of embodiment 1, wherein the antibody, or an antigen binding fragment thereof, comprises a variable heavy chain region comprising the amino acid sequence of SEQ ID NO: 342 and a light chain variable region comprising the sequence of SEQ ID NO 18.
24. The antibody of embodiment 23, wherein the antibody, or an antigen binding fragment thereof, comprises a heavy chain comprising the sequence of SEQ ID NO: 381 and a light chain comprising the sequence of SEQ ID NO: 236.
25. The antibody of embodiment 1, wherein the antibody, or an antigen binding fragment thereof, comprises a variable heavy chain region comprising the amino acid sequence of SEQ ID NO: 17 and a light chain variable region comprising the sequence of SEQ ID NO 18.
26. The antibody of embodiment 23, wherein the antibody, or an antigen binding fragment thereof, comprises a heavy chain comprising the sequence of SEQ ID NO: 185 and a light chain comprising the sequence of SEQ ID NO: 236.
27. An isolated nucleic acid molecule encoding an antibody, or an antigen binding fragment thereof, a heavy chain variable region, a light chain variable region, heavy chain, or light chain, of any of the preceding embodiments.
28. An expression vector comprising the nucleic acid molecule of embodiment 27.
29. A host cell comprising the nucleic acid molecule of embodiments 27 or the vector of embodiment 28.
30. A pharmaceutical composition comprising an antibody, or antigen binding fragment thereof, of any one of embodiments 1-26 and a pharmaceutically acceptable excipient.
31. The pharmaceutical composition of embodiment 30, wherein the composition is formulated for intravenous or subcutaneous injection.
32. A method of producing a polypeptide comprising a heavy chain variable region or light chain variable region, the method comprising:
   (a) growing the host cell of embodiment 29 under conditions so that the host cell expresses the polypeptide comprising the heavy chain variable region or the light chain variable region; and
   (b) purifying the polypeptide comprising the heavy chain variable region and/or the light chain variable region.
33. A method of treating a subject with C1s mediated disorder, the method comprising administering to the subject an antibody, or antigen binding fragment thereof, of any one of embodiments 1-26, or a pharmaceutical composition comprising the same.
34. The method of embodiments 33, wherein the disorder is Myasthenia Gravis, hemolysis, Cold Agglutinin Disease, Immune Thrombocytopenia (ITP), Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy.
35. A method of treating a subject with or Myasthenia Gravis or Cold Agglutinin Disease, the method comprising administering to the subject an antibody, or antigen binding fragment thereof, of any one of embodiments 1-26, or a pharmaceutical composition comprising the same.
36. The method of embodiment 35, wherein the antibody, or an antigen binding fragment thereof, comprises a variable heavy chain region comprising the amino acid sequence of SEQ ID NO: 342 and a light chain variable region comprising the sequence of SEQ ID NO 18.
37. The method of embodiment 35, wherein the antibody, or an antigen binding fragment thereof, comprises a heavy chain comprising the sequence of SEQ ID NO: 381 and a light chain comprising the sequence of SEQ ID NO: 236.
38. A method of inhibiting membrane attack complex (MAC) formation or deposition in a subject in need thereof (e.g. such as a patient with Myasthenia Gravis), the method comprising administering to the subject an antibody, or antigen binding fragment thereof, of any one of embodiments 1-26, or a pharmaceutical composition comprising the same.
39. A method of inhibiting or preventing neurotransmission impairment in a subject in need thereof, (e.g. such as a patient with Myasthenia Gravis), the method comprising administering to the subject an antibody, or antigen binding fragment thereof, of any one of embodiments 1-26, or a pharmaceutical composition comprising the same.
40. A method of enhancing, allowing, or permitting muscle contraction in a subject in need thereof, (e.g. such as a patient with Myasthenia Gravis), the method comprising administering to the subject an antibody, or antigen binding fragment thereof, of any one of embodiments 1-26, or a pharmaceutical composition comprising the same.
41. A method of ameliorating or alleviating muscle paralysis in a subject in need thereof, (e.g. such as a patient with Myasthenia Gravis), the method comprising administering to the subject an antibody, or antigen binding fragment thereof, embodiments 1-26, or a pharmaceutical composition comprising the same.
42. A method of inhibiting the loss of muscle tone or muscle mass in a subject in need thereof, (e.g. such as a patient with Myasthenia Gravis), the method comprising administering to the subject an antibody, or antigen binding fragment thereof, of any one of embodiments 1-26, or a pharmaceutical composition comprising the same.
43. A method of reducing the muscle fatigue index in a subject in need thereof, (e.g. such as a patient with Myasthenia Gravis), the method comprising administering to the subject an antibody, or antigen binding fragment thereof, of any one of embodiments 1-26, or a pharmaceutical composition comprising the same.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### Examples

### Example 1: Expression and Purification of hC1s RQ

For all work with active human C1s, material purified from human serum (CompTech, Tyler, TX, catalog number A104) was used. It was observed that human ProC1s purified from serum is unstable and appears to undergo cleavage, presumably at Arg437, the site cleaved by C1r (Budayova-Spano, M et al. EMBO J 21:231-239, 2002, using the C1s numbering in GenBank sequence AAA51852.1) either because of trace amounts of C1r in the material or a slow rate of autocleavage. To stabilize ProC1s for use in binding experiments, recombinant human ProC1s was expressed in mammalian cells in which Arg437 was mutated to glutamine (hProC1s_RQ).

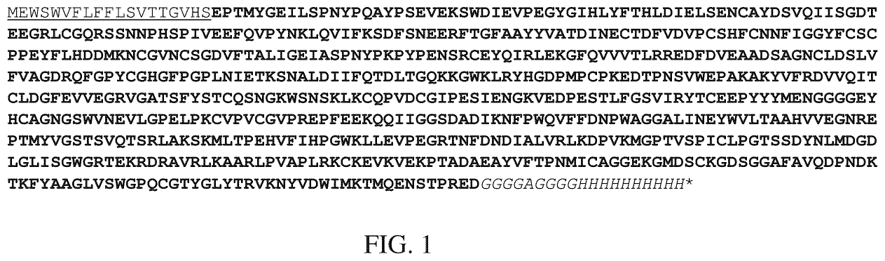

A gene sequence encoding the protein human ProC1s_RQ shown in FIG. 1 was cloned into a high expression mammalian plasmid vector Suspension Chinese hamster ovary (CHO) or human embryonic kidney (HEK293) cells were seeded in a shake flask and expanded using a serum-free and chemically defined medium. On the day of transfection, the expanded cells were seeded into a new vessel with fresh medium. After transfection with the plasmid expression vector for human ProC1s RQ, the cells were maintained as a batch-fed culture for 14 days. Next, the cells were removed from the media by centrifugation and the media containing human ProC1s_RQ was loaded onto an immobilized metal affinity column (Millipore Sigma, St. Louis, MO, catalog GE17-920-07) pre-equilibrated with binding buffer (20 mM sodium phosphate, 1M NaCl, pH 7.2). Washing buffer (20 mM sodium phosphate, 1M NaCl, 40 mM imidazole, pH 7.2) was passed through the column until the OD₂₈₀ value returned to baseline. Human ProC1s_RQ was eluted with a linear gradient of increasing imidazole concentration up to 0.5 M in 20 mM sodium phosphate pH 7.2. The eluate was collected in fractions, and the OD₂₈₀ value of each fraction was recorded. Denaturing capillary electrophoresis (CE-SDS, LabChip GXII, Perkin Elmer) of each fraction was performed and analyzed. Fractions containing the target protein were pooled and dialyzed into 30 mM HEPES, 150 mM NaCl, pH 7.0.

### Example 2: Human C1s and hProC1s_RQ ELISA

Human C1s and recombinant hC1s_RQ were biotinylated using the EZ link Sulfo NHS-LC-Biotin kit (ThermoFisher catalog #21327) as described by the vendor. 96-well plates (Maxisorp black 96-well immune plates (Thermofisher Scientific, Waltham, MA, Cat No 437111) were coated with 50 μl/well of a goat anti-human IgG (Jackson ImmunoResearch, West Grove, PA, catalog number 109-001-008) diluted to 2.5 μg/ml in phosphate buffered saline (PBS) and incubated at 4° C. overnight. Plates were washed with PBS and then blocked with 300 μl/well 3% (wt/vol) powdered milk in PBS (3% M-PBS) and incubated at room temperature for 1 hour with agitation and then washed with PBS. For each clone tested, 50 μl of the media from the expression cultures was added to an equal volume of 6% (wt/vol) powdered milk in PBS and added to a well in an anti-human IgG coated plate for 1 h at room temperature. The plate was then washed with PBS and then either 100 μl biotinylated-human C1s or biotinylated-hProC1s_RQ was added to each well and incubated for 1 h at room temperature. After washing the wells with PBS, 50 μl/well of Strep-Eu (Perkin-Elmer, Waltham, MA, Cat No 1244-360) diluted 500-fold in DELFIA® assay buffer (Perkin-Elmer, Waltham, MA, Cat. No 1244-111) was added to each well and incubated at room temperature for 1 h. After washing the wells 3 times with PBS, 50 μL of DELFIA enhancement solution (Perkin-Elmer, Waltham, MA, Cat. No 1244-105) was added to the wells. The plates were agitated gently for 3-5 minutes and read using a PiERAstar FS plate reader (BMG Labtech, Cary, NC), with excitation at 340 nm, emission at 615 nm with a delay time of 400 μsecs.

### Example 3: Optimization of Three Parental Anti-Human C1s Antibodies

Three antibody lineages, 4011, 191 and 5L3, that bound, as determined by surface plasmon resonance (SPR), to active human C1s with greater selectivity vs. human ProC1s RQ than recombinant sutimlimab/BIVV009/TNT009 (sequence contained in US patent application US2021/0115116A1), which binds with high affinity to both human C1s and human ProC1s, were identified (Table 11). Having low affinity for ProC1s reduces binding of these three antibodies to serum ProC1s versus BIVV009/TNT009 which should improve serum half-life. To further optimize the affinity for human C1s and selectivity versus human ProC1s, mutations were introduced into the CDRs of parental antibodies. Before optimization the amino acid sequences of the variable region heavy and light chains, outside of the CDR3 region, were changed to the sequences of the most closely related germline sequences when those changes did not significantly impact potency. Additionally, sequences that could potentially make the antibodies structurally unstable were also changed. For antibody 4011, four variable region heavy chain amino acids were changed (V32L, D55E, I55T and D82E) and three variable region light chain amino acids were changed (E13A, T31S and I62F). The resulting antibody is called 4011g. For antibody 191, four heavy chain amino acids were changed (V40A, K77N, D100E and G101T) and four light chain amino acids were changed (N45K, G65S, D81E and S80P). The resulting antibody is called 191g. For antibody 5L3 one heavy chain amino acid was changed (N54E) and one light chain amino acid was changed (E45K). The resulting antibody is called 5L3g.

TABLE 11
Affinity of 4O11, 191, 5L3 and TNT009 for human
C1s and human ProC1s RQ (as determined by SPR).

| | Human C1s | Human ProC1s |
|---|---|---|
| Antibody | K_{D} (nM) | RQ K_{D} (nM) |
| TNT009 (sutimlimab) | 0.25 | 0.4 |
| 4O11 | 0.3 | 3900 |
| 191 | 1.2 | >5000 |
| 5L3 | 3.7 | >5000 |

For each of the three antibodies, 4011g, 191g and 5L3g, heavy and light chain CDR3 sequences were diversified by mutating two residues at a time to all 17 alternate amino acids (excluding cysteine, methionine and the original amino acid) using synthetic primers. The resulting libraries were then enriched for C1s binding and counterselection against ProC1s RQ binding. The enriched sequences were then screened for: 1) high IgG expression level (for good developability); 2) high binding to labelled C1s at low concentrations (for high affinity and potency); and 3) low binding to labelled ProC1s_RQ at high concentrations (for high selectivity).

High scoring sequences from the screening were expressed at a small scale by conducting transfections into mammalian 293 cells in four 96-well plates for 5-7 days. An ELISA assay was used for initial ranking of affinity to C1s and selectivity against proC1s_RQ.

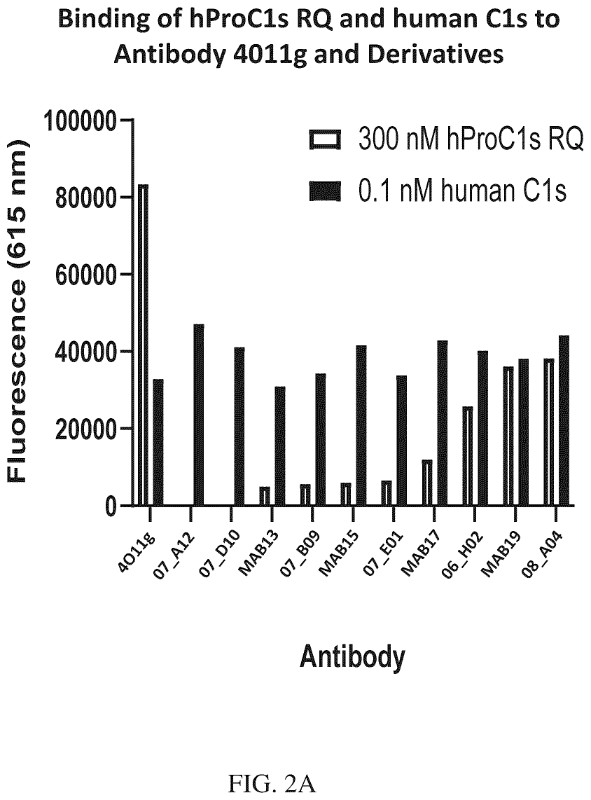

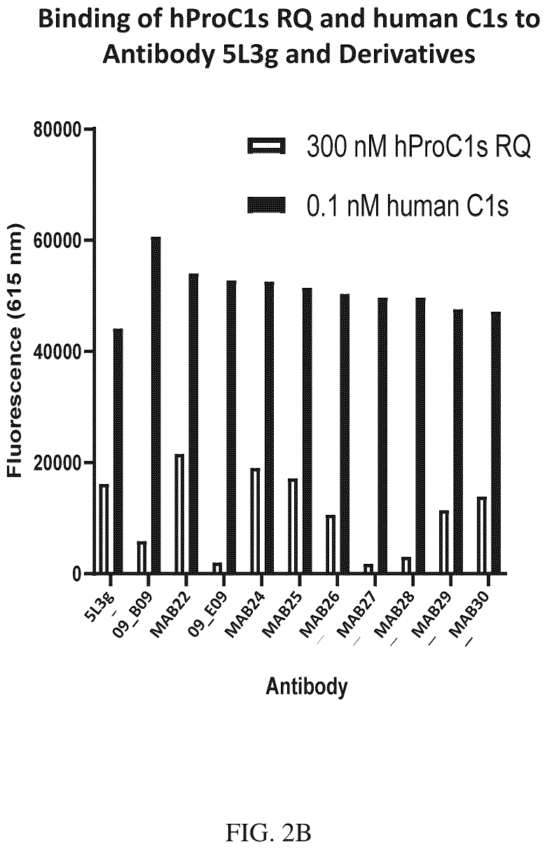

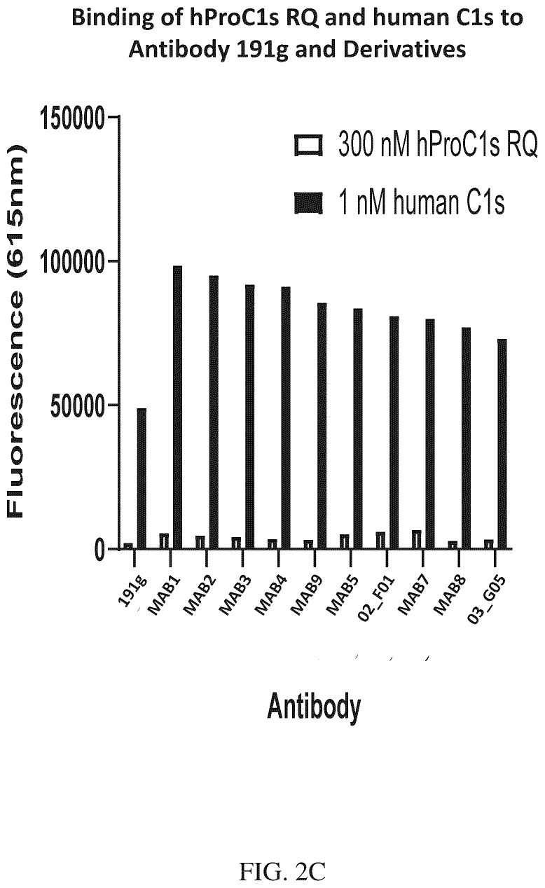

For the initial selection of clones, each was evaluated for binding to both biotinylated-human C1s (0.5 nM for 4011g and 5L3g clones; 1.0 nM for 191g clones) and hProC1s_RQ (500 nM) on separate plates. Clones with weak binding to hProC1s_RQ and high affinity binding to human C1s were rescreened by ELISA using biotinylated-human C1s (1 nM for 191g clones, 0.1 nM for 4011g and 5L3g clones) and 300 nM biotinylated-hProC1s_RQ and ten clones were chosen from each. See FIG. 2. For a subset of these 30 antibodies, we evaluated by surface plasmon resonance (SPR) the affinity to human C1s (Table 12). We also evaluated by SPR the binding to human ProC1s_RQ and found that for those marked with an asterisk (*) in Table 12 there was no detectable binding above background to 500 nM human ProC1s_RQ.

SPR methods: The binding assays were performed according to the following methods, which are described briefly.

### Antibody Immobilization

Affinity-purified goat anti-human Fc antibody (MP Biomedical, Irvine, CA, catalog #55071) was immobilized onto a high-capacity amine (HCA) sensor surface (Bruker, Billerica, MA catalog #1862614) at 1,000 RU-10,000 RU. HBS-EP+running buffer (10 mM HEPES, 150 mM NaCl, 0.05% v/v Tween-20, pH 7.4) was equilibrated with the system by performing a 3× Prime, then the baseline stability was observed over 30 min.

### Kinetics

Each antibody analyzed was diluted to 10 nM in HBS-EP+running buffer and captured by injecting at 10 μl/min for 3 minutes on a Sierra Sensors MASS-2 instrument, (Bruker, Billerica, MA).

Buffer was injected for 2 minutes at 25 μl/min. Varying concentrations of C1s or ProC1s RQ were injected at 35 μl/min for 2 minutes.

The surface was regenerated by injecting Regeneration solution (10 mM glycine-HCl, pH 1.7) at 35 μl/min for 30 seconds.

This was repeated for all concentrations of analyte tested and the kinetic parameters (on rate (Kₐ), off rate (K_{d}) and affinity K_{D} were calculated using instrument software. Table 12. SPR binding of selected clones and parental antibodies from each lineage and recombinant TNT020 to human C1s. Values for the association rate (Kₐ), dissociation rate (kd) and affinity (K_{D}) are shown. Antibodies with an asterisk (*) showed no detectable binding above background by SPR to human ProC1s_RQ. moles/liter (M)

TABLE 12

| Antibody | Kₐ (M⁻¹sec⁻¹) | Kₐ (sec⁻¹) | K_{D} (M) |
|---|---|---|---|
| 5L3g | 8.70 × 10⁴ | 4.85 × 10⁻⁴ | 5.58 × 10⁻⁹ |
| MAB22 | 8.04 × 10⁴ | 3.35 × 10⁻⁴ | 4.17 × 10⁻⁹ |
| MAB29 | 8.41 × 10⁴ | 5.90 × 10⁻⁴ | 7.02 × 10⁻⁹ |
| MAB26 | 8.45 × 10⁴ | 5.50 × 10⁻⁴ | 6.52 × 10⁻⁹ |
| MAB24 | 9.39 × 10⁴ | 3.28 × 10⁻⁴ | 3.49 × 10⁻⁹ |
| MAB25 | 1.11 × 10⁵ | 6.21 × 10⁻⁴ | 5.57 × 10⁻⁹ |
| MAB30 | 1.15 × 10⁵ | 6.88 × 10⁻⁴ | 6.01 × 10⁻⁹ |
| MAB28 | 1.41 × 10⁵ | 4.09 × 10⁻⁴ | 2.89 × 10⁻⁹ |
| MAB27 | 1.92 × 10⁵ | 2.64 × 10⁻⁴ | 1.38 × 10⁻⁹ |
| SCT191g* | 3.57 × 10⁵ | 1.345 × 10⁻³ | 3.77 × 10⁻⁹ |
| MAB9* | 4.14 × 10⁵ | 1.27 × 10⁻⁴ | 3.06 × 10⁻¹⁰ |
| MAB3* | 2.24 × 10⁵ | 2.09 × 10⁻⁴ | 9.32 × 10⁻¹⁰ |
| MAB1* | 2.68 × 10⁵ | 2.55 × 10⁻⁴ | 9.50 × 10⁻¹⁰ |
| MAB7* | 1.84 × 10⁵ | 2.23 × 10⁻⁴ | 1.22 × 10⁻⁹ |
| MAB4* | 1.90 × 10⁵ | 2.32 × 10⁻⁴ | 1.22 × 10⁻⁹ |
| MAB5* | 1.89 × 10⁵ | 2.64 × 10⁻⁴ | 1.40 × 10⁻⁹ |
| MAB2* | 1.18 × 10⁵ | 2.15 × 10⁻⁴ | 1.83 × 10⁻⁹ |
| MAB8* | 1.62 × 10⁵ | 3.45 × 10⁻⁴ | 2.13 × 10⁻⁹ |
| 4O11g | 1.52 × 10⁵ | 7.78 × 10⁻⁴ | 5.12 × 10⁻⁹ |
| MAB17* | 1.98 × 10⁵ | 4.14 × 10⁻⁴ | 2.09 × 10⁻⁹ |
| MAB13* | 2.04 × 10⁵ | 1.22 × 10⁻³ | 6.00 × 10⁻⁹ |
| MAB15* | 1.15 × 10⁵ | 7.37 × 10⁻⁴ | 6.39 × 10⁻⁹ |
| MAB19 | 1.06 × 10⁵ | 6.57 × 10⁻⁴ | 6.18 × 10⁻⁹ |
| TNT020* | 3.57 × 10⁵ | 1.34 × 10⁻³ | 3.77 × 10⁻⁹ |

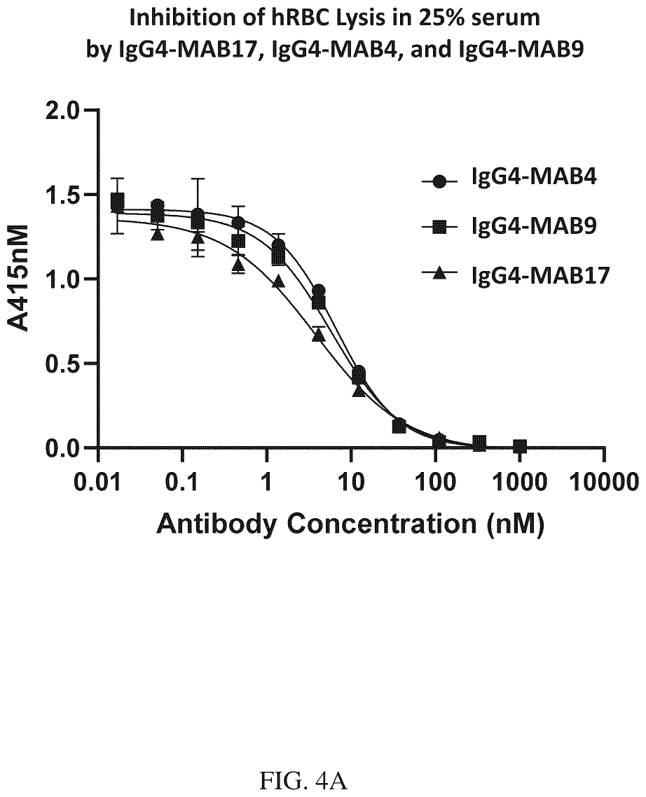

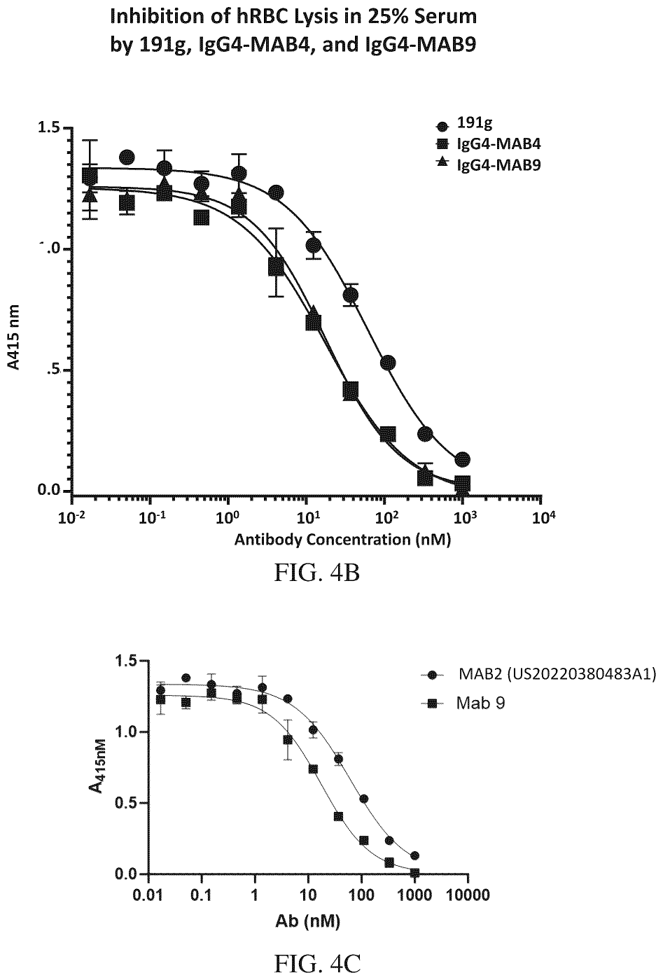

Of the 30 antibodies obtained from the screening process, we chose three for further characterization. MAB17 was chosen because it had reduced binding to human ProC1s_RQ, the precursor to active human C1s, as compared to the parental antibody 4O11g (FIG. 2A) and, therefore, might have a longer serum half-life due to reduced binding to the high level of ProC1s in serum (600 nM). Antibody MAB17 also had higher affinity to the target protein human C1s than its parental 4011g and recombinant TNT020, an antibody with selectivity for human C1s vs. human ProC1s (Patent application, US2020/0048332 A1) (Table 12). MAB4 and MAB9 were chosen because they have higher affinity to human C1s than their parental antibody 191g and recombinant TNT020 (FIG. 2C and Table 12). The higher affinity for human C1s also resulted in improved potency in inhibiting lysis of sensitized human red blood cells in 25% serum versus 191g (FIG. 4B and Table 14A).

The antibody as referred herein as MAB9 was also compared to a different antibody that binds to the active form of C1s. This comparator antibody is referred to as MAB2 in WO2022246154A2 and US20220380483A1, each of which is hereby incorporated by reference in its entirety (hereinafter referred to as “MAB2 (US20220380483A1)”. In the RBC lysis assay the IC50 of MAB2 (US20220380483A1) was calculated to be 61.2 nM and the IC50 of MAB9 (described herein) was calculated to be 17.6 nM. (FIG. 4C) Although a direct comparison of MAB39 to MAB2 (US20220380483A1) was not performed in the same assay, the data provided herein demonstrate that MAB39 and MAB9 have equivalent potency. Accordingly, the MAB9 antibody has over a 3× improvement in potency as compared to MAB2 (US20220380483A1) based upon the IC50 in the RBC lysis assay. This would also apply to MAB39, which would be expected to have more than 3× improvement over MAB2 (US20220380483A1).

We chose to further characterize and express MAB4, MAB9 and MAB17, in a human IgG4 backbone with the following modifications to the heavy chain; YTE: M252Y, S254T, T256E; Hinge region: S228P; CH2 region: L235E. The YTE modification was included to increase FcRn binding and serum half-life (Dall'Acqua, W F et al., J Immunol, 169: 5171-5180 (2002). The S228P modification was included to prevent Fab-arm exchange (Silva, J P et al., J Biol Chem, 290: 5462-5469 (2015). The L235E modification reduces binding to various FcgRs (Alegre, M L et al., J Immunol, 148: 3461-3468 (1992). To denote that these three antibodies are in this IgG4 background, we named these three antibodies IgG4-MAB4, IgG4-MAB9 and IgG4-MAB17.

### Example 4: Inhibition of the Complement Classical Pathway

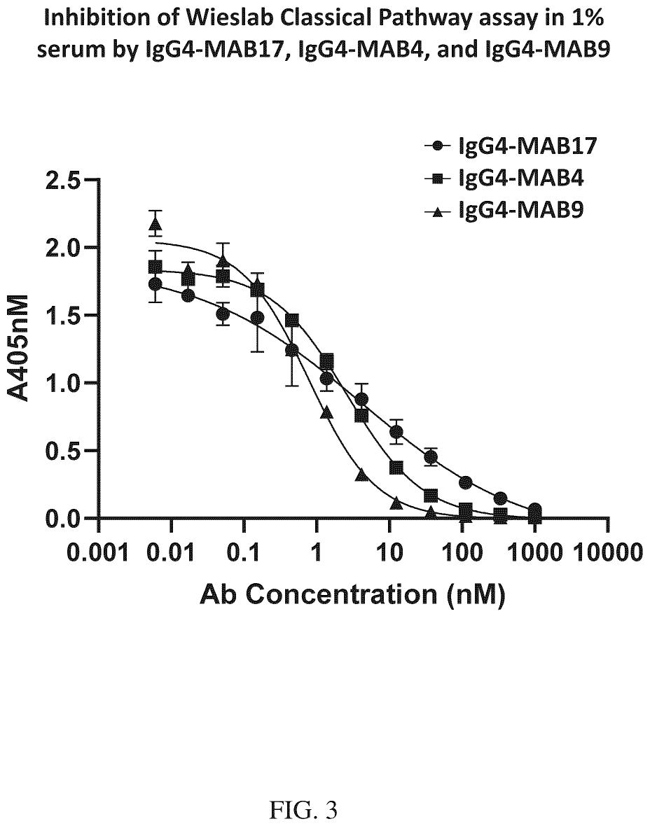

An adaptation of the Wieslab® assay produced by SVAR LifeSciences, Malmo, Sweden and purchased from Eagle Bioscience, Amherst, NH (catalog number COMPL CL310) was used to determine the IC₅₀ of the antibodies to block the complement classical pathway (Frederikson, G N et al., J. Imm Meth, 166: 263-270 (1993)). The Wieslab® assay used plate-bound IgM to activate the complement classical pathway when 1% human serum was added to the wells leading to deposition of components of the membrane attack complex (MAC) in the wells of the plate. After incubation at 37° C. for 1 hour, the wells were washed and the C5bC9 component of the MAC was detected by ELISA with an alkaline phosphatase labelled antibody specific to the neoantigen expressed during MAC formation as described by the makers of the kit. The assay was modified by preincubating aliquots of 1% human serum with varying amounts of inhibitory antibody for 15 minutes at 37° C. before initiating the assay so IC₅₀ could be determined for inhibition of C5bC9 formation. The C5bC9 ELISA signal (A405 nm) vs. inhibitory antibody concentration was plotted to determine an IC₅₀ value fitting the data to the equation Y=Bottom+(Top−Bottom)/(1+(IC₅₀/X)\hat{ }HillSlope) with GraphPad Prism software, version 9.2 (GraphPad Software, San Diego, CA). Results: The plots from the titration of IgG4-MAB4, IgG4-MAB9 and IgG4-MAB17 in the Wieslab Complement Classical Pathway Assay are shown in FIG. 3 and the IC₅₀ values are in Table 13.

TABLE 13
IC₅₀ values for inhibition of Wieslab complement classical
pathway by IgG4-MAB4, IgG4-MAB9 and IgG4-MAB17.

| Antibody | IC₅₀ (nM) | R² |
|---|---|---|
| IgG4-MAB4 | 2.6 | 0.995 |
| IgG4-MAB9 | 0.78 | 0.991 |
| IgG4-MAB17 | 4.1 | 0.967 |

All three antibodies, IgG4-MAB4, IgG4-MAB9 and IgG4-MAB17, completely inhibited the Wieslab complement classical pathway assay with IC₅₀ values of 0.78 to 4.1 nM. In addition to showing that IgG4-MAB4, IgG4-MAB9, IgG4-MAB17 are potent inhibitors of the complement classical pathway, we have also tested these antibodies for inhibition of the Wieslab complement alternative and lectin pathways using similar methods. A selective C1s inhibitory antibody would not be expected to inhibit either the complement alternative or lectin pathways as C1 complex activation is specific for the complement classical pathway. IgG4-MAB4, IgG4-MAB9, IgG4-MAB17 were tested at concentrations up to 1 micromolar and no dose-dependent inhibition of either the complement alternative or lectin pathways using the Wieslab complement alternative pathway and lectin pathway assays was observed (data not shown).

In three separate Wieslab complement classical pathway assays we also compared IgG4-MAB9 to IgG-MAB39. The variable heavy chain region of MAB9 AND MAB39 have the same CDRs, but differ only in the N-terminal amino acid of the variable heavy chain region. IgG4-MAB9 has a glutamine (Q) at this position while IgG-MAB39 has a glutamate (E). IgG4-MAB9 had an IC₅₀+standard deviation of 0.69+0.28 nM, while IgG4-MAB39 had an IC₅₀+standard deviation of 0.49+0.11 nM. The change of the glutamine at the N-terminus of the VH in MAB9 to the glutamate at the N-terminus of the VH in MAB39 was done for manufacturing purposes. Without wishing to be bound to any particular theory, glutamine at the N-terminus may lead to spontaneous cyclization of the residue to pyroglutamate (pE) in vitro. This process is slower with a glutamate as compared to glutamine. Therefore, the manufacturing of MAB39 is advantageous as compared to MAB9. The antibody referenced in this example and the examples below as MAB9 comprises a VL of SEQ ID NO: 18, a light chain of SEQ ID NO: 236, a VH of SEQ ID NO: 17 and a heavy chain of SEQ ID NO 185, which has the “YTE” mutations in the constant domain. The antibody referenced in this example and the examples below as MAB39 comprises a VL of SEQ ID NO: 18, a light chain of SEQ ID NO: 236, a VH of SEQ ID NO: 342 and a heavy chain of SEQ ID NO 381, which has the “YTE” mutations in the constant domain.

### Example 5: Inhibition of Human Serum Complement-Mediated Human RBC Lysis

To evaluate inhibition of MAC formation stimulated by the complement classical pathway at a higher, more physiologically relevant, serum concentration than used in the Wieslab® assay, the ability of the antibodies to inhibit lysis of sensitized human RBCs (hRBCs) was tested in an assay using 25% human serum. Methods: hRBCs were sensitized with a rabbit IgG to hRBCs (LS Bio, Seattle, WA, catalog #LS-C63000-2). Serial dilutions of the anti-C1s antibodies were preincubated with aliquots of human serum diluted in 0.1% gelatin, 5 mM veronal, 145 mM NaCl, 0.15 mM CaCl₂, 0.5 mM MgCl₂, 0.025% NaN₃, pH 7.3 (GVB++), added to sensitized hRBCs in GVB++ at a final human serum concentration of 25% and incubated at 37° C. in microtiter plates. After 1 hour, EDTA was added to 20 mM, the plates centrifuged at 500×g for 5 minutes at 4° C. and the supernatants assayed for absorbance at 415 nm. The data were plotted and used to calculate an IC₅₀ value fitting the data to the equation Y=Bottom+(Top−Bottom)/(1+(IC₅₀/X)\hat{ }HillSlope) with GraphPad Prism software, version 9.2 (GraphPad Software, San Diego, CA). hRBCs were obtained from three different donors and each were tested independently. FIG. 4A shows the plotted data from Donor 3 and Table 14 the IC₅₀ values for experiments in which inhibition of lysis of hRBCs from three different donors were tested in the presence of IgG4-MAB4, IgG4-MAB9, IgG4-MAB17 and recombinant TNT020. IgG4-MAB4, IgG4-MAB9 and IgG4-MAB17 showed complete inhibition of complement-mediated hRBC lysis in 25% human serum with IC₅₀ values of 3.8-22.1 nM from the three independent hRBC donors. In all cases, the IC₅₀ values for IgG4-MAB4, IgG4-MAB9 and IgG4-MAB17 were 1.39-3.84 times lower than the IC₅₀ values for recombinant TNT020.

TABLE 14
IC₅₀ values for inhibition of hRBC lysis in 25% human serum
by IgG4-MAB4, IgG4-MAB9, IgG4-MAB17 and recombinant
TNT020 of hRBCs from three independent donors.

| | Donor 1 | | Donor 2 | | Donor 3 | |
|---|---|---|---|---|---|---|
| | IC₅₀ | | IC₅₀ | | IC₅₀ | |
| Antibody | [nM] | R² | [nM] | R² | [nM] | R² |
| IgG4-MAB4 | 14.9 | 0.9953 | 17.1 | 0.9858 | 6.6 | 0.9909 |
| IgG4-MAB9 | 12.9 | 0.9966 | 22.1 | 0.9914 | 5.8 | 0.9919 |
| IgG4-MAB17 | 11.0 | 0.9938 | 20.5 | 0.9979 | 3.8 | 0.9868 |
| TNT020 | 20.9 | 0.9932 | 30.7 | 0.9839 | 14.6 | 0.9848 |

We also compared parental antibody 191g to two derivatives of 191g, MAB4 and MAB9, using the inhibition of hRBC lysis assay in 25% human serum as shown in FIG. 4B and Table 14A, which illustrates that MAB4 and MAB9 completely inhibited hRBC lysis in 25% serum and had lower IC₅₀ values than the parental antibody 191g.

TABLE 14A
IC₅₀ values for inhibition of hRBC lysis in 25% human
serum by 191g, IgG4-MAB4 and IgG4-MAB9.

| Antibody | IC₅₀ [nM] | R² |
|---|---|---|
| 191g | 61.2 | 0.989 |
| IgG4-MAB4 | 17.5 | 0.988 |
| IgG4-MAB9 | 17.6 | 0.985 |

### Example 6: Inhibition of Human Serum C3b Deposition on hRBCs

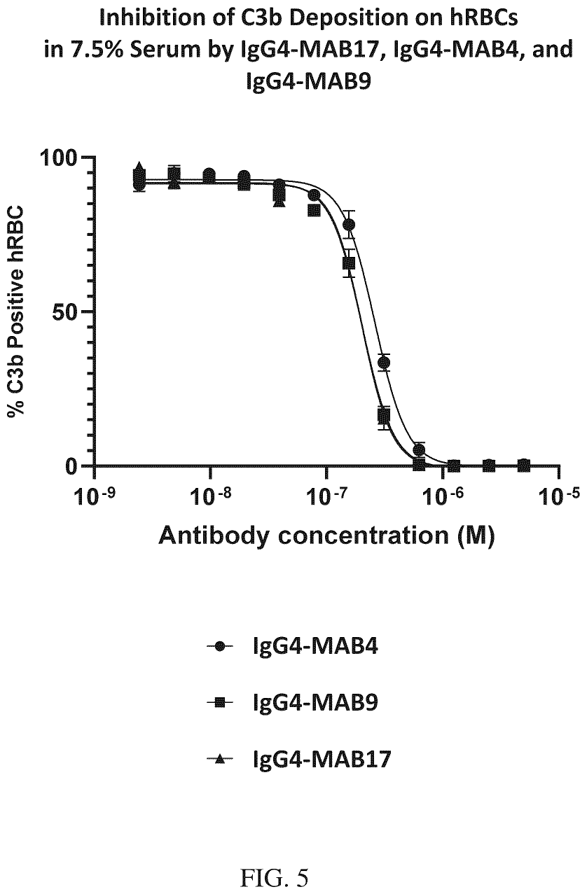

In addition to causing cell lysis, complement classical pathway activation also leads to deposition of C3b on sensitized cells which can lead to phagocytosis by macrophages (Brown, Proc Natl Acad Sci USA 99:16969, 2002). The ability of the antibodies to inhibit this endpoint of the complement classical pathway was evaluated using flow cytometry to examine C3b deposition from human serum on hRBCs sensitized with the same rabbit IgG to hRBCs used for hRBC lysis. Methods: hRBCs were sensitized with rabbit anti-hRBCs IgG (LS Bio, Seattle, WA, catalog #LS-C63000-2), using a higher dilution (less antibody) than in the hRBC lysis experiments to avoid too much lysis but still leading to C3b deposition. Human serum pre-incubated with serial dilutions of IgG4-MAB4, IgG4-MAB9, IgG4-MAB17 and recombinant TNT020 in GVB++ was added to the sensitized hRBCs at a final human serum concentration of 7.5%, incubated at 37° C. for 1 hour and then EDTA was added to 10 mM. The hRBCs were pelleted by centrifugation, washed with GVB without MgCl₂ or CaCl₂ and pelleted again. The resulting hRBCs were stained with a fluorescein isothiocyanate (FITC) conjugated antibody that binds to C3c/C3b (Abcam catalog number ab4212), counterstained with 7 amino-actinomycin D (7AAD) and sorted using an Intellicyte iQue3 to determine the percentage of hRBCs staining positive for C3b. The data were plotted and used to calculate an IC₅₀ value fitting the data to the equation Y=Bottom+(Top−Bottom)/(1+(IC50/X)\hat{ }HillSlope) with GraphPad Prism software version 9.2 (GraphPad Software, San Diego, CA). hRBCs from two different donors were tested in this assay. Donor A was tested once and Donor B was tested twice. FIG. 5 shows the plotted data for inhibition of C3b-deposition 7.5% human serum by IgG4-MAB4, IgG4-MAB9, IgG4-MAB17 and TNT020 from Donor B (2) and Table 15 the IC₅₀ values for all three experiments.

TABLE 15
C3b deposition IC₅₀ values for IgG4-MAB4, IgG4-MAB9,
IgG4-MAB17 and TNT020. RBCs from donor A were tested
once while RBCs from donor B were tested twice (1) and (2).

| | Donor A | Donor B (1) | Donor B (2) |
|---|---|---|---|
| Antibody | IC₅₀ (nM) [R2] | IC₅₀ (nM) [R2] | IC₅₀ (nM) [R2] |
| IgG4-MAB4 | 69.2 [0.994] | 193 [0.994] | 262 [0.998] |
| IgG4-MAB9 | 64.1 [0.983] | 124 [0.997] | 202 [0.997] |
| IgG4-MAB17 | 40.7 [0.996] | 112 [0.995] | 202 [0.995] |
| TNT020 | 52.9 [0.980] | 238 [0.988] | 688 [0.995] |

In summary, IgG4-MAB4, IgG4-MAB9 and IgG4-MAB17 showed complete inhibition of C3b deposition on sensitized hRBCs in 7.5% human serum with IC₅₀s of 40.7-262 nM.

### Example 7: Pharmacokinetics of IgG4-MAB4, IgG4-MAB9 and IgG4-MAB17 Dosed Intravenously (IV) in Cynomolgus Monkeys

We dosed IgG4-MAB4, IgG4-MAB9 and IgG4-MAB17 IV in cynomolgus monkeys to understand the pharmacokinetics of the antibodies in a primate species where the amino acid sequence of C1s is 94% identical to human C1s.

IgG4-MAB4, IgG4-MAB9 and IgG4-MAB17 were dosed intravenously in cynomolgus monkeys to understand the pharmacokinetics of the antibodies in a primate species. Two different doses of the antibodies were evaluated, 3 and 30 mpk. Methods: Male cynomolgus monkeys (Macaca fascicularus) weighing approximately 2-4 kg each were dosed IV in the cephalic vein with either 3 mg antibody/kg body weight (mpk) or 30 mpk of IgG4-MAB4, IgG4-MAB9 and IgG4-MAB17 in 20 mM Histidine, 8% (weight/volume) sucrose. The antibody solutions were prepared immediately before dosing and a volume of 4 ml/kg body weight was administered over 10 minutes to cohorts of three animals for each of the two doses. Blood was collected and placed in tubes without anticoagulant to obtain serum at the following time points: prior to dosing (pre-dose), and 2 hours (h), 8 h, 24 h, day 2, day 3, day 7, day 10, day 14, day 21, day 28, day 42, day 56 and day 70 post dosing. At the end of the study the serum concentration of the dosed antibodies was determined for each sample using an ELISA to detect total human IgG.

ELISA Materials: High Bind Microplate, Corning (Glendale, CA), Cat #: 9018 Tween-20: Sigma (Burlington, MA), Cat #: P7949-100 mL Proclin300, Sigma (Burlington, MA), Cat #: 48912-U Goat anti-Human Antibody, Southern Biotech (Birmingham, AL), Cat #: 2049-01 Human IgG-heavy and light chain monkey-adsorbed Antibody, Bethyl (Montgomery, TX), Cat #A80-319P TMB substrate solutions (A and B), InnoReagents, Cat #: TMB-S-003 10× phosphate buffered saline (PBS): 1370 mM NaCl, 100 mM Na₂HPO₄, 27 mM KCl, 20 mM KH₂PO₄ 1×PBS: 137 mM NaCl, 10 mM Na₂HPO₄, 2.7 mM KCl, 2 mM KH₂PO₄ (Dilute from 10×PBS using Milli Q water) Assay Diluent: 1×PBS, 1% BSA, 0.05% Tween-20, 0.05% proclin 300, Washing Buffer (PBST): 1×PBS with 0.05% Tween-20 Stop solution: 2N H₂SO₄ Male Cynomolgus monkey serum, ELISA plate reader: SpectraMax M2, Molecular Devices (San Jose, CA) SoftMax Pro GxP 5.4.4 software, Molecular Devices (San Jose, CA)

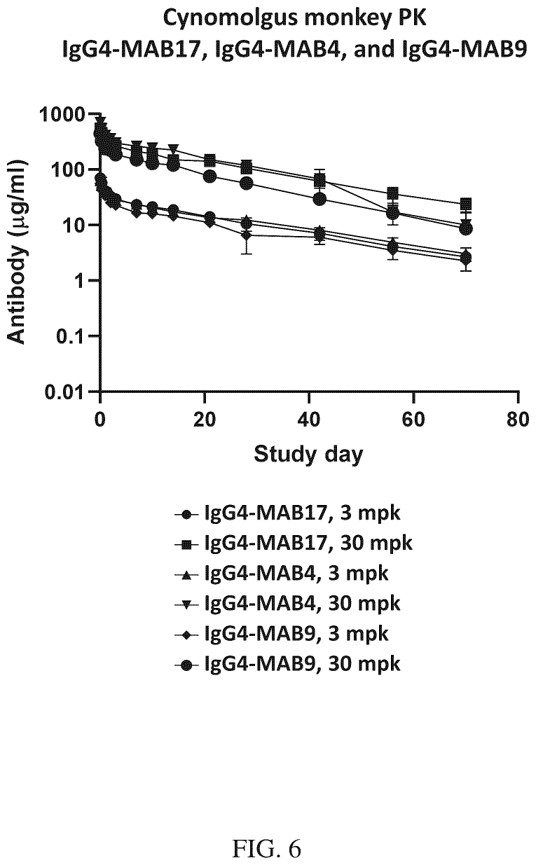

For assay plate coating, goat anti-human antibody was diluted in PBS. 50 μL/well was added into the ELISA plate. The plate was sealed and incubated overnight in 4° C. The plate was washed 3 times with 300 μL of washing buffer, then blocked with 200 μL/well blocking buffer. The plate was sealed and incubated at RT for 1-3 hr, constantly shaking at 300 rpm. The plate was then washed 3 times with 300 μL of washing buffer. For preparation of the anti-C1s antibody standard, a 2-fold serial dilution was made in 5% male monkey serum. Eight standards ranged from 50 to 0.391 ng/ml of IgG4-MAB4, IgG4-MAB9 and IgG4-MAB17. For QC preparation, two sets of QCs were prepared in 5% pooled monkey serum for each antibody: HQC (40 ng/mL), MQC (8 ng/mL) and LQC (2 ng/mL). For sample preparation: All serum samples were diluted at 20-fold in Assay Diluent first. Additional dilutions were made in 5% male monkey serum. For sample incubation, the diluted standards/QCs/samples were added at 50 μL/well into the plate. The plate was sealed and incubated at 37° C. for 1 hr. For the detection reagent reaction, the plate was first washed 3 times with 300 L of washing buffer. Human IgG-heavy and light chain monkey-adsorbed Antibody (A80-319P) was diluted in assay diluent. The diluted detection antibody was added into the assay plate at 100 μL/well. The assay plate was then incubated at 37° C. for 30 min. For substrate reaction and plate reading, the plate was first washed 3 times with 300 μL of washing buffer. TMB working solution was prepared by bringing the substrate to room temperature 30 min before use, mixing equal volume of substrate A and substrate B. 100 μL/well of premixed TMB substrate was added to each well and then incubated at room temperature for about 10 min. 100 μL/well of ELISA stopping solution was then added. The plate was then mixed and read at 450/630 nm wavelength using SpectraMax M2. Data was processed using Soft Max Pro GxP, and the standard curve was fit using 4-PL model. PK parameters were calculated using WinNonlin version 8.2 software, Certara (Princeton, NJ) Results: A plot of the mean IgG4-MAB4, IgG4-MAB9 and IgG4-MAB17 concentrations (n=3 animals/treatment group) for the cynomolgus monkeys dosed with 3 and 30 mg/kg body weight (mpk) are plotted in FIG. 6. The calculated pharmacokinetic parameters are listed in Table 16.

TABLE 16
Pharmacokinetic parameters of IgG4-MAB4, IgG4-MAB9 and IgG4-MAB17 when
dosed at 3 or 30 mpk in cynomolgus monkeys.

| | 3 mpk | 30 mpk | 3 mpk | 30 mpk | 3 mpk | 30 mpk |
|---|---|---|---|---|---|---|
| | IgG4- | IgG4- | IgG4- | IgG4- | IgG4- | IgG4- |
| Dose and Ab | MAB17 IV | MAB17 IV | MAB9 IV | MAB9 IV | MAB4 IV | MAB4 IV |
| Mean | 3.38 ± 0.58 | 3.78 ± 0.65 | 3.16 ± 0.18 | 3.43 ± 0.76 | 4.19 ± 0.33 | 6.22 ± 0.96 |
| clearance ± | | | | | | |
| SD | | | | | | |
| (ml/day/kg) | | | | | | |
| Mean | 89 ± 11 | 100 ± 7.2 | 92 ± 9.9 | 75.4 ± 4.8 | 124 ± 16 | 139 ± 14 |
| volume of | | | | | | |
| distribution | | | | | | |
| steady state ± | | | | | | |
| SD (ml/kg) | | | | | | |
| Mean serum | 19.8 ± 4.4 | 19.2 ± 2.7 | 20.7 ± 2.8 | 15.6 ± 3.2 | 21.7 ± 1.3 | 18.3 ± 8.2 |
| half life ± | | | | | | |
| SD (days) | | | | | | |
| Area under | 824 ± 98 | 7407 ± 1027 | 857 ± 20 | 7373 ± 2652 | 556 ± 195 | 4538 ± 542 |
| the curve | | | | | | |
| through last | | | | | | |
| day | | | | | | |
| (day*mg/ml) | | | | | | |

### Example 8: Inhibition of the Complement Classical Pathway is Sustained Over Time in Cynomolgus Monkeys Treated with IgG4-MAB39 Dosed Subcutaneously (SC)

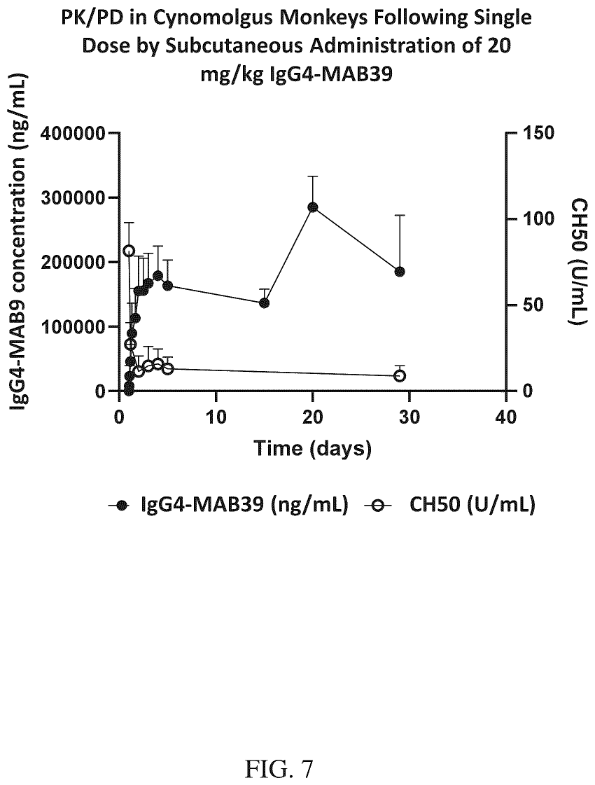

The ability of IgG4-MAB39 to sustain classical pathway inhibition over time was evaluated. Serum from cynomolgus monkeys was obtained for up to 30 days following the administration of a single subcutaneous dose of 20 mg/kg of IgG4-MAB39 (n=10 animals). The dilution factor of the serum necessary to obtain 50% lysis of sensitized sheep RBCs (CH50) was determined at multiple time points using the Complement CH50 Test Kit (Haemoscan. Groningen, The Netherlands) according to the manufacturer's instructions. The serum concentration of IgG4-MAB39 was quantified using an electrochemiluminescent immunoassay at the dilution factor of CH50 is depicted over time by the filled circles shown in FIG. 7. In summary, a single subcutaneous dose of IgG4-MAB39 at 20 mg/kg in cynomolgus monkeys led to a rapid and sustained inhibition of complement classical pathway.

### Example 9: Comparative Biophysical and Functional Studies of the Anti-Active C1s Antibody MAB39 and its Preclinical Precursor MAB9

Three comparative studies were performed to determine if there are differences between MAB39 and its precursor MAB9 in terms of biophysical and functional properties: Surface Plasmon Resonance analysis to assess binding affinity to human active C1s, Wieslab Classical Pathway assay to assess potency, and Classical pathway mediated cell lysis assay using antibody-sensitized sheep red blood cells to assess potency. Assays assessing potency used 1% human sera.

### Materials and Methods

Binding of MAB39 and MAB9 to Human Active C1s at 37° C./pH 7.4 Measured by Surface Plasmon Resonance. Human active C1s purified from serum was procured from Complement Technologies, Tyler, TX (catalog number A104). Studies to evaluate MAB39 and MAB9 binding to human active C1s at 37° C. were performed on a Biacore 4000 and Biacore T100 (Cytiva, Marlborough, MA), respectively. Goat anti-human antibody (ThermoFisher, Waltham, MA, USA, catalog number H10500) was amine-coupled to a C1 sensor chip (Cytiva, Marlborough, MA, USA, catalog number BR100535). MAB39 was diluted to 6.67 nM and MAB9 was diluted to 10 nM in 50 mM HEPES pH 7.4, 150 mM NaCl, 1.3 mM Ca, 0.02% Tween-20, 1 mg/mL BSA (HBS). MAB39 was captured for 60 seconds to produce surface densities of 200 RU and MAB9 was captured for 30 seconds to produce surface densities of 10 to 20 RU. Human active C1s was tested for binding using a 3-fold titration series in HBS with a maximum concentration of 12.3 nM for MAB39 and 16 nM for MAB9. Data were processed by subtracting responses from a reference surface as well as a buffer injection. The processed data were fitted with a 1:1 interaction model including baseline offset and bulk refractive index shifts in the association phase. Surfaces were regenerated with a 12 second injection of 1/100 dilution of H3PO4 for MAB39 and 12 second injection of 1/50 dilution of H3PO4 for MAB9. Replicate experiments were performed (n=2 for MAB9 and n=4 for MAB39).

Potency of MAB39 and MAB9 in the Wieslab Classical Pathway Assay. Inhibition of complement classical pathway by MAB39 and MAB9 was assessed by using the Wieslab Classical Pathway ELISA kit (SVAR LifeSciences, Malmo, Sweden, catalog number COMPLCP310), which measures the amount of C5b9 formed on IgM-coated plates upon addition of human serum. Serial dilutions of MAB39 and MAB9 were prepared using diluent buffer contained in the kit then combined with normal human serum (Complement Technologies, Tyler, TX, USA, catalog number NHS), with final antibody concentrations ranging from 0.01 to 600 nM with a 3-fold sequential serial dilution and final normal human serum concentration of 1%. Test samples, along with reconstituted positive and negative control human sera contained in the kit, diluent alone, and 0 nM antibody NHS control, were added to the plate. Then the plate was sealed with a plate sealer and incubated for 1 hour at 37° C. The plate was processed according to the manufacturer's protocol with reagents contained in the kit. Briefly, the plate was washed 3 times with wash buffer, followed by the addition of anti-C5b9 alkaline phosphatase conjugated detection antibody for 30 minutes at room temperature, washed 3 times with wash buffer, incubated with substrate solution for 30 minutes at ambient temperature, and finally the reaction was quenched with the addition of 5 mM EDTA stop solution. The plate was then read at absorbance 405 nm (SpectraMax 250, Molecular Devices, San Jose, CA, USA). For data analysis, the diluent alone value (blank control) was subtracted from all samples. The % complement classical pathway CCP activity was calculated using the following formula: % CCP activity=(Sample−Negative Control)/(Positive Control−Negative Control)×100. Next, the % CCP activity was normalized using the following formula: (% CCP activity of sample)/(mean of % CCP activity at lowest concentration of each data set)×100. The normalized % complement classical pathway activity was then plotted against inhibitor concentration and fitted to a standard 4-parameter dose-response inhibition using GraphPad (GraphPad Software, San Diego, CA, USA, version 9) to calculate the IC50 values. Three independent experiments were performed. Each experiment had 2 replicates. IgG4 isotype (recombinantly produced at Atum, Newark, CA, USA) served as a negative control antibody.

Potency of MAB39 and MAB9 in an Antibody-sensitized Sheep Red Blood Cell Lysis Assay. Inhibition of complement classical pathway by MAB39 and MAB9 was also determined with a hemolysis assay that utilizes antibody-sensitized sheep red blood cells. In this assay, complement classical pathway and MAC-mediated lysis are induced on antibody-coated sheep red blood cells upon exposure to human sera. Serial dilutions of MAB39 and MAB9 were prepared using GVB++ buffer containing calcium and magnesium (Complement Technologies, Tyler, Texas, USA, catalog number B102) then combined with normal human serum (Quidel, San Diego, CA, USA, catalog number 185487) and antibody-sensitized sheep red blood cells (5×107 cells per well, Complement Technologies, Tyler, Texas, USA, catalog number B202) in a tissue culture treated round bottom 96-well plate (Sarstedt, Numbrecht, Germany, catalog number 82.1582001). The final antibody concentrations ranged from 0.05 to 1000 nM with a 3-fold serial dilution and final human serum concentration was 1%. Controls included GVB++ buffer alone (blank), antibody-sensitized sheep red blood cells in GVB++ with 40% water (total lysis control), antibody-sensitized sheep red blood cells in GVB++ with 1% normal human serum final concentration (0 nM antibody NHS control), antibody-sensitized sheep red blood cells in GVB++, antibody-sensitized sheep red blood cells in GVB++ with 10 mM EDTA final concentration, and antibody-sensitized sheep red blood cells in GVB++ with 1% normal human serum final concentration and 10 mM EDTA final concentration. The plate was incubated for 1 hour at 37° C., centrifuged at 1000× gravity (g) for 3 minutes to pellet intact cells, and the supernatants were collected and transferred to a flat bottom 96-well plate (Corning, Corning, NY, USA, catalog number 3596). Hemoglobin release from lysed red blood cells in the supernatant was detected by measuring the optical density (OD) at 412 nm using a spectrophotometer (PHERAStar, BMG Lab Tech, Ortenberg, Germany). For data analysis, the GVB++ alone value (blank control) was subtracted from all samples. The % sheep red blood cell (sRBC) hemolysis was calculated using the following formula: % sRBC hemolysis=(ODtest)−(ODblank)/(ODtotal lysis)−(ODblank)×100. Next, the % sRBC hemolysis was normalized using the following formula: (% sRBC hemolysis of sample/mean of % sRBC hemolysis at lowest concentration of each data set)×100. The normalized % sRBC hemolysis was then plotted against inhibitor concentration and fitted to a standard 4-parameter dose-response inhibition using GraphPad (GraphPad Software, San Diego, CA, version 9) to calculate the IC50 values. Three independent experiments were performed. Each experiment had 2 replicates. IgG4 isotype (recombinantly produced at Atum, Newark, CA, USA) served as a negative control antibody.

### Results

Comparison of Binding Affinities of MAB39 and MAB9 to Human Active C1s by Surface Plasmon Resonance. The human active C1s association constant (ka), disassociation constant (kd) and affinity (KD) for MAB39 and MAB9 were determined in separate experiments (Table 17). The KD values±SD of MAB39 and MAB9 for human active C1s were 0.0105±0.0046 nM and 0.0071±0.00054 nM, respectively, and no statistical difference in binding affinity to human active C1s was observed between MAB39 and MAB9. Statistical analysis was performed using paired t-test, with p value of 0.6168.

TABLE 17
Binding Affinity of MAB39 and MAB9 to Human Active C1s

| | | Ka (M⁻¹sec⁻¹) ± | Kd (sec⁻¹) ± | KD (nM) ± |
|---|---|---|---|---|
| Antibody | Ligand | SD | SD | SD |
| MAB39 | Human | 1.19 ± 0.07 × 10⁷ | 1.25 ± | 0.0105 ± |
| | active C1s | | 0.62 × 10⁻⁴ | 0.0046 |
| MAB9 | Human | 1.69 ± 0.01 × 10⁷ | 1.20 ± | 0.0071 ± |
| | active C1s | | 0.08 × 10⁻⁴ | 0.0005 |

These very high affinities represent a significant improvement as compared to a parental antibody, referred to as MAB2 in WO2022246154A2. In a direct comparison between MAB2 (US20220380483A1) and MAB9 in a Surface Plasmon Resonance (SPR) assay and MAB9 was found to have an affinity that is about 10× higher than MAB2 (US20220380483A1). Thus, based on the KD reported above for MAB39 and MAB9, MAB39 would also be expected to have a significant improvement in affinity as compared to MAB2.

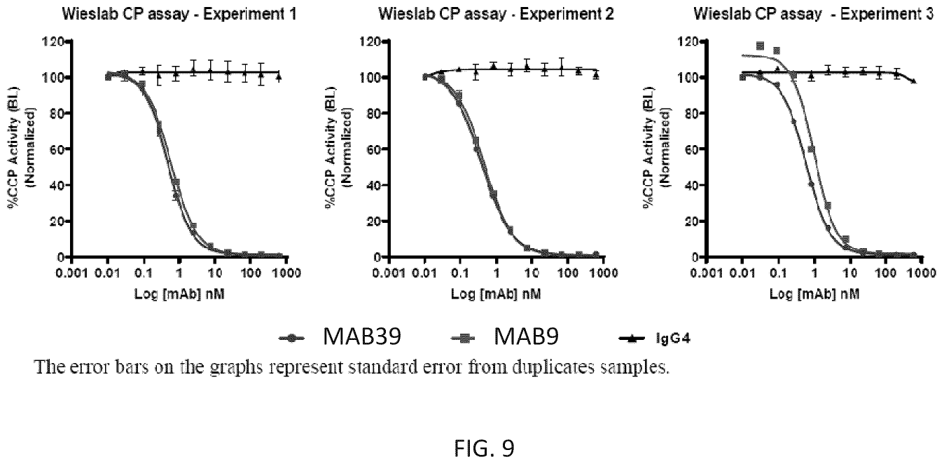

Comparison of Potency Between MAB39 and MAB9 in the Wieslab Classical Pathway Assay. Complement classical pathway activity versus antibody concentrations were plotted and the IC50 values were calculated. FIG. 9 shows the results from all 3 independent experiments evaluating the potency of MAB39 and MAB9 in the Wieslab Classical Pathway assay. MAB39 and MAB9 exhibited similar dose-dependent and complete inhibition of the classical pathway in the Wieslab ELISA assay at 1% human serum concentration. In contrast, an IgG4 isotype control antibody had minimal effect and did not inhibit the classical pathway. The IC50 value for MAB39 was 0.49±0.11 nM and MAB9 was 0.69±0.28 nM, representing the mean and standard deviation from the 3 independent experiments (Table 18). Thus, no statistical difference in potency was observed between MAB39 and MAB9 in the Wieslab Classical Pathway assay

TABLE 18
IC50 values for MAB39 and MAB9 in the Wieslab
Classical Pathway Assay with 1%
Human Serum

| Antibody | IC50 (nM) |
|---|---|
| MAB39 | 0.49 ± 0.11 |
| MAB9 | 0.69 ± 0.28 |

The values represent the mean and standard deviation from three independent experiments performed in duplicates.

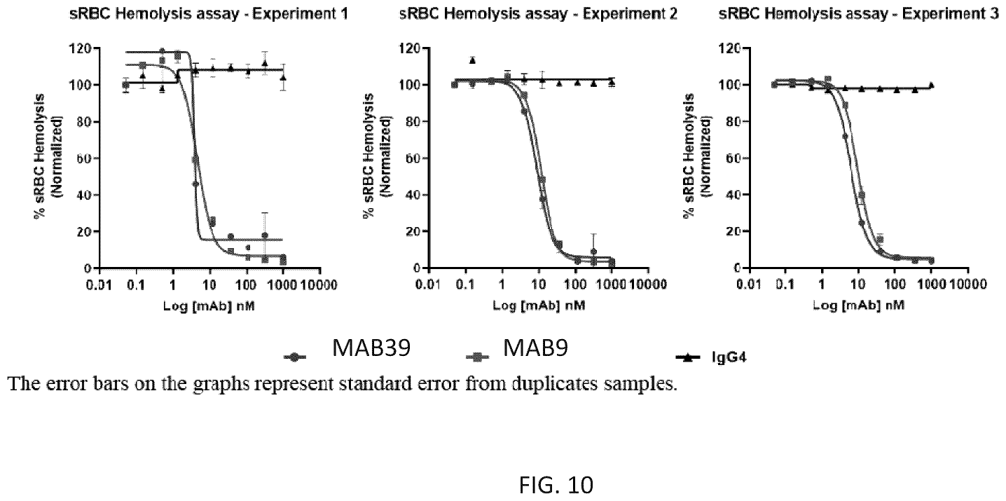

Comparison of Potency Between MAB39 and MAB9 in an Antibody-sensitized Sheep Red Blood Cell Lysis Assay. The percentage hemolysis versus antibody concentrations were plotted and the IC50 values were calculated. FIG. 10 shows the graphs from all 3 independent experiments evaluating the potency of MAB39 and MAB9 in the antibody-sensitized sheep red blood cell lysis assay. MAB39 and MAB9 exhibited similar dose-dependent and complete inhibition of the classical pathway in the antibody-sensitized sheep red blood cell lysis assay at 1% human serum concentration. The isotype control antibody did not inhibit the classical pathway mediated cell lysis. The IC50 value for MAB39 was 6.30±2.51 nM and MAB9 was 8.57±3.57 nM, representing the mean and standard deviation from the 3 independent experiments (Table 19). Consistent with the findings in the Wieslab Classical Pathway assay, no statistical difference in potency was observed between MAB39 and MAB9 in the antibody-sensitized sheep red blood cell lysis assay.

TABLE 19
IC50 values for MAB39 and MAB9 in the
antibody-sensitized sheep red blood cell lysis assay

| Antibody | IC50 (nM) |
|---|---|
| MAB39 | 6.30 ± 2.51 |
| MAB9 | 8.57 ± 3.57 |

The values represent the mean an standard deviation rom independent experiments performed in duplicates.

Results: MAB9 and MAB39 exhibited comparable binding affinities to the target protein human active C1s. The KD values±SD of MAB39 and MAB9 for human active C1s were 0.0105±0.0046 nM and 0.0071±0.00054 nM, respectively. Both antibodies demonstrated comparable potency, with complete inhibition of the classical pathway in the Wieslab and antibody-sensitized red blood cell lysis assays. For the Wieslab Classical Pathway assay using 1% human sera, the IC50 values±standard deviation (SD) for MAB39 was 0.49±0.11 nM and MAB9 was 0.69±0.28 nM. For the antibody-sensitized sheep red blood cell lysis assay using 1% human sera, the IC50 values±SD for MAB39 was 6.30±2.51 nM and MAB9 was 8.57±3.57 nM.

Conclusions: The single amino acid substitution from glutamine (MAB9) to glutamate (MAB39) had no statistically observable effect on the binding affinity to human active C1s by MAB9 or MAB39, and both molecules showed equivalent in vitro inhibition (i.e., IC50 values) of classical pathway activity in 2 well-established complement classical pathway assays. These findings demonstrate that MAB39 and its analog and precursor, MAB9, exhibit indistinguishable biophysical and functional properties, and that preclinical studies conducted with MAB9 can be used to support the clinical evaluation of MAB39.

### Example 10: Anti-Active C1s Antibodies Prevent Neurotransmission Impairment in a Functional In Vitro Model of Myasthenia Gravis (MG)

A functional human on-chip model of MG was used to interrogate the effect of IgG4-MAB9 on physiologically relevant readouts that include deposition of the pore-forming membrane attack complex (MAC), neurotransmission and muscle fatigability.

The effect of IgG4-MAB9 on neurotransmission and muscle fatigue was assessed using an in vitro human on-chip model of MG composed of human iPSC-derived motor neurons and primary human skeletal muscle cells in NHS-supplemented AChR+ MG patient sera.

Human iPSC-derived motor neurons and primary human skeletal muscle cells were cultured in separate chambers of a two-chamber compartmentalized microfluidic device for one month. On the day of the experiment, 10% AChR+ MG patient sera or healthy volunteer sera each containing 1% NHS-supplemented sera was added to the wells. The cells were treated with IgG4-MAB9, recombinant ravulizumab, or left untreated for three hours. Muscle contraction fatigue was assessed by stimulating cells in the motor neuron chamber with electrical stimulation at 2 Hz for 2 minutes and recording the resulting muscle contractions in the skeletal muscle cell chamber. Muscle contraction fatigue index score was calculated based on the area under the curve of the region of interest divided by tetanic response from individual myotubes.

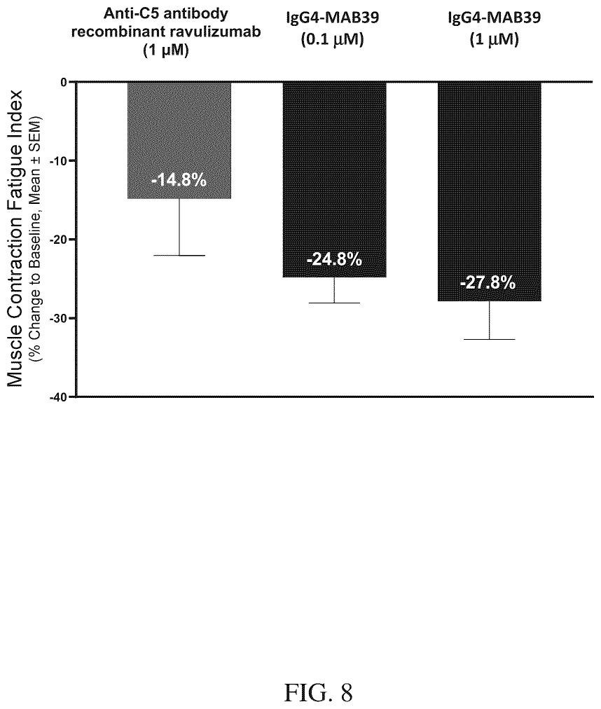

The effect of IgG4-MAB39 and anti-C5 antibody recombinant ravulizumab on the muscle fatigue index in cells incubated in AChR+ MG serum from three individual patients is shown in FIG. 8.

Treatment of the cells incubated in AChR+ MG patient sera with 0.1 μM and 1 μM IgG4-MAB39 resulted in a reduction of muscle contraction fatigue by 24.8% and 27.8%, respectively, as compared to baseline. Reduced muscle contraction fatigue was also observed in cells incubated in AChR+ MG patient sera treated with anti-C5 antibody recombinant ravulizumab, as compared to baseline. This finding provides a scientific rationale for targeting complement classical pathway in AChR+ MG.

IgG4-MAB39 was found to be a potent and selective anti-active C1s antibody that prevented complement-mediated MAC formation and complement-mediated hRBC lysis, and blocked activity of the classical complement pathway, but not the alternative or lectin complement pathways for immune surveillance, as discussed in Examples 3, 4, and 5, above. AChR+ MG patient sera induced neurotransmission impairment and muscle fatigue in a human on-chip model of MG. Treatment with IgG4-MAB39 inhibited active C1s and prevented neurotransmission impairment and muscle fatigue in a cell-based human on-chip model of MG, allowing for muscle contraction and reducing the muscle fatigue index, providing proof of efficacy data in this indication.

### Example 11: MAB39 does not Inhibit N. meningitidis Bacteria Killing

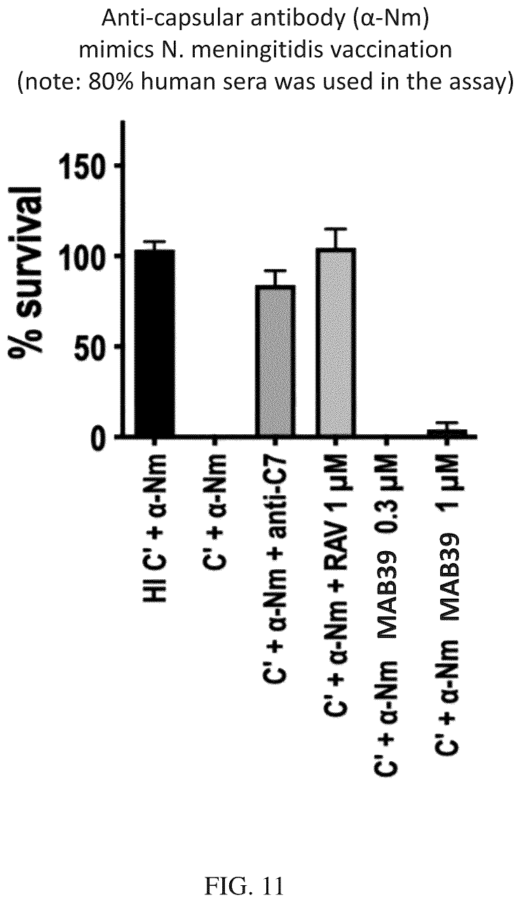

It can be important that the anti-C1S antibodies provided herein are specific and do not inhibit other immune pathways that are necessary for a subject's health, such as against bacterial infections. Accordingly, the ability of bacteria to survive in the presence of antibodies specific for the active form of C1S was tested. Briefly, N. meningitidis bacteria survival was measured in a culture of complement-containing sera (80% human sera) and anticapsular antibody (α-Nm), which mimics N. meningitidis vaccination, with and without test reagents. Anti-C7 antibody and heat-inactivation (HI), were used as controls that inactivate complement and demonstrated that the bacteria survived (see, FIG. 11). Ravulizumab (RAV), a non-specific humanized monoclonal antibody complement inhibitor, resulted in bacterial survival (i.e., no complement-mediated killing). In contrast to ravulizumab, no bacterial survival was observed when MAB39 was added to the complement-containing sera (C′) and anticapsular antibody (α-Nm) mixture, which means that MAB39 does not inhibit N. meningitidis bacteria complement mediated killing. (FIG. 11) The lack of inhibition of meningitidis bacteria complement mediated killing was observed at both 0.3 uM and 1 uM concentrations. (FIG. 11) Accordingly, the antibodies provided herein do not inhibit complement mediated bacteria killing as compared to the non-specific complement antibody inhibitor ravulizumab.

### Example 12: Proteolytic Cleavage of Non-Complement Substrates by Active C1s

Several in vitro studies have demonstrated an emerging role of active C1s in the proteolytic cleavage of non-complement substrates that include, for example, collagen, HMC I, HMGB1, IGFBP5, LRP6, NCL, and NPM1. Without being bound to any particular theory, these potential non-complement substrates of active C1s are postulated to be important in the clearance of antigens on apoptotic cells or in the hemostasis of bone, muscle, and connective tissues. Accordingly, it would be preferable for an antibody that targets the active form of C1S not inhibit or not inhibit significantly inhibit the cleavage of these non-complement substrates.

To test this, non-complement substrates in PBS (phosphate buffer saline) are incubated with active C1s (Complement Technologies, Tyler, TX, Catalog A104) in the absence or presence of varying concentrations of MAB39, MAB9, or isotype IgG4 antibody at 37° C. for 30 minutes. Complement component C4 is used a positive control. The reaction is stopped with 10×SDS-PAGE loading buffer with reducing agent DTT (dithiothreitol) and heated to 95° C. for about 10 minutes. Samples are then subjected to SDS-PAGE and cleavage products are detected by Coomassie Blue staining (for native non-complement substrate proteins) or alternatively, Western blot analysis (for recombinant non-complement substrate proteins with His- or Flag-tags).

No significant cleavage of non-complement substrates HMC I, HMGB1, IGFBP5, LRP6, NCL, and NPM1 by MAB39 or MAB9 is observed.

Thus, the antibodies provided for herein can be used as inhibitors of active C1s to treat C1s mediated disorders, such as cold agglutinin disease and Myasthenia Gravis. Due to the specificity of the antibodies to active C1s, the antibodies provided herein do not inhibit complement mediated bacterial killing and are not expected to inhibit the proteolytic cleavage of non-complement substrates of active C1S.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description and the accompanying FIGs. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

1-26. (canceled)

27. An antibody, or an antigen binding fragment thereof, wherein the antibody or antibody fragment comprises:
(a) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62, and the heavy chain HCDR3 sequence has the amino acid sequence of SEQ ID NO: 63, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 66; or variants or convention equivalents of any of the foregoing;
(b) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 67, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 68; or variants or convention equivalents of any of the foregoing;
(c) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 69, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 68; or variants or convention equivalents of any of the foregoing;
(d) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 70, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65, or SEQ ID NO: 134; and the light chain LCDR3 sequence has the amino acid sequence of SEQ ID NO: 71; or variants or convention equivalents of any of the foregoing;
(e) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 72, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 71; or variants or convention equivalents of any of the foregoing;
(f) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 73, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 71, or variants or convention equivalents of any of the foregoing;
(g) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 74, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 75; or variants or convention equivalents of any of the foregoing;
(h) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 76, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64; the light chain LCDR2 sequence has the amino acid sequence of SEQ ID NO: 65; and the light chain LCDR3 sequence has the amino acid sequence of SEQ ID NO: 77; or variants or convention equivalents of any of the foregoing;
(i) a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62, and the heavy chain HCDR3 sequence has the amino acid sequence of SEQ ID NO: 80, or SEQ ID NO: 143, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 64, or SEQ ID NO 133; the light chain LCDR2 sequence has the amino acid sequence of SEQ ID NO: 65, or SEQ ID NO: 134; and the light chain LCDR3 sequence has the amino acid sequence of SEQ ID NO: 81; or variants or convention equivalents of any of the foregoing;
(j) a heavy chain variable region comprising heavy chain HCDR1, HCDR2, and HCDR3 sequences, wherein the heavy chain HCDR1 sequence has the amino acid sequence of SEQ ID NO: 82N; the heavy chain HCDR2 has the amino acid sequence of SEQ ID NO: 83; and the heavy chain HCDR3 sequence has the amino acid sequence of SEQ ID NO: 84, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 87; or variants or convention equivalents of any of the foregoing;
(k) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 88, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 89; or variants or convention equivalents of any of the foregoing;
(l) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 90, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 89; or variants or convention equivalents of any of the foregoing;
(m) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 91, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 92; or variants or convention equivalents of any of the foregoing;
(n) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 93, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 94; or variants or convention equivalents of any of the foregoing;
(o) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 84, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 95; or variants or convention equivalents of any of the foregoing;
(p) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 96, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 97; or variants or convention equivalents of any of the foregoing;
(q) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 98, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 99; or variants or convention equivalents of any of the foregoing;
(r) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 100, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 101; or variants or convention equivalents of any of the foregoing;
(s) a heavy chain variable region comprising heavy chain HCDR1, HCDR2, and HCDR3 sequences, wherein the heavy chain HCDR1 sequence has the amino acid sequence of SEQ ID NO: 82; the heavy chain HCDR2 has the amino acid sequence of SEQ ID NO: 83; and the heavy chain HCDR3 sequence has the amino acid sequence of SEQ ID NO: 100, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 85; the light chain LCDR2 sequence has the amino acid sequence of SEQ ID NO: 86; and the light chain LCDR3 sequence has the amino acid sequence of SEQ ID NO: 102; or variants or convention equivalents of any of the foregoing;
(t) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 105, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 108; or variants or convention equivalents of any of the foregoing;
(u) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 109, or SEQ ID NO: 161, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 110; or variants or convention equivalents of any of the foregoing;
(v) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 111, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 112; or variants or convention equivalents of any of the foregoing;
(w) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 105, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 113; or variants or convention equivalents of any of the foregoing;
(x) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 114, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 115; or variants or convention equivalents of any of the foregoing;
(y) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 116, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 113; or variants or convention equivalents of any of the foregoing;
(z) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 117, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 118; or variants or convention equivalents of any of the foregoing;
(aa) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 116, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 119; or variants or convention equivalents of any of the foregoing;
(ab) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 120, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 121; or variants or convention equivalents of any of the foregoing; or
(ac) a heavy chain variable region comprising heavy chain HCDR1, HCDR2, and HCDR3 sequences, wherein the heavy chain HCDR1 sequence has the amino acid sequence of SEQ ID NO: 103; the heavy chain HCDR2 has the amino acid sequence of SEQ ID NO: 104; and the heavy chain HCDR3 sequence has the amino acid sequence of SEQ ID NO: 122, or variants or convention equivalents of any of the foregoing; and (ii) a light chain variable region comprising light chain LCDR1, LCDR2, and LCDR3 sequences, wherein the light chain LCDR1 sequence has the amino acid sequence of SEQ ID NO: 106; the light chain LCDR2 sequence has the amino acid sequence of SEQ ID NO: 107; and the light chain LCDR3 sequence has the amino acid sequence of SEQ ID NO: 123; or variants or convention equivalents of any of the foregoing.

28. The antibody, or antigen-binding fragment thereof, of claim 27, wherein the antibody, or antigen binding fragment thereof, comprises:
(a) a light chain variable region of the light chain having at least 90% sequence identity to SEQ ID NO: 2, and a heavy chain variable region of the heavy chain having at least 90% sequence identity to SEQ ID NO: 1, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 63 the LCDR1 having the amino acid sequence of SEQ ID NO: 64, the LCDR2 having the amino acid sequence of SEQ ID NO: 65, and the LCDR3 having the amino acid sequence of SEQ ID NO: 66;
(b) a light chain variable region of the light chain having at least 90% sequence identity to SEQ ID NO: 4, and a heavy chain variable region of the heavy chain having at least 90% sequence identity to SEQ ID NO: 3, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 67 the LCDR1 having the amino acid sequence of SEQ ID NO: 64, the LCDR2 having the amino acid sequence of SEQ ID NO: 65, and the LCDR3 having the amino acid sequence of SEQ ID NO: 68;
(c) a light chain variable region of the light chain having at least 90% sequence identity to SEQ ID NO: 6, and a heavy chain variable region of the heavy chain having at least 90% sequence identity to SEQ ID NO: 5, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 69 the LCDR1 having the amino acid sequence of SEQ ID NO: 64, the LCDR2 having the amino acid sequence of SEQ ID NO: 65, and the LCDR3 having the amino acid sequence of SEQ ID NO: 68;
(d) a light chain variable region of the light chain having at least 90% sequence identity to SEQ ID NO: 8, and a heavy chain variable region of the heavy chain having at least 90% sequence identity to SEQ ID NO: 7, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 70 the LCDR1 having the amino acid sequence of SEQ ID NO: 64, the LCDR2 having the amino acid sequence of SEQ ID NO: 65, and the LCDR3 having the amino acid sequence of SEQ ID NO: 71;
(e) a light chain variable region of the light chain having at least 90% sequence identity to SEQ ID NO: 10, and a heavy chain variable region of the heavy chain having at least 90% sequence identity to SEQ ID NO: 9, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 72 the LCDR1 having the amino acid sequence of SEQ ID NO: 64, the LCDR2 having the amino acid sequence of SEQ ID NO: 65, and the LCDR3 having the amino acid sequence of SEQ ID NO: 71;
(f) a light chain variable region of the light chain having at least 90% sequence identity to SEQ ID NO: 12, and a heavy chain variable region of the heavy chain having at least 90% sequence identity to SEQ ID NO: 11, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 73 the LCDR1 having the amino acid sequence of SEQ ID NO: 64, the LCDR2 having the amino acid sequence of SEQ ID NO: 65, and the LCDR3 having the amino acid sequence of SEQ ID NO: 71;
(g) a light chain variable region of the light chain having at least 90% sequence identity to SEQ ID NO: 14, and a heavy chain variable region of the heavy chain having at least 90% sequence identity to SEQ ID NO: 13, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 74 the LCDR1 having the amino acid sequence of SEQ ID NO: 64, the LCDR2 having the amino acid sequence of SEQ ID NO: 65, and the LCDR3 having the amino acid sequence of SEQ ID NO: 75;
(h) a light chain variable region of the light chain having at least 90% sequence identity to SEQ ID NO: 16, and a heavy chain variable region of the heavy chain having at least 90% sequence identity to SEQ ID NO: 15, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 76 the LCDR1 having the amino acid sequence of SEQ ID NO: 64, the LCDR2 having the amino acid sequence of SEQ ID NO: 65 and the LCDR3 having the amino acid sequence of SEQ ID NO: 77;
(i) a light chain variable region of the light chain having at least 90% sequence identity to SEQ ID NO: 20, and a heavy chain variable region of the heavy chain having at least 90% sequence identity to SEQ ID NO: 19, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 80 the LCDR1 having the amino acid sequence of SEQ ID NO: 64, the LCDR2 having the amino acid sequence of SEQ ID NO: 65, and the LCDR3 having the amino acid sequence of SEQ ID NO: 81;
(j) a light chain variable region of the light chain having at least 90% sequence identity to SEQ ID NO: 22, and a heavy chain variable region of the heavy chain having at least 90% sequence identity to SEQ ID NO: 21, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 84 the LCDR1 having the amino acid sequence of SEQ ID NO: 85, the LCDR2 having the amino acid sequence of SEQ ID NO: 86, and the LCDR3 having the amino acid sequence of SEQ ID NO: 87;
(k) a light chain variable region of the light chain having at least 90% sequence identity to SEQ ID NO: 24, and a heavy chain variable region of the heavy chain having at least 90% sequence identity to SEQ ID NO: 23, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 88, the LCDR1 having the amino acid sequence of SEQ ID NO: 85, the LCDR2 having the amino acid sequence of SEQ ID NO: 86, and the LCDR3 having the amino acid sequence of SEQ ID NO: 89;
(l) a light chain variable region of the light chain having at least 90% sequence identity to SEQ ID NO: 26, and a heavy chain variable region of the heavy chain having at least 90% sequence identity to SEQ ID NO: 25, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 90, the LCDR1 having the amino acid sequence of SEQ ID NO: 85, the LCDR2 having the amino acid sequence of SEQ ID NO: 86, and the LCDR3 having the amino acid sequence of SEQ ID NO: 89;
(m) a light chain variable region of the light chain having at least 90% sequence identity to SEQ ID NO: 28, and a heavy chain variable region of the heavy chain having at least 90% sequence identity to SEQ ID NO: 27, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 91, the LCDR1 having the amino acid sequence of SEQ ID NO: 85, the LCDR2 having the amino acid sequence of SEQ ID NO: 86, and the LCDR3 having the amino acid sequence of SEQ ID NO: 92;
(n) a light chain variable region of the light chain having at least 90% sequence identity to SEQ ID NO: 30, and a heavy chain variable region of the heavy chain having at least 90% sequence identity to SEQ ID NO: 29, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 93 the LCDR1 having the amino acid sequence of SEQ ID NO: 85, the LCDR2 having the amino acid sequence of SEQ ID NO: 86, and the LCDR3 having the amino acid sequence of SEQ ID NO: 94;
(o) a light chain variable region of the light chain having at least 90% sequence identity to SEQ ID NO: 32, and a heavy chain variable region of the heavy chain having at least 90% sequence identity to SEQ ID NO: 31, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 84 the LCDR1 having the amino acid sequence of SEQ ID NO: 85, the LCDR2 having the amino acid sequence of SEQ ID NO: 86, and the LCDR3 having the amino acid sequence of SEQ ID NO: 95;
(p) a light chain variable region of the light chain having at least 90% sequence identity to SEQ ID NO: 34, and a heavy chain variable region of the heavy chain having at least 90% sequence identity to SEQ ID NO: 33, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 96 the LCDR1 having the amino acid sequence of SEQ ID NO: 85, the LCDR2 having the amino acid sequence of SEQ ID NO: 86, and the LCDR3 having the amino acid sequence of SEQ ID NO: 97;
(q) a light chain variable region of the light chain having at least 90% sequence identity to SEQ ID NO: 36, and a heavy chain variable region of the heavy chain having at least 90% sequence identity to SEQ ID NO: 35, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 98, the LCDR1 having the amino acid sequence of SEQ ID NO: 85, the LCDR2 having the amino acid sequence of SEQ ID NO: 86, and the LCDR3 having the amino acid sequence of SEQ ID NO: 99;
(r) a light chain variable region of the light chain having at least 90% sequence identity to SEQ ID NO: 38, and a heavy chain variable region of the heavy chain having at least 90% sequence identity to SEQ ID NO: 37, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 100, the LCDR1 having the amino acid sequence of SEQ ID NO: 85, the LCDR2 having the amino acid sequence of SEQ ID NO: 86, and the LCDR3 having the amino acid sequence of SEQ ID NO: 101;
(s) a light chain variable region of the light chain having at least 90% sequence identity to SEQ ID NO: 40, and a heavy chain variable region of the heavy chain having at least 90% sequence identity to SEQ ID NO: 39, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 100, the LCDR1 having the amino acid sequence of SEQ ID NO: 85, the LCDR2 having the amino acid sequence of SEQ ID NO: 86, and the LCDR3 having the amino acid sequence of SEQ ID NO: 102;
(t) a light chain variable region of the light chain having at least 90% sequence identity to SEQ ID NO: 42, and a heavy chain variable region of the heavy chain having at least 90% sequence identity to SEQ ID NO: 41, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 105, the LCDR1 having the amino acid sequence of SEQ ID NO: 106, the LCDR2 having the amino acid sequence of SEQ ID NO: 107, and the LCDR3 having the amino acid sequence of SEQ ID NO: 108;
(u) a light chain variable region of the light chain having at least 90% sequence identity to SEQ ID NO: 44, and a heavy chain variable region of the heavy chain having at least 90% sequence identity to SEQ ID NO: 43, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 109, the LCDR1 having the amino acid sequence of SEQ ID NO: 106, the LCDR2 having the amino acid sequence of SEQ ID NO: 107, and the LCDR3 having the amino acid sequence of SEQ ID NO: 110;
(v) a light chain variable region of the light chain having at least 90% sequence identity to SEQ ID NO: 46, and a heavy chain variable region of the heavy chain having at least 90% sequence identity to SEQ ID NO: 45, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 111, the LCDR1 having the amino acid sequence of SEQ ID NO: 106, the LCDR2 having the amino acid sequence of SEQ ID NO: 107, and the LCDR3 having the amino acid sequence of SEQ ID NO: 112;
(w) a light chain variable region of the light chain having at least 90% sequence identity to SEQ ID NO: 48, and a heavy chain variable region of the heavy chain having at least 90% sequence identity to SEQ ID NO: 47, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 105, the LCDR1 having the amino acid sequence of SEQ ID NO: 106, the LCDR2 having the amino acid sequence of SEQ ID NO: 107, and the LCDR3 having the amino acid sequence of SEQ ID NO: 113;
(x) a light chain variable region of the light chain having at least 90% sequence identity to SEQ ID NO: 50, and a heavy chain variable region of the heavy chain having at least 90% sequence identity to SEQ ID NO: 49, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 114, the LCDR1 having the amino acid sequence of SEQ ID NO: 106, the LCDR2 having the amino acid sequence of SEQ ID NO: 107, and the LCDR3 having the amino acid sequence of SEQ ID NO: 115;
(y) a light chain variable region of the light chain having at least 90% sequence identity to SEQ ID NO: 52, and a heavy chain variable region of the heavy chain having at least 90% sequence identity to SEQ ID NO: 51, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 116, the LCDR1 having the amino acid sequence of SEQ ID NO: 106, the LCDR2 having the amino acid sequence of SEQ ID NO: 107, and the LCDR3 having the amino acid sequence of SEQ ID NO: 113;
(z) a light chain variable region of the light chain having at least 90% sequence identity to SEQ ID NO: 54, and a heavy chain variable region of the heavy chain having at least 90% sequence identity to SEQ ID NO: 53, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 117, the LCDR1 having the amino acid sequence of SEQ ID NO: 106, the LCDR2 having the amino acid sequence of SEQ ID NO: 107, and the LCDR3 having the amino acid sequence of SEQ ID NO: 118;
(aa) a light chain variable region of the light chain having at least 90% sequence identity to SEQ ID NO: 56, and a heavy chain variable region of the heavy chain having at least 90% sequence identity to SEQ ID NO: 55, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 116, the LCDR1 having the amino acid sequence of SEQ ID NO: 106, the LCDR2 having the amino acid sequence of SEQ ID NO: 107, and the LCDR3 having the amino acid sequence of SEQ ID NO: 119;
(ab) a light chain variable region of the light chain having at least 90% sequence identity to SEQ ID NO: 58, and a heavy chain variable region of the heavy chain having at least 90% sequence identity to SEQ ID NO: 57, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 120, the LCDR1 having the amino acid sequence of SEQ ID NO: 106, the LCDR2 having the amino acid sequence of SEQ ID NO: 107, and the LCDR3 having the amino acid sequence of SEQ ID NO: 121;
(c) a light chain variable region of the light chain having at least 90% sequence identity to SEQ ID NO: 60, and a heavy chain variable region of the heavy chain having at least 90% sequence identity to SEQ ID NO: 59, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 122, the LCDR1 having the amino acid sequence of SEQ ID NO: 106, the LCDR2 having the amino acid sequence of SEQ ID NO: 107, and the LCDR3 having the amino acid sequence of SEQ ID NO: 123;
(ad) a light chain variable region of the light chain having at least 90% sequence identity to SEQ ID NO: 2, and a heavy chain variable region of the heavy chain having at least 90% sequence identity to SEQ ID NO: 334, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 63, the LCDR1 having the amino acid sequence of SEQ ID NO: 64, the LCDR2 having the amino acid sequence of SEQ ID NO: 65, and the LCDR3 having the amino acid sequence of SEQ ID NO: 66;
(ag) a light chain variable region of the light chain having at least 90% sequence identity to SEQ ID NO: 4, and a heavy chain variable region of the heavy chain having at least 90% sequence identity to SEQ ID NO: 335, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 67, the LCDR1 having the amino acid sequence of SEQ ID NO: 64, the LCDR2 having the amino acid sequence of SEQ ID NO: 65, and the LCDR3 having the amino acid sequence of SEQ ID NO: 68;
(af) a light chain variable region of the light chain having at least 90% sequence identity to SEQ ID NO: 6, and a heavy chain variable region of the heavy chain having at least 90% sequence identity to SEQ ID NO: 336, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 69, the LCDR1 having the amino acid sequence of SEQ ID NO: 64, the LCDR2 having the amino acid sequence of SEQ ID NO: 65, and the LCDR3 having the amino acid sequence of SEQ ID NO: 68;
(ag) a light chain variable region of the light chain having at least 90% sequence identity to SEQ ID NO: 8, and a heavy chain variable region of the heavy chain having at least 90% sequence identity to SEQ ID NO: 337, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 70, the LCDR1 having the amino acid sequence of SEQ ID NO: 64, the LCDR2 having the amino acid sequence of SEQ ID NO: 65, and the LCDR3 having the amino acid sequence of SEQ ID NO: 71;
(ah) a light chain variable region of the light chain having at least 90% sequence identity to SEQ ID NO: 10, and a heavy chain variable region of the heavy chain having at least 90% sequence identity to SEQ ID NO: 338, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 72, the LCDR1 having the amino acid sequence of SEQ ID NO: 64, the LCDR2 having the amino acid sequence of SEQ ID NO: 65, and the LCDR3 having the amino acid sequence of SEQ ID NO: 71;
(ai) a light chain variable region of the light chain having at least 90% sequence identity to SEQ ID NO: 12, and a heavy chain variable region of the heavy chain having at least 90% sequence identity to SEQ ID NO: 339, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 73, the LCDR1 having the amino acid sequence of SEQ ID NO: 64, the LCDR2 having the amino acid sequence of SEQ ID NO: 65, and the LCDR3 having the amino acid sequence of SEQ ID NO: 71;
(aj) a light chain variable region of the light chain having at least 90% sequence identity to SEQ ID NO: 14, and a heavy chain variable region of the heavy chain having at least 90% sequence identity to SEQ ID NO: 340, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 74, the LCDR1 having the amino acid sequence of SEQ ID NO: 64, the LCDR2 having the amino acid sequence of SEQ ID NO: 65, and the LCDR3 having the amino acid sequence of SEQ ID NO: 75;
(ak) a light chain variable region of the light chain having at least 90% sequence identity to SEQ ID NO: 16, and a heavy chain variable region of the heavy chain having at least 90% sequence identity to SEQ ID NO: 341, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 76, the LCDR1 having the amino acid sequence of SEQ ID NO: 64, the LCDR2 having the amino acid sequence of SEQ ID NO: 65, and the LCDR3 having the amino acid sequence of SEQ ID NO: 77;
(al) a light chain variable region of the light chain having at least 90% sequence identity to SEQ ID NO: 20, and a heavy chain variable region of the heavy chain having at least 90% sequence identity to SEQ ID NO: 343, wherein the antibody, or antigen binding fragment thereof, maintains the HCDR1 having the amino acid sequence of SEQ ID NO: 61, the HCDR2 having the amino acid sequence of SEQ ID NO: 62, the HCDR3 having the amino acid sequence of SEQ ID NO: 80, the LCDR1 having the amino acid sequence of SEQ ID NO: 64, the LCDR2 having the amino acid sequence of SEQ ID NO: 65, and the LCDR3 having the amino acid sequence of SEQ ID NO: 81;
(am) a light chain variable region of the light chain having at least 90% sequence identity to SEQ ID NO: 22, and a heavy chain variable region of the heavy chain having at least 90% sequence identity to SEQ ID NO: 344, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 84, the LCDR1 having the amino acid sequence of SEQ ID NO: 85, the LCDR2 having the amino acid sequence of SEQ ID NO: 86, and the LCDR3 having the amino acid sequence of SEQ ID NO: 87;
(an) a light chain variable region of the light chain having at least 90% sequence identity to SEQ ID NO: 24, and a heavy chain variable region of the heavy chain having at least 90% sequence identity to SEQ ID NO: 345, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 88, the LCDR1 having the amino acid sequence of SEQ ID NO: 85, the LCDR2 having the amino acid sequence of SEQ ID NO: 86, and the LCDR3 having the amino acid sequence of SEQ ID NO: 89;
(ao) a light chain variable region of the light chain having at least 90% sequence identity to SEQ ID NO: 26, and a heavy chain variable region of the heavy chain having at least 90% sequence identity to SEQ ID NO: 346, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 90, the LCDR1 having the amino acid sequence of SEQ ID NO: 85, the LCDR2 having the amino acid sequence of SEQ ID NO: 86, and the LCDR3 having the amino acid sequence of SEQ ID NO: 89;
(ap) a light chain variable region of the light chain having at least 90% sequence identity to SEQ ID NO: 28, and a heavy chain variable region of the heavy chain having at least 90% sequence identity to SEQ ID NO: 347, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 91, the LCDR1 having the amino acid sequence of SEQ ID NO: 85, the LCDR2 having the amino acid sequence of SEQ ID NO: 86, and the LCDR3 having the amino acid sequence of SEQ ID NO: 92;
(aq) a light chain variable region of the light chain having at least 90% sequence identity to SEQ ID NO: 30, and a heavy chain variable region of the heavy chain having at least 90% sequence identity to SEQ ID NO: 348, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 93, the LCDR1 having the amino acid sequence of SEQ ID NO: 85, the LCDR2 having the amino acid sequence of SEQ ID NO: 86, and the LCDR3 having the amino acid sequence of SEQ ID NO: 94;
(ar) a light chain variable region of the light chain having at least 90% sequence identity to SEQ ID NO: 32, and a heavy chain variable region of the heavy chain having at least 90% sequence identity to SEQ ID NO: 349, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 84, the LCDR1 having the amino acid sequence of SEQ ID NO: 85, the LCDR2 having the amino acid sequence of SEQ ID NO: 86, and the LCDR3 having the amino acid sequence of SEQ ID NO: 95;
(as) a light chain variable region of the light chain having at least 90% sequence identity to SEQ ID NO: 34, and a heavy chain variable region of the heavy chain having at least 90% sequence identity to SEQ ID NO: 350, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 96, the LCDR1 having the amino acid sequence of SEQ ID NO: 85, the LCDR2 having the amino acid sequence of SEQ ID NO: 86, and the LCDR3 having the amino acid sequence of SEQ ID NO: 97;
(at) a light chain variable region of the light chain having at least 90% sequence identity to SEQ ID NO: 36, and a heavy chain variable region of the heavy chain having at least 90% sequence identity to SEQ ID NO: 351, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 98, the LCDR1 having the amino acid sequence of SEQ ID NO: 85, the LCDR2 having the amino acid sequence of SEQ ID NO: 86, and the LCDR3 having the amino acid sequence of SEQ ID NO: 99;
(au) a light chain variable region of the light chain having at least 90% sequence identity to SEQ ID NO: 38, and a heavy chain variable region of the heavy chain having at least 90% sequence identity to SEQ ID NO: 352, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 100, the LCDR1 having the amino acid sequence of SEQ ID NO: 85, the LCDR2 having the amino acid sequence of SEQ ID NO: 86, and the LCDR3 having the amino acid sequence of SEQ ID NO: 101;
(av) a light chain variable region of the light chain having at least 90% sequence identity to SEQ ID NO: 40, and a heavy chain variable region of the heavy chain having at least 90% sequence identity to SEQ ID NO: 353, wherein the antibody, or antigen binding fragment thereof, maintains the HCDR1 having the amino acid sequence of SEQ ID NO: 82, the HCDR2 having the amino acid sequence of SEQ ID NO: 83, the HCDR3 having the amino acid sequence of SEQ ID NO: 100, the LCDR1 having the amino acid sequence of SEQ ID NO: 85, the LCDR2 having the amino acid sequence of SEQ ID NO: 86, and the LCDR3 having the amino acid sequence of SEQ ID NO: 102;
(aw) a light chain variable region of the light chain having at least 90% sequence identity to SEQ ID NO: 42, and a heavy chain variable region of the heavy chain having at least 90% sequence identity to SEQ ID NO: 354, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 105, the LCDR1 having the amino acid sequence of SEQ ID NO: 106, the LCDR2 having the amino acid sequence of SEQ ID NO: 107, and the LCDR3 having the amino acid sequence of SEQ ID NO: 108;
(x) a light chain variable region of the light chain having at least 90% sequence identity to SEQ ID NO: 44, and a heavy chain variable region of the heavy chain having at least 90% sequence identity to SEQ ID NO: 355, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 109, the LCDR1 having the amino acid sequence of SEQ ID NO: 106, the LCDR2 having the amino acid sequence of SEQ ID NO: 107, and the LCDR3 having the amino acid sequence of SEQ ID NO: 110;
(ay) a light chain variable region of the light chain having at least 90% sequence identity to SEQ ID NO: 46, and a heavy chain variable region of the heavy chain having at least 90% sequence identity to SEQ ID NO: 356, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 111, the LCDR1 having the amino acid sequence of SEQ ID NO: 106, the LCDR2 having the amino acid sequence of SEQ ID NO: 107, and the LCDR3 having the amino acid sequence of SEQ ID NO: 112;
(w) a light chain variable region of the light chain having at least 90% sequence identity to SEQ ID NO: 48, and a heavy chain variable region of the heavy chain having at least 90% sequence identity to SEQ ID NO: 357, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 105, the LCDR1 having the amino acid sequence of SEQ ID NO: 106, the LCDR2 having the amino acid sequence of SEQ ID NO: 107, and the LCDR3 having the amino acid sequence of SEQ ID NO: 113;
(ba) a light chain variable region of the light chain having at least 90% sequence identity to SEQ ID NO: 50, and a heavy chain variable region of the heavy chain having at least 90% sequence identity to SEQ ID NO: 358, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 114, the LCDR1 having the amino acid sequence of SEQ ID NO: 106, the LCDR2 having the amino acid sequence of SEQ ID NO: 107, and the LCDR3 having the amino acid sequence of SEQ ID NO: 115;
(b) a light chain variable region of the light chain having at least 90% sequence identity to SEQ ID NO: 52, and a heavy chain variable region of the heavy chain having at least 90% sequence identity to SEQ ID NO: 359, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 116, the LCDR1 having the amino acid sequence of SEQ ID NO: 106, the LCDR2 having the amino acid sequence of SEQ ID NO: 107, and the LCDR3 having the amino acid sequence of SEQ ID NO: 113;
(bc) a light chain variable region of the light chain having at least 90% sequence identity to SEQ ID NO: 54, and a heavy chain variable region of the heavy chain having at least 90% sequence identity to SEQ ID NO: 360, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 117, the LCDR1 having the amino acid sequence of SEQ ID NO: 106, the LCDR2 having the amino acid sequence of SEQ ID NO: 107, and the LCDR3 having the amino acid sequence of SEQ ID NO: 118;
(bd) a light chain variable region of the light chain having at least 90% sequence identity to SEQ ID NO: 56, and a heavy chain variable region of the heavy chain having at least 90% sequence identity to SEQ ID NO: 361, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 116, the LCDR1 having the amino acid sequence of SEQ ID NO: 106, the LCDR2 having the amino acid sequence of SEQ ID NO: 107, and the LCDR3 having the amino acid sequence of SEQ ID NO: 119;
(be) a light chain variable region of the light chain having at least 90% sequence identity to SEQ ID NO: 58, and a heavy chain variable region of the heavy chain having at least 90% sequence identity to SEQ ID NO: 362, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 120, the LCDR1 having the amino acid sequence of SEQ ID NO: 106, the LCDR2 having the amino acid sequence of SEQ ID NO: 107, and the LCDR3 having the amino acid sequence of SEQ ID NO: 121; or
(bf) a light chain variable region of the light chain having at least 90% sequence identity to SEQ ID NO: 60, and a heavy chain variable region of the heavy chain having at least 90% sequence identity to SEQ ID NO: 363, wherein the antibody, or antigen binding fragment thereof, maintains the HCDR1 having the amino acid sequence of SEQ ID NO: 103, the HCDR2 having the amino acid sequence of SEQ ID NO: 104, the HCDR3 having the amino acid sequence of SEQ ID NO: 122, the LCDR1 having the amino acid sequence of SEQ ID NO: 106, the LCDR2 having the amino acid sequence of SEQ ID NO: 107, and the LCDR3 having the amino acid sequence of SEQ ID NO: 123.

29. The antibody, or antigen-binding fragment thereof, of claim 28, wherein;
(a) the light chain variable region comprises SEQ ID NO: 2, and the heavy chain variable region comprises SEQ ID NO: 1;
(b) the light chain variable region comprises SEQ ID NO: 4, and the heavy chain variable region comprises SEQ ID NO: 3;
(c) the light chain variable region comprises SEQ ID NO: 6, and the heavy chain variable region comprises SEQ ID NO: 5
(d) the light chain variable region comprises SEQ ID NO: 8, and the heavy chain variable region comprises SEQ ID NO: 7
(e) the light chain variable region comprises SEQ ID NO: 10, and the heavy chain variable region comprises SEQ ID NO: 9
(f) the light chain variable region comprises SEQ ID NO: 12, and the heavy chain variable region comprises SEQ ID NO: 11;
(g) the light chain variable region comprises SEQ ID NO: 14, and the heavy chain variable region comprises SEQ ID NO: 13;
(h) the light chain variable region comprises SEQ ID NO: 16, and the heavy chain variable region comprises SEQ ID NO: 15;
(i) the light chain variable region comprises SEQ ID NO: 20, and the heavy chain variable region comprises SEQ ID NO: 19;
(j) the light chain variable region comprises SEQ ID NO: 22, and the heavy chain variable region comprises SEQ ID NO: 21;
(k) the light chain variable region comprises SEQ ID NO: 24, and the heavy chain variable region comprises SEQ ID NO: 23;
(l) the light chain variable region comprises SEQ ID NO: 26, and the heavy chain variable region comprises SEQ ID NO: 25;
(m) the light chain variable region comprises SEQ ID NO: 28, and the heavy chain variable region comprises SEQ ID NO: 27;
(n) the light chain variable region comprises SEQ ID NO: 30, and the heavy chain variable region comprises SEQ ID NO: 29;
(o) the light chain variable region comprises SEQ ID NO: 32, and the heavy chain variable region comprises SEQ ID NO: 31;
(p) the light chain variable region comprises SEQ ID NO: 34, and the heavy chain variable region comprises SEQ ID NO: 33;
(q) the light chain variable region comprises SEQ ID NO: 36, and the heavy chain variable region comprises SEQ ID NO: 35;
(r) the light chain variable region comprises SEQ ID NO: 38, and the heavy chain variable region comprises SEQ ID NO: 37;
(s) the light chain variable region comprises SEQ ID NO: 40, and the heavy chain variable region comprises SEQ ID NO: 39;
(t) the light chain variable region comprises SEQ ID NO: 42, and the heavy chain variable region comprises SEQ ID NO: 41;
(u) the light chain variable region comprises SEQ ID NO: 44, and the heavy chain variable region comprises SEQ ID NO: 43;
(v) the light chain variable region comprises SEQ ID NO: 46, and the heavy chain variable region comprises SEQ ID NO: 45;
(w) the light chain variable region comprises SEQ ID NO: 48, and the heavy chain variable region comprises SEQ ID NO: 47;
(x) the light chain variable region comprises SEQ ID NO: 50, and the heavy chain variable region comprises SEQ ID NO: 49;
(v) the light chain variable region comprises SEQ ID NO: 52, and the heavy chain variable region comprises SEQ ID NO: 51;
(z) the light chain variable region comprises SEQ ID NO: 54, and the heavy chain variable region comprises SEQ ID NO: 53;
(aa) the light chain variable region comprises SEQ ID NO: 56, and the heavy chain variable region comprises SEQ ID NO: 55;
(ab) the light chain variable region comprises SEQ ID NO: 58, and the heavy chain variable region comprises SEQ ID NO: 57;
(ac) the light chain variable region comprises SEQ ID NO: 60, and the heavy chain variable region comprises SEQ ID NO: 59; or
(ad) the light chain variable region comprises SEQ ID NO: 2, and the heavy chain variable region comprises SEQ ID NO: 334;
(ae) the light chain variable region comprises SEQ ID NO: 4, and the heavy chain variable region comprises SEQ ID NO: 335;
(a) the light chain variable region comprises SEQ ID NO: 6, and the heavy chain variable region comprises SEQ ID NO: 336;
(ag) the light chain variable region comprises SEQ ID NO: 8, and the heavy chain variable region comprises SEQ ID NO: 337;
(ah) the light chain variable region comprises SEQ ID NO: 10, and the heavy chain variable region comprises SEQ ID NO: 338;
(ai) the light chain variable region comprises SEQ ID NO: 12, and the heavy chain variable region comprises SEQ ID NO: 339;
(aj) the light chain variable region comprises SEQ ID NO: 14, and the heavy chain variable region comprises SEQ ID NO: 340;
(ak) the light chain variable region comprises SEQ ID NO: 16, and the heavy chain variable region comprises SEQ ID NO: 341;
(al) the light chain variable region comprises SEQ ID NO: 20, and the heavy chain variable region comprises SEQ ID NO: 343;
(am) the light chain variable region comprises SEQ ID NO: 22, and the heavy chain variable region comprises SEQ ID NO: 344;
(an) the light chain variable region comprises SEQ ID NO: 24, and the heavy chain variable region comprises SEQ ID NO: 345;
(ao) the light chain variable region comprises SEQ ID NO: 26, and the heavy chain variable region comprises SEQ ID NO: 346;
(ap) the light chain variable region comprises SEQ ID NO: 28, and the heavy chain variable region comprises SEQ ID NO: 347;
(aq) the light chain variable region comprises SEQ ID NO: 30, and the heavy chain variable region comprises SEQ ID NO: 348;
(ar) the light chain variable region comprises SEQ ID NO: 32, and the heavy chain variable region comprises SEQ ID NO: 349;
(as) the light chain variable region comprises SEQ ID NO: 34, and the heavy chain variable region comprises SEQ ID NO: 350;
(at) the light chain variable region comprises SEQ ID NO: 36, and the heavy chain variable region comprises SEQ ID NO: 351;
(au) the light chain variable region comprises SEQ ID NO: 38, and the heavy chain variable region comprises SEQ ID NO: 352;
(av) the light chain variable region comprises SEQ ID NO: 40, and the heavy chain variable region comprises SEQ ID NO: 353;
(aw) the light chain variable region comprises SEQ ID NO: 42, and the heavy chain variable region comprises SEQ ID NO: 354;
(ax) the light chain variable region comprises SEQ ID NO: 44, and the heavy chain variable region comprises SEQ ID NO: 355;
(ay) the light chain variable region comprises SEQ ID NO: 46, and the heavy chain variable region comprises SEQ ID NO: 356;
(z) the light chain variable region comprises SEQ ID NO: 48, and the heavy chain variable region comprises SEQ ID NO: 357;
(ba) the light chain variable region comprises SEQ ID NO: 50, and the heavy chain variable region comprises SEQ ID NO: 358;
(bb) the light chain variable region comprises SEQ ID NO: 52, and the heavy chain variable region comprises SEQ ID NO: 359;
(bc) the light chain variable region comprises SEQ ID NO: 54, and the heavy chain variable region comprises SEQ ID NO: 360;
(bd) the light chain variable region comprises SEQ ID NO: 56, and the heavy chain variable region comprises SEQ ID NO: 361;
(be) the light chain variable region comprises SEQ ID NO: 58, and the heavy chain variable region comprises SEQ ID NO: 362; or
(bf) the light chain variable region comprises SEQ ID NO: 60, and the heavy chain variable region comprises SEQ ID NO: 363.

30. The antibody, or antigen-binding fragment thereof, of claim 29, wherein:
(a) the light chain comprises SEQ ID NO: 228, and the heavy chain comprises SEQ ID NO: 168 or SEQ ID NO: 169; or
(b) the light chain comprises SEQ ID NO: 229, and the heavy chain comprises SEQ ID NO: 170 or SEQ ID NO: 171; or
(c) the light chain comprises SEQ ID NO: 230, and the heavy chain comprises SEQ ID NO: 172 or SEQ ID NO: 173; or
(d) the light chain comprises SEQ ID NO: 231, and the heavy chain comprises SEQ ID NO: 174 or SEQ ID NO: 175; or
(e) the light chain comprises SEQ ID NO: 232, and the heavy chain comprises SEQ ID NO: 176 or SEQ ID NO: 177; or
(f) the light chain comprises SEQ ID NO: 233, and the heavy chain comprises SEQ ID NO: 178 or SEQ ID NO: 179; or
(g) the light chain comprises SEQ ID NO: 234, and the heavy chain comprises SEQ ID NO: 180 or SEQ ID NO: 181; or
(h) the light chain comprises SEQ ID NO: 235, and the heavy chain comprises SEQ ID NO: 182 or SEQ ID NO: 183; or
(i) the light chain comprises SEQ ID NO: 237, and the heavy chain comprises SEQ ID NO: 186 or SEQ ID NO: 187; or
(j) the light chain comprises SEQ ID NO: 238, and the heavy chain comprises SEQ ID NO: 188 or SEQ ID NO: 189; or
(k) the light chain comprises SEQ ID NO: 239, and the heavy chain comprises SEQ ID NO: 190 or SEQ ID NO: 191; or
(l) the light chain comprises SEQ ID NO: 240, and the heavy chain comprises SEQ ID NO: 192 or SEQ ID NO: 193; or
(m) the light chain comprises SEQ ID NO: 241, and the heavy chain comprises SEQ ID NO: 194 or SEQ ID NO: 195; or
(n) the light chain comprises SEQ ID NO: 242, and the heavy chain comprises SEQ ID NO: 196 or SEQ ID NO: 197; or
(o) the light chain comprises SEQ ID NO: 243, and the heavy chain comprises SEQ ID NO: 198 or SEQ ID NO: 199; or
(p) the light chain comprises SEQ ID NO: 244, and the heavy chain comprises SEQ ID NO: 200 or SEQ ID NO: 201; or
(q) the light chain comprises SEQ ID NO: 245, and the heavy chain comprises SEQ ID NO: 202 or SEQ ID NO: 203; or
(r) the light chain comprises SEQ ID NO: 246, and the heavy chain comprises SEQ ID NO: 204 or SEQ ID NO: 205; or
(s) the light chain comprises SEQ ID NO: 247, and the heavy chain comprises SEQ ID NO: 206 or SEQ ID NO: 207; or
(l) the light chain comprises SEQ ID NO: 248, and the heavy chain comprises SEQ ID NO: 208 or SEQ ID NO: 209; or
(u) the light chain comprises SEQ ID NO: 249, and the heavy chain comprises SEQ ID NO: 210 or SEQ ID NO: 211; or
(v) the light chain comprises SEQ ID NO: 250, and the heavy chain comprises SEQ ID NO: 212 or SEQ ID NO: 213; or
(w) the light chain comprises SEQ ID NO: 251, and the heavy chain comprises SEQ ID NO: 214 or SEQ ID NO: 215; or
(x) the light chain comprises SEQ ID NO: 252, and the heavy chain comprises SEQ ID NO: 216 or SEQ ID NO: 217; or
(y) the light chain comprises SEQ ID NO: 253, and the heavy chain comprises SEQ ID NO: 218 or SEQ ID NO: 219; or
(z) the light chain comprises SEQ ID NO: 254, and the heavy chain comprises SEQ ID NO: 220 or SEQ ID NO: 221; or
(aa) the light chain comprises SEQ ID NO: 255, and the heavy chain comprises SEQ ID NO: 222 or SEQ ID NO: 223; or
(ab) the light chain comprises SEQ ID NO: 256, and the heavy chain comprises SEQ ID NO: 224 or SEQ ID NO: 225; or
(ac) the light chain comprises SEQ ID NO: 257, and the heavy chain comprises SEQ ID NO: 226 or SEQ ID NO: 227; or
(ad) the light chain comprises SEQ ID NO: 228, and the heavy chain comprises SEQ ID NO: 364 or SEQ ID NO: 365; or
(ae) the light chain comprises SEQ ID NO: 229, and the heavy chain comprises SEQ ID NO: 366 or SEQ ID NO: 367; or
(af) the light chain comprises SEQ ID NO: 230, and the heavy chain comprises SEQ ID NO: 368 or SEQ ID NO: 369; or
(ag) the light chain comprises SEQ ID NO: 231, and the heavy chain comprises SEQ ID NO: 370 or SEQ ID NO: 371; or
(ah) the light chain comprises SEQ ID NO: 232, and the heavy chain comprises SEQ ID NO: 372 or SEQ ID NO: 373; or
(ai) the light chain comprises SEQ ID NO: 233, and the heavy chain comprises SEQ ID NO: 374 or SEQ ID NO: 375; or
(aj) the light chain comprises SEQ ID NO: 234, and the heavy chain comprises SEQ ID NO: 376 or SEQ ID NO: 377; or
(ak) the light chain comprises SEQ ID NO: 235, and the heavy chain comprises SEQ ID NO: 378 or SEQ ID NO: 379; or
(al) the light chain comprises SEQ ID NO: 237, and the heavy chain comprises SEQ ID NO: 382 or SEQ ID NO: 383; or
(am) the light chain comprises SEQ ID NO: 238, and the heavy chain comprises SEQ ID NO: 384 or SEQ ID NO: 385; or
(an) the light chain comprises SEQ ID NO: 239, and the heavy chain comprises SEQ ID NO: 386 or SEQ ID NO: 387; or
(ao) the light chain comprises SEQ ID NO: 240, and the heavy chain comprises SEQ ID NO: 388 or SEQ ID NO: 389; or
(ap) the light chain comprises SEQ ID NO: 241, and the heavy chain comprises SEQ ID NO: 390 or SEQ ID NO: 391; or
(aq) the light chain comprises SEQ ID NO: 242, and the heavy chain comprises SEQ ID NO: 392 or SEQ ID NO: 393; or
(ar) the light chain comprises SEQ ID NO: 243, and the heavy chain comprises SEQ ID NO: 394 or SEQ ID NO: 395; or
(as) the light chain comprises SEQ ID NO: 244, and the heavy chain comprises SEQ ID NO: 396 or SEQ ID NO: 397; or
(at) the light chain comprises SEQ ID NO: 245, and the heavy chain comprises SEQ ID NO: 398 or SEQ ID NO: 399; or
(au) the light chain comprises SEQ ID NO: 246, and the heavy chain comprises SEQ ID NO: 400 or SEQ ID NO: 401; or
(av) the light chain comprises SEQ ID NO: 247, and the heavy chain comprises SEQ ID NO: 402 or SEQ ID NO: 403; or
(aw) the light chain comprises SEQ ID NO: 248, and the heavy chain comprises SEQ ID NO: 404 or SEQ ID NO: 405; or
(ax) the light chain comprises SEQ ID NO: 249, and the heavy chain comprises SEQ ID NO: 406 or SEQ ID NO: 407; or
(ay) the light chain comprises SEQ ID NO: 250, and the heavy chain comprises SEQ ID NO: 408 or SEQ ID NO: 409; or
(z) the light chain comprises SEQ ID NO: 251, and the heavy chain comprises SEQ ID NO: 410 or SEQ ID NO: 411; or
(ba) the light chain comprises SEQ ID NO: 252, and the heavy chain comprises SEQ ID NO: 412 or SEQ ID NO: 413; or
(bb) the light chain comprises SEQ ID NO: 253, and the heavy chain comprises SEQ ID NO: 414 or SEQ ID NO: 415; or
(bc) the light chain comprises SEQ ID NO: 254, and the heavy chain comprises SEQ ID NO: 416 or SEQ ID NO: 417; or
(bd) the light chain comprises SEQ ID NO: 255, and the heavy chain comprises SEQ ID NO: 418 or SEQ ID NO: 419; or
(be) the light chain comprises SEQ ID NO: 256, and the heavy chain comprises SEQ ID NO: 420 or SEQ ID NO: 421; or
(bf) the light chain comprises SEQ ID NO: 257, and the heavy chain comprises SEQ ID NO: 422 or SEQ ID NO: 423.

31. An antibody, or antigen binding fragment thereof, wherein the antibody comprises:
a variable heavy chain comprising a HCDR1 of SEQ ID NO: 61, a HCDR2 of SEQ ID NO: 62, and a HCDR3 of DX₁₀X₁₁X₁₂YX₁₃X₁₄DX₁₅ (SEQ ID NO: 329), wherein: X₁₀ is E, Q, or N; X₁₁ is T, E, S, or N; X₁₂ is D or Q; X₁₃ is A or G; X₁₄ is L, W, F, or Y; and X₁₅ is Y, E, L, N, F, or W; and
a variable light chain comprising a LCDR1 of SEQ ID NO: 64, a LCDR2 of SEQ ID NO: 65, and a LCDR3 comprising an amino acid sequence of X₁₆X₁₇X₁₈EDX₁₉X₂₀X₂₁X₂₂ (SEQ ID NO: 328), wherein X₁₆ is Q, E, K, or H; X₁₇ is Q or H; X₁₈ is Y or H, X₁₉ is L or Y; X₂₀ is P or I; X₂₁ is L or P, and X₂₂ is T or V.

32. An isolated nucleic acid molecule encoding an antibody, or an antigen binding fragment thereof, of claim 27.

33. An expression vector comprising the nucleic acid molecule of claim 32.

34. A host cell comprising the nucleic acid molecule of claim 32.

35. A pharmaceutical composition comprising an antibody, or antigen binding fragment thereof, of claim 27 and a pharmaceutically acceptable excipient.

36-38. (canceled)

39. The pharmaceutical composition of claim 35, wherein the composition is formulated for intravenous or subcutaneous injection.

40. A method of producing a polypeptide comprising a heavy chain variable region or light chain variable region, the method comprising:
(a) growing the host cell of claim 34 under conditions so that the host cell expresses the polypeptide comprising the heavy chain variable region or the light chain variable region;
and
(b) purifying the polypeptide comprising the heavy chain variable region and/or the light chain variable region.

41. A method of treating a subject with C1s mediated disorder, the method comprising administering to the subject the antibody, or antigen binding fragment thereof, of claim 27, or a pharmaceutical composition comprising the same.

42. (canceled)

43. The method of claim 41, wherein the disorder is Myasthenia Gravis, hemolysis, Cold Agglutinin Disease, Immune Thrombocytopenia (ITP), Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy.

44. A method of treating a subject with or Myasthenia Gravis or Cold Agglutinin Disease, the method comprising administering to the subject the subject an antibody, or antigen binding fragment thereof, of claim 27, or a pharmaceutical composition comprising the same.

45-46. (canceled)

47. A method of inhibiting membrane attack complex (MAC) formation or deposition in a subject in need thereof, the method comprising administering to the subject the antibody, or antigen binding fragment thereof, of claim 27, or a pharmaceutical composition comprising the same.

48. A method of inhibiting or preventing neurotransmission impairment in a subject in need thereof, the method comprising administering to the subject the antibody, or antigen binding fragment thereof, of claim 27, or a pharmaceutical composition comprising the same.

49. A method of enhancing, allowing, or permitting muscle contraction in a subject in need thereof, the method comprising administering to the subject the antibody, or antigen binding fragment thereof, of claim 27, or a pharmaceutical composition comprising the same.

50. A method of ameliorating or alleviating muscle paralysis in a subject in need thereof, the method comprising administering to the subject the antibody, or antigen binding fragment thereof, of claim 27, or a pharmaceutical composition comprising the same.

51. A method of inhibiting the loss of muscle tone or muscle mass in a subject in need thereof, the method comprising administering to the subject the antibody, or antigen binding fragment thereof, of claim 27, or a pharmaceutical composition comprising the same.

52. A method of reducing the muscle fatigue index in a subject in need thereof, the method comprising administering to the subject the antibody, or antigen binding fragment thereof, of claim 27, or a pharmaceutical composition comprising the same.


SEQUENCE LISTING (455 sequences)

SEQ ID NO: 1 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSS

SEQ ID NO: 2 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCEQYEDYPLTFGGGTKVEIK

SEQ ID NO: 3 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSS

SEQ ID NO: 4 - protein, 107 aa, synthetic construct; identical to SEQ ID NO: 2

SEQ ID NO: 5 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSS

SEQ ID NO: 6 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCKQYEDYPLTFGGGTKVEIK

SEQ ID NO: 7 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSS

SEQ ID NO: 8 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCHQYEDYPLTFGGGTKVEIK

SEQ ID NO: 9 - protein, 118 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSS

SEQ ID NO: 10 - protein, 107 aa, synthetic construct; identical to SEQ ID NO: 8

SEQ ID NO: 11 - protein, 118 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSS

SEQ ID NO: 12 - protein, 107 aa, synthetic construct; identical to SEQ ID NO: 8

SEQ ID NO: 13 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSS

SEQ ID NO: 14 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDYPLVFGGGTKVEIK

SEQ ID NO: 15 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSS

SEQ ID NO: 16 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQHEDYPLTFGGGTKVEIK

SEQ ID NO: 17 - protein, 117 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 18 - protein, 107 aa, synthetic construct; identical to SEQ ID NO: 16

SEQ ID NO: 19 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSS

SEQ ID NO: 20 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDLIPTFGGGTKVEIK

SEQ ID NO: 21 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSS

SEQ ID NO: 22 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQSYNSYVWTFGQGTKVEIK

SEQ ID NO: 23 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSS

SEQ ID NO: 24 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSYDWTFGQGTKVEIK

SEQ ID NO: 25 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSS

SEQ ID NO: 26 - protein, 107 aa, synthetic construct; identical to SEQ ID NO: 24

SEQ ID NO: 27 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSS

SEQ ID NO: 28 - protein, 107 aa, synthetic construct; identical to SEQ ID NO: 24

SEQ ID NO: 29 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSS

SEQ ID NO: 30 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQARSYSWTFGQGTKVEIK

SEQ ID NO: 31 - protein, 119 aa, synthetic construct; identical to SEQ ID NO: 21

SEQ ID NO: 32 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQHNSYRWTFGQGTKVEIK

SEQ ID NO: 33 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSS

SEQ ID NO: 34 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCVQYPSYSWTFGQGTKVEIK

SEQ ID NO: 35 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSS

SEQ ID NO: 36 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYKLTFGQGTKVEIK

SEQ ID NO: 37 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSS

SEQ ID NO: 38 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSPSWLFGQGTKVEIK

SEQ ID NO: 39 - protein, 119 aa, synthetic construct; identical to SEQ ID NO: 37

SEQ ID NO: 40 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSLSWTFGQGTKVEIK

SEQ ID NO: 41 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSS

SEQ ID NO: 42 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCFQYNSYPLGFGGGTKVEIK

SEQ ID NO: 43 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSS

SEQ ID NO: 44 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYRSHPLTFGGGTKVEIK

SEQ ID NO: 45 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSS

SEQ ID NO: 46 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNQVPLTFGGGTKVEIK

SEQ ID NO: 47 - protein, 125 aa, synthetic construct; identical to SEQ ID NO: 41

SEQ ID NO: 48 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQTNIYPLTFGGGTKVEIK

SEQ ID NO: 49 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSS

SEQ ID NO: 50 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYKQYPLTFGGGTKVEIK

SEQ ID NO: 51 - protein, 125 aa, synthetic construct
EVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSS

SEQ ID NO: 52 - protein, 107 aa, synthetic construct; identical to SEQ ID NO: 48

SEQ ID NO: 53 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSS

SEQ ID NO: 54 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNSYPLAFGGGTKVEIK

SEQ ID NO: 55 - protein, 125 aa, synthetic construct; identical to SEQ ID NO: 51

SEQ ID NO: 56 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYHSYPLRFGGGTKVEIK

SEQ ID NO: 57 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSS

SEQ ID NO: 58 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYASYPLKFGGGTKVEIK

SEQ ID NO: 59 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSS

SEQ ID NO: 60 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNAYPLIFGGGTKVEIK

SEQ ID NO: 61 - protein, 5 aa, synthetic construct
DYYMS

SEQ ID NO: 62 - protein, 17 aa, synthetic construct
YISRSGSTKYYADSVKG

SEQ ID NO: 63 - protein, 9 aa, synthetic construct
DQEDYALDY

SEQ ID NO: 64 - protein, 11 aa, synthetic construct
QASQDISNYLN

SEQ ID NO: 65 - protein, 7 aa, synthetic construct
DASNLET

SEQ ID NO: 66 - protein, 9 aa, synthetic construct
EQYEDYPLT

SEQ ID NO: 67 - protein, 9 aa, synthetic construct
DETDYGWDY

SEQ ID NO: 68 - protein, 9 aa, synthetic construct
KQYEDYPLT

SEQ ID NO: 69 - protein, 9 aa, synthetic construct
DETDYAFDE

SEQ ID NO: 70 - protein, 9 aa, synthetic construct
DESQYALDY

SEQ ID NO: 71 - protein, 9 aa, synthetic construct
HQYEDYPLT

SEQ ID NO: 72 - protein, 9 aa, synthetic construct
DNTDYALDL

SEQ ID NO: 73 - protein, 9 aa, synthetic construct
DETDYAYDE

SEQ ID NO: 74 - protein, 9 aa, synthetic construct
DETDYAYDN

SEQ ID NO: 75 - protein, 9 aa, synthetic construct
QQYEDYPLV

SEQ ID NO: 76 - protein, 9 aa, synthetic construct
DESDYAYDY

SEQ ID NO: 77 - protein, 8 aa, synthetic construct
QQHEDYPL

SEQ ID NO: 78 - protein, 8 aa, synthetic construct
DENYALDW

SEQ ID NO: 79 - protein, 8 aa, synthetic construct
QHYEDYPL

SEQ ID NO: 80 - protein, 9 aa, synthetic construct
DESDYALDF

SEQ ID NO: 81 - protein, 9 aa, synthetic construct
QQYEDLIPT

SEQ ID NO: 82 - protein, 5 aa, synthetic construct
ELSMH

SEQ ID NO: 83 - protein, 17 aa, synthetic construct
TFDPEEGETIYAQKFQG

SEQ ID NO: 84 - protein, 10 aa, synthetic construct
EGLAGVPFDL

SEQ ID NO: 85 - protein, 11 aa, synthetic construct
RASQSISSWLA

SEQ ID NO: 86 - protein, 7 aa, synthetic construct
KASSLES

SEQ ID NO: 87 - protein, 9 aa, synthetic construct
QSYNSYVWT

SEQ ID NO: 88 - protein, 10 aa, synthetic construct
EGLGGRPFDH

SEQ ID NO: 89 - protein, 9 aa, synthetic construct
QQVNSYDWT

SEQ ID NO: 90 - protein, 10 aa, synthetic construct
EGLAGFPFDI

SEQ ID NO: 91 - protein, 10 aa, synthetic construct
EGLAWRPTDS

SEQ ID NO: 92 - protein, 9 aa, synthetic construct
QQYSSYAWT

SEQ ID NO: 93 - protein, 10 aa, synthetic construct
KGLAWLPYYS

SEQ ID NO: 94 - protein, 9 aa, synthetic construct
QQARSYSWT

SEQ ID NO: 95 - protein, 9 aa, synthetic construct
QQHNSYRWT

SEQ ID NO: 96 - protein, 10 aa, synthetic construct
EGEAGRPFDA

SEQ ID NO: 97 - protein, 9 aa, synthetic construct
VQYPSYSWT

SEQ ID NO: 98 - protein, 10 aa, synthetic construct
EGLAGRPYDV

SEQ ID NO: 99 - protein, 9 aa, synthetic construct
QQYNSYKLT

SEQ ID NO: 100 - protein, 11 aa, synthetic construct
EGLAGIPFDSW

SEQ ID NO: 101 - protein, 9 aa, synthetic construct
QQYNSPSWL

SEQ ID NO: 102 - protein, 9 aa, synthetic construct
QQVNSLSWT

SEQ ID NO: 103 - protein, 5 aa, synthetic construct
DYGMS

SEQ ID NO: 104 - protein, 17 aa, synthetic construct
GINWEGGSTGYADSVKG

SEQ ID NO: 105 - protein, 16 aa, synthetic construct
DEQLGGTTYYYYYMDV

SEQ ID NO: 106 - protein, 11 aa, synthetic construct
RASQGIRNDLG

SEQ ID NO: 107 - protein, 7 aa, synthetic construct
TASNLQS

SEQ ID NO: 108 - protein, 9 aa, synthetic construct
FQYNSYPLG

SEQ ID NO: 109 - protein, 16 aa, synthetic construct
DEQLGGKYYYYYYMDV

SEQ ID NO: 110 - protein, 9 aa, synthetic construct
LQYRSHPLT

SEQ ID NO: 111 - protein, 16 aa, synthetic construct
DEQLGGQTYYYYYMDV

SEQ ID NO: 112 - protein, 9 aa, synthetic construct
LQYNQVPLT

SEQ ID NO: 113 - protein, 9 aa, synthetic construct
LQTNIYPLT

SEQ ID NO: 114 - protein, 16 aa, synthetic construct
DEQLGGIKYYYYYMDV

SEQ ID NO: 115 - protein, 9 aa, synthetic construct
LQYKQYPLT

SEQ ID NO: 116 - protein, 16 aa, synthetic construct
DEQLGGLKYYYYYMDV

SEQ ID NO: 117 - protein, 16 aa, synthetic construct
DEQLGGKVYYYYYMDV

SEQ ID NO: 118 - protein, 9 aa, synthetic construct
LQYNSYPLA

SEQ ID NO: 119 - protein, 9 aa, synthetic construct
LQYHSYPLR

SEQ ID NO: 120 - protein, 16 aa, synthetic construct
DEQLGGNHYYYYYMDA

SEQ ID NO: 121 - protein, 9 aa, synthetic construct
LQYASYPLK

SEQ ID NO: 122 - protein, 16 aa, synthetic construct
DEQLGGRHYYYYYMDV

SEQ ID NO: 123 - protein, 9 aa, synthetic construct
LQYNAYPLI

SEQ ID NO: 124 - protein, 7 aa, synthetic construct
GFTFSDY

SEQ ID NO: 125 - protein, 6 aa, synthetic construct
SRSGST

SEQ ID NO: 126 - protein, 7 aa, synthetic construct
GDTLTEL

SEQ ID NO: 127 - protein, 6 aa, synthetic construct
DPEEGE

SEQ ID NO: 128 - protein, 7 aa, synthetic construct
GFTFDDY

SEQ ID NO: 129 - protein, 6 aa, synthetic construct
NWEGGS

SEQ ID NO: 130 - protein, 8 aa, synthetic construct
GFTFSDYY

SEQ ID NO: 131 - protein, 8 aa, synthetic construct
ISRSGSTK

SEQ ID NO: 132 - protein, 11 aa, synthetic construct
ARDQEDYALDY

SEQ ID NO: 133 - protein, 6 aa, synthetic construct
QDISNY

SEQ ID NO: 134 - skipped

SEQ ID NO: 135 - protein, 11 aa, synthetic construct
ARDETDYGWDY

SEQ ID NO: 136 - protein, 11 aa, synthetic construct
ARDETDYAFDE

SEQ ID NO: 137 - protein, 11 aa, synthetic construct
ARDESQYALDY

SEQ ID NO: 138 - protein, 11 aa, synthetic construct
ARDNTDYALDL

SEQ ID NO: 139 - protein, 11 aa, synthetic construct
ARDETDYAYDE

SEQ ID NO: 140 - protein, 11 aa, synthetic construct
ARDETDYAYDN

SEQ ID NO: 141 - protein, 11 aa, synthetic construct
ARDESDYAYDY

SEQ ID NO: 142 - protein, 10 aa, synthetic construct
ARDENYALDW

SEQ ID NO: 143 - protein, 11 aa, synthetic construct
ARDESDYALDF

SEQ ID NO: 144 - protein, 8 aa, synthetic construct
GDTLTELS

SEQ ID NO: 145 - protein, 8 aa, synthetic construct
FDPEEGET

SEQ ID NO: 146 - protein, 12 aa, synthetic construct
VTEGLAGVPFDL

SEQ ID NO: 147 - protein, 6 aa, synthetic construct
QSISSW

SEQ ID NO: 148 - skipped

SEQ ID NO: 149 - protein, 12 aa, synthetic construct
VTEGLGGRPFDH

SEQ ID NO: 150 - protein, 12 aa, synthetic construct
VTEGLAGFPFDI

SEQ ID NO: 151 - protein, 12 aa, synthetic construct
VTEGLAWRPTDS

SEQ ID NO: 152 - protein, 12 aa, synthetic construct
VTKGLAWLPYYS

SEQ ID NO: 153 - protein, 12 aa, synthetic construct
VTEGEAGRPFDA

SEQ ID NO: 154 - protein, 12 aa, synthetic construct
VTEGLAGRPYDV

SEQ ID NO: 155 - protein, 13 aa, synthetic construct
VTEGLAGIPFDSW

SEQ ID NO: 156 - protein, 8 aa, synthetic construct
GFTFDDYG

SEQ ID NO: 157 - protein, 8 aa, synthetic construct
INWEGGST

SEQ ID NO: 158 - protein, 18 aa, synthetic construct
ARDEQLGGTTYYYYYMDV

SEQ ID NO: 159 - protein, 6 aa, synthetic construct
QGIRND

SEQ ID NO: 160 - skipped

SEQ ID NO: 161 - protein, 17 aa, synthetic construct
ARDEQLGGKYYYYYMDV

SEQ ID NO: 162 - protein, 18 aa, synthetic construct
ARDEQLGGQTYYYYYMDV

SEQ ID NO: 163 - protein, 18 aa, synthetic construct
ARDEQLGGIKYYYYYMDV

SEQ ID NO: 164 - protein, 18 aa, synthetic construct
ARDEQLGGLKYYYYYMDV

SEQ ID NO: 165 - protein, 18 aa, synthetic construct
ARDEQLGGKVYYYYYMDV

SEQ ID NO: 166 - protein, 18 aa, synthetic construct
ARDEQLGGNHYYYYYMDA

SEQ ID NO: 167 - protein, 18 aa, synthetic construct
ARDEQLGGRHYYYYYMDV

SEQ ID NO: 168 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 169 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 170 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 171 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 172 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 173 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 174 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 175 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 176 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 177 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 178 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 179 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 180 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 181 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 182 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 183 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 184 - protein, 443 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 185 - protein, 443 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 186 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 187 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 188 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 189 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 190 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 191 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 192 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 193 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 194 - protein, 444 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSL

SEQ ID NO: 195 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 196 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 197 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 198 - protein, 445 aa, synthetic construct; identical to SEQ ID NO: 188

SEQ ID NO: 199 - protein, 445 aa, synthetic construct; identical to SEQ ID NO: 189

SEQ ID NO: 200 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 201 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 202 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 203 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 204 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 205 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 206 - protein, 445 aa, synthetic construct; identical to SEQ ID NO: 204

SEQ ID NO: 207 - protein, 445 aa, synthetic construct; identical to SEQ ID NO: 205

SEQ ID NO: 208 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 209 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 210 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 211 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 212 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 213 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 214 - protein, 451 aa, synthetic construct; identical to SEQ ID NO: 208

SEQ ID NO: 215 - protein, 451 aa, synthetic construct; identical to SEQ ID NO: 209

SEQ ID NO: 216 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 217 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 218 - protein, 451 aa, synthetic construct
EVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 219 - protein, 451 aa, synthetic construct
EVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 220 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 221 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 222 - protein, 451 aa, synthetic construct; identical to SEQ ID NO: 218

SEQ ID NO: 223 - protein, 451 aa, synthetic construct; identical to SEQ ID NO: 219

SEQ ID NO: 224 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 225 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 226 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 227 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 228 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCEQYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCKQYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 214 aa, synthetic construct; identical to SEQ ID NO: 229

SEQ ID NO: 231 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCHQYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 214 aa, synthetic construct; identical to SEQ ID NO: 231

SEQ ID NO: 233 - protein, 214 aa, synthetic construct; identical to SEQ ID NO: 231

SEQ ID NO: 234 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDYPLVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQHEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDLIPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQSYNSYVWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSYDWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 214 aa, synthetic construct; identical to SEQ ID NO: 239

SEQ ID NO: 241 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSYAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQARSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQHNSYRWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCVQYPSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYKLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSPSWLFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSLSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCFQYNSYPLGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYRSHPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNQVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQTNIYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYKQYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 214 aa, synthetic construct; identical to SEQ ID NO: 251

SEQ ID NO: 254 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNSYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYHSYPLRFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYASYPLKFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNAYPLIFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 259 - protein, 15 aa, synthetic construct
MWCIVLFSLLAWVYA

SEQ ID NO: 260 - protein, 673 aa, Homo sapiens
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 261 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 262 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 263 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 264 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 265 - protein, 107 aa, Homo sapiens [sequence omitted]

SEQ ID NO: 266 - protein, 5 aa, synthetic construct
  REPEAT 1..5: note = The sequence can be repeated 1 time, 2 times, 3 times, 4 times, or 5 times
GGGGS

SEQ ID NO: 267 - protein, 5 aa, synthetic construct
  REPEAT 1..5: note = The sequence can be repeated 1 time, 2 times, 3 times, 4 times, or 5 times
GGGGA

SEQ ID NO: 268 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagaccaggaagattacgctcttgactactggggccaaggtacgctggttacggtc

SEQ ID NO: 269 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtgaacagtatgaggactaccctcttaccttcggcggtggaacgaaagttgagattaagcgaacc

SEQ ID NO: 270 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacggctgggactactggggccaaggtacgctggttacggtc

SEQ ID NO: 271 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtaagcagtatgaggactaccctcttaccttcggcggtggaacgaaagttgagattaagcgaacc

SEQ ID NO: 272 - DNA, 327 bp, synthetic construct; identical to SEQ ID NO: 271
  source 1..327: mol_type = other DNA

SEQ ID NO: 273 - DNA, 327 bp, synthetic construct; identical to SEQ ID NO: 271
  source 1..327: mol_type = other DNA

SEQ ID NO: 274 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtcagtacgctcttgactactggggccaaggtacgctggttacggtc

SEQ ID NO: 275 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcaccagtatgaggactaccctcttaccttcggcggtggaacgaaagttgagattaagcgaacc

SEQ ID NO: 276 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacaacaccgattacgctcttgacctgtggggccaaggtacgctggttacggtc

SEQ ID NO: 277 - DNA, 327 bp, synthetic construct; identical to SEQ ID NO: 275
  source 1..327: mol_type = other DNA

SEQ ID NO: 278 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacgagaccgattacgcttacgacgaatggggccaaggtacgctggttacggtc

SEQ ID NO: 279 - DNA, 327 bp, synthetic construct; identical to SEQ ID NO: 275
  source 1..327: mol_type = other DNA

SEQ ID NO: 280 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgcttacgacaactggggccaaggtacgctggttacggtc

SEQ ID NO: 281 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcagtatgaggactaccctcttgtgttcggcggtggaacgaaagttgagattaagcgaacc

SEQ ID NO: 282 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgcttacgactactggggccaaggtacgctggttacggtc

SEQ ID NO: 283 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcagcacgaggactaccctcttaccttcggcggtggaacgaaagttgagattaagcgaacc

SEQ ID NO: 284 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaattacgctcttgactggtggggccaaggtacgctggttacggtctcg

SEQ ID NO: 285 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcactatgaggactaccctcttaccttcggcggtggaacgaaagttgagattaagcgaacc

SEQ ID NO: 286 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgctcttgacttctggggccaaggtacgctggttacggtc

SEQ ID NO: 287 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcagtatgaggacctcatccccaccttcggcggtggaacgaaagttgagattaagcgaacc

SEQ ID NO: 288 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccggggtgccgtttgatctgtgggggcaggggacgctggtaacggtctcgagt

SEQ ID NO: 289 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagtggatcaggatctggtactgaattcactctgaccatttcaagtcttcagcctgatgactttgcgacctattattgccagagctacaatagctacgtgtggacgttcgggcagggtactaaagtcgagattaaacgaacc

SEQ ID NO: 290 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttgggcgggcgcccgtttgatcactgggggcaggggacgctggtaacggtctcgagt

SEQ ID NO: 291 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagtggatcaggatctggtactgaattcactctgaccatttcaagtcttcagcctgatgactttgcgacctattattgccagcaggtgaatagctacgactggacgttcgggcagggtactaaagtcgagattaaacgaacc

SEQ ID NO: 292 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggttcccgtttgatatctgggggcaggggacgctggtaacggtctcgagt

SEQ ID NO: 293 - DNA, 327 bp, synthetic construct; identical to SEQ ID NO: 291
  source 1..327: mol_type = other DNA

SEQ ID NO: 294 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggcctggcgcccgaccgattcatgggggcaggggacgctggtaacggtctcgagt

SEQ ID NO: 295 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagtacagcagctacgcctggacgttcgggcagggtactaaagtcgagattaaacgaacc

SEQ ID NO: 296 - DNA, 357 bp, synthetic construct
  source 1..357: mol_type = other DNA
caagttcaactggttcaaagcggggccgaggtaaagaagtcaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccaagggtttggccgggctgccgtactactcatgggggcaggggacgctggtaacggtctcgagt

SEQ ID NO: 297 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcaggcccgcagctactcatggacgttcgggcagggtactaaagtcgagattaaacgaacc

SEQ ID NO: 298 - DNA, 357 bp, synthetic construct; identical to SEQ ID NO: 288
  source 1..357: mol_type = other DNA

SEQ ID NO: 299 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagcacaatagctaccgctggacgttcgggcagggtactaaagtcgagattaaacgaacc

SEQ ID NO: 300 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtgaggccgggcgcccgtttgatgcctgggggcaggggacgctggtaacggtctcgagt

SEQ ID NO: 301 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgcgtgcagtaccccagctactcatggacgttcgggcagggtactaaagtcgagattaaacgaacc

SEQ ID NO: 302 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggcgcccgtacgatgtgtgggggcaggggacgctggtaacggtctcgagt

SEQ ID NO: 303 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagtacaatagctacaagctgacgttcgggcagggtactaaagtcgagattaaacgaacc

SEQ ID NO: 304 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctacctcagaagtgaagatactgccgtctactactgtgtgaccgagggtttggccgggatcccgtttgattcatgggggcaggggacgctggtaacggtctcgagt

SEQ ID NO: 305 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagtacaatagcccctcatggctgttcgggcagggtactaaagtcgagattaaacgaacc

SEQ ID NO: 306 - DNA, 357 bp, synthetic construct; identical to SEQ ID NO: 304
  source 1..357: mol_type = other DNA

SEQ ID NO: 307 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcaggtgaatagcctgtcatggacgttcgggcagggtactaaagtcgagattaaacgaacc

SEQ ID NO: 308 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggactacttattattactattacatggacgtgtggggtaaaggaacgaccgtcacagtc

SEQ ID NO: 309 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttccagtataatagttatcctctcggctttggcggtggcaccaaagtggagattaagcgaacc

SEQ ID NO: 310 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaagtattactattactattacatggacgtgtggggtaaaggaacgaccgtcacagtc

SEQ ID NO: 311 - DNA, 330 bp, synthetic construct
  source 1..330: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtaccgcagtcactatcctctcacttttggcggtggcaccaaagtggagattaagcgaacc

SEQ ID NO: 312 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcagacttattattactattacatggacgtgtggggtaaaggaacgaccgtcacagtc

SEQ ID NO: 313 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataatcaggtgcctctcacttttggcggtggcaccaaagtggagattaagcgaacc

SEQ ID NO: 314 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaccacctattattactattacatggacgtgtggggtaaaggaacgaccgtcacagtc

SEQ ID NO: 315 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagaccaatatctatcctctcacttttggcggtggcaccaaagtggagattaagcgaacc

SEQ ID NO: 316 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggatcaagtattattactattacatggacgtgtggggtaaaggaacgaccgtcacagtc

SEQ ID NO: 317 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataagcagtatcctctcacttttggcggtggcaccaaagtggagattaagcgaacc

SEQ ID NO: 318 - DNA, 369 bp, synthetic construct
  source 1..369: mol_type = other DNA
gaagtccagcttgttgagtctggcggaggggtggttaggccaggaggttcccttaggctttcctgtgcggcatcaggatttactttcgatgactacggaatgtcatgggtacgacaagctcccggcaaaggccttgaatgggtttctggcatcaactgggagggcggttccactggctacgcggactcagttaagggtaggttcagcataagcagggacaatgcgaagaactcactgtatctccaaatgaacagtctccgcgccgaggatacagccctttactattgtgcacgagatgagcaattgggggggctgaagtattattactattacatggacgtgtggggtaaaggaacgaccgtcacagtc

SEQ ID NO: 319 - DNA, 327 bp, synthetic construct; identical to SEQ ID NO: 315
  source 1..327: mol_type = other DNA

SEQ ID NO: 320 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaaggtgtattattactattacatggacgtgtggggtaaaggaacgaccgtcacagtc

SEQ ID NO: 321 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataatagttatcctctcgcctttggcggtggcaccaaagtggagattaagcgaacc

SEQ ID NO: 322 - DNA, 369 bp, synthetic construct; identical to SEQ ID NO: 318
  source 1..369: mol_type = other DNA

SEQ ID NO: 323 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtatcacagttatcctctccgctttggcggtggcaccaaagtggagattaagcgaacc

SEQ ID NO: 324 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaatcactattattactattacatggacgcttggggtaaaggaacgaccgtcacagtc

SEQ ID NO: 325 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtatgccagttatcctctcaagtttggcggtggcaccaaagtggagattaagcgaacc

SEQ ID NO: 326 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcgccactattattactattacatggacgtgtggggtaaaggaacgaccgtcacagtc

SEQ ID NO: 327 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataatgcctatcctctcatctttggcggtggcaccaaagtggagattaagcgaacc

SEQ ID NO: 328-332 - skipped (5 entries)

SEQ ID NO: 333 - protein, 16 aa, synthetic construct
  VARIANT 7: note = X can be N, T, I, Q, L, K, or R
  VARIANT 8: note = X can be Y, T, K, H, or V
  VARIANT 16: note = X can be V or A
DEQLGGXXYYYYYMDX

SEQ ID NO: 334 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSS

SEQ ID NO: 335 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSS

SEQ ID NO: 336 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSS

SEQ ID NO: 337 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSS

SEQ ID NO: 338 - protein, 118 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSS

SEQ ID NO: 339 - protein, 118 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSS

SEQ ID NO: 340 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSS

SEQ ID NO: 341 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSS

SEQ ID NO: 342 - protein, 117 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 343 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSS

SEQ ID NO: 344 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSS

SEQ ID NO: 345 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSS

SEQ ID NO: 346 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSS

SEQ ID NO: 347 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSS

SEQ ID NO: 348 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSS

SEQ ID NO: 349 - protein, 119 aa, synthetic construct; identical to SEQ ID NO: 344

SEQ ID NO: 350 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSS

SEQ ID NO: 351 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSS

SEQ ID NO: 352 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSS

SEQ ID NO: 353 - protein, 119 aa, synthetic construct; identical to SEQ ID NO: 352

SEQ ID NO: 354 - protein, 124 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVS

SEQ ID NO: 355 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSS

SEQ ID NO: 356 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSS

SEQ ID NO: 357 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSS

SEQ ID NO: 358 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSS

SEQ ID NO: 359 - protein, 125 aa, synthetic construct
QVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSS

SEQ ID NO: 360 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSS

SEQ ID NO: 361 - protein, 125 aa, synthetic construct; identical to SEQ ID NO: 359

SEQ ID NO: 362 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSS

SEQ ID NO: 363 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSS

SEQ ID NO: 364 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 365 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 366 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 367 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 368 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 369 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 370 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 371 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 372 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 373 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 374 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 375 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 376 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 377 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 378 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 379 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 380 - protein, 443 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 381 - protein, 443 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 382 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 383 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 384 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 385 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 386 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 387 - protein, 483 aa, synthetic construct
MABWITHCONSTANTDOMAINPEANDYTEMUTATIONSEVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 388 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 389 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 390 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 391 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 392 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 393 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 394 - protein, 445 aa, synthetic construct; identical to SEQ ID NO: 384

SEQ ID NO: 395 - protein, 445 aa, synthetic construct; identical to SEQ ID NO: 385

SEQ ID NO: 396 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 397 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 398 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 399 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 400 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 401 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 402 - protein, 445 aa, synthetic construct; identical to SEQ ID NO: 400

SEQ ID NO: 403 - protein, 445 aa, synthetic construct; identical to SEQ ID NO: 401

SEQ ID NO: 404 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 405 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 406 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 407 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 408 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 409 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 410 - protein, 451 aa, synthetic construct; identical to SEQ ID NO: 404

SEQ ID NO: 411 - protein, 451 aa, synthetic construct; identical to SEQ ID NO: 405

SEQ ID NO: 412 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 413 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 414 - protein, 451 aa, synthetic construct
QVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 415 - protein, 451 aa, synthetic construct
QVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 416 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 417 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 418 - protein, 451 aa, synthetic construct; identical to SEQ ID NO: 414

SEQ ID NO: 419 - protein, 451 aa, synthetic construct; identical to SEQ ID NO: 415

SEQ ID NO: 420 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 421 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 422 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 423 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 424 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagaccaggaagattacgctcttgactactggggccaaggtacgctggttacggtc

SEQ ID NO: 425 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacggctgggactactggggccaaggtacgctggttacggtc

SEQ ID NO: 426 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgctttcgacgaatggggccaaggtacgctggttacggtc

SEQ ID NO: 427 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtcagtacgctcttgactactggggccaaggtacgctggttacggtc

SEQ ID NO: 428 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacaacaccgattacgctcttgacctgtggggccaaggtacgctggttacggtc

SEQ ID NO: 429 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacgagaccgattacgcttacgacgaatggggccaaggtacgctggttacggtc

SEQ ID NO: 430 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgcttacgacaactggggccaaggtacgctggttacggtc

SEQ ID NO: 431 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgcttacgactactggggccaaggtacgctggttacggtc

SEQ ID NO: 432 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaattacgctcttgactggtggggccaaggtacgctggttacggtctcg

SEQ ID NO: 433 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgctcttgacttctggggccaaggtacgctggttacggtc

SEQ ID NO: 434 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccggggtgccgtttgatctgtgggggcaggggacgctggtaacggtctcgagt

SEQ ID NO: 435 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttgggcgggcgcccgtttgatcactgggggcaggggacgctggtaacggtctcgagt

SEQ ID NO: 436 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggttcccgtttgatatctgggggcaggggacgctggtaacggtctcgagt

SEQ ID NO: 437 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggcctggcgcccgaccgattcatgggggcaggggacgctggtaacggtctcgagt

SEQ ID NO: 438 - DNA, 357 bp, synthetic construct
  source 1..357: mol_type = other DNA
gaagttcaactggttcaaagcggggccgaggtaaagaagtcaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccaagggtttggccgggctgccgtactactcatgggggcaggggacgctggtaacggtctcgagt

SEQ ID NO: 439 - DNA, 357 bp, synthetic construct; identical to SEQ ID NO: 434
  source 1..357: mol_type = other DNA

SEQ ID NO: 440 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtgaggccgggcgcccgtttgatgcctgggggcaggggacgctggtaacggtctcgagt

SEQ ID NO: 441 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggcgcccgtacgatgtgtgggggcaggggacgctggtaacggtctcgagt

SEQ ID NO: 442 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctacctcagaagtgaagatactgccgtctactactgtgtgaccgagggtttggccgggatcccgtttgattcatgggggcaggggacgctggtaacggtctcgagt

SEQ ID NO: 443 - DNA, 357 bp, synthetic construct; identical to SEQ ID NO: 442
  source 1..357: mol_type = other DNA

SEQ ID NO: 444 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggactacttattattactattacatggacgtgtggggtaaaggaacgaccgtcacagtc

SEQ ID NO: 445 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaagtattactattactattacatggacgtgtggggtaaaggaacgaccgtcacagtc

SEQ ID NO: 446 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcagacttattattactattacatggacgtgtggggtaaaggaacgaccgtcacagtc

SEQ ID NO: 447 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaccacctattattactattacatggacgtgtggggtaaaggaacgaccgtcacagtc

SEQ ID NO: 448 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggatcaagtattattactattacatggacgtgtggggtaaaggaacgaccgtcacagtc

SEQ ID NO: 449 - DNA, 369 bp, synthetic construct
  source 1..369: mol_type = other DNA
caagtccagcttgttgagtctggcggaggggtggttaggccaggaggttcccttaggctttcctgtgcggcatcaggatttactttcgatgactacggaatgtcatgggtacgacaagctcccggcaaaggccttgaatgggtttctggcatcaactgggagggcggttccactggctacgcggactcagttaagggtaggttcagcataagcagggacaatgcgaagaactcactgtatctccaaatgaacagtctccgcgccgaggatacagccctttactattgtgcacgagatgagcaattgggggggctgaagtattattactattacatggacgtgtggggtaaaggaacgaccgtcacagtc

SEQ ID NO: 450 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaaggtgtattattactattacatggacgtgtggggtaaaggaacgaccgtcacagtc

SEQ ID NO: 451 - DNA, 369 bp, synthetic construct; identical to SEQ ID NO: 449
  source 1..369: mol_type = other DNA

SEQ ID NO: 452 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaatcactattattactattacatggacgcttggggtaaaggaacgaccgtcacagtc

SEQ ID NO: 453 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcgccactattattactattacatggacgtgtggggtaaaggaacgaccgtcacagtc

SEQ ID NO: 454 - protein, 116 aa, synthetic construct
VQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 455 - protein, 442 aa, synthetic construct
VQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG